SEQUENCE LISTING (38 sequences)

SEQ ID NO: 1 - RNA, 30 nt, synthetic construct
  misc_feature 1..30: note = Synthetic: WV-46312
  misc_feature 1..30: note = Synthetic: ADR-0100541
  modified_base 1: mod_base = OTHER; note = triantennary GalNAc with C12 linker linked to 6-aminohexanol linked by phosphate to the 5' end of 2-O-methylribose cytosine
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = cm
  modified_base 7: mod_base = OTHER; note = 2-O-methylribose adenine
  modified_base 7..8: mod_base = OTHER; note = S-phosphorothioate
  modified_base 8: mod_base = OTHER; note = 2-fluororibose guanine
  modified_base 8..9: mod_base = OTHER; note = S-phosphorothioate
  modified_base 9: mod_base = OTHER; note = 2-fluororibose cytosine
  modified_base 10: mod_base = um
  modified_base 10..11: mod_base = OTHER; note = S-phosphorothioate
  modified_base 11: mod_base = OTHER; note = 2-fluororibose uracil
  modified_base 11..12: mod_base = OTHER; note = R-N-(1,3-dimethylimidazolidin-2-ylidene)phosphorami date
  modified_base 12: mod_base = cm
  modified_base 13: mod_base = OTHER; note = 2-fluororibose adenine
  modified_base 13..14: mod_base = OTHER; note = S-phosphorothioate
  modified_base 14: mod_base = OTHER; note = 2-fluororibose guanine
  modified_base 14..15: mod_base = OTHER; note = R-N-(1,3-dimethylimidazolidin-2-ylidene)phosphorami date
  modified_base 15: mod_base = OTHER; note = 2-fluororibose uracil
  modified_base 16: mod_base = cm
  modified_base 16..17: mod_base = OTHER; note = S-phosphorothioate
  modified_base 17: mod_base = OTHER; note = 2-fluororibose cytosine
  modified_base 17..18: mod_base = OTHER; note = S-phosphorothioate
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = S-phosphorothioate
  modified_base 20: mod_base = um
  modified_base 21: mod_base = um
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA
cccagcagcttcagtccctttcttgtcgat

SEQ ID NO: 2 - RNA, 30 nt, synthetic construct; identical to SEQ ID NO: 1
  misc_feature 1..30: note = Synthetic: WV-44515
  misc_feature 1..30: note = Synthetic: ADR-0104180
  modified_base 1: mod_base = cm
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = cm
  modified_base 7: mod_base = OTHER; note = 2-O-methylribose adenine
  modified_base 7..8: mod_base = OTHER; note = S-phosphorothioate
  modified_base 8: mod_base = OTHER; note = 2-fluororibose guanine
  modified_base 8..9: mod_base = OTHER; note = S-phosphorothioate
  modified_base 9: mod_base = OTHER; note = 2-fluororibose cytosine
  modified_base 10: mod_base = um
  modified_base 10..11: mod_base = OTHER; note = S-phosphorothioate
  modified_base 11: mod_base = OTHER; note = 2-fluororibose uracil
  modified_base 11..12: mod_base = OTHER; note = R-N-(1,3-dimethylimidazolidin-2-ylidene)phosphorami date
  modified_base 12: mod_base = cm
  modified_base 13: mod_base = OTHER; note = 2-fluororibose adenine
  modified_base 13..14: mod_base = OTHER; note = S-phosphorothioate
  modified_base 14: mod_base = OTHER; note = 2-fluororibose guanine
  modified_base 14..15: mod_base = OTHER; note = R-N-(1,3-dimethylimidazolidin-2-ylidene)phosphorami date
  modified_base 15: mod_base = OTHER; note = 2-fluororibose uracil
  modified_base 16: mod_base = cm
  modified_base 16..17: mod_base = OTHER; note = S-phosphorothioate
  modified_base 17: mod_base = OTHER; note = 2-fluororibose cytosine
  modified_base 17..18: mod_base = OTHER; note = S-phosphorothioate
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = S-phosphorothioate
  modified_base 20: mod_base = um
  modified_base 21: mod_base = um
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 3 - RNA, 30 nt, synthetic construct; identical to SEQ ID NO: 1
  misc_feature 1..30: note = Synthetic: WV-47606
  misc_feature 1..30: note = Synthetic: ADR-0100564
  modified_base 1: mod_base = OTHER; note = triantennary GalNAc with C12 linker linked to 6-aminohexanol linked by phosphate to the 5' end of 2-O-methylribose cytosine
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = OTHER; note = 2-O-methoxyethylribose 5-methyl-cytidine
  modified_base 7: mod_base = OTHER; note = 2-O-methoxyethylribose adenine
  modified_base 8: mod_base = OTHER; note = 2-fluororibose guanine
  modified_base 8..9: mod_base = OTHER; note = S-phosphorothioate
  modified_base 9: mod_base = OTHER; note = 2-fluororibose cytosine
  modified_base 9..10: mod_base = OTHER; note = S-phosphorothioate
  modified_base 10: mod_base = OTHER; note = 2-O-methoxyethylribose thymine
  modified_base 11: mod_base = OTHER; note = 2-fluororibose uracil
  modified_base 11..12: mod_base = OTHER; note = R-N-(1,3-dimethylimidazolidin-2-ylidene)phosphorami date
  modified_base 12: mod_base = cm
  modified_base 13: mod_base = OTHER; note = 2-fluororibose adenine
  modified_base 13..14: mod_base = OTHER; note = S-phosphorothioate
  modified_base 14: mod_base = OTHER; note = 2-fluororibose guanine
  modified_base 14..15: mod_base = OTHER; note = R-N-(1,3-dimethylimidazolidin-2-ylidene)phosphorami date
  modified_base 15: mod_base = OTHER; note = 2-fluororibose uracil
  modified_base 16: mod_base = cm
  modified_base 16..17: mod_base = OTHER; note = S-phosphorothioate
  modified_base 17: mod_base = OTHER; note = 2-fluororibose cytosine
  modified_base 17..18: mod_base = OTHER; note = S-phosphorothioate
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = R-N-(1,3-dimethylimidazolidin-2-ylidene)phosphorami date
  modified_base 20: mod_base = OTHER; note = 2-O-methoxyethylribose thymine
  modified_base 21: mod_base = OTHER; note = 2-O-methoxyethylribose thymine
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 4 - RNA, 30 nt, synthetic construct; identical to SEQ ID NO: 1
  misc_feature 1..30: note = Synthetic: WV-46460
  misc_feature 1..30: note = Synthetic: ADR-0104089
  modified_base 1: mod_base = cm
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = OTHER; note = 2-O-methoxyethylribose 5-methyl-cytidine
  modified_base 7: mod_base = OTHER; note = 2-O-methoxyethylribose adenine
  modified_base 8: mod_base = OTHER; note = 2-fluororibose guanine
  modified_base 8..9: mod_base = OTHER; note = S-phosphorothioate
  modified_base 9: mod_base = OTHER; note = 2-fluororibose cytosine
  modified_base 9..10: mod_base = OTHER; note = S-phosphorothioate
  modified_base 10: mod_base = OTHER; note = 2-O-methoxyethylribose thymine
  modified_base 11: mod_base = OTHER; note = 2-fluororibose uracil
  modified_base 11..12: mod_base = OTHER; note = R-N-(1,3-dimethylimidazolidin-2-ylidene)phosphorami date
  modified_base 12: mod_base = cm
  modified_base 13: mod_base = OTHER; note = 2-fluororibose adenine
  modified_base 13..14: mod_base = OTHER; note = S-phosphorothioate
  modified_base 14: mod_base = OTHER; note = 2-fluororibose guanine
  modified_base 14..15: mod_base = OTHER; note = R-N-(1,3-dimethylimidazolidin-2-ylidene)phosphorami date
  modified_base 15: mod_base = OTHER; note = 2-fluororibose uracil
  modified_base 16: mod_base = cm
  modified_base 16..17: mod_base = OTHER; note = S-phosphorothioate
  modified_base 17: mod_base = OTHER; note = 2-fluororibose cytosine
  modified_base 17..18: mod_base = OTHER; note = S-phosphorothioate
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = R-N-(1,3-dimethylimidazolidin-2-ylidene)phosphorami date
  modified_base 20: mod_base = OTHER; note = 2-O-methoxyethylribose thymine
  modified_base 21: mod_base = OTHER; note = 2-O-methoxyethylribose thymine
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 5 - RNA, 30 nt, synthetic construct; identical to SEQ ID NO: 1
  misc_feature 1..30: note = Synthetic: WV-47608
  misc_feature 1..30: note = Synthetic: ADR-0100566
  modified_base 1: mod_base = OTHER; note = triantennary GalNAc with C12 linker linked to 6-aminohexanol linked by phosphate to the 5' end of 2-O-methylribose cytosine
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = cm
  modified_base 7: mod_base = OTHER; note = 2-O-methylribose adenine
  modified_base 8: mod_base = OTHER; note = 2-fluororibose guanine
  modified_base 8..9: mod_base = OTHER; note = S-phosphorothioate
  modified_base 9: mod_base = OTHER; note = 2-fluororibose cytosine
  modified_base 9..10: mod_base = OTHER; note = S-phosphorothioate
  modified_base 10: mod_base = um
  modified_base 11: mod_base = OTHER; note = 2-fluororibose uracil
  modified_base 11..12: mod_base = OTHER; note = R-N-(1,3-dimethylimidazolidin-2-ylidene)phosphorami date
  modified_base 12: mod_base = cm
  modified_base 13: mod_base = OTHER; note = 2-fluororibose adenine
  modified_base 13..14: mod_base = OTHER; note = S-phosphorothioate
  modified_base 14: mod_base = gm
  modified_base 14..15: mod_base = OTHER; note = R-N-(1,3-dimethylimidazolidin-2-ylidene)phosphorami date
  modified_base 15: mod_base = OTHER; note = 2-fluororibose uracil
  modified_base 16: mod_base = cm
  modified_base 16..17: mod_base = OTHER; note = S-phosphorothioate
  modified_base 17: mod_base = OTHER; note = 2-fluororibose cytosine
  modified_base 17..18: mod_base = OTHER; note = S-phosphorothioate
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = R-N-(1,3-dimethylimidazolidin-2-ylidene)phosphorami date
  modified_base 20: mod_base = um
  modified_base 21: mod_base = um
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 6 - RNA, 30 nt, synthetic construct; identical to SEQ ID NO: 1
  misc_feature 1..30: note = Synthetic: WV-47037
  misc_feature 1..30: note = Synthetic: ADR-0104273
  modified_base 1: mod_base = cm
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = cm
  modified_base 7: mod_base = OTHER; note = 2-O-methylribose adenine
  modified_base 8: mod_base = OTHER; note = 2-fluororibose guanine
  modified_base 8..9: mod_base = OTHER; note = S-phosphorothioate
  modified_base 9: mod_base = OTHER; note = 2-fluororibose cytosine
  modified_base 9..10: mod_base = OTHER; note = S-phosphorothioate
  modified_base 10: mod_base = um
  modified_base 11: mod_base = OTHER; note = 2-fluororibose uracil
  modified_base 11..12: mod_base = OTHER; note = R-N-(1,3-dimethylimidazolidin-2-ylidene)phosphorami date
  modified_base 12: mod_base = cm
  modified_base 13: mod_base = OTHER; note = 2-fluororibose adenine
  modified_base 13..14: mod_base = OTHER; note = S-phosphorothioate
  modified_base 14: mod_base = gm
  modified_base 14..15: mod_base = OTHER; note = R-N-(1,3-dimethylimidazolidin-2-ylidene)phosphorami date
  modified_base 15: mod_base = OTHER; note = 2-fluororibose uracil
  modified_base 16: mod_base = cm
  modified_base 16..17: mod_base = OTHER; note = S-phosphorothioate
  modified_base 17: mod_base = OTHER; note = 2-fluororibose cytosine
  modified_base 17..18: mod_base = OTHER; note = S-phosphorothioate
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = R-N-(1,3-dimethylimidazolidin-2-ylidene)phosphorami date
  modified_base 20: mod_base = um
  modified_base 21: mod_base = um
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 7 - RNA, 30 nt, synthetic construct; identical to SEQ ID NO: 1
  misc_feature 1..30: note = Synthetic: WV-49085
  misc_feature 1..30: note = Synthetic: ADR-0100585
  modified_base 1: mod_base = OTHER; note = triantennary GalNAc with C12 linker linked to 6-aminohexanol linked by phosphate to the 5' end of 2-O-methylribose cytosine
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = OTHER; note = 2-O-methoxyethylribose 5-methyl-cytidine
  modified_base 7: mod_base = OTHER; note = 2-O-methoxyethylribose adenine
  modified_base 8: mod_base = OTHER; note = 2-fluororibose guanine
  modified_base 8..9: mod_base = OTHER; note = S-phosphorothioate
  modified_base 9: mod_base = OTHER; note = 2-fluororibose cytosine
  modified_base 9..10: mod_base = OTHER; note = S-phosphorothioate
  modified_base 10: mod_base = OTHER; note = 2-O-methoxyethylribose thymine
  modified_base 11: mod_base = OTHER; note = 2-fluororibose uracil
  modified_base 11..12: mod_base = OTHER; note = R-N-(1,3-dimethylimidazolidin-2-ylidene)phosphorami date
  modified_base 12: mod_base = cm
  modified_base 13: mod_base = OTHER; note = 2-fluororibose adenine
  modified_base 13..14: mod_base = OTHER; note = S-phosphorothioate
  modified_base 14: mod_base = gm
  modified_base 14..15: mod_base = OTHER; note = R-N-(1,3-dimethylimidazolidin-2-ylidene)phosphorami date
  modified_base 15: mod_base = OTHER; note = 2-fluororibose uracil
  modified_base 16: mod_base = cm
  modified_base 16..17: mod_base = OTHER; note = S-phosphorothioate
  modified_base 17: mod_base = OTHER; note = 2-fluororibose cytosine
  modified_base 17..18: mod_base = OTHER; note = S-phosphorothioate
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = R-N-(1,3-dimethylimidazolidin-2-ylidene)phosphorami date
  modified_base 20: mod_base = OTHER; note = 2-O-methoxyethylribose thymine
  modified_base 21: mod_base = OTHER; note = 2-O-methoxyethylribose thymine
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 8 - RNA, 30 nt, synthetic construct; identical to SEQ ID NO: 1
  misc_feature 1..30: note = Synthetic: WV-49086
  misc_feature 1..30: note = Synthetic: ADR-0100586
  modified_base 1: mod_base = OTHER; note = triantennary GalNAc with C12 linker linked to 6-aminohexanol linked by phosphate to the 5' end of 2-O-methylribose cytosine
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = OTHER; note = 2-O-methoxyethylribose 5-methyl-cytidine
  modified_base 7: mod_base = OTHER; note = 2-O-methoxyethylribose adenine
  modified_base 8: mod_base = OTHER; note = 2-fluororibose guanine
  modified_base 8..9: mod_base = OTHER; note = S-phosphorothioate
  modified_base 9: mod_base = OTHER; note = 2-fluororibose cytosine
  modified_base 9..10: mod_base = OTHER; note = S-phosphorothioate
  modified_base 10: mod_base = OTHER; note = 2-O-methoxyethylribose thymine
  modified_base 11: mod_base = OTHER; note = 2-fluororibose uracil
  modified_base 11..12: mod_base = OTHER; note = R-N-(1,3-dimethylimidazolidin-2-ylidene)phosphorami date
  modified_base 12: mod_base = cm
  modified_base 13: mod_base = OTHER; note = 2-fluororibose adenine
  modified_base 13..14: mod_base = OTHER; note = S-phosphorothioate
  modified_base 14: mod_base = gm
  modified_base 14..15: mod_base = OTHER; note = R-N-(1,3-dimethylimidazolidin-2-ylidene)phosphorami date
  modified_base 15: mod_base = OTHER; note = 2-fluororibose uracil
  modified_base 16: mod_base = OTHER; note = 2-O-methoxyethylribose 5-methyl-cytidine
  modified_base 16..17: mod_base = OTHER; note = S-phosphorothioate
  modified_base 17: mod_base = OTHER; note = 2-fluororibose cytosine
  modified_base 17..18: mod_base = OTHER; note = S-phosphorothioate
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = R-N-(1,3-dimethylimidazolidin-2-ylidene)phosphorami date
  modified_base 20: mod_base = OTHER; note = 2-O-methoxyethylribose thymine
  modified_base 21: mod_base = OTHER; note = 2-O-methoxyethylribose thymine
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 9 - RNA, 30 nt, synthetic construct; identical to SEQ ID NO: 1
  misc_feature 1..30: note = Synthetic: WV-49087
  misc_feature 1..30: note = Synthetic: ADR-0100587
  modified_base 1: mod_base = OTHER; note = triantennary GalNAc with C12 linker linked to 6-aminohexanol linked by phosphate to the 5' end of 2-O-methylribose cytosine
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = OTHER; note = 2-O-methoxyethylribose 5-methyl-cytidine
  modified_base 7: mod_base = OTHER; note = 2-O-methoxyethylribose adenine
  modified_base 8: mod_base = OTHER; note = 2-fluororibose guanine
  modified_base 8..9: mod_base = OTHER; note = S-phosphorothioate
  modified_base 9: mod_base = cm
  modified_base 10: mod_base = OTHER; note = 2-O-methoxyethylribose thymine
  modified_base 10..11: mod_base = OTHER; note = S-phosphorothioate
  modified_base 11: mod_base = um
  modified_base 11..12: mod_base = OTHER; note = R-N-(1,3-dimethylimidazolidin-2-ylidene)phosphorami date
  modified_base 12: mod_base = cm
  modified_base 13: mod_base = OTHER; note = 2-fluororibose adenine
  modified_base 13..14: mod_base = OTHER; note = S-phosphorothioate
  modified_base 14: mod_base = OTHER; note = 2-fluororibose guanine
  modified_base 14..15: mod_base = OTHER; note = R-N-(1,3-dimethylimidazolidin-2-ylidene)phosphorami date
  modified_base 15: mod_base = um
  modified_base 16: mod_base = cm
  modified_base 17: mod_base = cm
  modified_base 17..18: mod_base = OTHER; note = S-phosphorothioate
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = S-phosphorothioate
  modified_base 20: mod_base = OTHER; note = 2-O-methoxyethylribose thymine
  modified_base 21: mod_base = OTHER; note = 2-O-methoxyethylribose thymine
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 10 - RNA, 30 nt, synthetic construct; identical to SEQ ID NO: 1
  misc_feature 1..30: note = Synthetic: WV-49088
  misc_feature 1..30: note = Synthetic: ADR-0100588
  modified_base 1: mod_base = OTHER; note = triantennary GalNAc with C12 linker linked to 6-aminohexanol linked by phosphate to the 5' end of 2-O-methylribose cytosine
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = OTHER; note = 2-O-methoxyethylribose 5-methyl-cytidine
  modified_base 7: mod_base = OTHER; note = 2-O-methoxyethylribose adenine
  modified_base 8: mod_base = OTHER; note = 2-fluororibose guanine
  modified_base 8..9: mod_base = OTHER; note = S-phosphorothioate
  modified_base 9: mod_base = cm
  modified_base 10: mod_base = OTHER; note = 2-O-methoxyethylribose thymine
  modified_base 10..11: mod_base = OTHER; note = S-phosphorothioate
  modified_base 11: mod_base = um
  modified_base 11..12: mod_base = OTHER; note = R-N-(1,3-dimethylimidazolidin-2-ylidene)phosphorami date
  modified_base 12: mod_base = cm
  modified_base 13: mod_base = OTHER; note = 2-fluororibose adenine
  modified_base 13..14: mod_base = OTHER; note = S-phosphorothioate
  modified_base 14: mod_base = OTHER; note = 2-fluororibose guanine
  modified_base 14..15: mod_base = OTHER; note = R-N-(1,3-dimethylimidazolidin-2-ylidene)phosphorami date
  modified_base 15: mod_base = um
  modified_base 16: mod_base = OTHER; note = 2-O-methoxyethylribose 5-methyl-cytidine
  modified_base 17: mod_base = cm
  modified_base 17..18: mod_base = OTHER; note = S-phosphorothioate
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = S-phosphorothioate
  modified_base 20: mod_base = OTHER; note = 2-O-methoxyethylribose thymine
  modified_base 21: mod_base = OTHER; note = 2-O-methoxyethylribose thymine
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 11 - RNA, 30 nt, synthetic construct; identical to SEQ ID NO: 1
  misc_feature 1..30: note = Synthetic: WV-50496
  misc_feature 1..30: note = Synthetic: ADR-0104091
  modified_base 1: mod_base = cm
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = OTHER; note = 2-O-methoxyethylribose 5-methyl-cytidine
  modified_base 7: mod_base = OTHER; note = 2-O-methoxyethylribose adenine
  modified_base 8: mod_base = OTHER; note = 2-fluororibose guanine
  modified_base 8..9: mod_base = OTHER; note = S-phosphorothioate
  modified_base 9: mod_base = cm
  modified_base 10: mod_base = OTHER; note = 2-O-methoxyethylribose thymine
  modified_base 10..11: mod_base = OTHER; note = S-phosphorothioate
  modified_base 11: mod_base = um
  modified_base 11..12: mod_base = OTHER; note = R-N-(1,3-dimethylimidazolidin-2-ylidene)phosphorami date
  modified_base 12: mod_base = cm
  modified_base 13: mod_base = OTHER; note = 2-fluororibose adenine
  modified_base 13..14: mod_base = OTHER; note = S-phosphorothioate
  modified_base 14: mod_base = OTHER; note = 2-fluororibose guanine
  modified_base 14..15: mod_base = OTHER; note = R-N-(1,3-dimethylimidazolidin-2-ylidene)phosphorami date
  modified_base 15: mod_base = um
  modified_base 16: mod_base = OTHER; note = 2-O-methoxyethylribose 5-methyl-cytidine
  modified_base 17: mod_base = cm
  modified_base 17..18: mod_base = OTHER; note = S-phosphorothioate
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = S-phosphorothioate
  modified_base 20: mod_base = OTHER; note = 2-O-methoxyethylribose thymine
  modified_base 21: mod_base = OTHER; note = 2-O-methoxyethylribose thymine
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 12 - RNA, 30 nt, synthetic construct; identical to SEQ ID NO: 1
  misc_feature 1..30: note = Synthetic: WV-49089
  misc_feature 1..30: note = Synthetic: ADR-0100589
  modified_base 1: mod_base = OTHER; note = triantennary GalNAc with C12 linker linked to 6-aminohexanol linked by phosphate to the 5' end of 2-O-methylribose cytosine
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = OTHER; note = 2-O-methoxyethylribose 5-methyl-cytidine
  modified_base 7: mod_base = OTHER; note = 2-O-methoxyethylribose adenine
  modified_base 8: mod_base = OTHER; note = 2-fluororibose guanine
  modified_base 8..9: mod_base = OTHER; note = S-phosphorothioate
  modified_base 9: mod_base = OTHER; note = 2-O-methoxyethylribose 5-methyl-cytidine
  modified_base 10: mod_base = OTHER; note = 2-O-methoxyethylribose thymine
  modified_base 10..11: mod_base = OTHER; note = S-phosphorothioate
  modified_base 11: mod_base = um
  modified_base 11..12: mod_base = OTHER; note = R-N-(1,3-dimethylimidazolidin-2-ylidene)phosphorami date
  modified_base 12: mod_base = OTHER; note = 2-O-methoxyethylribose 5-methyl-cytidine
  modified_base 13: mod_base = OTHER; note = 2-fluororibose adenine
  modified_base 13..14: mod_base = OTHER; note = S-phosphorothioate
  modified_base 14: mod_base = OTHER; note = 2-fluororibose guanine
  modified_base 14..15: mod_base = OTHER; note = R-N-(1,3-dimethylimidazolidin-2-ylidene)phosphorami date
  modified_base 15: mod_base = um
  modified_base 16: mod_base = OTHER; note = 2-O-methoxyethylribose 5-methyl-cytidine
  modified_base 17: mod_base = OTHER; note = 2-O-methoxyethylribose 5-methyl-cytidine
  modified_base 17..18: mod_base = OTHER; note = S-phosphorothioate
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = S-phosphorothioate
  modified_base 20: mod_base = OTHER; note = 2-O-methoxyethylribose thymine
  modified_base 21: mod_base = OTHER; note = 2-O-methoxyethylribose thymine
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 13 - RNA, 30 nt, synthetic construct; identical to SEQ ID NO: 1
  misc_feature 1..30: note = Synthetic: WV-49090
  misc_feature 1..30: note = Synthetic: ADR-0100590
  modified_base 1: mod_base = OTHER; note = triantennary GalNAc with C12 linker linked to 6-aminohexanol linked by phosphate to the 5' end of 2-O-methylribose cytosine
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = OTHER; note = 2-O-methoxyethylribose 5-methyl-cytidine
  modified_base 7: mod_base = OTHER; note = 2-O-methoxyethylribose adenine
  modified_base 8: mod_base = OTHER; note = 2-fluororibose guanine
  modified_base 8..9: mod_base = OTHER; note = S-phosphorothioate
  modified_base 9: mod_base = OTHER; note = 2-fluororibose cytosine
  modified_base 9..10: mod_base = OTHER; note = S-phosphorothioate
  modified_base 10: mod_base = um
  modified_base 11: mod_base = um
  modified_base 11..12: mod_base = OTHER; note = R-N-(1,3-dimethylimidazolidin-2-ylidene)phosphorami date
  modified_base 12: mod_base = cm
  modified_base 13: mod_base = OTHER; note = 2-fluororibose adenine
  modified_base 13..14: mod_base = OTHER; note = S-phosphorothioate
  modified_base 14: mod_base = OTHER; note = 2-fluororibose guanine
  modified_base 14..15: mod_base = OTHER; note = R-N-(1,3-dimethylimidazolidin-2-ylidene)phosphorami date
  modified_base 15: mod_base = OTHER; note = 2-fluororibose uracil
  modified_base 16: mod_base = OTHER; note = 2-O-methoxyethylribose 5-methyl-cytidine
  modified_base 16..17: mod_base = OTHER; note = S-phosphorothioate
  modified_base 17: mod_base = OTHER; note = 2-fluororibose cytosine
  modified_base 17..18: mod_base = OTHER; note = S-phosphorothioate
  modified_base 18: mod_base = cm
  modified_base 19: mod_base = um
  modified_base 19..20: mod_base = OTHER; note = R-N-(1,3-dimethylimidazolidin-2-ylidene)phosphorami date
  modified_base 20: mod_base = um
  modified_base 21: mod_base = OTHER; note = 2-O-methoxyethylribose thymine
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 14 - RNA, 30 nt, synthetic construct; identical to SEQ ID NO: 1
  misc_feature 1..30: note = Synthetic: WV-50497
  misc_feature 1..30: note = Synthetic: ADR-0104090
  modified_base 1: mod_base = cm
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = OTHER; note = 2-O-methoxyethylribose 5-methyl-cytidine
  modified_base 7: mod_base = OTHER; note = 2-O-methoxyethylribose adenine
  modified_base 8: mod_base = OTHER; note = 2-fluororibose guanine
  modified_base 8..9: mod_base = OTHER; note = S-phosphorothioate
  modified_base 9: mod_base = OTHER; note = 2-fluororibose cytosine
  modified_base 9..10: mod_base = OTHER; note = S-phosphorothioate
  modified_base 10: mod_base = um
  modified_base 11: mod_base = um
  modified_base 11..12: mod_base = OTHER; note = R-N-(1,3-dimethylimidazolidin-2-ylidene)phosphorami date
  modified_base 12: mod_base = cm
  modified_base 13: mod_base = OTHER; note = 2-fluororibose adenine
  modified_base 13..14: mod_base = OTHER; note = S-phosphorothioate
  modified_base 14: mod_base = OTHER; note = 2-fluororibose guanine
  modified_base 14..15: mod_base = OTHER; note = R-N-(1,3-dimethylimidazolidin-2-ylidene)phosphorami date
  modified_base 15: mod_base = OTHER; note = 2-fluororibose uracil
  modified_base 16: mod_base = OTHER; note = 2-O-methoxyethylribose 5-methyl-cytidine
  modified_base 16..17: mod_base = OTHER; note = S-phosphorothioate
  modified_base 17: mod_base = OTHER; note = 2-fluororibose cytosine
  modified_base 17..18: mod_base = OTHER; note = S-phosphorothioate
  modified_base 18: mod_base = cm
  modified_base 19: mod_base = um
  modified_base 19..20: mod_base = OTHER; note = R-N-(1,3-dimethylimidazolidin-2-ylidene)phosphorami date
  modified_base 20: mod_base = um
  modified_base 21: mod_base = OTHER; note = 2-O-methoxyethylribose thymine
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 15 - RNA, 30 nt, synthetic construct; identical to SEQ ID NO: 1
  misc_feature 1..30: note = Synthetic: WV-49092
  misc_feature 1..30: note = Synthetic: ADR-0100592
  modified_base 1: mod_base = OTHER; note = triantennary GalNAc with C12 linker linked to 6-aminohexanol linked by phosphate to the 5' end of 2-O-methylribose cytosine
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = OTHER; note = 2-O-methoxyethylribose 5-methyl-cytidine
  modified_base 7: mod_base = OTHER; note = 2-O-methoxyethylribose adenine
  modified_base 8: mod_base = OTHER; note = 2-fluororibose guanine
  modified_base 8..9: mod_base = OTHER; note = S-phosphorothioate
  modified_base 9: mod_base = OTHER; note = 2-fluororibose cytosine
  modified_base 9..10: mod_base = OTHER; note = S-phosphorothioate
  modified_base 10: mod_base = OTHER; note = 2-O-methoxyethylribose thymine
  modified_base 11: mod_base = OTHER; note = 2-fluororibose uracil
  modified_base 11..12: mod_base = OTHER; note = R-N-(1,3-dimethylimidazolidin-2-ylidene)phosphorami date
  modified_base 12: mod_base = cm
  modified_base 13: mod_base = OTHER; note = 2-fluororibose adenine
  modified_base 13..14: mod_base = OTHER; note = S-phosphorothioate
  modified_base 14: mod_base = OTHER; note = 2-fluororibose guanine
  modified_base 14..15: mod_base = OTHER; note = R-N-(1,3-dimethylimidazolidin-2-ylidene)phosphorami date
  modified_base 15: mod_base = OTHER; note = 2-fluororibose uracil
  modified_base 16: mod_base = OTHER; note = 2-O-methoxyethylribose 5-methyl-cytidine
  modified_base 16..17: mod_base = OTHER; note = S-phosphorothioate
  modified_base 17: mod_base = OTHER; note = 2-fluororibose cytosine
  modified_base 17..18: mod_base = OTHER; note = S-phosphorothioate
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = R-N-(1,3-dimethylimidazolidin-2-ylidene)phosphorami date
  modified_base 20: mod_base = OTHER; note = 2-O-methoxyethylribose thymine
  modified_base 21: mod_base = OTHER; note = 2-O-methoxyethylribose thymine
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 16 - RNA, 30 nt, synthetic construct; identical to SEQ ID NO: 1
  misc_feature 1..30: note = Synthetic: WV-50498
  misc_feature 1..30: note = Synthetic: ADR-0104087
  modified_base 1: mod_base = cm
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = OTHER; note = 2-O-methoxyethylribose 5-methyl-cytidine
  modified_base 7: mod_base = OTHER; note = 2-O-methoxyethylribose adenine
  modified_base 8: mod_base = OTHER; note = 2-fluororibose guanine
  modified_base 8..9: mod_base = OTHER; note = S-phosphorothioate
  modified_base 9: mod_base = OTHER; note = 2-fluororibose cytosine
  modified_base 9..10: mod_base = OTHER; note = S-phosphorothioate
  modified_base 10: mod_base = OTHER; note = 2-O-methoxyethylribose thymine
  modified_base 11: mod_base = OTHER; note = 2-fluororibose uracil
  modified_base 11..12: mod_base = OTHER; note = R-N-(1,3-dimethylimidazolidin-2-ylidene)phosphorami date
  modified_base 12: mod_base = cm
  modified_base 13: mod_base = OTHER; note = 2-fluororibose adenine
  modified_base 13..14: mod_base = OTHER; note = S-phosphorothioate
  modified_base 14: mod_base = OTHER; note = 2-fluororibose guanine
  modified_base 14..15: mod_base = OTHER; note = R-N-(1,3-dimethylimidazolidin-2-ylidene)phosphorami date
  modified_base 15: mod_base = OTHER; note = 2-fluororibose uracil
  modified_base 16: mod_base = OTHER; note = 2-O-methoxyethylribose 5-methyl-cytidine
  modified_base 16..17: mod_base = OTHER; note = S-phosphorothioate
  modified_base 17: mod_base = OTHER; note = 2-fluororibose cytosine
  modified_base 17..18: mod_base = OTHER; note = S-phosphorothioate
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = R-N-(1,3-dimethylimidazolidin-2-ylidene)phosphorami date
  modified_base 20: mod_base = OTHER; note = 2-O-methoxyethylribose thymine
  modified_base 21: mod_base = OTHER; note = 2-O-methoxyethylribose thymine
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 17 - RNA, 30 nt, synthetic construct
  misc_feature 1..30: note = Synthetic: WV-44464
  misc_feature 1..30: note = Synthetic: ADR-0100529
  modified_base 1: mod_base = OTHER; note = triantennary GalNAc with C12 linker linked to 6-aminohexanol linked by phosphate to the 5' end of 2-O-methylribose cytosine
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = cm
  modified_base 7: mod_base = OTHER; note = 2-fluororibose adenine
  modified_base 7..8: mod_base = OTHER; note = S-phosphorothioate
  modified_base 8: mod_base = OTHER; note = 2-fluororibose guanine
  modified_base 8..9: mod_base = OTHER; note = S-phosphorothioate
  modified_base 9: mod_base = OTHER; note = 2-fluororibose cytosine
  modified_base 9..10: mod_base = OTHER; note = S-phosphorothioate
  modified_base 10: mod_base = OTHER; note = 2-fluororibose uracil
  modified_base 10..11: mod_base = OTHER; note = S-phosphorothioate
  modified_base 11: mod_base = OTHER; note = 2-fluororibose uracil
  modified_base 11..12: mod_base = OTHER; note = R-N-(1,3-dimethylimidazolidin-2-ylidene)phosphorami date
  modified_base 12: mod_base = OTHER; note = 2-fluororibose cytosine
  modified_base 12..13: mod_base = OTHER; note = S-phosphorothioate
  modified_base 13: mod_base = OTHER; note = 2-fluororibose adenine
  modified_base 14: mod_base = OTHER; note = 2-fluororibose guanine
  modified_base 14..15: mod_base = OTHER; note = R-N-(1,3-dimethylimidazolidin-2-ylidene)phosphorami date
  modified_base 15: mod_base = OTHER; note = 2-fluororibose uracil
  modified_base 15..16: mod_base = OTHER; note = S-phosphorothioate
  modified_base 16: mod_base = OTHER; note = 2-fluororibose cytosine
  modified_base 16..17: mod_base = OTHER; note = S-phosphorothioate
  modified_base 17: mod_base = OTHER; note = 2-fluororibose cytosine
  modified_base 17..18: mod_base = OTHER; note = S-phosphorothioate
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = S-phosphorothioate
  modified_base 20: mod_base = um
  modified_base 21: mod_base = OTHER; note = 2-fluororibose uracil
  modified_base 21..22: mod_base = OTHER; note = S-phosphorothioate
  modified_base 22: mod_base = OTHER; note = 2-fluororibose cytosine
  modified_base 22..23: mod_base = OTHER; note = S-phosphorothioate
  modified_base 23: mod_base = OTHER; note = 2-fluororibose uracil
  modified_base 23..24: mod_base = OTHER; note = S-phosphorothioate
  modified_base 24: mod_base = OTHER; note = deoxyribose 6-amino-9-substituted 8,9-dihydro-7H-purin-8-one
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA
cccagcagcttcagtccctttctagtcgat

SEQ ID NO: 18 - RNA, 30 nt, synthetic construct; identical to SEQ ID NO: 1
  misc_feature 1..30: note = Synthetic: WV-46323
  misc_feature 1..30: note = Synthetic: ADR-0100552
  modified_base 1: mod_base = OTHER; note = triantennary GalNAc with C12 linker linked to 6-aminohexanol linked by phosphate to the 5' end of 2-O-methylribose cytosine
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = OTHER; note = 2-fluororibose cytosine
  modified_base 7: mod_base = OTHER; note = 2-O-methylribose adenine
  modified_base 7..8: mod_base = OTHER; note = S-phosphorothioate
  modified_base 8: mod_base = OTHER; note = 2-fluororibose guanine
  modified_base 8..9: mod_base = OTHER; note = S-phosphorothioate
  modified_base 9: mod_base = OTHER; note = 2-fluororibose cytosine
  modified_base 10: mod_base = um
  modified_base 10..11: mod_base = OTHER; note = S-phosphorothioate
  modified_base 11: mod_base = OTHER; note = 2-fluororibose uracil
  modified_base 11..12: mod_base = OTHER; note = R-N-(1,3-dimethylimidazolidin-2-ylidene)phosphorami date
  modified_base 12: mod_base = cm
  modified_base 13: mod_base = OTHER; note = 2-fluororibose adenine
  modified_base 13..14: mod_base = OTHER; note = S-phosphorothioate
  modified_base 14: mod_base = OTHER; note = 2-fluororibose guanine
  modified_base 14..15: mod_base = OTHER; note = R-N-(1,3-dimethylimidazolidin-2-ylidene)phosphorami date
  modified_base 15: mod_base = OTHER; note = 2-fluororibose uracil
  modified_base 16: mod_base = cm
  modified_base 16..17: mod_base = OTHER; note = S-phosphorothioate
  modified_base 17: mod_base = OTHER; note = 2-fluororibose cytosine
  modified_base 17..18: mod_base = OTHER; note = S-phosphorothioate
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = S-phosphorothioate
  modified_base 20: mod_base = OTHER; note = 2-fluororibose uracil
  modified_base 20..21: mod_base = OTHER; note = S-phosphorothioate
  modified_base 21: mod_base = um
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 19 - RNA, 30 nt, synthetic construct; identical to SEQ ID NO: 1
  misc_feature 1..30: note = Synthetic: WV-46313
  misc_feature 1..30: note = Synthetic: ADR-0100542
  modified_base 1: mod_base = OTHER; note = triantennary GalNAc with C12 linker linked to 6-aminohexanol linked by phosphate to the 5' end of 2-O-methylribose cytosine
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = cm
  modified_base 7: mod_base = OTHER; note = 2-O-methylribose adenine
  modified_base 8: mod_base = OTHER; note = 2-fluororibose guanine
  modified_base 8..9: mod_base = OTHER; note = S-phosphorothioate
  modified_base 9: mod_base = OTHER; note = 2-fluororibose cytosine
  modified_base 9..10: mod_base = OTHER; note = S-phosphorothioate
  modified_base 10: mod_base = um
  modified_base 11: mod_base = OTHER; note = 2-fluororibose uracil
  modified_base 11..12: mod_base = OTHER; note = R-N-(1,3-dimethylimidazolidin-2-ylidene)phosphorami date
  modified_base 12: mod_base = cm
  modified_base 13: mod_base = OTHER; note = 2-fluororibose adenine
  modified_base 13..14: mod_base = OTHER; note = S-phosphorothioate
  modified_base 14: mod_base = OTHER; note = 2-fluororibose guanine
  modified_base 14..15: mod_base = OTHER; note = R-N-(1,3-dimethylimidazolidin-2-ylidene)phosphorami date
  modified_base 15: mod_base = OTHER; note = 2-fluororibose uracil
  modified_base 16: mod_base = cm
  modified_base 16..17: mod_base = OTHER; note = S-phosphorothioate
  modified_base 17: mod_base = OTHER; note = 2-fluororibose cytosine
  modified_base 17..18: mod_base = OTHER; note = S-phosphorothioate
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = R-N-(1,3-dimethylimidazolidin-2-ylidene)phosphorami date
  modified_base 20: mod_base = um
  modified_base 21: mod_base = um
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 20 - RNA, 30 nt, synthetic construct; identical to SEQ ID NO: 1
  misc_feature 1..30: note = Synthetic: WV-46314
  misc_feature 1..30: note = Synthetic: ADR-0100543
  modified_base 1: mod_base = OTHER; note = triantennary GalNAc with C12 linker linked to 6-aminohexanol linked by phosphate to the 5' end of 2-O-methylribose cytosine
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = cm
  modified_base 7: mod_base = OTHER; note = 2-O-methylribose adenine
  modified_base 7..8: mod_base = OTHER; note = S-phosphorothioate
  modified_base 8: mod_base = OTHER; note = 2-fluororibose guanine
  modified_base 8..9: mod_base = OTHER; note = S-phosphorothioate
  modified_base 9: mod_base = OTHER; note = 2-fluororibose cytosine
  modified_base 10: mod_base = um
  modified_base 10..11: mod_base = OTHER; note = S-phosphorothioate
  modified_base 11: mod_base = OTHER; note = 2-fluororibose uracil
  modified_base 11..12: mod_base = OTHER; note = R-N-(1,3-dimethylimidazolidin-2-ylidene)phosphorami date
  modified_base 12: mod_base = cm
  modified_base 13: mod_base = OTHER; note = 2-fluororibose adenine
  modified_base 13..14: mod_base = OTHER; note = S-phosphorothioate
  modified_base 14: mod_base = OTHER; note = 2-fluororibose guanine
  modified_base 14..15: mod_base = OTHER; note = R-N-(1,3-dimethylimidazolidin-2-ylidene)phosphorami date
  modified_base 15: mod_base = OTHER; note = 2-fluororibose uracil
  modified_base 16: mod_base = cm
  modified_base 16..17: mod_base = OTHER; note = S-phosphorothioate
  modified_base 17: mod_base = OTHER; note = 2-fluororibose cytosine
  modified_base 17..18: mod_base = OTHER; note = S-phosphorothioate
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = R-N-(1,3-dimethylimidazolidin-2-ylidene)phosphorami date
  modified_base 20: mod_base = um
  modified_base 20..21: mod_base = OTHER; note = S-phosphorothioate
  modified_base 21: mod_base = um
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 21 - RNA, 30 nt, synthetic construct; identical to SEQ ID NO: 1
  misc_feature 1..30: note = Synthetic: WV-46315
  misc_feature 1..30: note = Synthetic: ADR-0100544
  modified_base 1: mod_base = OTHER; note = triantennary GalNAc with C12 linker linked to 6-aminohexanol linked by phosphate to the 5' end of 2-O-methylribose cytosine
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = cm
  modified_base 7: mod_base = OTHER; note = 2-O-methylribose adenine
  modified_base 8: mod_base = OTHER; note = 2-fluororibose guanine
  modified_base 8..9: mod_base = OTHER; note = S-phosphorothioate
  modified_base 9: mod_base = OTHER; note = 2-fluororibose cytosine
  modified_base 9..10: mod_base = OTHER; note = S-phosphorothioate
  modified_base 10: mod_base = OTHER; note = 2-fluororibose uracil
  modified_base 10..11: mod_base = OTHER; note = S-phosphorothioate
  modified_base 11: mod_base = OTHER; note = 2-fluororibose uracil
  modified_base 11..12: mod_base = OTHER; note = R-N-(1,3-dimethylimidazolidin-2-ylidene)phosphorami date
  modified_base 12: mod_base = OTHER; note = 2-fluororibose cytosine
  modified_base 12..13: mod_base = OTHER; note = S-phosphorothioate
  modified_base 13: mod_base = OTHER; note = 2-fluororibose adenine
  modified_base 14: mod_base = OTHER; note = 2-fluororibose guanine
  modified_base 14..15: mod_base = OTHER; note = R-N-(1,3-dimethylimidazolidin-2-ylidene)phosphorami date
  modified_base 15: mod_base = OTHER; note = 2-fluororibose uracil
  modified_base 16: mod_base = cm
  modified_base 17: mod_base = cm
  modified_base 17..18: mod_base = OTHER; note = S-phosphorothioate
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = S-phosphorothioate
  modified_base 20: mod_base = um
  modified_base 21: mod_base = um
  modified_base 21..22: mod_base = OTHER; note = S-phosphorothioate
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 22 - RNA, 30 nt, synthetic construct; identical to SEQ ID NO: 1
  misc_feature 1..30: note = Synthetic: WV-46316
  misc_feature 1..30: note = Synthetic: ADR-0100545
  modified_base 1: mod_base = OTHER; note = triantennary GalNAc with C12 linker linked to 6-aminohexanol linked by phosphate to the 5' end of 2-O-methylribose cytosine
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = cm
  modified_base 7: mod_base = OTHER; note = 2-O-methylribose adenine
  modified_base 8: mod_base = OTHER; note = 2-fluororibose guanine
  modified_base 8..9: mod_base = OTHER; note = S-phosphorothioate
  modified_base 9: mod_base = OTHER; note = 2-fluororibose cytosine
  modified_base 9..10: mod_base = OTHER; note = S-phosphorothioate
  modified_base 10: mod_base = OTHER; note = 2-fluororibose uracil
  modified_base 10..11: mod_base = OTHER; note = S-phosphorothioate
  modified_base 11: mod_base = OTHER; note = 2-fluororibose uracil
  modified_base 11..12: mod_base = OTHER; note = R-N-(1,3-dimethylimidazolidin-2-ylidene)phosphorami date
  modified_base 12: mod_base = OTHER; note = 2-fluororibose cytosine
  modified_base 12..13: mod_base = OTHER; note = S-phosphorothioate
  modified_base 13: mod_base = OTHER; note = 2-fluororibose adenine
  modified_base 14: mod_base = OTHER; note = 2-fluororibose guanine
  modified_base 14..15: mod_base = OTHER; note = R-N-(1,3-dimethylimidazolidin-2-ylidene)phosphorami date
  modified_base 15: mod_base = OTHER; note = 2-fluororibose uracil
  modified_base 15..16: mod_base = OTHER; note = S-phosphorothioate
  modified_base 16: mod_base = OTHER; note = 2-fluororibose cytosine
  modified_base 16..17: mod_base = OTHER; note = S-phosphorothioate
  modified_base 17: mod_base = OTHER; note = 2-fluororibose cytosine
  modified_base 17..18: mod_base = OTHER; note = S-phosphorothioate
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = S-phosphorothioate
  modified_base 20: mod_base = um
  modified_base 21: mod_base = um
  modified_base 21..22: mod_base = OTHER; note = S-phosphorothioate
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 23 - RNA, 30 nt, synthetic construct; identical to SEQ ID NO: 1
  misc_feature 1..30: note = Synthetic: WV-46317
  misc_feature 1..30: note = Synthetic: ADR-0100546
  modified_base 1: mod_base = OTHER; note = triantennary GalNAc with C12 linker linked to 6-aminohexanol linked by phosphate to the 5' end of 2-O-methylribose cytosine
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = cm
  modified_base 7: mod_base = OTHER; note = 2-O-methylribose adenine
  modified_base 8: mod_base = OTHER; note = 2-fluororibose guanine
  modified_base 8..9: mod_base = OTHER; note = S-phosphorothioate
  modified_base 9: mod_base = OTHER; note = 2-fluororibose cytosine
  modified_base 9..10: mod_base = OTHER; note = S-phosphorothioate
  modified_base 10: mod_base = um
  modified_base 10..11: mod_base = OTHER; note = S-phosphorothioate
  modified_base 11: mod_base = OTHER; note = 2-fluororibose uracil
  modified_base 11..12: mod_base = OTHER; note = R-N-(1,3-dimethylimidazolidin-2-ylidene)phosphorami date
  modified_base 12: mod_base = cm
  modified_base 13: mod_base = OTHER; note = 2-fluororibose adenine
  modified_base 13..14: mod_base = OTHER; note = S-phosphorothioate
  modified_base 14: mod_base = OTHER; note = 2-fluororibose guanine
  modified_base 14..15: mod_base = OTHER; note = R-N-(1,3-dimethylimidazolidin-2-ylidene)phosphorami date
  modified_base 15: mod_base = OTHER; note = 2-fluororibose uracil
  modified_base 16: mod_base = cm
  modified_base 16..17: mod_base = OTHER; note = S-phosphorothioate
  modified_base 17: mod_base = OTHER; note = 2-fluororibose cytosine
  modified_base 17..18: mod_base = OTHER; note = S-phosphorothioate
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = R-N-(1,3-dimethylimidazolidin-2-ylidene)phosphorami date
  modified_base 20: mod_base = um
  modified_base 21: mod_base = um
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 24 - RNA, 30 nt, synthetic construct; identical to SEQ ID NO: 1
  misc_feature 1..30: note = Synthetic: WV-46318
  misc_feature 1..30: note = Synthetic: ADR-0100547
  modified_base 1: mod_base = OTHER; note = triantennary GalNAc with C12 linker linked to 6-aminohexanol linked by phosphate to the 5' end of 2-O-methylribose cytosine
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = cm
  modified_base 7: mod_base = OTHER; note = 2-O-methylribose adenine
  modified_base 7..8: mod_base = OTHER; note = S-phosphorothioate
  modified_base 8: mod_base = OTHER; note = 2-fluororibose guanine
  modified_base 8..9: mod_base = OTHER; note = S-phosphorothioate
  modified_base 9: mod_base = OTHER; note = 2-fluororibose cytosine
  modified_base 10: mod_base = um
  modified_base 10..11: mod_base = OTHER; note = S-phosphorothioate
  modified_base 11: mod_base = OTHER; note = 2-fluororibose uracil
  modified_base 11..12: mod_base = OTHER; note = R-N-(1,3-dimethylimidazolidin-2-ylidene)phosphorami date
  modified_base 12: mod_base = cm
  modified_base 13: mod_base = OTHER; note = 2-fluororibose adenine
  modified_base 13..14: mod_base = OTHER; note = S-phosphorothioate
  modified_base 14: mod_base = OTHER; note = 2-fluororibose guanine
  modified_base 14..15: mod_base = OTHER; note = R-N-(1,3-dimethylimidazolidin-2-ylidene)phosphorami date
  modified_base 15: mod_base = OTHER; note = 2-fluororibose uracil
  modified_base 16: mod_base = cm
  modified_base 16..17: mod_base = OTHER; note = S-phosphorothioate
  modified_base 17: mod_base = OTHER; note = 2-fluororibose cytosine
  modified_base 17..18: mod_base = OTHER; note = S-phosphorothioate
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = R-N-(1,3-dimethylimidazolidin-2-ylidene)phosphorami date
  modified_base 20: mod_base = um
  modified_base 21: mod_base = um
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 25 - RNA, 30 nt, synthetic construct; identical to SEQ ID NO: 1
  misc_feature 1..30: note = Synthetic: WV-46319
  misc_feature 1..30: note = Synthetic: ADR-0100548
  modified_base 1: mod_base = OTHER; note = triantennary GalNAc with C12 linker linked to 6-aminohexanol linked by phosphate to the 5' end of 2-O-methylribose cytosine
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = cm
  modified_base 7: mod_base = OTHER; note = 2-O-methylribose adenine
  modified_base 8: mod_base = OTHER; note = 2-fluororibose guanine
  modified_base 8..9: mod_base = OTHER; note = S-phosphorothioate
  modified_base 9: mod_base = OTHER; note = 2-fluororibose cytosine
  modified_base 10: mod_base = um
  modified_base 10..11: mod_base = OTHER; note = S-phosphorothioate
  modified_base 11: mod_base = OTHER; note = 2-fluororibose uracil
  modified_base 11..12: mod_base = OTHER; note = R-N-(1,3-dimethylimidazolidin-2-ylidene)phosphorami date
  modified_base 12: mod_base = cm
  modified_base 13: mod_base = OTHER; note = 2-fluororibose adenine
  modified_base 13..14: mod_base = OTHER; note = S-phosphorothioate
  modified_base 14: mod_base = OTHER; note = 2-fluororibose guanine
  modified_base 14..15: mod_base = OTHER; note = R-N-(1,3-dimethylimidazolidin-2-ylidene)phosphorami date
  modified_base 15: mod_base = OTHER; note = 2-fluororibose uracil
  modified_base 16: mod_base = cm
  modified_base 16..17: mod_base = OTHER; note = S-phosphorothioate
  modified_base 17: mod_base = OTHER; note = 2-fluororibose cytosine
  modified_base 17..18: mod_base = OTHER; note = S-phosphorothioate
  modified_base 18: mod_base = cm
  modified_base 19: mod_base = OTHER; note = 2-fluororibose uracil
  modified_base 19..20: mod_base = OTHER; note = R-N-(1,3-dimethylimidazolidin-2-ylidene)phosphorami date
  modified_base 20: mod_base = um
  modified_base 21: mod_base = um
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 26 - RNA, 30 nt, synthetic construct; identical to SEQ ID NO: 1
  misc_feature 1..30: note = Synthetic: WV-46320
  misc_feature 1..30: note = Synthetic: ADR-0100549
  modified_base 1: mod_base = OTHER; note = triantennary GalNAc with C12 linker linked to 6-aminohexanol linked by phosphate to the 5' end of 2-O-methylribose cytosine
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = OTHER; note = 2-fluororibose cytosine
  modified_base 7: mod_base = OTHER; note = 2-O-methylribose adenine
  modified_base 7..8: mod_base = OTHER; note = S-phosphorothioate
  modified_base 8: mod_base = OTHER; note = 2-fluororibose guanine
  modified_base 8..9: mod_base = OTHER; note = S-phosphorothioate
  modified_base 9: mod_base = cm
  modified_base 10: mod_base = um
  modified_base 10..11: mod_base = OTHER; note = S-phosphorothioate
  modified_base 11: mod_base = OTHER; note = 2-fluororibose uracil
  modified_base 11..12: mod_base = OTHER; note = R-N-(1,3-dimethylimidazolidin-2-ylidene)phosphorami date
  modified_base 12: mod_base = cm
  modified_base 13: mod_base = OTHER; note = 2-fluororibose adenine
  modified_base 13..14: mod_base = OTHER; note = S-phosphorothioate
  modified_base 14: mod_base = OTHER; note = 2-fluororibose guanine
  modified_base 14..15: mod_base = OTHER; note = R-N-(1,3-dimethylimidazolidin-2-ylidene)phosphorami date
  modified_base 15: mod_base = OTHER; note = 2-fluororibose uracil
  modified_base 16: mod_base = cm
  modified_base 16..17: mod_base = OTHER; note = S-phosphorothioate
  modified_base 17: mod_base = OTHER; note = 2-fluororibose cytosine
  modified_base 17..18: mod_base = OTHER; note = S-phosphorothioate
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = R-N-(1,3-dimethylimidazolidin-2-ylidene)phosphorami date
  modified_base 20: mod_base = OTHER; note = 2-fluororibose uracil
  modified_base 20..21: mod_base = OTHER; note = S-phosphorothioate
  modified_base 21: mod_base = um
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 27 - RNA, 30 nt, synthetic construct; identical to SEQ ID NO: 1
  misc_feature 1..30: note = Synthetic: WV-46321
  misc_feature 1..30: note = Synthetic: ADR-0100550
  modified_base 1: mod_base = OTHER; note = triantennary GalNAc with C12 linker linked to 6-aminohexanol linked by phosphate to the 5' end of 2-O-methylribose cytosine
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = OTHER; note = 2-fluororibose cytosine
  modified_base 7: mod_base = OTHER; note = 2-O-methylribose adenine
  modified_base 7..8: mod_base = OTHER; note = S-phosphorothioate
  modified_base 8: mod_base = OTHER; note = 2-fluororibose guanine
  modified_base 8..9: mod_base = OTHER; note = S-phosphorothioate
  modified_base 9: mod_base = OTHER; note = 2-fluororibose cytosine
  modified_base 10: mod_base = um
  modified_base 10..11: mod_base = OTHER; note = S-phosphorothioate
  modified_base 11: mod_base = OTHER; note = 2-fluororibose uracil
  modified_base 11..12: mod_base = OTHER; note = R-N-(1,3-dimethylimidazolidin-2-ylidene)phosphorami date
  modified_base 12: mod_base = cm
  modified_base 13: mod_base = OTHER; note = 2-fluororibose adenine
  modified_base 13..14: mod_base = OTHER; note = S-phosphorothioate
  modified_base 14: mod_base = OTHER; note = 2-fluororibose guanine
  modified_base 14..15: mod_base = OTHER; note = R-N-(1,3-dimethylimidazolidin-2-ylidene)phosphorami date
  modified_base 15: mod_base = OTHER; note = 2-fluororibose uracil
  modified_base 16: mod_base = cm
  modified_base 16..17: mod_base = OTHER; note = S-phosphorothioate
  modified_base 17: mod_base = cm
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = R-N-(1,3-dimethylimidazolidin-2-ylidene)phosphorami date
  modified_base 20: mod_base = OTHER; note = 2-fluororibose uracil
  modified_base 20..21: mod_base = OTHER; note = S-phosphorothioate
  modified_base 21: mod_base = um
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 28 - RNA, 30 nt, synthetic construct; identical to SEQ ID NO: 1
  misc_feature 1..30: note = Synthetic: WV-46322
  misc_feature 1..30: note = Synthetic: ADR-0100551
  modified_base 1: mod_base = OTHER; note = triantennary GalNAc with C12 linker linked to 6-aminohexanol linked by phosphate to the 5' end of 2-O-methylribose cytosine
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = cm
  modified_base 7: mod_base = OTHER; note = 2-O-methylribose adenine
  modified_base 8: mod_base = OTHER; note = 2-fluororibose guanine
  modified_base 8..9: mod_base = OTHER; note = S-phosphorothioate
  modified_base 9: mod_base = OTHER; note = 2-fluororibose cytosine
  modified_base 9..10: mod_base = OTHER; note = S-phosphorothioate
  modified_base 10: mod_base = OTHER; note = 2-fluororibose uracil
  modified_base 10..11: mod_base = OTHER; note = S-phosphorothioate
  modified_base 11: mod_base = OTHER; note = 2-fluororibose uracil
  modified_base 11..12: mod_base = OTHER; note = R-N-(1,3-dimethylimidazolidin-2-ylidene)phosphorami date
  modified_base 12: mod_base = OTHER; note = 2-fluororibose cytosine
  modified_base 12..13: mod_base = OTHER; note = S-phosphorothioate
  modified_base 13: mod_base = OTHER; note = 2-fluororibose adenine
  modified_base 14: mod_base = OTHER; note = 2-fluororibose guanine
  modified_base 14..15: mod_base = OTHER; note = R-N-(1,3-dimethylimidazolidin-2-ylidene)phosphorami date
  modified_base 15: mod_base = OTHER; note = 2-fluororibose uracil
  modified_base 16: mod_base = cm
  modified_base 17: mod_base = cm
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = S-phosphorothioate
  modified_base 20: mod_base = um
  modified_base 21: mod_base = um
  modified_base 21..22: mod_base = OTHER; note = S-phosphorothioate
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 29 - RNA, 30 nt, synthetic construct; identical to SEQ ID NO: 1
  misc_feature 1..30: note = Synthetic: WV-49085b
  modified_base 1: mod_base = cm
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = OTHER; note = 2-O-methoxyethylribose 5-methyl-cytidine
  modified_base 7: mod_base = OTHER; note = 2-O-methoxyethylribose adenine
  modified_base 8: mod_base = OTHER; note = 2-fluororibose guanine
  modified_base 8..9: mod_base = OTHER; note = S-phosphorothioate
  modified_base 9: mod_base = OTHER; note = 2-fluororibose cytosine
  modified_base 9..10: mod_base = OTHER; note = S-phosphorothioate
  modified_base 10: mod_base = OTHER; note = 2-O-methoxyethylribose thymine
  modified_base 11: mod_base = OTHER; note = 2-fluororibose uracil
  modified_base 11..12: mod_base = OTHER; note = R-N-(1,3-dimethylimidazolidin-2-ylidene)phosphorami date
  modified_base 12: mod_base = cm
  modified_base 13: mod_base = OTHER; note = 2-fluororibose adenine
  modified_base 13..14: mod_base = OTHER; note = S-phosphorothioate
  modified_base 14: mod_base = gm
  modified_base 14..15: mod_base = OTHER; note = R-N-(1,3-dimethylimidazolidin-2-ylidene)phosphorami date
  modified_base 15: mod_base = OTHER; note = 2-fluororibose uracil
  modified_base 16: mod_base = cm
  modified_base 16..17: mod_base = OTHER; note = S-phosphorothioate
  modified_base 17: mod_base = OTHER; note = 2-fluororibose cytosine
  modified_base 17..18: mod_base = OTHER; note = S-phosphorothioate
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = R-N-(1,3-dimethylimidazolidin-2-ylidene)phosphorami date
  modified_base 20: mod_base = OTHER; note = 2-O-methoxyethylribose thymine
  modified_base 21: mod_base = OTHER; note = 2-O-methoxyethylribose thymine
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 30 - RNA, 30 nt, synthetic construct; identical to SEQ ID NO: 1
  misc_feature 1..30: note = Synthetic: WV-49086b
  modified_base 1: mod_base = cm
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = OTHER; note = 2-O-methoxyethylribose 5-methyl-cytidine
  modified_base 7: mod_base = OTHER; note = 2-O-methoxyethylribose adenine
  modified_base 8: mod_base = OTHER; note = 2-fluororibose guanine
  modified_base 8..9: mod_base = OTHER; note = S-phosphorothioate
  modified_base 9: mod_base = OTHER; note = 2-fluororibose cytosine
  modified_base 9..10: mod_base = OTHER; note = S-phosphorothioate
  modified_base 10: mod_base = OTHER; note = 2-O-methoxyethylribose thymine
  modified_base 11: mod_base = OTHER; note = 2-fluororibose uracil
  modified_base 11..12: mod_base = OTHER; note = R-N-(1,3-dimethylimidazolidin-2-ylidene)phosphorami date
  modified_base 12: mod_base = cm
  modified_base 13: mod_base = OTHER; note = 2-fluororibose adenine
  modified_base 13..14: mod_base = OTHER; note = S-phosphorothioate
  modified_base 14: mod_base = gm
  modified_base 14..15: mod_base = OTHER; note = R-N-(1,3-dimethylimidazolidin-2-ylidene)phosphorami date
  modified_base 15: mod_base = OTHER; note = 2-fluororibose uracil
  modified_base 16: mod_base = OTHER; note = 2-O-methoxyethylribose 5-methyl-cytidine
  modified_base 16..17: mod_base = OTHER; note = S-phosphorothioate
  modified_base 17: mod_base = OTHER; note = 2-fluororibose cytosine
  modified_base 17..18: mod_base = OTHER; note = S-phosphorothioate
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = R-N-(1,3-dimethylimidazolidin-2-ylidene)phosphorami date
  modified_base 20: mod_base = OTHER; note = 2-O-methoxyethylribose thymine
  modified_base 21: mod_base = OTHER; note = 2-O-methoxyethylribose thymine
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 31 - RNA, 30 nt, synthetic construct; identical to SEQ ID NO: 1
  misc_feature 1..30: note = Synthetic: WV-49087b
  modified_base 1: mod_base = cm
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = OTHER; note = 2-O-methoxyethylribose 5-methyl-cytidine
  modified_base 7: mod_base = OTHER; note = 2-O-methoxyethylribose adenine
  modified_base 8: mod_base = OTHER; note = 2-fluororibose guanine
  modified_base 8..9: mod_base = OTHER; note = S-phosphorothioate
  modified_base 9: mod_base = cm
  modified_base 10: mod_base = OTHER; note = 2-O-methoxyethylribose thymine
  modified_base 10..11: mod_base = OTHER; note = S-phosphorothioate
  modified_base 11: mod_base = um
  modified_base 11..12: mod_base = OTHER; note = R-N-(1,3-dimethylimidazolidin-2-ylidene)phosphorami date
  modified_base 12: mod_base = cm
  modified_base 13: mod_base = OTHER; note = 2-fluororibose adenine
  modified_base 13..14: mod_base = OTHER; note = S-phosphorothioate
  modified_base 14: mod_base = OTHER; note = 2-fluororibose guanine
  modified_base 14..15: mod_base = OTHER; note = R-N-(1,3-dimethylimidazolidin-2-ylidene)phosphorami date
  modified_base 15: mod_base = um
  modified_base 16: mod_base = cm
  modified_base 17: mod_base = cm
  modified_base 17..18: mod_base = OTHER; note = S-phosphorothioate
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = S-phosphorothioate
  modified_base 20: mod_base = OTHER; note = 2-O-methoxyethylribose thymine
  modified_base 21: mod_base = OTHER; note = 2-O-methoxyethylribose thymine
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 32 - RNA, 30 nt, synthetic construct; identical to SEQ ID NO: 1
  misc_feature 1..30: note = Synthetic: WV-49089b
  modified_base 1: mod_base = cm
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = OTHER; note = 2-O-methoxyethylribose 5-methyl-cytidine
  modified_base 7: mod_base = OTHER; note = 2-O-methoxyethylribose adenine
  modified_base 8: mod_base = OTHER; note = 2-fluororibose guanine
  modified_base 8..9: mod_base = OTHER; note = S-phosphorothioate
  modified_base 9: mod_base = OTHER; note = 2-O-methoxyethylribose 5-methyl-cytidine
  modified_base 10: mod_base = OTHER; note = 2-O-methoxyethylribose thymine
  modified_base 10..11: mod_base = OTHER; note = S-phosphorothioate
  modified_base 11: mod_base = um
  modified_base 11..12: mod_base = OTHER; note = R-N-(1,3-dimethylimidazolidin-2-ylidene)phosphorami date
  modified_base 12: mod_base = OTHER; note = 2-O-methoxyethylribose 5-methyl-cytidine
  modified_base 13: mod_base = OTHER; note = 2-fluororibose adenine
  modified_base 13..14: mod_base = OTHER; note = S-phosphorothioate
  modified_base 14: mod_base = OTHER; note = 2-fluororibose guanine
  modified_base 14..15: mod_base = OTHER; note = R-N-(1,3-dimethylimidazolidin-2-ylidene)phosphorami date
  modified_base 15: mod_base = um
  modified_base 16: mod_base = OTHER; note = 2-O-methoxyethylribose 5-methyl-cytidine
  modified_base 17: mod_base = OTHER; note = 2-O-methoxyethylribose 5-methyl-cytidine
  modified_base 17..18: mod_base = OTHER; note = S-phosphorothioate
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = S-phosphorothioate
  modified_base 20: mod_base = OTHER; note = 2-O-methoxyethylribose thymine
  modified_base 21: mod_base = OTHER; note = 2-O-methoxyethylribose thymine
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 33 - RNA, 30 nt, synthetic construct; identical to SEQ ID NO: 1
  modified_base 1: mod_base = OTHER; note = triantennary GalNAc with C12 linker linked to 6-aminohexanol linked by phosphate to the 5' end of 2-O-methylribose cytosine
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = cm
  modified_base 7: mod_base = OTHER; note = 2-O-methylribose adenine
  modified_base 7..8: mod_base = OTHER; note = S-phosphorothioate
  modified_base 8: mod_base = OTHER; note = 2-fluororibose guanine
  modified_base 8..9: mod_base = OTHER; note = S-phosphorothioate
  modified_base 9: mod_base = OTHER; note = 2-fluororibose cytosine
  modified_base 10: mod_base = um
  modified_base 10..11: mod_base = OTHER; note = S-phosphorothioate
  modified_base 11: mod_base = OTHER; note = 2-fluororibose uracil
  modified_base 11..12: mod_base = OTHER; note = R-N-(1,3-dimethylimidazolidin-2-ylidene)phosphorami date
  modified_base 12: mod_base = cm
  modified_base 13: mod_base = OTHER; note = 2-fluororibose adenine
  modified_base 13..14: mod_base = OTHER; note = S-phosphorothioate
  modified_base 14: mod_base = OTHER; note = 2-fluororibose guanine
  modified_base 14..15: mod_base = OTHER; note = R-N-(1,3-dimethylimidazolidin-2-ylidene)phosphorami date
  modified_base 15: mod_base = OTHER; note = 2-fluororibose uracil
  modified_base 16: mod_base = cm
  modified_base 16..17: mod_base = OTHER; note = S-phosphorothioate
  modified_base 17: mod_base = OTHER; note = 2-fluororibose cytosine
  modified_base 17..18: mod_base = OTHER; note = S-phosphorothioate
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = S-phosphorothioate
  modified_base 20: mod_base = um
  modified_base 21: mod_base = um
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 34 - RNA, 30 nt, synthetic construct; identical to SEQ ID NO: 1
  modified_base 1: mod_base = OTHER; note = triantennary GalNAc with C12 linker linked to 6-aminohexanol linked by phosphate to the 5' end of 2-O-methylribose cytosine
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = OTHER; note = 2-O-methoxyethylribose 5-methyl-cytidine
  modified_base 7: mod_base = OTHER; note = 2-O-methoxyethylribose adenine
  modified_base 8: mod_base = OTHER; note = 2-fluororibose guanine
  modified_base 8..9: mod_base = OTHER; note = S-phosphorothioate
  modified_base 9: mod_base = OTHER; note = 2-fluororibose cytosine
  modified_base 9..10: mod_base = OTHER; note = S-phosphorothioate
  modified_base 10: mod_base = um
  modified_base 11: mod_base = um
  modified_base 11..12: mod_base = OTHER; note = R-N-(1,3-dimethylimidazolidin-2-ylidene)phosphorami date
  modified_base 12: mod_base = cm
  modified_base 13: mod_base = OTHER; note = 2-fluororibose adenine
  modified_base 13..14: mod_base = OTHER; note = S-phosphorothioate
  modified_base 14: mod_base = OTHER; note = 2-fluororibose guanine
  modified_base 14..15: mod_base = OTHER; note = R-N-(1,3-dimethylimidazolidin-2-ylidene)phosphorami date
  modified_base 15: mod_base = OTHER; note = 2-fluororibose uracil
  modified_base 16: mod_base = OTHER; note = 2-O-methoxyethylribose 5-methyl-cytidine
  modified_base 16..17: mod_base = OTHER; note = S-phosphorothioate
  modified_base 17: mod_base = OTHER; note = 2-fluororibose cytosine
  modified_base 17..18: mod_base = OTHER; note = S-phosphorothioate
  modified_base 18: mod_base = cm
  modified_base 19: mod_base = um
  modified_base 19..20: mod_base = OTHER; note = R-N-(1,3-dimethylimidazolidin-2-ylidene)phosphorami date
  modified_base 20: mod_base = um
  modified_base 21: mod_base = OTHER; note = 2-O-methoxyethylribose thymine
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 35 - RNA, 30 nt, synthetic construct; identical to SEQ ID NO: 1
  modified_base 1: mod_base = OTHER; note = triantennary GalNAc with C12 linker linked to 6-aminohexanol linked by phosphate to the 5' end of 2-O-methylribose cytosine
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = OTHER; note = 2-O-methoxyethylribose 5-methyl-cytidine
  modified_base 7: mod_base = OTHER; note = 2-O-methoxyethylribose adenine
  modified_base 8: mod_base = OTHER; note = 2-fluororibose guanine
  modified_base 8..9: mod_base = OTHER; note = S-phosphorothioate
  modified_base 9: mod_base = OTHER; note = 2-fluororibose cytosine
  modified_base 9..10: mod_base = OTHER; note = S-phosphorothioate
  modified_base 10: mod_base = OTHER; note = 2-O-methoxyethylribose thymine
  modified_base 11: mod_base = OTHER; note = 2-fluororibose uracil
  modified_base 11..12: mod_base = OTHER; note = R-N-(1,3-dimethylimidazolidin-2-ylidene)phosphorami date
  modified_base 12: mod_base = cm
  modified_base 13: mod_base = OTHER; note = 2-fluororibose adenine
  modified_base 13..14: mod_base = OTHER; note = S-phosphorothioate
  modified_base 14: mod_base = OTHER; note = 2-fluororibose guanine
  modified_base 14..15: mod_base = OTHER; note = R-N-(1,3-dimethylimidazolidin-2-ylidene)phosphorami date
  modified_base 15: mod_base = OTHER; note = 2-fluororibose uracil
  modified_base 16: mod_base = OTHER; note = 2-O-methoxyethylribose 5-methyl-cytidine
  modified_base 16..17: mod_base = OTHER; note = S-phosphorothioate
  modified_base 17: mod_base = OTHER; note = 2-fluororibose cytosine
  modified_base 17..18: mod_base = OTHER; note = S-phosphorothioate
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = R-N-(1,3-dimethylimidazolidin-2-ylidene)phosphorami date
  modified_base 20: mod_base = OTHER; note = 2-O-methoxyethylribose thymine
  modified_base 21: mod_base = OTHER; note = 2-O-methoxyethylribose thymine
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 36 - RNA, 30 nt, synthetic construct; identical to SEQ ID NO: 1
  modified_base 1: mod_base = cm
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = cm
  modified_base 7: mod_base = OTHER; note = 2-O-methylribose adenine
  modified_base 7..8: mod_base = OTHER; note = S-phosphorothioate
  modified_base 8: mod_base = OTHER; note = 2-fluororibose guanine
  modified_base 8..9: mod_base = OTHER; note = S-phosphorothioate
  modified_base 9: mod_base = OTHER; note = 2-fluororibose cytosine
  modified_base 10: mod_base = um
  modified_base 10..11: mod_base = OTHER; note = S-phosphorothioate
  modified_base 11: mod_base = OTHER; note = 2-fluororibose uracil
  modified_base 11..12: mod_base = OTHER; note = R-N-(1,3-dimethylimidazolidin-2-ylidene)phosphorami date
  modified_base 12: mod_base = cm
  modified_base 13: mod_base = OTHER; note = 2-fluororibose adenine
  modified_base 13..14: mod_base = OTHER; note = S-phosphorothioate
  modified_base 14: mod_base = OTHER; note = 2-fluororibose guanine
  modified_base 14..15: mod_base = OTHER; note = R-N-(1,3-dimethylimidazolidin-2-ylidene)phosphorami date
  modified_base 15: mod_base = OTHER; note = 2-fluororibose uracil
  modified_base 16: mod_base = cm
  modified_base 16..17: mod_base = OTHER; note = S-phosphorothioate
  modified_base 17: mod_base = OTHER; note = 2-fluororibose cytosine
  modified_base 17..18: mod_base = OTHER; note = S-phosphorothioate
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = S-phosphorothioate
  modified_base 20: mod_base = um
  modified_base 21: mod_base = um
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 37 - RNA, 30 nt, synthetic construct; identical to SEQ ID NO: 1
  modified_base 1: mod_base = cm
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = OTHER; note = 2-O-methoxyethylribose 5-methyl-cytidine
  modified_base 7: mod_base = OTHER; note = 2-O-methoxyethylribose adenine
  modified_base 8: mod_base = OTHER; note = 2-fluororibose guanine
  modified_base 8..9: mod_base = OTHER; note = S-phosphorothioate
  modified_base 9: mod_base = OTHER; note = 2-fluororibose cytosine
  modified_base 9..10: mod_base = OTHER; note = S-phosphorothioate
  modified_base 10: mod_base = um
  modified_base 11: mod_base = um
  modified_base 11..12: mod_base = OTHER; note = R-N-(1,3-dimethylimidazolidin-2-ylidene)phosphorami date
  modified_base 12: mod_base = cm
  modified_base 13: mod_base = OTHER; note = 2-fluororibose adenine
  modified_base 13..14: mod_base = OTHER; note = S-phosphorothioate
  modified_base 14: mod_base = OTHER; note = 2-fluororibose guanine
  modified_base 14..15: mod_base = OTHER; note = R-N-(1,3-dimethylimidazolidin-2-ylidene)phosphorami date
  modified_base 15: mod_base = OTHER; note = 2-fluororibose uracil
  modified_base 16: mod_base = OTHER; note = 2-O-methoxyethylribose 5-methyl-cytidine
  modified_base 16..17: mod_base = OTHER; note = S-phosphorothioate
  modified_base 17: mod_base = OTHER; note = 2-fluororibose cytosine
  modified_base 17..18: mod_base = OTHER; note = S-phosphorothioate
  modified_base 18: mod_base = cm
  modified_base 19: mod_base = um
  modified_base 19..20: mod_base = OTHER; note = R-N-(1,3-dimethylimidazolidin-2-ylidene)phosphorami date
  modified_base 20: mod_base = um
  modified_base 21: mod_base = OTHER; note = 2-O-methoxyethylribose thymine
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

SEQ ID NO: 38 - RNA, 30 nt, synthetic construct; identical to SEQ ID NO: 1
  modified_base 1: mod_base = cm
  modified_base 1..2: mod_base = OTHER; note = R-N-(1,3-dimethylimidazolidin-2-ylidene)phosphorami date
  modified_base 2: mod_base = cm
  modified_base 2..3: mod_base = OTHER; note = S-phosphorothioate
  modified_base 3: mod_base = cm
  modified_base 3..4: mod_base = OTHER; note = S-phosphorothioate
  modified_base 4: mod_base = OTHER; note = 2-fluororibose adenine
  modified_base 4..5: mod_base = OTHER; note = S-phosphorothioate
  modified_base 5: mod_base = OTHER; note = 2-fluororibose guanine
  modified_base 5..6: mod_base = OTHER; note = S-phosphorothioate
  modified_base 6: mod_base = OTHER; note = 2-O-methoxyethylribose 5-methyl-cytidine
  modified_base 7: mod_base = OTHER; note = 2-O-methoxyethylribose adenine
  modified_base 8: mod_base = OTHER; note = 2-fluororibose guanine
  modified_base 8..9: mod_base = OTHER; note = S-phosphorothioate
  modified_base 9: mod_base = OTHER; note = 2-fluororibose cytosine
  modified_base 9..10: mod_base = OTHER; note = S-phosphorothioate
  modified_base 10: mod_base = OTHER; note = 2-O-methoxyethylribose thymine
  modified_base 11: mod_base = OTHER; note = 2-fluororibose uracil
  modified_base 11..12: mod_base = OTHER; note = R-N-(1,3-dimethylimidazolidin-2-ylidene)phosphorami date
  modified_base 12: mod_base = cm
  modified_base 13: mod_base = OTHER; note = 2-fluororibose adenine
  modified_base 13..14: mod_base = OTHER; note = S-phosphorothioate
  modified_base 14: mod_base = OTHER; note = 2-fluororibose guanine
  modified_base 14..15: mod_base = OTHER; note = R-N-(1,3-dimethylimidazolidin-2-ylidene)phosphorami date
  modified_base 15: mod_base = OTHER; note = 2-fluororibose uracil
  modified_base 16: mod_base = OTHER; note = 2-O-methoxyethylribose 5-methyl-cytidine
  modified_base 16..17: mod_base = OTHER; note = S-phosphorothioate
  modified_base 17: mod_base = OTHER; note = 2-fluororibose cytosine
  modified_base 17..18: mod_base = OTHER; note = S-phosphorothioate
  modified_base 18: mod_base = OTHER; note = 2-fluororibose cytosine
  modified_base 18..19: mod_base = OTHER; note = S-phosphorothioate
  modified_base 19: mod_base = OTHER; note = 2-fluororibose uracil
  modified_base 19..20: mod_base = OTHER; note = R-N-(1,3-dimethylimidazolidin-2-ylidene)phosphorami date
  modified_base 20: mod_base = OTHER; note = 2-O-methoxyethylribose thymine
  modified_base 21: mod_base = OTHER; note = 2-O-methoxyethylribose thymine
  modified_base 22: mod_base = OTHER; note = 2-fluororibose cytosine
  modified_base 22..23: mod_base = OTHER; note = S-phosphorothioate
  modified_base 23: mod_base = OTHER; note = deoxyribose thymine
  modified_base 23..24: mod_base = OTHER; note = S-phosphorothioate
  modified_base 24: mod_base = OTHER; note = deoxyribose uracil (position 3 connectivity)
  modified_base 24..25: mod_base = OTHER; note = S-phosphorothioate
  modified_base 25: mod_base = OTHER; note = deoxyribose inosine
  modified_base 25..26: mod_base = OTHER; note = S-N-(1,3-dimethylimidazolidin-2-ylidene)phosphorami date
  modified_base 26: mod_base = um
  modified_base 27: mod_base = OTHER; note = 2-fluororibose cytosine
  modified_base 27..28: mod_base = OTHER; note = S-phosphorothioate
  modified_base 28: mod_base = gm
  modified_base 28..29: mod_base = OTHER; note = S-phosphorothioate
  modified_base 29: mod_base = OTHER; note = 2-O-methylribose adenine
  modified_base 29..30: mod_base = OTHER; note = R-N-(1,3-dimethylimidazolidin-2-ylidene)phosphorami date
  modified_base 30: mod_base = um
  source 1..30: mol_type = other RNA

(19) United States
(12) Patent Application Publication
(10) Pub. No.: US 2025/0333740 A1
(43) Pub. Date: Oct. 30, 2025

(54) OLIGONUCLEOTIDE COMPOSITIONS AND METHODS THEREOF

(71) Applicant: WAVE LIFE SCIENCES LTD., Singapore (SG)

(72) Inventors: Prashant Monian, Arlington, MA (US); Chikdu Shakti Shivalila, Woburn, MA (US); Genliang Lu, Winchester, MA (US); Chandra Vargeese, Schwenksville, PA (US); Paloma Hoban Giangrande, Belmont, MA (US); Pachamuthu Kandasamy, Lexington, MA (US); Naoki Iwamoto, Boston, MA (US); Mamoru Shimizu, Arlington, MA (US); Hui Yu, Arlington, MA (US); Hailin Yang, West Roxbury, MA (US); Sarah Diane Lamore, Burlington, MA (US); Fengjiao Zhang, Lexington, MA (US); Padmakumar Narayanan, Carlsbad, CA (US)

(21) Appl. No.: 18/695,346

(22) PCT Filed: Sep. 26, 2022
(86) PCT No.: PCT/US2022/044765

§ 371 Date: Mar. 25, 2024

Related U.S. Application Data
(60) Provisional application No. 63/248,520, filed on Sep. 26, 2021.
(60) Provisional application No. 63/331,756, filed on Apr. 15, 2022.
(60) Provisional application No. 63/397,320, filed on Aug. 11, 2022.

(30) Foreign Application Priority Data
Nov. 8, 2021 (WO) PCT/US2021/058495

(51) Int. Cl. C12N 15/113; A61P 11/00

(52) U.S. Cl. CPC C12N 15/1137; A61P 11/00; C12N 2310/14; C12N 2310/315; C12N 2310/321; C12N 2310/322; C12N 2310/351; C12N 2320/32; C12Y 304/21011

(57) ABSTRACT

Among other things, the present disclosure provides designed oligonucleotides and compositions thereof. In some embodiments, oligonucleotides and compositions of the present disclosure can provide high levels of adenosine editing. In some embodiments, oligonucleotides and compositions of the present disclosure are useful for treating various conditions, disorders or diseases, e.g., alpha-1 antitrypsin deficiency. In some embodiments, the present disclosure provides methods for treating various conditions, disorders or diseases that can benefit from adenosine editing.

### CROSS-REFERENCE TO RELATED APPLICATIONS

This application is a National Stage Application of PCT International Application No. PCT/US2022/044765, filed on Sep. 26, 2022 and published as WO 2023/049475 on Mar. 30, 3023, which claims priority to United States Provisional Application Nos 63/248,520, filed Sep. 26, 2021, 63/331,756, filed Apr. 15, 2022, and 63/397,320, filed Aug. 11, 2022, and PCT Application No. PCT/US2021/058495, filed Nov. 8, 2021 and published as WO 2022/099159 May 12, 2022, the entirety of each of which is incorporated herein by reference.

### SEQUENCE LISTING

The instant application contains a Sequence Listing which has been submitted electronically in XML file format and is hereby incorporated by reference in its entirety. Said XML copy, created on Nov. 25, 2024, is named 2010581-1301.xml and is 1,068,543 bytes in size.

### BACKGROUND

Oligonucleotides are useful in various applications, e.g., therapeutic, diagnostic, and/or research applications. For example, oligonucleotides targeting various genes can be useful for treatment of conditions, disorders or diseases related to such target genes. The SERPINA1 gene encodes serine protease inhibitor alpha-1 antitrypsin (A1AT). It has been reported that A1AT protects tissues from certain inflammatory enzymes, including neutrophil elastase. A deficiency in A1AT (alpha 1 antitrypsin deficiency, A1AD) can lead to excessive break down of elastin in the lungs by neutrophil elastase. This may lead to reduced elasticity in the lungs and subsequent respiratory complications, including emphysema and chronic obstructive lung disease (COPD). Mutant A1AT can also build up in liver, resulting in cirrhosis and liver failure.

### SUMMARY

Among other things, the present disclosure recognizes a need for new treatments and therapies to correct pathogenic mutations in SERPINA1, e.g., 1024 G>A (E342K in A1AT), to treat alpha 1 antitrypsin deficiency (A1AD) which may result in hepatic failure and/or emphysema. In some embodiments, the present disclosure provides technologies, e.g., oligonucleotides, compounds, compositions, methods, etc., for preventing or treating conditions, disorders or diseases associated with 1024 G>A (E342K in A1AT) in SERPINA1.

Among other things, the present disclosure provides designed oligonucleotides and compositions thereof which oligonucleotides comprise modifications (e.g., modifications to nucleobases sugars, and/or internucleotidic linkages, and patterns thereof) as described herein. In some embodiments herein are compounds and methods for selectively and efficiently editing the SERPINA1 gene and correcting pathogenic mutations in the gene in order to treat alpha 1 antitrypsin deficiency (A1AD). Also provided are methods useful for preventing or ameliorating at least one symptom of a condition, disorder or disease associated with a SERPINA1 mutation. In some embodiments, technologies (compounds (e.g., oligonucleotides), compositions, methods, etc.) of the present disclosure (e.g., oligonucleotides, oligonucleotide compositions, methods, etc.) are particularly useful for editing nucleic acids, e.g., site-directed editing in nucleic acids (e.g., editing of target adenosine). In some embodiments, as demonstrated herein, provided technologies can significantly improve efficiency of nucleic acid editing, e.g., modification of one or more A residues, such as conversion of A to I. In some embodiments, the present disclosure provides technologies for editing (e.g., for modifying an A residue, e.g., converting an A to I) in an RNA. In some embodiments, the present disclosure provides technologies for editing (e.g., for modifying an A residue, e.g., converting an A to an I) in a transcript, e.g., mRNA. Among other things, provided technologies provide the benefits of utilization of endogenous proteins such as ADAR (Adenosine Deaminases Acting on RNA) proteins (e.g., ADAR1 and/or ADAR2), for editing nucleic acids, e.g., for modifying an A (e.g., as a result of G to A mutation). Those skilled in the art will appreciates that such utilization of endogenous proteins can avoid a number of challenges and/or provide various benefits compared to those technologies that require the delivery of exogenous components (e.g., proteins (e.g., those engineered to bind to oligonucleotides (and/or duplexes thereof with target nucleic acids) to provide desired activities), nucleic acids encoding proteins, viruses, etc.). In some embodiments, the present invention provides oligonucleotides, compounds, compositions and methods for editing a SERPINA1 transcript and/or for treating or preventing a condition, disorder or disease associated with a SERPINA1 mutation, e.g., 1024 G>A, in a subject. In some embodiments, an oligonucleotide, compound or composition is capable of effecting an adenosine deaminase acting on RNA (ADAR)-mediated adenosine to inosine alteration in a transcript. In some embodiments, the deamination correcting the pathogenic mutation 1024 G>A in SERPINA1, reversing a E342K mutation in an A1AT polypeptide back to wild-type, and/or reversing or slowing 1024 G>A-associated condition, disorder or disease and related symptoms experienced by the patient.

Particularly, in some embodiments, oligonucleotides of provided technologies comprise useful sugar modifications and/or patterns thereof (e.g., presence and/or absence of certain modifications), nucleobase modifications and/or patterns thereof (e.g., presence and/or absence of certain modifications), internucleotidic linkages modifications and/or stereochemistry and/or patterns thereof [e.g., types, modifications, and/or configuration (Rp or Sp) of chiral linkage phosphorus, etc.], etc., which, when combined with one or more other structural elements described herein (e.g., additional chemical moieties) can provide high activities and/or various desired properties, e.g., high efficiency of nucleic acid editing, high selectivity, high stability, high cellular uptake, low immune stimulation, low toxicity, improved distribution, improved affinity, etc. In some embodiments, provided oligonucleotides provide high stability, e.g., when compared to oligonucleotides having a high percentage of natural RNA sugars and/or 2′-F modified sugars utilized for adenosine editing. In some embodiments, provided oligonucleotides provide high activities, e.g., adenosine editing activity. In some embodiments, provided oligonucleotides provide high selectivity, for example, in some embodiments, provided oligonucleotides provide selective modification of a target adenosine in a target nucleic acid over other adenosine in the same target nucleic acid (e.g., 2, 3, 4, 5, 6, 7, 8, 9, 10, 11, 12, 13, 14, 15, 16, 17, 18, 19, 20 fold or more modification at the target adenosine than another adenosine, or all other adenosine, in a target nucleic acid).

In some embodiments, stereochemistry of one or more chiral linkage phosphorus of provided oligonucleotides are controlled in a composition. In some embodiments, the present disclosure provides a composition comprising a plurality of oligonucleotides, wherein oligonucleotides of a plurality share a common base sequence, and the same configuration of linkage phosphorus (e.g., all are Rp or all are Sp for the chiral linkage phosphorus) independently at one or more (e.g., about 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more, or at least 50%, 60%, 70%, 75%, 80%, 85%, 90%, 95%, or 99% of all chiral internucleotidic linkages) chiral internucleotidic linkages (“chirally controlled internucleotidic linkages”). In some embodiments, they share the same stereochemistry at each chiral linkage phosphorus. In some embodiments, oligonucleotides of a plurality share the same constitution. In some embodiments, oligonucleotides of a plurality are structurally identical except the internucleotidic linkages. In some embodiments, oligonucleotides of a plurality are structurally identical. In some embodiments, at least about 50%, 60%, 65%, 70%, 75%, 80%, 85%, 90%, or 95% of all oligonucleotides in a composition, or of all oligonucleotides sharing the common base sequence, share the pattern of backbone chiral centers of oligonucleotides of the plurality. In some embodiments, at least about 50%, 60%, 65%, 70%, 75%, 80%, 85%, 90%, or 95% of all oligonucleotides in a composition, or of all oligonucleotides sharing the common base sequence, are oligonucleotides of the plurality. In some embodiments, at least about 50%, 60%, 65%, 70%, 75%, 80%, 85%, 90%, or 95% of all oligonucleotides in a composition, or of all oligonucleotides sharing the common base sequence, are oligonucleotides of the plurality.

In some embodiments, the present disclosure provides a chirally controlled oligonucleotide composition of an oligonucleotide, wherein at least about 50%, 60%, 65%, 70%, 75%, 80%, 85%, 90%, or 95% of all oligonucleotides of the same constitution as the oligonucleotide, are one or more forms of the oligonucleotide (e.g., acid forms, salt forms (e.g. pharmaceutically acceptable salt forms; as appreciated by those skilled in the art, in case the oligonucleotide is a salt, other salt forms of the corresponding acid or base form of the oligonucleotide), etc.).

In some embodiments, the present disclosure provides technologies for preparing oligonucleotides and compositions thereof, particularly chirally controlled oligonucleotide compositions. In some embodiments, provided oligonucleotides, compounds and compositions thereof are of high purity. In some embodiments, oligonucleotides of the present disclosure are at least 50%, 60%, 70%, 75%, 80%, 85%, 90%, 95%, or 99% stereochemically pure at linkage phosphorus of chiral internucleotidic linkages. In some embodiments, oligonucleotides of the present disclosure are prepared stereoselectively and are substantially free of stereoisomers. In some embodiments, in provided compositions comprising a plurality of oligonucleotides which share the same base sequence of the same pattern of chiral linkage phosphorus stereochemistry (e.g., comprising one or more of Rp and/or Sp, wherein each chiral linkage phosphorus is independently Rp or Sp), at least 50%, 60%, 70%, 75%, 80%, 85%, 90%, 95%, or 99% of all oligonucleotides in the composition that share the same base sequence as oligonucleotides of the plurality share the same pattern of chiral linkage phosphorus stereochemistry or are oligonucleotides of the plurality. In some embodiments, in provided compositions comprising a plurality of oligonucleotides which share the same base sequence of the same pattern of chiral linkage phosphorus stereochemistry, at least 50%, 60%, 70%, 75%, 80%, 85%, 90%, 95%, or 99% of all oligonucleotides in the composition that share the same constitution as oligonucleotides of the plurality share the same pattern of chiral linkage phosphorus stereochemistry or are oligonucleotides of the plurality. In some embodiments, diastereomeric excess of each chiral phosphorus is independently about or at least about 90%. In some embodiments, diastereomeric excess of each chiral phosphorus is independently about or at least about 95%. In some embodiments, diastereomeric excess of each chiral phosphorus is independently about or at least about 97%. In some embodiments, diastereomeric excess of each chiral phosphorus is independently about or at least about 98%. In some embodiments, diastereomeric purity is about or at least about (DS)^{nc}, wherein DS is about 90-100%, and nc is the number of chiral linkage phosphorus. In some embodiments, DS is about 90% or more. In some embodiments, DS is about 95% or more. In some embodiments, DS is about 96% or more. In some embodiments, DS is about 97% or more. In some embodiments, DS is about 98% or more. In some embodiments, DS is about 99% or more.

In some embodiments, an oligonucleotide is WV-46312, WV-47606, WV-47608, WV-49085, WV-49086, WV-49087, WV-49088, WV-49089, WV-49090 or WV-49092. In some embodiments, an oligonucleotide is WV-46312. In some embodiments, an oligonucleotide is WV-47606. In some embodiments, an oligonucleotide is WV-47608. In some embodiments, an oligonucleotide is WV-49085. In some embodiments, an oligonucleotide is WV-49086. In some embodiments, an oligonucleotide is WV-49087. In some embodiments, an oligonucleotide is WV-49088. In some embodiments, an oligonucleotide is WV-49089. In some embodiments, an oligonucleotide is WV-49090. In some embodiments, an oligonucleotide is WV-49092.

In some embodiments, an oligomeric compound comprising an oligonucleotide or a pharmaceutically acceptable salt thereof, wherein the oligonucleotide is of formula:

(SEQ ID NO: 1)
Mod001L001mCn001RmC*SmC*SfA*SfG*SmCmA*SfG*SfCmU*SfUn001RmCfA*SfGn001RfUmCSfC*
SfC*SfU*SmUmUfC*ST*Sb008U*SIn001SmUfC*SmG*SmAn001RmU;
(SEQ ID NO: 15)
Mod001L001mCn001RmC*SmC*SfA*SfG*Sm5CeoAcofG*SfC*STeofUn001RmCfA*SfGn001RfUm5Ceo*
SfC*SfC*SfUn001RTeoTeofC*ST*Sb008U*SIn001SmUfC*SmG*SmAn001RmU
(SEQ ID NO: 13)
Mod001L001mCn001RmC*SmC*SfA*SfG*Sm5CeoAeofG*SfC*SmUmUn001RmCfA*SfGn001RfUm5Ceo*
SfC*SmCmUn001RmUTeofC*ST*Sb008U*SIn001SmUfC*SmG*SmAn001RmU;
(SEQ ID NO: 12)
Mod001L001mCh001RmC*SmC*SfA*SfG*Sm5CeoAcofG*Sm5CeoTeo*SmUn001Rm5CcofA*
SfGn001RmUm5Ceom5Ceo*SfC*SfU*STeoTeofC*ST*Sb008U*SIn001SmUfC*SmG*SmAn001RmU;
(SEQ ID NO: 10)
Mod001L001mCn001RmC*SmC*SfA*SfG*Sm5CeoAeofG*SmCTeo*SmUn001RmCfA*SfGn001RmUm5CcomC*
SfC*SfU*STeoTeofC*ST*Sb008U*SIn001SmUfC*SmG*SmAn001RmU;
(SEQ ID NO: 9)
Mod001L001mCn001RmC*SmC*SfA*SfG*Sm5CeoAeofG*SmCTeo*SmUn001RmCfA*SfGn001RmUmCmC*
SfC*SfU*STeoTeofC*ST*Sb008U*SIn001SmUfC*SmG*SmAn001RmU;
(SEQ ID NO: 8)
Mod001L001mCn001RmC*SmC*SfA*SfG*Sm5CeoAcofG*SfC*STeofUn001RmCfA*SmGn001RfUm5Cco*
SfC*SfC*SfUn001RTcoTeofC*ST*Sb008U*SIn001SmUfC*SmG*SmAn001RmU;
(SEQ ID NO: 7)
Mod001L001mCn001RmC*SmC*SfA*SfG*Sm5CeoAcofG*SfC*STeofUn001RmCfA*SmGn001RfUmC*SfC*
SfC*SfUn001RTeoTeofC*ST*Sb008U*SIn001SmUfC*SmG*SmAn001RmU;
(SEQ ID NO: 5)
Mod001L001mCn001RmC*SmC*SfA*SfG*SmCmAfG*SfC*SmUfUn001RmCfA*SmGn001RfUmC*SfC*SfC*
SfUn001RmUmUfC*ST*Sb008U*SIn001SmUfC*SmG*SmAn001RmU
or
(SEQ ID NO: 3)
Mod001L001mCn001RmC*SmC*SfA*SfG*Sm5CeoAeofG*SfC*STeofUn001RmCfA*SfGn001RfUmC*SfC*
SfC*SfUn001RTeoTeofC*ST*Sb008U*SIn001SmUfC*SmG*SmAn001RmU.

In some embodiments, an oligomeric compound comprising an oligonucleotide or a pharmaceutically acceptable salt thereof, wherein the oligonucleotide is of formula:

(SEQ ID NO: 2)
mCn001RmC*SmC*SfA*SfG*SmCmA*SfG*SfCmU*SfUn001RmCfA*SfGn001RfUmC*SfC*SfC*
SfU*SmUmUfC*ST*Sb008U*SIn001SmUfC*SmG*SmAn001RmU;
(SEQ ID NO: 16)
mCn001RmC*SmC*SfA*SfG*Sm5CeoAcofG*SfC*STeofUn001RmCfA*SfGn001RfUm5Ceo*SfC*
SfC*SfUn001RTeoTeofC*ST*Sb008U*SIn001SmUfC*SmG*SmAn001RmU;
(SEQ ID NO: 14)
mCn001RmC*SmC*SfA*SfG*Sm5CeoAcofG*SfC*SmUmUn001RmCfA*SfGn001RfUm5Ceo*SfC*
SmCmUn001RmUTeofC*ST*Sb008U*SIn001SmUfC*SmG*SmAn001RmU;
(SEQ ID NO: 32)
mCn001RmC*SmC*SfA*SfG*Sm5CeoAeofG*Sm5CeoTeo*SmUn001Rm5CeofA*SfGn001RmUm5Ceom5Ceo*
SfC*SfU*STeoTeofC*ST*Sb008U*SIn001Sm UfC*SmG*SmAn001RmU;
(SEQ ID NO: 11)
mCn001RmC*SmC*SfA*SfG*Sm5CeoAeofG*SmCTeo*SmUn001RmCfA*SfGn001RmUm5CeomC*
SfC*SfU*STeoTeofC*ST*Sb008U*SIn001SmUfC*SmG*SmAn001RmU;
(SEQ ID NO: 31)
mCn001RmC*SmC*SfA*SfG*Sm5CeoAeofG*SmCTeo*SmUn001RmCfA*SfGn001RmUmCmC*SfC*
SfU*STeoTeofC*ST*Sb008U*SIn001SmUfC*SmG*SmAn001RmU;
(SEQ ID NO: 30)
mCn001RmC*SmC*SfA*SfG*Sm5CeoAcofG*SfC*STeofUn001RmCfA*SmGn001RfUm5Ceo*SfC*
SfC*SfUn001RTeoTeofC*ST*Sb008U*SIn001SmUfC*SmG*SmAn001RmU;
(SEQ ID NO: 29)
mCn001RmC*SmC*SfA*SfG*Sm5CeoAeofG*SfC*STeofUn001RmCfA*SmGn001RfUmC*SfC*SfC*
SfUn001RTeoTeofC*ST*Sb008U*SIn001SmUfC*SmG*SmAn001RmU;
(SEQ ID NO: 6)
mCn001RmC*SmC*SfA*SfG*SmCmAfG*SfC*SmUfUn001RmCfA*SmGn001RfUmC*SfC*SfC*
SfUn001RmUmUfC*ST*Sb008U*SIn001SmUfC*SmG*SmAn001RmU
or
(SEQ ID NO: 4)
mCn001RmC*SmC*SfA*SfG*Sm5CeoAeofG*SfC*STeofUn001RmCfA*SfGn001RfUmC*SfC*SfC*
SfUn001RTeoTeofC*ST*Sb008U*SIn001SmUfC*SmG*SmAn001RmU.

As described herein, oligonucleotides and compositions of the present disclosure may be provided/utilized in various forms. In some embodiments, the present disclosure provides compositions comprising one or more forms of oligonucleotides, e.g., acid forms (e.g., in which natural phosphate linkages exist as —O(P(O)(OH)—O—, phosphorothioate internucleotidic linkages exist as —O(P(O)(SH)—O—), base forms, salt forms (e.g., in which natural phosphate linkages exist as salt forms (e.g., sodium salt (—O(P(O)(O⁻Na⁺)—O—), phosphorothioate internucleotidic linkages exist as salt forms (e.g., sodium salt (—O(P(O)(S⁻Na⁺)—O—) etc. As appreciated by those skilled in the art, oligonucleotides can exist in various salt forms, including pharmaceutically acceptable salts, and in solutions (e.g., various aqueous buffering system), cations may dissociate from anions. In some embodiments, the present disclosure provides a pharmaceutical composition comprising a provided oligonucleotide and/or one or more pharmaceutically acceptable salts thereof, and a pharmaceutically acceptable carrier. In some embodiments, pharmaceutical compositions are chirally controlled oligonucleotide compositions.

As appreciated by those skilled in the art, an oligonucleotide may be provided, administered or delivered as various forms, including various salts forms such as pharmaceutically acceptable salt forms. In some embodiments, an oligonucleotide is provided, administered or delivered in a salt form. In some embodiments, an oligonucleotide is provided, administered or delivered in a pharmaceutically acceptable salt forms. In some embodiments, an oligonucleotide is provided, administered or delivered in multiple forms. In some embodiments, an oligonucleotide is provided, administered or delivered in multiple salt forms. In some embodiments, an oligonucleotide is provided, administered or delivered in multiple pharmaceutically acceptable salt forms. In some embodiments, together the multiple forms amount to an effective amount of an oligonucleotide.

In some embodiments, provided oligonucleotides comprise an additional moiety, e.g., a targeting moiety, a carbohydrate moiety, etc. In some embodiments, an additional moiety is or comprises a ligand for an asialoglycoprotein receptor. In some embodiments, an additional moiety is or comprises GalNAc or derivatives thereof. In some embodiments, an additional moiety is or comprises GalNAc. Among other things, additional moieties may facilitate delivery to certain target locations, e.g., cells, tissues, organs, etc. (e.g., locations comprising receptors that interact with additional moieties). In some embodiments, additional moieties facilitate delivery to liver. In some embodiments, to deliver an oligonucleotide, a conjugate oligonucleotide comprising such an oligonucleotide with an additional chemical moiety is administered. In some embodiments, an oligonucleotide is delivered through administering a conjugate of such an oligonucleotide with one or more additional chemical moieties optionally through one or more linkers.

In some embodiments, an oligonucleotide is mCn001RmC*SmC*SfA*SfG*SmCmA*SfG*SfCmU*SfUn001RmCfA*SfGn001RfUmC*SfC*SfC*SfU*SmUmUfC*ST*Sb008U*SIn001SmUfC*SmG*SmAn001RmU (SEQ ID NO: 2) or a salt thereof. In some embodiments, the present disclosure provides an oligonucleotide which is a conjugate of such an oligonucleotide with one or more additional chemical moieties optionally through one or more linkers. In some embodiments, an oligonucleotide is Mod001L001mCn001RmC*SmC*SfA*SfG*SmCmA*SfG*SfCmU*SfUn001RmCfA*SfGn001RfUmC*SfC*SfC*SfU*SmUmUfC*ST*Sb008U*SIn001SmUfC*SmG*SmAn001RmU (SEQ ID NO: 1) or a salt thereof.

In some embodiments, an oligonucleotide is mCn001RmC*SmC*SfA*SfG*Sm5CeoAeofG*SfC*STeofUn001RmCfA*SfGn001RfUmC*SfC*SfC*SfUn001RTeoTeofC*ST*Sb008U*SIn001SmUfC*SmG*SmAn001RmU (SEQ ID NO: 4) or a salt thereof. In some embodiments, the present disclosure provides an oligonucleotide which is a conjugate of such an oligonucleotide with one or more additional chemical moieties optionally through one or more linkers. In some embodiments, an oligonucleotide is Mod001L001mCn001RmC*SmC*SfA*SfG*Sm5CeoAeofG*SfC*STeofUn001RmCfA*SfGn001RfUmC*SfC*SfC*SfUn001RTeoTeofC*ST*Sb008U*SIn001SmUfC*SmG*SmAn001RmU (SEQ ID NO: 3) or a salt thereof.

In some embodiments, an oligonucleotide is mCn001RmC*SmC*SfA*SfG*SmCmAfG*SfC*SmUfUn001RmCfA*SmGn001RfUmC*SfC*SfC*SfUn001RmUmUfC*ST*Sb008U*SIn001SmUfC*SmG*SmAn001RmU (SEQ ID NO: 6) or a salt thereof.

In some embodiments, the present disclosure provides an oligonucleotide which is a conjugate of such an oligonucleotide with one or more additional chemical moieties optionally through one or more linkers. In some embodiments, an oligonucleotide is Mod001L001mCn001RmC*SmC*SfA*SfG*SmCmAfG*SfC*SmUfUn001RmCfA*SmGn001RfUmC*SfC*SfC*SfUn001RmUmUfC*ST*Sb008U*SIn001SmUfC*SmG*SmAn001RmU (SEQ ID NO: 5) or a salt thereof.

In some embodiments, an oligonucleotide is mCn001RmC*SmC*SfA*SfG*Sm5CeoAeofG*SfC*STeofUn001RmCfA*SmGn001RfUmC*SfC*SfC*SfUn001RTeoTeofC*ST*Sb008U*SIn001SmUfC*SmG*SmAn001RmU (SEQ ID NO: 29) or a salt thereof. In some embodiments, the present disclosure provides an oligonucleotide which is a conjugate of such an oligonucleotide with one or more additional chemical moieties optionally through one or more linkers. In some embodiments, an oligonucleotide is Mod001L001mCn001RmC*SmC*SfA*SfG*Sm5CeoAeofG*SfC*STeofUn001RmCfA*SmGn001RfUmC*SfC*SfC*SfUn001RTeoTeofC*ST*Sb008U*SIn001SmUfC*SmG*SmAn001RmU (SEQ ID NO: 7) or a salt thereof.

In some embodiments, an oligonucleotide is mCn001RmC*SmC*SfA*SfG*Sm5CeoAeofG*SfC*STeofUn001RmCfA*SmGn001RfUm5Ceo*SfC*SfC*SfUn001RTeoTeofC*ST*Sb008U*SIn001SmUfC*SmG*SmAn001RmU (SEQ ID NO: 30) or a salt thereof. In some embodiments, the present disclosure provides an oligonucleotide which is a conjugate of such an oligonucleotide with one or more additional chemical moieties optionally through one or more linkers. In some embodiments, an oligonucleotide is Mod001L001mCn001RmC*SmC*SfA*SfG*Sm5CeoAeofG*SfC*STeofUn001RmCfA*SmGn001RfUm5Ceo*SfC*SfC*SfUn001RTeoTeofC*ST*Sb008U*SIn001SmUfC*SmG*SmAn001RmU (SEQ ID NO: 8) or a salt thereof.

In some embodiments, an oligonucleotide is mCn001RmC*SmC*SfA*SfG*Sm5CeoAeofG*SmCTeo*SmUn001 RmCfA*SfGn001RmUmCmC*SfC*SfU*STeoTeofC*ST*Sb008U*SIn000SmUfC*SmG*SmAn001RmU (SEQ ID NO: 31) or a salt thereof. In some embodiments, the present disclosure provides an oligonucleotide which is a conjugate of such an oligonucleotide with one or more additional chemical moieties optionally through one or more linkers. In some embodiments, an oligonucleotide is Mod001L001 mCn001RmC*SmC*SfA*SfG*Sm5CeoAeofG*SmCTeo*SmUn001RmCfA*SfGn001RmUmCmC*SfC*SfU*STeoTeofC*ST*Sb008U*SIn001SmUfC*SmG*SmAn001RmU (SEQ ID NO: 9) or a salt thereof.

In some embodiments, an oligonucleotide is mCn001RmC*SmC*SfA*SfG*Sm5CeoAeofG*SmCTeo*SmUn001RmCfA*SfGn001RmUm5CeomC*SfC*SfU*STeoTeofaC*ST*Sb008U*SIn001SmUf*SmG*SmAn001RmU (SEQ ID NO: 11) or a salt thereof. In some embodiments, the present disclosure provides an oligonucleotide which is a conjugate of such an oligonucleotide with one or more additional chemical moieties optionally through one or more linkers. In some embodiments, an oligonucleotide is Mod001L001mCn001RmC*SmC*SfA*SfG*Sm5CeoAeofG*SmCTeo*SmUn001RmCfA*SfGn001RmUm5CeomC*SfC*SfU*STeoTeofC*ST*Sb008U*SIn001SmUfC*SmG*SmAn001RmU (SEQ ID NO: 10) or a salt thereof.

In some embodiments, an oligonucleotide is mCn001RmC*SmC*SfA*SfG*Sm5CeoAeofG*Sm5CeoTeo*SmUn001Rm5CeofA*SfGn001RmUm5Ceom5Ceo*SfC*SfU*STeoTeofC*ST*Sb008U*SIn001SmUfC*SmG*SmAn001RmU (SEQ ID NO: 32) or a salt thereof. In some embodiments, the present disclosure provides an oligonucleotide which is a conjugate of such an oligonucleotide with one or more additional chemical moieties optionally through one or more linkers. In some embodiments, an oligonucleotide is Mod001L001mCn001RmC*SmC*SfA*SfG*Sm5CeoAeofG*Sm5CeoTeo*SmUn001Rm5CeofA*SfGn001RmUm5Ceom5Ceo*SfC*SfU*STeoTeofC*ST*Sb008U*SIn001SmUfC*SmG*SmAn001RmU (SEQ ID NO: 12) or a salt thereof.

In some embodiments, an oligonucleotide is mCn001RmC*SmC*SfA*SfG*Sm5CeoAeofG*SfC*SmUmUn001RmCfA*SfGn001RfUm5Ceo*SfC*SmCmUn001RmUTeofC*ST*Sb008U*SIn001SmUfC*SmG*SmAn001RmU (SEQ ID NO: 14) or a salt thereof. In some embodiments, the present disclosure provides an oligonucleotide which is a conjugate of such an oligonucleotide with one or more additional chemical moieties optionally through one or more linkers. In some embodiments, an oligonucleotide is Mod001L001mCn001RmC*SmC*SfA*SfG*Sm5CeoAeofG*Sf C*SmUmUn001RmCfA*SfGn001RfUm5Ceo*SfC*SmCmUn001RmUTeofC*ST*Sb008U*SIn001SmUfC*SmG*SmAn001RmU (SEQ ID NO: 13) or a salt thereof.

In some embodiments, an oligonucleotide is mCn001RmC*SmC*SfA*SfG*Sm5CeoAeofG*SfC*STeofUn001RmCfA*SfGn001RfUm5Ceo*SfC*Sf C*SfJn001RTeoTeofC*ST*Sb008U*SIn00SmUfC*SmG*SmAn001RmU (SEQ ID NO: 16) or a salt thereof. In some embodiments, the present disclosure provides an oligonucleotide which is a conjugate of such an oligonucleotide with one or more additional chemical moieties optionally through one or more linkers. In some embodiments, an oligonucleotide is Mod001L001mCn001 RmC*SmC*SfA*SfG*Sm5CeoAeofG*SfC*STeofUn001RmCfA*SfGn001 RfUm5 Ceo*SfC*SfC*SfUn001RTeoTeofC*ST*Sb008U*SIn001SmUfC*SmG*SmAn001RmU (SEQ ID NO: 15) or a salt thereof.

Provided technologies can be utilized for various purposes. For example, those skilled in the art will appreciate that provided technologies are useful for many purposes involving modification of adenosine, e.g., correction of G to A mutations, modulate levels of certain nucleic acids and/or products encoded thereby, etc.

In some embodiments, the present disclosure provides technologies for preventing or treating a condition, disorder or disease that is amenable to an adenosine modification, e.g. conversion of A to I or G. As appreciated by those skilled in the art, I may perform one or more functions of G, e.g., in base pairing, translation, etc. In some embodiments, a G to A mutation may be corrected through conversion of A to I so that one or more products, e.g., proteins, of the G-version nucleic acid can be produced. In some embodiments, the present disclosure provides technologies for preventing or treating a condition, disorder or disease associated with a mutation, comprising administering to a subject susceptible thereto or suffering therefrom a provided oligonucleotide or composition thereof, which oligonucleotide or composition can edit a mutation. In some embodiments, the present disclosure provides technologies for preventing or treating a condition, disorder or disease associated with a G to A mutation, comprising administering to a subject susceptible thereto or suffering therefrom a provided oligonucleotide or composition thereof, which oligonucleotide or composition can modify an A. In some embodiments, provided technologies modify an A in a transcript, e.g., RNA transcript. In some embodiments, an A is converted into an I. In some embodiments, during translation protein synthesis machineries read I as G. In some embodiments, an A form encodes one or more proteins that have one or more higher desired activities and/or one or more better desired properties compared those encoded by its corresponding G form. In some embodiments, an A form provides higher levels, compared to its corresponding G form, of one or more proteins that have one or more higher desired activities and/or one or more better desired properties. In some embodiments, products encoded by an A form are structurally different (e.g., longer, in some embodiments, full length proteins) from those encoded by its corresponding G form. In some embodiments, an A form provides structurally identical products (e.g., proteins) compared to its corresponding G form. In some embodiments, a mutation is 1024 G>A in SERPINA1. In some embodiments, a condition, disorder or disease is associated with 1024 G>A in SERPINA1.

This application incorporates herein by reference United States Provisional Application Nos. 63/111,079, filed Nov. 8, 2020, 63/175,036, filed Apr. 14, 2021, 63/188,415, filed May 13, 2021, 63/196,178, filed Jun. 2, 2021, 63/248,520, filed Sep. 26, 2021, 63/331,756, filed Apr. 15, 2022, and 63/397,320, filed Aug. 11, 2022, and WO 2021/071858 and WO 2022/099159.

### BRIEF DESCRIPTION OF THE DRAWINGS

FIG. 1. Provided technologies can provide durable editing in vivo. Mice transgenic for hADAR and SERPINA1-Z allele were treated with oligonucleotide compositions targeting SERPINA1-Z allele at 10 mg/kg doses on days 0, 2, and 4 via subcutaneous administration. Mouse serum was collected through weekly blood draws on indicated days post-treatment. (a) Levels of human AAT protein were measured by ELISA. Data are presented as mean±sem. Stats: Matched 2-way ANOVA: ns: non-significant, **: P<0.01, ***: P<0.001. (b) Mass spectrometry and ELISA were used to determine relative proportions of wild-type (W^{T}/M-AAT) and mutant (Z-AAT/Mutant) AAT protein.

FIG. 2. Provided technologies can provide editing. Primary mouse hepatocytes transgenic for hADARp110 and SERPINA1-Z allele were treated with oligonucleotide compositions comprising indicated GalNAc-conjugated oligonucleotides targeting SERPINA1-Z allele at indicated concentrations. RNA was isolated 48 hours post-treatment and RNA editing was measured by Sanger sequeneing (n=2 biological replicates).

FIG. 3. Provided technologies can provide editing in vivo. Mice transgenic for hADAR and SERPINA1-Z allele were treated with oligonucleotide compositions targeting SERPINA1-Z allele at 5 mg/kg doses on days 0, 2, and 4 via subcutaneous administration. Mouse liver biopsies were collected on day 7 post-treatment. RNA editing was measured by Sanger sequeneing in male (left bar) and female (right bar) mice (n=3 animals per gender).

FIG. 4. Provided technologies can provide editing. Primary mouse hepatocytes transgenic for hADARp110 and SERPINA1-Z allele were treated with oligonucleotide compositions targeting SERPINA-Z allele at indicated concentrations. RNA was isolated 48 hours post-treatment and RNA editing was measured by Sanger sequeneing (n=3 biological replicates).

FIG. 5. Provided technologies can provide functional edited polypeptides in vivo. Mice transgenic for hADAR and SERPINA1-Z allele were treated with oligonucleotide compositions targeting SERPINA1-Z allele at 10 mg/kg doses on days 0, 2, and 4 via subcutaneous administration. Mouse serum was collected through weekly blood draws on indicated days. Levels of human AAT protein was quantified by ELISA and mass spectrometry to assess relative proportions of wild-type (PiM/WT, left bar) and mutant (PiZ/Mutant, right bar) AAT protein.

FIG. 6. Provided technologies can provide editing. Compositions of oligonucleotides comprising various modifications, such as base modifications (e.g., b008U, etc.), linkage modifications (e.g., PS (phosphorothioate), PN (e.g., phosphoryl guanidine linkages such as n001), etc.), sugar modifications (e.g., 2′-F, 2′-OMe, 2′-MOE, etc.), etc., were prepared and assessed. Editing of target adenosines in SERPINA1-Z allele in primary mouse hepatocytes transgenic for humanADARp110 and SERPINA1-Z allele was confirmed (N=2 biological replicates).

FIG. 7. Provided technologies can provide editing. Compositions of oligonucleotides comprising various modifications, such as base modifications (e.g., b008U, etc.), linkage modifications (e.g., PS (phosphorothioate), PN (e.g., phosphoryl guanidine linkages such as n001), etc.), sugar modifications (e.g., 2′-F, 2′-OMe, 2′-MOE, etc.), etc., were prepared and assessed. Editing of target adenosines in SERPINA1-Z allele in primary mouse hepatocytes transgenic for humanADARp110 and SERPINA1-Z allele was confirmed (N=2 biological replicates).

FIG. 8. Provided technologies can provide editing in vivo. In vivo editing of target adenosines in SERPINA1-Z allele in mice transgenic for human ADAR and SERPINA1-Z allele was confirmed. Serum levels of AAT in treated mice were also increased.

FIG. 9. Provided technologies can provide editing in vivo. Oligonucleotides comprising various nucleobases (e.g., b008U, hypoxanthine, etc.), linkages (e.g., PO, PS, PN (e.g., phosphoryl guanidine linkages such as n001), etc.), sugar modifications (e.g., 2′-F, 2′-OMe, 2′-MOE, etc.), etc., and patterns thereof were prepared. Editing of target adenosines and increase of serum AAT was confirmed (N=4 animals per group). Top: SERPINA1 editing at day 10. Bottom: serum AAT fold change.

FIG. 10. Provided technologies can provide editing of target transcripts. Editing of SERPINA1-Z allele was confirmed (N=2 biological replicates). Human patient iPSC-derived hepatocytes with the ZZ genotype were plated on day 0 and treated on day 2 with the indicated oligonucleotides (e.g., WV-46312, WV-49090, WV-49092) at various concentrations (from left to right for each oligonucleotide, 5, 1.25, 0.31, and 0.08 uM). Media was refreshed every 2 days (e.g., on days 4, 6, 8). RNA was collected on day 10 and RNA editing was quantified by Sanger sequeneing. Error bars represent standard error of the mean (SEM).

FIG. 11. Provided technologies can provide editing of target transcripts. Editing of SERPINA1-Z allele was confirmed (N=2 biological replicates). Human patient iPSC-derived hepatocytes with the ZZ genotype were plated on day 0 and treated on day 2 with the indicated oligonucleotides (WV-46312 on left. WV-44515 on right) at various concentrations (e.g., 5, 1.25, 0.31, and 0.08 uM). Media was changed every 2 days (e.g., on days 4, 6, 8) and indicated oligonucleotides were redosed every 2 days (e.g., on days 4, 6, 8). RNA was collected on day 10 and RNA editing was quantified by Sanger sequeneing. Error bars represent standard error of the mean (SEM).

FIG. 12. Provided technologies can provide editing in vivo. Editing of transcripts from SERPINA1 PiZ allele was confirmed. Seven-week-old NSG-PiZ mice (JAX stock #028842: N=5 per treatment group) were dosed subcutaneously with indicated oligonucleotide compositions (e.g., WV-49090) at 10 mg/kg per dose. One group of mice received loading doses during week 1 (on days 0, 2, 4), while the other group received a single dose during week 1 (on day 0) (no loading dose). The groups subsequently received additional doses every 2 weeks (e.g., during week 2, 4, 6, 8, 10, 12). A control group of mice received PBS. Mouse liver biopsies were collected on week 13 following treatment. RNA was collected from the liver biopsies and RNA editing was quantified by Sanger sequeneing. Error bars represent standard error of the mean (SEM). One-way ANOVA with adjustment for multiple comparisons (Tukey) was used to test for differences in % editing between loading dose and no loading dose (ns: not significant).

FIG. 13. Provided technologies can increase SERPINA1 mRNA levels in vivo. Seven-week-old NSG-PiZ mice (JAX stock #028842; N=5 per treatment group) were dosed subcutaneously with indicated oligonucleotide compositions (e.g., WV-49090) at 10 mg/kg per dose. One group of mice received loading doses during week 1 (on days 0, 2, 4), while the other group received a single dose during week 1 (on day 0) (no loading dose). The groups subsequently received additional doses every 2 weeks (e.g., during week 2, 4, 6, 8, 10, 12). Mouse liver biopsies were collected on week 13 following treatment. A control group of mice received PBS. RNA was collected from the liver biopsies and relative SERPINA1 mRNA levels (SERPINA1/HPRT) were quantified using qPCR. Baseline measure of relative SERPINA1 mRNA levels were determined from mice liver biopsies collected pre-dosing (week 0). Error bars represent standard error of the mean (SEM). One-way ANOVA with adjustments for repeated measures and multiple comparisons (Dunnett) was used to test for differences in relative SERPINA1 mRNA levels (****: P-value<0.001; ns: not significant).

FIG. 14. Provided technologies can decrease mutant Z-AAT protein levels and increase wild-type (M) AAT protein levels in serum. Seven-week-old NSG-PiZ mice (JAX stock #028842; N=5 per treatment group) were dosed subcutaneously with indicated oligonucleotide compositions (e.g., WV-49090) at 10 mg/kg per dose. One group of mice received loading doses during week 1 (on days 0, 2, 4), while the other group received a single dose during week 1 (on day 0) (no loading dose). The groups subsequently received additional doses every 2 weeks (e.g., during week 2, 4, 6, 8, 10, 12). A control group of mice received PBS. Serum was collected from mice at week 13 following treatment. Relative abundance of Z (mutant) vs. M (wild-type) AAT isoforms was determined by liquid chromatography-mass spectrometry (LC-MS). Error bars represent standard error of the mean (SEM).

FIG. 15. Editing by various provided oligonucleotide compositions can result in functional wild-type AAT protein. Seven-week-old NSG-PiZ mice (JAX stock #028842; N=5 per treatment group) were dosed subcutaneously with indicated oligonucleotide compositions at 10 mg/kg per dose. One group of mice received loading doses during week 1 (on days 0, 2, 4), while the other group received a single dose during week 1 (on day 0). The groups subsequently received additional doses every 2 weeks (e.g., during week 2, 4, 6, 8, 10, 12). A control group of mice received PBS. Serum was collected from mice prior to dosing and at week 13 following treatment. Relative elastase inhibition activity in serum was determined in an in vitro reaction using a commercially available kit. Treatment groups were, from left to right for each time point: PBS control, WV-49090 (with loading doses on days 0, 2, 4), WV-49090 (without loading doses). Error bars represent standard error of the mean (SEM). Two-way ANOVA with adjustment for multiple comparisons (Bonferroni) was used to test for differences in elastase inhibition activity in serum for the treatment groups receiving indicated oligonucleotide compositions versus the PBS control. (****: P-value<0.001; ns: not significant).

### DETAILED DESCRIPTION OF CERTAIN EMBODIMENTS

Technologies of the present disclosure may be understood more readily by reference to the following detailed description of certain embodiments.

### Definitions

As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell. University Science Books. Sausalito: 1999, and “March's Advanced Organic Chemistry”, 5th Ed., Ed.: Smith, M. B, and March, J., John Wiley & Sons, New York: 2001.

As used herein in the present disclosure, unless otherwise clear from context, (i) the term “a” or “an” may be understood to mean “at least one”; (ii) the term “or” may be understood to mean “and/or”; (iii) the terms “comprising”, “comprise”, “including” (whether used with “not limited to” or not), and “include” (whether used with “not limited to” or not) may be understood to encompass itemized components or steps whether presented by themselves or together with one or more additional components or steps; (iv) the term “another” may be understood to mean at least an additional/second one or more; (v) the terms “about” and “approximately” may be understood to permit standard variation as would be understood by those of ordinary skill in the art; and (vi) where ranges are provided, endpoints are included.

Unless otherwise specified, description of oligonucleotides and elements thereof (e.g., base sequence, sugar modifications, internucleotidic linkages, linkage phosphorus stereochemistry, patterns thereof, etc.) is from 5′ to 3′. As those skilled in the art will appreciate, in some embodiments, oligonucleotides may be provided and/or utilized as salt forms, particularly pharmaceutically acceptable salt forms, e.g., sodium salts. As those skilled in the art will also appreciate, in some embodiments, individual oligonucleotides within a composition may be considered to be of the same constitution and/or structure even though, within such composition (e.g., a liquid composition), particular such oligonucleotides might be in different salt form(s) (and may be dissolved and the oligonucleotide chain may exist as an anion form when, e.g., in a liquid composition) at a particular moment in time. For example, those skilled in the art will appreciate that, at a given pH, individual internucleotidic linkages along an oligonucleotide chain may be in an acid (H) form, or in one of a plurality of possible salt forms (e.g., a sodium salt, or a salt of a different cation, depending on which ions might be present in the preparation or composition), and will understand that, so long as their acid forms (e.g., replacing all cations, if any, with H⁺) are of the same constitution and/or structure, such individual oligonucleotides may properly be considered to be of the same constitution and/or structure.

Aliphatic: As used herein, “aliphatic” means a straight-chain (i.e., unbranched) or branched, substituted or unsubstituted hydrocarbon chain that is completely saturated or that contains one or more units of unsaturation (but not aromatic), or a substituted or unsubstituted monocyclic, bicyclic, or polycyclic hydrocarbon ring that is completely saturated or that contains one or more units of unsaturation (but not aromatic), or combinations thereof. In some embodiments, aliphatic groups contain 1-50 aliphatic carbon atoms. In some embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1-6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

Alkenyl: As used herein, the term “alkenyl” refers to an aliphatic group, as defined herein, having one or more double bonds.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, cycloalkyl rings have from about 3-10 carbon atoms in their ring structure where such rings are monocyclic, bicyclic, or polycyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

Alkynyl: As used herein, the term “alkynyl” refers to an aliphatic group, as defined herein, having one or more triple bonds.

Analog: The term “analog” includes any chemical moiety which differs structurally from a reference chemical moiety or class of moieties, but which is capable of performing at least one function of such a reference chemical moiety or class of moieties. As non-limiting examples, a nucleotide analog differs structurally from a nucleotide but performs at least one function of a nucleotide; a nucleobase analog differs structurally from a nucleobase but performs at least one function of a nucleobase: etc.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate and/or a pig). In some embodiments, animals include, but are not limited to, mammals, birds, reptiles, amphibians, fish and/or worms. In some embodiments, an animal may be a transgenic animal, a genetically-engineered animal and/or a clone.

Aryl: The term “aryl”, as used herein, used alone or as part of a larger moiety as in “aralkyl,” “aralkoxy,” or “aryloxyalkyl,” refers to monocyclic, bicyclic or polycyclic ring systems having a total of five to thirty ring members, wherein at least one ring in the system is aromatic. In some embodiments, an aryl group is a monocyclic, bicyclic or polycyclic ring system having a total of five to fourteen ring members, wherein at least one ring in the system is aromatic, and wherein each ring in the system contains 3 to 7 ring members. In some embodiments, each monocyclic ring unit is aromatic. In some embodiments, an aryl group is a biaryl group. The term “aryl” may be used interchangeably with the term “aryl ring.” In certain embodiments of the present disclosure, “aryl” refers to an aromatic ring system which includes, but is not limited to, phenyl, biphenyl, naphthyl, binaphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

Characteristic portion: As used herein, the term “characteristic portion”, in the broadest sense, refers to a portion of a substance whose presence (or absence) correlates with presence (or absence) of a particular feature, attribute, or activity of the substance. In some embodiments, a characteristic portion of a substance is a portion that is found in the substance and in related substances that share the particular feature, attribute or activity, but not in those that do not share the particular feature, attribute or activity. In certain embodiments, a characteristic portion shares at least one functional characteristic with the intact substance. For example, in some embodiments, a “characteristic portion” of a protein or polypeptide is one that contains a continuous stretch of amino acids, or a collection of continuous stretches of amino acids, that together are characteristic of a protein or polypeptide. In some embodiments, each such continuous stretch generally contains at least 2, 5, 10, 15, 20, 50, or more amino acids. In general, a characteristic portion of a substance (e.g., of a protein, antibody, etc.) is one that, in addition to the sequence and/or structural identity specified above, shares at least one functional characteristic with the relevant intact substance. In some embodiments, a characteristic portion may be biologically active.

Chiral control: As used herein, “chiral control” refers to control of the stereochemical designation of the chiral linkage phosphorus in a chiral internucleotidic linkage within an oligonucleotide. As used herein, a chiral internucleotidic linkage is an internucleotidic linkage whose linkage phosphorus is chiral. In some embodiments, a control is achieved through a chiral element that is absent from the sugar and base moieties of an oligonucleotide, for example, in some embodiments, a control is achieved through use of one or more chiral auxiliaries during oligonucleotide preparation, which chiral auxiliaries often are part of chiral phosphoramidites used during oligonucleotide preparation. In contrast to chiral control, a person having ordinary skill in the art will appreciate that conventional oligonucleotide synthesis which does not use chiral auxiliaries cannot control stereochemistry at a chiral internucleotidic linkage if such conventional oligonucleotide synthesis is used to form the chiral internucleotidic linkage. In some embodiments, the stereochemical designation of each chiral linkage phosphorus in each chiral internucleotidic linkage within an oligonucleotide is controlled.

Chirally controlled oligonucleotide composition: The terms “chirally controlled oligonucleotide composition”, “chirally controlled nucleic acid composition”, and the like, as used herein, refers to a composition that comprises a plurality of oligonucleotides (or nucleic acids) which share a common base sequence, wherein the plurality of oligonucleotides (or nucleic acids) share the same linkage phosphorus stereochemistry at one or more chiral internucleotidic linkages (chirally controlled or stereodefined internucleotidic linkages, whose chiral linkage phosphorus is Rp or Sp in the composition (“stereodefined”), not a random Rp and Sp mixture as non-chirally controlled internucleotidic linkages). In some embodiments, a chirally controlled oligonucleotide composition comprises a plurality of oligonucleotides (or nucleic acids) that share: 1) a common base sequence, 2) a common pattern of backbone linkages, and 3) a common pattern of backbone phosphorus modifications, wherein the plurality of oligonucleotides (or nucleic acids) share the same linkage phosphorus stereochemistry at one or more chiral internucleotidic linkages (chirally controlled or stereodefined internucleotidic linkages, whose chiral linkage phosphorus is Rp or Sp in the composition (“stereodefined”), not a random Rp and Sp mixture as non-chirally controlled internucleotidic linkages). Level of the plurality of oligonucleotides (or nucleic acids) in a chirally controlled oligonucleotide composition is pre-determined/controlled or enriched (e.g., through chirally controlled oligonucleotide preparation to stereoselectively form one or more chiral internucleotidic linkages) compared to a random level in a non-chirally controlled oligonucleotide composition. In some embodiments, about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition are oligonucleotides of the plurality. In some embodiments, about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition that share the common base sequence, the common pattern of backbone linkages, and the common pattern of backbone phosphorus modifications are oligonucleotides of the plurality. In some embodiments, a level is about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a composition, or of all oligonucleotides in a composition that share a common base sequence (e.g., of a plurality of oligonucleotide or an oligonucleotide type), or of all oligonucleotides in a composition that share a common base sequence, a common pattern of backbone linkages, and a common pattern of backbone phosphorus modifications, or of all oligonucleotides in a composition that share a common base sequence, a common patter of base modifications, a common pattern of sugar modifications, a common pattern of internucleotidic linkage types, and/or a common pattern of internucleotidic linkage modifications. In some embodiments, the plurality of oligonucleotides share the same stereochemistry at about 1-50 (e.g., about 1-10, 1-20, 5-10, 5-20, 10-15, 10-20, 10-25, 10-30, or about 1, 2, 3, 4, 5, 6, 7, 8, 9, 10, 11, 12, 13, 14, 15, 16, 17, 18, 19, or 20, or at least 1, 2, 3, 4, 5, 6, 7, 8, 9, 10, 11, 12, 13, 14, 15, 16, 17, 18, 19, or 20) chiral internucleotidic linkages. In some embodiments, the plurality of oligonucleotides share the same stereochemistry at about 1%-100% (e.g., about 5%-100%, 10%-100%, 20%-100%, 30%-100%, 40%-100%, 50%-100%, 60%-100%, 70%-100%, 80-100%, 90-100%, 95-100%, 50%-90%, about 5%, 10%, 15%, 20%, 25%, 30%, 35%, 40%, 45%, 50%, 55%, 60%, 65%, 70%, 75%, 80%, 85%, 90%, 95%, or 100%, or at least 5%, 10%, 15%, 20%, 25%, 30%, 35%, 40%, 45%, 50%, 55%, 60%, 65%, 70%, 75%, 80%, 85%, 90%, 95%, or 99%) of chiral internucleotidic linkages. In some embodiments, oligonucleotides (or nucleic acids) of a plurality share the same pattern of sugar and/or nucleobase modifications, in any. In some embodiments, oligonucleotides (or nucleic acids) of a plurality are various forms of the same oligonucleotide (e.g., acid and/or various salts of the same oligonucleotide). In some embodiments, oligonucleotides (or nucleic acids) of a plurality are of the same constitution. In some embodiments, level of the oligonucleotides (or nucleic acids) of the plurality is about 1%-100%, (e.g., about 5%-100%, 10%-100%, 20%-100%, 30%-100%, 40%-100%, 50%-100%, 60%-100%, 70%-100%, 80-100/0, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides (or nucleic acids) in a composition that share the same constitution as the oligonucleotides (or nucleic acids) of the plurality. In some embodiments, each chiral internucleotidic linkage is a chiral controlled internucleotidic linkage, and the composition is a completely chirally controlled oligonucleotide composition. In some embodiments, oligonucleotides (or nucleic acids) of a plurality are structurally identical. In some embodiments, a chirally controlled internucleotidic linkage has a diastereopurity of at least 80%, 85%, 90%, 91%, 92%, 93%, 94%, 95%, 96%, 97%, 98%, 99% or 99.5%, typically at least 90%, 91%, 92%, 93%, 94%, 95%, 96%, 97%, 98%, 99% or 99.5%. In some embodiments, a chirally controlled internucleotidic linkage has a diastereopurity of at least 95%. In some embodiments, a chirally controlled internucleotidic linkage has a diastereopurity of at least 96%. In some embodiments, a chirally controlled internucleotidic linkage has a diastereopurity of at least 97%. In some embodiments, a chirally controlled internucleotidic linkage has a diastereopurity of at least 98%. In some embodiments, a chirally controlled internucleotidic linkage has a diastereopurity of at least 99%. In some embodiments, a percentage of a level is or is at least (DS)^{nc}, wherein DS is a diastereopurity as described in the present disclosure (e.g., 90%, 91%, 92%, 93%, 94%, 95%, 96%, 97%, 98%, 99% or 99.5% or more) and nc is the number of chiral linkage phosphorus as described in the present disclosure (e.g., 1-50, 1-40, 1-30, 1-25, 1-20, 5-50, 5-40, 5-30, 5-25, 5-20, 1, 2, 3, 4, 5, 6, 7, 8, 9, 10, 11, 12, 13, 14, 15, 16, 17, 18, 19, 20, 21, 22, 23, 24, 25 or more). In some embodiments, a percentage of a level is or is at least (DS)^{nc}, wherein DS is a diastereopurity as described in the present disclosure (e.g., 90%, 91%, 92%, 93%, 94%, 95%, 96%, 97%, 98%, 99% or 99.5% or more) and nc is the number of chirally controlled internucleotidic linkages as described in the present disclosure (e.g., 1-50, 1-40, 1-30, 1-25, 1-20, 5-50, 5-40, 5-30, 5-25, 5-20, 1, 2, 3, 4, 5, 6, 7, 8, 9, 10, 11, 12, 13, 14, 15, 16, 17, 18, 19, 20, 21, 22, 23, 24, 25 or more). In some embodiments, a percentage of a level is or is at least (DS)^{nc}, wherein DS is 95%-100%. For example, when DS is 99% and nc is 10, the percentage is or is at least 90% ((99%)¹⁰≈0.90=90%). In some embodiments, level of a plurality of oligonucleotides in a composition is represented as the product of diastereopurity of each chiral linkage phosphorus in the oligonucleotides. In some embodiments, level of a plurality of oligonucleotides in a composition is represented as the product of diastereopurity of each chirally controlled internucleotidic linkage in the oligonucleotides. In some embodiments, diastereopurity of an internucleotidic linkage connecting two nucleosides in an oligonucleotide (or nucleic acid) is represented by the diastereopurity of an internucleotidic linkage of a dimer connecting the same two nucleosides, wherein the dimer is prepared using comparable conditions, in some instances, identical synthetic cycle conditions (e.g., for the linkage between Nx and Ny in an oligonucleotide . . . . NxNy . . . , the dimer is NxNy). In some embodiments, not all chiral internucleotidic linkages are chiral controlled internucleotidic linkages, and the composition is a partially chirally controlled oligonucleotide composition. In some embodiments, a non-chirally controlled internucleotidic linkage has a diastereopurity of less than about 80%, 75%, 70%, 65%, 60%, 55%, or of about 50%, as typically observed in stereorandom oligonucleotide compositions (e.g., as appreciated by those skilled in the art, from traditional oligonucleotide synthesis, e.g., the phosphoramidite method). In some embodiments, oligonucleotides (or nucleic acids) of a plurality are of the same type. In some embodiments, a chirally controlled oligonucleotide composition comprises non-random or controlled levels of individual oligonucleotide or nucleic acids types. For instance, in some embodiments a chirally controlled oligonucleotide composition comprises one and no more than one oligonucleotide type. In some embodiments, a chirally controlled oligonucleotide composition comprises more than one oligonucleotide type. In some embodiments, a chirally controlled oligonucleotide composition comprises multiple oligonucleotide types. In some embodiments, a chirally controlled oligonucleotide composition is a composition of oligonucleotides of an oligonucleotide type, which composition comprises a non-random or controlled level of a plurality of oligonucleotides of the oligonucleotide type.

Comparable: The term “comparable” is used herein to describe two (or more) sets of conditions or circumstances that are sufficiently similar to one another to permit comparison of results obtained or phenomena observed. In some embodiments, comparable sets of conditions or circumstances are characterized by a plurality of substantially identical features and one or a small number of varied features. Those of ordinary skill in the art will appreciate that sets of conditions are comparable to one another when characterized by a sufficient number and type of substantially identical features to warrant a reasonable conclusion that differences in results obtained or phenomena observed under the different sets of conditions or circumstances are caused by or indicative of the variation in those features that are varied.

Cycloaliphatic: The term “cycloaliphatic,” “carbocycle,” “carbocyclyl,” “carbocyclic radical,” and “carbocyclic ring,” are used interchangeably, and as used herein, refer to saturated or partially unsaturated, but non-aromatic, cyclic aliphatic monocyclic, bicyclic, or polycyclic ring systems, as described herein, having, unless otherwise specified, from 3 to 30 ring members. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, a cycloaliphatic group has 3-6 carbons. In some embodiments, a cycloaliphatic group is saturated and is cycloalkyl. The term “cycloaliphatic” may also include aliphatic rings that are fused to one or more aromatic or nonaromatic rings, such as decahydronaphthyl or tetrahydronaphthyl. In some embodiments, a cycloaliphatic group is bicyclic. In some embodiments, a cycloaliphatic group is tricyclic. In some embodiments, a cycloaliphatic group is polycyclic. In some embodiments, “cycloaliphatic” refers to C₃-C₆ monocyclic hydrocarbon, or C₈-C₁₀ bicyclic or polycyclic hydrocarbon, that is completely saturated or that contains one or more units of unsaturation, but which is not aromatic, that has a single point of attachment to the rest of the molecule, or a C₉-C₁₆ polycyclic hydrocarbon that is completely saturated or that contains one or more units of unsaturation, but which is not aromatic, that has a single point of attachment to the rest of the molecule.

Heteroaliphatic: The term “heteroaliphatic”, as used herein, is given its ordinary meaning in the art and refers to aliphatic groups as described herein in which one or more carbon atoms are independently replaced with one or more heteroatoms (e.g., oxygen, nitrogen, sulfur, silicon, phosphorus, and the like). In some embodiments, one or more units selected from C, CH, CH₂, and CH₃ are independently replaced by one or more heteroatoms (including oxidized and/or substituted forms thereof). In some embodiments, a heteroaliphatic group is heteroalkyl. In some embodiments, a heteroaliphatic group is heteroalkenyl.

Heteroalkyl: The term “heteroalkyl”, as used herein, is given its ordinary meaning in the art and refers to alkyl groups as described herein in which one or more carbon atoms are independently replaced with one or more heteroatoms (e.g., oxygen, nitrogen, sulfur, silicon, phosphorus, and the like). Examples of heteroalkyl groups include, but are not limited to, alkoxy, poly(ethylene glycol)-, alkyl-substituted amino, tetrahydrofuranyl, piperidinyl, morpholinyl, etc.

Heteroaryl: The terms “heteroaryl” and “heteroar-”, as used herein, used alone or as part of a larger moiety, e.g., “heteroaralkyl,” or “heteroaralkoxy,” refer to monocyclic, bicyclic or polycyclic ring systems having a total of five to thirty ring members, wherein at least one ring in the system is aromatic and at least one aromatic ring atom is a heteroatom. In some embodiments, a heteroaryl group is a group having 5 to 10 ring atoms (i.e., monocyclic, bicyclic or polycyclic), in some embodiments 5, 6, 9, or 10 ring atoms. In some embodiments, each monocyclic ring unit is aromatic. In some embodiments, a heteroaryl group has 6, 10, or 14 a electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic or polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl group, wherein the alkyl and heteroaryl portions independently are optionally substituted.

Heteroatom: The term “heteroatom”, as used herein, means an atom that is not carbon or hydrogen. In some embodiments, a heteroatom is boron, oxygen, sulfur, nitrogen, phosphorus, or silicon (including oxidized forms of nitrogen, sulfur, phosphorus, or silicon, charged forms of nitrogen (e.g., quaternized forms, forms as in iminium groups, etc.), phosphorus, sulfur, oxygen: etc.). In some embodiments, a heteroatom is silicon, phosphorus, oxygen, sulfur or nitrogen. In some embodiments, a heteroatom is silicon, oxygen, sulfur or nitrogen. In some embodiments, a heteroatom is oxygen, sulfur or nitrogen.

Heterocycle: As used herein, the terms “heterocycle,” “heterocyclyl,” “heterocyclic radical,” and “heterocyclic ring”, as used herein, are used interchangeably and refer to a monocyclic, bicyclic or polycyclic ring moiety (e.g., 3-30 membered) that is saturated or partially unsaturated and has one or more heteroatom ring atoms. In some embodiments, a heterocyclyl group is a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes substituted nitrogen. As an example, in a saturated or partially unsaturated ring having 0-3 heteroatoms selected from oxygen, sulfur and nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle, “heterocyclyl,” “heterocyclyl ring,”-heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic or polycyclic. The term “heterocyclylalkyl” refers to an alkyl group substituted by a heterocyclyl, wherein the alkyl and heterocyclyl portions independently are optionally substituted.

Identity: As used herein, the term “identity” refers to the overall relatedness between polymeric molecules, e.g., between nucleic acid molecules (e.g., oligonucleotides, DNA, RNA, etc.) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Internucleotidic linkage: As used herein, the phrase “internucleotidic linkage” refers generally to a linkage linking nucleoside units of an oligonucleotide or a nucleic acid. In some embodiments, an internucleotidic linkage is a phosphodiester linkage, as extensively found in naturally occurring DNA and RNA molecules (natural phosphate linkage (—OP(═O)(OH)O—), which as appreciated by those skilled in the art may exist as a salt form). In some embodiments, an internucleotidic linkage is a modified internucleotidic linkage (not a natural phosphate linkage). In some embodiments, an internucleotidic linkage is a “modified internucleotidic linkage” wherein at least one oxygen atom or —OH of a phosphodiester linkage is replaced by a different organic or inorganic moiety. In some embodiments, such an organic or inorganic moiety is selected from ═S, ═Se, =NR′, —SR′, —SeR′, —N(R′)₂, B(R′)₃, —S—, —Se—, and —N(R′)—, wherein each R′ is independently as defined and described in the present disclosure. In some embodiments, an internucleotidic linkage is a phosphotriester linkage, phosphorothioate linkage (or phosphorothioate diester linkage, —OP(═O)(SH)O—, which as appreciated by those skilled in the art may exist as a salt form), or phosphorothioate triester linkage. In some embodiments, a modified internucleotidic linkage is a phosphorothioate linkage. In some embodiments, an internucleotidic linkage is one of, e.g., PNA (peptide nucleic acid) or PMO (phosphorodiamidate Morpholino oligomer) linkage. In some embodiments, a modified internucleotidic linkage is a non-negatively charged internucleotidic linkage. In some embodiments, a modified internucleotidic linkage is a neutral internucleotidic linkage (e.g., n001 in certain provided oligonucleotides). It is understood by a person of ordinary skill in the art that an internucleotidic linkage may exist as an anion or cation at a given pH due to the existence of acid or base moieties in the linkage. In some embodiments, a modified internucleotidic linkages is a modified internucleotidic linkages designated as s, s1, s2, s3, s4, s5, s6, s7, s8, s9, s10, s11, s12, s13, s14, s15, s16, s17 and s18 as described in WO 2017/210647.

In vitro: As used herein, the term “in vitro” refers to events that occur in an artificial environment, e.g., in a test tube or reaction vessel, in cell culture, etc., rather than within an organism (e.g., animal, plant and/or microbe).

In vivo: As used herein, the term “in vivo” refers to events that occur within an organism (e.g., animal, plant and/or microbe).

Linkage phosphorus: as defined herein, the phrase “linkage phosphorus” is used to indicate that the particular phosphorus atom being referred to is the phosphorus atom present in the internucleotidic linkage, which phosphorus atom corresponds to the phosphorus atom of a phosphodiester internucleotidic linkage as occurs in naturally occurring DNA and RNA. In some embodiments, a linkage phosphorus atom is in a modified internucleotidic linkage, wherein each oxygen atom of a phosphodiester linkage is optionally and independently replaced by an organic or inorganic moiety. In some embodiments, a linkage phosphorus atom is chiral (e.g., as in phosphorothioate internucleotidic linkages). In some embodiments, a linkage phosphorus atom is achiral (e.g., as in natural phosphate linkages).

Modified nucleobase: The terms “modified nucleobase”, “modified base” and the like refer to a chemical moiety which is chemically distinct from a nucleobase, but which is capable of performing at least one function of a nucleobase. In some embodiments, a modified nucleobase is a nucleobase which comprises a modification. In some embodiments, a modified nucleobase is capable of at least one function of a nucleobase, e.g., forming a moiety in a polymer capable of base-pairing to a nucleic acid comprising an at least complementary sequence of bases. In some embodiments, a modified nucleobase is substituted A, T, C, G, or U, or a substituted tautomer of A, T, C, G, or U. In some embodiments, a modified nucleobase in the context of oligonucleotides refer to a nucleobase that is not A, T, C, G or U.

Modified nucleoside: The term “modified nucleoside” refers to a moiety derived from or chemically similar to a natural nucleoside, but which comprises a chemical modification which differentiates it from a natural nucleoside. Non-limiting examples of modified nucleosides include those which comprise a modification at the base and/or the sugar. Non-limiting examples of modified nucleosides include those with a 2′ modification at a sugar. Non-limiting examples of modified nucleosides also include abasic nucleosides (which lack a nucleobase). In some embodiments, a modified nucleoside is capable of at least one function of a nucleoside, e.g., forming a moiety in a polymer capable of base-pairing to a nucleic acid comprising an at least complementary sequence of bases.

Modified nucleotide: The term “modified nucleotide” includes any chemical moiety which differs structurally from a natural nucleotide but is capable of performing at least one function of a natural nucleotide. In some embodiments, a modified nucleotide comprises a modification at a sugar, base and/or internucleotidic linkage. In some embodiments, a modified nucleotide comprises a modified sugar, modified nucleobase and/or modified internucleotidic linkage. In some embodiments, a modified nucleotide is capable of at least one function of a nucleotide, e.g., forming a subunit in a polymer capable of base-pairing to a nucleic acid comprising an at least complementary sequence of bases.

Modified sugar: The term “modified sugar” refers to a moiety that can replace a sugar. A modified sugar mimics the spatial arrangement, electronic properties, or some other physicochemical property of a sugar. In some embodiments, as described in the present disclosure, a modified sugar is substituted ribose or deoxyribose. In some embodiments, a modified sugar comprises a 2′-modification. Examples of useful 2′-modification are widely utilized in the art and described herein. In some embodiments, a 2′-modification is 2′-F. In some embodiments, a 2′-modification is 2′-OR, wherein R is optionally substituted C₁₋₁₀ aliphatic. In some embodiments, a 2′-modification is 2′-OMe. In some embodiments, a 2′-modification is 2′-MOE. In some embodiments, a modified sugar is a bicyclic sugar (e.g., a sugar used in LNA, BNA, etc.). In some embodiments, in the context of oligonucleotides, a modified sugar is a sugar that is not ribose or deoxyribose as typically found in natural RNA or DNA.

Nucleic acid: The term “nucleic acid”, as used herein, includes any nucleotides and polymers thereof. The term “polynucleotide”, as used herein, refers to a polymeric form of nucleotides of any length, either ribonucleotides (RNA) or deoxyribonucleotides (DNA) or a combination thereof. These terms refer to the primary structure of the molecules and, thus, include double- and single-stranded DNA, and double- and single-stranded RNA. These terms include, as equivalents, analogs of either RNA or DNA comprising modified nucleotides and/or modified polynucleotides, such as, though not limited to, methylated, protected and/or capped nucleotides or polynucleotides. The terms encompass poly- or oligo-ribonucleotides (RNA) and poly- or oligo-deoxyribonucleotides (DNA); RNA or DNA derived from N-glycosides or C-glycosides of nucleobases and/or modified nucleobases; nucleic acids derived from sugars and/or modified sugars; and nucleic acids derived from phosphate bridges and/or modified internucleotidic linkages. The term encompasses nucleic acids containing any combinations of nucleobases, modified nucleobases, sugars, modified sugars, phosphate bridges or modified internucleotidic linkages. Examples include, and are not limited to, nucleic acids containing ribose moieties, nucleic acids containing deoxy-ribose moieties, nucleic acids containing both ribose and deoxyribose moieties, nucleic acids containing ribose and modified ribose moieties. Unless otherwise specified, the prefix poly- refers to a nucleic acid containing 2 to about 10,000 nucleotide monomer units and wherein the prefix oligo- refers to a nucleic acid containing 2 to about 200 nucleotide monomer units.

Nucleobase: The term “nucleobase” refers to the parts of nucleic acids that are involved in the hydrogen-bonding that binds one nucleic acid strand to another complementary strand in a sequence specific manner. The most common naturally-occurring nucleobases are adenine (A), guanine (G), uracil (U), cytosine (C), and thymine (T). In some embodiments, a naturally-occurring nucleobases are modified adenine, guanine, uracil, cytosine, or thymine. In some embodiments, a naturally-occurring nucleobases are methylated adenine, guanine, uracil, cytosine, or thymine. In some embodiments, a nucleobase comprises a heteroaryl ring wherein a ring atom is nitrogen, and when in a nucleoside, the nitrogen is bonded to a sugar moiety. In some embodiments, a nucleobase comprises a heterocyclic ring wherein a ring atom is nitrogen, and when in a nucleoside, the nitrogen is bonded to a sugar moiety. In some embodiments, a nucleobase is a “modified nucleobase,” a nucleobase other than adenine (A), guanine (G), uracil (U), cytosine (C), and thymine (T). In some embodiments, a modified nucleobase is substituted A, T, C, G or U. In some embodiments, a modified nucleobase is a substituted tautomer of A, T, C, G, or U. In some embodiments, a modified nucleobases is methylated adenine, guanine, uracil, cytosine, or thymine. In some embodiments, a modified nucleobase mimics the spatial arrangement, electronic properties, or some other physicochemical property of the nucleobase and retains the property of hydrogen-bonding that binds one nucleic acid strand to another in a sequence specific manner. In some embodiments, a modified nucleobase can pair with all of the five naturally occurring bases (uracil, thymine, adenine, cytosine, or guanine) without substantially affecting the melting behavior, recognition by intracellular enzymes or activity of the oligonucleotide duplex. As used herein, the term “nucleobase” also encompasses structural analogs used in lieu of natural or naturally-occurring nucleotides, such as modified nucleobases and nucleobase analogs. In some embodiments, a nucleobase is optionally substituted A, T, C, G, or U, or an optionally substituted tautomer of A, T, C, G, or U. In some embodiments, a “nucleobase” refers to a nucleobase unit in an oligonucleotide or a nucleic acid (e.g., A, T, C, G or U as in an oligonucleotide or a nucleic acid).

Nucleoside: The term “nucleoside” refers to a moiety wherein a nucleobase or a modified nucleobase is covalently bound to a sugar or a modified sugar. In some embodiments, a nucleoside is a natural nucleoside, e.g., adenosine, deoxyadenosine, guanosine, deoxyguanosine, thymidine, uridine, cytidine, or deoxycytidine. In some embodiments, a nucleoside is a modified nucleoside, e.g., a substituted natural nucleoside selected from adenosine, deoxyadenosine, guanosine, deoxyguanosine, thymidine, uridine, cytidine, and deoxycytidine. In some embodiments, a nucleoside is a modified nucleoside, e.g., a substituted tautomer of a natural nucleoside selected from adenosine, deoxyadenosine, guanosine, deoxyguanosine, thymidine, uridine, cytidine, and deoxycytidine. In some embodiments, a “nucleoside” refers to a nucleoside unit in an oligonucleotide or a nucleic acid.

Nucleotide: The term “nucleotide” as used herein refers to a monomeric unit of a polynucleotide that consists of a nucleobase, a sugar, and one or more internucleotidic linkages (e.g., phosphate linkages in natural DNA and RNA). The naturally occurring bases [guanine, (G), adenine, (A), cytosine, (C), thymine, (T), and uracil (U)] are derivatives of purine or pyrimidine, though it should be understood that naturally and non-naturally occurring base analogs are also included. The naturally occurring sugar is the pentose (five-carbon sugar) deoxyribose (which forms DNA) or ribose (which forms RNA), though it should be understood that naturally and non-naturally occurring sugar analogs are also included. Nucleotides are linked via internucleotidic linkages to form nucleic acids, or polynucleotides. Many internucleotidic linkages are known in the art (such as, though not limited to, phosphate, phosphorothioates, boranophosphates and the like). Artificial nucleic acids include PNAs (peptide nucleic acids), phosphotriesters, phosphorothionates, H-phosphonates, phosphoramidates, boranophosphates, methylphosphonates, phosphonoacetates, thiophosphonoacetates and other variants of the phosphate backbone of native nucleic acids, such as those described herein. In some embodiments, a natural nucleotide comprises a naturally occurring base, sugar and internucleotidic linkage. As used herein, the term “nucleotide” also encompasses structural analogs used in lieu of natural or naturally-occurring nucleotides, such as modified nucleotides and nucleotide analogs. In some embodiments, a “nucleotide” refers to a nucleotide unit in an oligonucleotide or a nucleic acid.

Oligonucleotide: The term “oligonucleotide” refers to a polymer or oligomer of nucleotides, and may contain any combination of natural and non-natural nucleobases, sugars, and internucleotidic linkages.

Oligonucleotides can be single-stranded or double-stranded. A single-stranded oligonucleotide can have double-stranded regions (formed by two portions of the single-stranded oligonucleotide) and a double-stranded oligonucleotide, which comprises two oligonucleotide chains, can have single-stranded regions for example, at regions where the two oligonucleotide chains are not complementary to each other. Example oligonucleotides include, but are not limited to structural genes, genes including control and termination regions, self-replicating systems such as viral or plasmid DNA, single-stranded and double-stranded RNAi agents and other RNA interference reagents (RNAi agents or iRNA agents), shRNA, antisense oligonucleotides, ribozymes, microRNAs, microRNA mimics, supermirs, aptamers, antimirs, antagomirs, Ul adaptors, triplex-forming oligonucleotides, G-quadruplex oligonucleotides, RNA activators, immuno-stimulatory oligonucleotides, and decoy oligonucleotides.

Oligonucleotides of the present disclosure can be of various lengths. In particular embodiments, oligonucleotides can range from about 2 to about 200 nucleosides in length. In various related embodiments, oligonucleotides, single-stranded, double-stranded, or triple-stranded, can range in length from about 4 to about 10 nucleosides, from about 10 to about 50 nucleosides, from about 20 to about 50 nucleosides, from about 15 to about 30 nucleosides, from about 20 to about 30 nucleosides in length. In some embodiments, an oligonucleotide is from about 9 to about 39 nucleosides in length. In some embodiments, an oligonucleotide is from about 25 to about 70 nucleosides in length. In some embodiments, an oligonucleotide is from about 26 to about 70 nucleosides in length. In some embodiments, an oligonucleotide is from about 27 to about 70 nucleosides in length. In some embodiments, an oligonucleotide is from about 28 to about 70 nucleosides in length. In some embodiments, an oligonucleotide is from about 29 to about 70 nucleosides in length. In some embodiments, an oligonucleotide is from about 30 to about 70 nucleosides in length. In some embodiments, an oligonucleotide is from about 31 to about 70 nucleosides in length. In some embodiments, an oligonucleotide is from about 32 to about 70 nucleosides in length. In some embodiments, an oligonucleotide is from about 25 to about 60 nucleosides in length. In some embodiments, an oligonucleotide is from about 25 to about 50 nucleosides in length. In some embodiments, an oligonucleotide is from about 25 to about 40 nucleosides in length. In some embodiments, an oligonucleotide is from about 30 to about 40 nucleosides in length. In some embodiments, the oligonucleotide is at least 4, 5, 6, 7, 8, 9, 10, 11, 12, 13, 14, 15, 16, 17, 18, 19, 20, 21, 22, 23, 24, 25, 26, 27, 28, 29 or 30 nucleosides in length. In some embodiments, an oligonucleotide is at least 4 nucleosides in length. In some embodiments, an oligonucleotide is at least 5 nucleosides in length. In some embodiments, an oligonucleotide is at least 6 nucleosides in length. In some embodiments, an oligonucleotide is at least 7 nucleosides in length. In some embodiments, an oligonucleotide is at least 8 nucleosides in length. In some embodiments, an oligonucleotide is at least 9 nucleosides in length. In some embodiments, an oligonucleotide is at least 10 nucleosides in length. In some embodiments, an oligonucleotide is at least 11 nucleosides in length. In some embodiments, an oligonucleotide is at least 12 nucleosides in length. In some embodiments, an oligonucleotide is at least 15 nucleosides in length. In some embodiments, an oligonucleotide is at least 15 nucleosides in length. In some embodiments, an oligonucleotide is at least 16 nucleosides in length. In some embodiments, an oligonucleotide is at least 17 nucleosides in length. In some embodiments, an oligonucleotide is at least 18 nucleosides in length. In some embodiments, an oligonucleotide is at least 19 nucleosides in length. In some embodiments, an oligonucleotide is at least 20 nucleosides in length. In some embodiments, an oligonucleotide is at least 25 nucleosides in length. In some embodiments, an oligonucleotide is at least 26 nucleosides in length. In some embodiments, an oligonucleotide is at least 27 nucleosides in length. In some embodiments, an oligonucleotide is at least 28 nucleosides in length. In some embodiments, an oligonucleotide is at least 29 nucleosides in length. In some embodiments, an oligonucleotide is at least 30 nucleosides in length. In some embodiments, an oligonucleotide is at least 31 nucleosides in length. In some embodiments, an oligonucleotide is at least 32 nucleosides in length. In some embodiments, an oligonucleotide is at least 33 nucleosides in length. In some embodiments, an oligonucleotide is at least 34 nucleosides in length. In some embodiments, an oligonucleotide is at least 35 nucleosides in length. In some embodiments, an oligonucleotide is at least 36 nucleosides in length. In some embodiments, an oligonucleotide is at least 37 nucleosides in length. In some embodiments, an oligonucleotide is at least 38 nucleosides in length. In some embodiments, an oligonucleotide is at least 39 nucleosides in length. In some embodiments, an oligonucleotide is at least 40 nucleosides in length. In some embodiments, an oligonucleotide is 25 nucleosides in length. In some embodiments, an oligonucleotide is 26 nucleosides in length. In some embodiments, an oligonucleotide is 27 nucleosides in length. In some embodiments, an oligonucleotide is 28 nucleosides in length. In some embodiments, an oligonucleotide is 29 nucleosides in length. In some embodiments, an oligonucleotide is 30 nucleosides in length. In some embodiments, an oligonucleotide is 31 nucleosides in length. In some embodiments, an oligonucleotide is 32 nucleosides in length. In some embodiments, an oligonucleotide is 33 nucleosides in length. In some embodiments, an oligonucleotide is 34 nucleosides in length. In some embodiments, an oligonucleotide is 35 nucleosides in length. In some embodiments, an oligonucleotide is 36 nucleosides in length. In some embodiments, an oligonucleotide is 37 nucleosides in length. In some embodiments, an oligonucleotide is 38 nucleosides in length. In some embodiments, an oligonucleotide is 39 nucleosides in length. In some embodiments, an oligonucleotide is 40 nucleosides in length. In some embodiments, each nucleoside counted in an oligonucleotide length independently comprises a nucleobase comprising a ring having at least one nitrogen ring atom. In some embodiments, each nucleoside counted in an oligonucleotide length independently comprises A, T, C, G, or U, or optionally substituted A, T, C, G, or U, or an optionally substituted tautomer of A, T, C, G or U.

Oligonucleotide type: As used herein, the phrase “oligonucleotide type” is used to define an oligonucleotide that has a particular base sequence, pattern of backbone linkages (i.e., pattern of internucleotidic linkage types, for example, phosphate, phosphorothioate, phosphorothioate triester, etc.), pattern of backbone chiral centers [i.e., pattern of linkage phosphorus stereochemistry (Rp/Sp)], and pattern of backbone phosphorus modifications. In some embodiments, oligonucleotides of a common designated “type” are structurally identical to one another.

One of skill in the art will appreciate that synthetic methods of the present disclosure provide for a degree of control during the synthesis of an oligonucleotide strand such that each nucleotide unit of the oligonucleotide strand can be designed and/or selected in advance to have a particular stereochemistry at the linkage phosphorus and/or a particular modification at the linkage phosphorus, and/or a particular base, and/or a particular sugar. In some embodiments, an oligonucleotide strand is designed and/or selected in advance to have a particular combination of stereocenters at the linkage phosphorus. In some embodiments, an oligonucleotide strand is designed and/or determined to have a particular combination of modifications at the linkage phosphorus. In some embodiments, an oligonucleotide strand is designed and/or selected to have a particular combination of bases. In some embodiments, an oligonucleotide strand is designed and/or selected to have a particular combination of one or more of the above structural characteristics. In some embodiments, the present disclosure provides compositions comprising or consisting of a plurality of oligonucleotide molecules (e.g., chirally controlled oligonucleotide compositions). In some embodiments, all such molecules are of the same type (i.e., are structurally identical to one another). In some embodiments, however, provided compositions comprise a plurality of oligonucleotides of different types, typically in pre-determined relative amounts.

Optionally Substituted: As described herein, compounds, e.g., oligonucleotides, of the disclosure may contain optionally substituted and/or substituted moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. In some embodiments, an optionally substituted group is unsubstituted. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. Certain substituents are described below.

Suitable monovalent substituents on a substitutable atom, e.g., a suitable carbon atom, are independently halogen: —(CH₂)₀₋₄R^{∘}; —(CH₂)₀₋₄OR^{∘}; —O(CH₂)₀₋₄R, —O—(CH₂)₀₋₄C(O)OR^{∘}; —(CH₂)₀₋₄CH(OR^{∘})₂; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, —SC(S)SR^{∘}; —(CH₂)₀₋₄SC(O)R^{∘}; —(CH₂)₀₋₄C(O)NR^{∘}₂; —C(S)NR^{∘}2; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —Si(R^{∘})₃; —OSi(R^{∘})₃; —B(R^{∘})₂; —OB(R^{∘})₂; —OB(OR^{∘})₂; —P(R^{∘})₂; —P(OR^{∘})₂; —P(R^{∘})(OR^{∘}); —OP(R^{∘})₂; —OP(OR^{∘})₂; —OP(R^{∘})(OR^{∘}); —P(O)(R^{∘})₂; —P(O)(OR^{∘})₂; —OP(O)(R^{∘})₂; —OP(O)(OR^{∘})₂; —OP(O)(OR^{∘})(SR^{∘}); —SP(O)(R^{∘})₂; —SP(O)(OR^{∘})₂; —N(R^{∘})P(O)(R^{∘})₂; —N(R^{∘})P(O)(OR^{∘})₂; —P(R^{∘})₂[B(R^{∘})₃]; —P(OR^{∘}):[B(R^{∘})₃]; —OP(R^{∘})₂[B(R^{∘})₃]; —OP(OR^{∘})₂[B(R^{∘})₃]; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined herein and is independently hydrogen, C₁₋₂₀ aliphatic, C₁₋₂₀ heteroaliphatic having 1-5 heteroatoms independently selected from nitrogen, oxygen, sulfur, silicon and phosphorus, —CH₂—(C₆₋₁₄ aryl), —O(CH₂)₀₋₁(C₆₋₁₄ aryl), —CH₂-(5-14 membered heteroaryl ring), a 5-20 membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of R^{∘}, taken together with their intervening atom(s), form a 5-20 membered, monocyclic, bicyclic, or polycyclic, saturated, partially unsaturated or aryl ring having 0-5 heteroatoms independently selected from nitrogen, oxygen, sulfur, silicon and phosphorus, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂))₂C(O)OR^{●}, —(CH₂)₀₋₂SR^{●}, —(CH₂)₀₋₂SH, —(CH₂))₂NH₂, —(CH₂)₀₋₂NHR^{●}, —(CH₂)₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, and a 5-6-membered saturated, partially unsaturated, or aryl ring having 0-4 heteroatoms independently selected from nitrogen, oxygen, and sulfur. Suitable divalent substituents on a saturated carbon atom of R^{∘} include ═O and ═S.

Suitable divalent substituents, e.g., on a suitable carbon atom, are independently the following: ═O, ═S, ═NNR*₂, =NNHC(O)R*, =NNHC(O)OR*, =NNHS(O)₂R*, =NR*, =NOR*, —O(C(R*₂)₂₋₃O—, or —S(C(R*₂))₂₋₃S—, wherein each independent occurrence of R* is selected from hydrogen, C₁₋₆ aliphatic which may be substituted as defined below, and an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, and an unsubstituted 5-6-membered saturated, partially unsaturated, and aryl ring having 0-4 heteroatoms independently selected from nitrogen, oxygen, and sulfur.

Suitable substituents on the aliphatic group of R* are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

In some embodiments, suitable substituents on a substitutable nitrogen are independently —R^{†}, —NR^{†}2, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†)S(O)}₂R^{†}; wherein each R^{† is independently hydrogen, C}₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, and sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, and sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

P-modification: as used herein, the term “P-modification” refers to any modification at the linkage phosphorus other than a stereochemical modification. In some embodiments, a P-modification comprises addition, substitution, or removal of a pendant moiety covalently attached to a linkage phosphorus.

Partially unsaturated: As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

Pharmaceutical composition: As used herein, the term “pharmaceutical composition” refers to an active agent, formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation: topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions and/or dosage forms which arc, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil: glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salt include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, malcate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, a provided compound comprises one or more acidic groups, e.g., an oligonucleotide, and a pharmaceutically acceptable salt is an alkali, alkaline earth metal, or ammonium (e.g., an ammonium salt of N(R)₃, wherein each R is independently defined and described in the present disclosure) salt. Representative alkali or alkaline earth metal salts include sodium, lithium, potassium, calcium, magnesium, and the like. In some embodiments, a pharmaceutically acceptable salt is a sodium salt. In some embodiments, a pharmaceutically acceptable salt is a potassium salt. In some embodiments, a pharmaceutically acceptable salt is a calcium salt. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate. In some embodiments, a provided compound comprises more than one acid groups, for example, an oligonucleotide may comprise two or more acidic groups (e.g., in natural phosphate linkages and/or modified internucleotidic linkages). In some embodiments, a pharmaceutically acceptable salt, or generally a salt, of such a compound comprises two or more cations, which can be the same or different. In some embodiments, in a pharmaceutically acceptable salt (or generally, a salt), all ionizable hydrogen (e.g., in an aqueous solution with a pKa no more than about 11, 10, 9, 8, 7, 6, 5, 4, 3, or 2; in some embodiments, no more than about 7; in some embodiments, no more than about 6; in some embodiments, no more than about 5; in some embodiments, no more than about 4: in some embodiments, no more than about 3) in the acidic groups are replaced with cations. In some embodiments, each phosphorothioate and phosphate group independently exists in its salt form (e.g., if sodium salt, —O—P(O)(SNa)—O— and —O—P(O)(ONa)—O—, respectively). In some embodiments, each phosphorothioate and phosphate internucleotidic linkage independently exists in its salt form (e.g., if sodium salt, —O—P(O)(SNa)—O— and —O—P(O)(ONa)—O—, respectively). In some embodiments, a pharmaceutically acceptable salt is a sodium salt of an oligonucleotide. In some embodiments, a pharmaceutically acceptable salt is a sodium salt of an oligonucleotide, wherein each acidic phosphate and modified phosphate group (e.g., phosphorothioate, phosphate, etc.), if any, exists as a salt form (all sodium salt).

Predetermined: By predetermined (or pre-determined) is meant deliberately selected or non-random or controlled, for example as opposed to randomly occurring, random, or achieved without control. Those of ordinary skill in the art, reading the present specification, will appreciate that the present disclosure provides technologies that permit selection of particular chemistry and/or stereochemistry features to be incorporated into oligonucleotide compositions, and further permits controlled preparation of oligonucleotide compositions having such chemistry and/or stereochemistry features. Such provided compositions are “predetermined” as described herein. Compositions that may contain certain oligonucleotides because they happen to have been generated through a process that are not controlled to intentionally generate the particular chemistry and/or stereochemistry features are not “predetermined” compositions. In some embodiments, a predetermined composition is one that can be intentionally reproduced (e.g., through repetition of a controlled process). In some embodiments, a predetermined level of a plurality of oligonucleotides in a composition means that the absolute amount, and/or the relative amount (ratio, percentage, etc.) of the plurality of oligonucleotides in the composition is controlled. In some embodiments, a predetermined level of a plurality of oligonucleotides in a composition is achieved through chirally controlled oligonucleotide preparation.

Protecting group: The term “protecting group,” as used herein, is well known in the art and includes those described in detail in Protecting Groups in Organic Synthesis, T. W. Greene and P. G. M. Wuts, 3^{rd} edition, John Wiley & Sons, 1999, the entirety of which is incorporated herein by reference. Also included are those protecting groups specially adapted for nucleoside and nucleotide chemistry described in Current Protocols in Nucleic Acid Chemistry, edited by Serge L. Beaucage et al. 06/2012, the entirety of Chapter 2 is incorporated herein by reference. Suitable amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), I-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Teroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dis), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-<dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pine), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), p-trimethylsilylethanesulfonamide (SES), 9-anthracenesulfonamide, 4-(4′8′-dimethoxynaphthylmethyl)benzenesulfonamide (DNMBS), benzylsulfonamide, trifluoromethylsulfonamide, and phenacylsulfonamide.

Suitably protected carboxylic acids further include, but are not limited to, silyl-, alkyl-, alkenyl-, aryl-, and arylalkyl-protected carboxylic acids. Examples of suitable silyl groups include trimethylsilyl, triethylsilyl, t-butyldimethylsilyl, t-butyldiphenylsilyl, triisopropylsilyl, and the like. Examples of suitable alkyl groups include methyl, benzyl, p-methoxybenzyl, 3,4-dimethoxybenzyl, trityl, t-butyl, tetrahydropyran-2-yl. Examples of suitable alkenyl groups include allyl. Examples of suitable aryl groups include optionally substituted phenyl, biphenyl, or naphthyl. Examples of suitable arylalkyl groups include optionally substituted benzyl (e.g., p-methoxybenzyl (MPM), 3,4-dimethoxybenzyl, O-nitrobenzyl, p-nitrobenzyl, p-halobenzyl, 2,6-dichlorobenzyl, p-cyanobenzyl), and 2- and 4-picolyl.

Suitable hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a, 4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-<dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4,4″-tris(levulinoyloxyphenyl)methyl, 4,4′,4″-tris(benzoyloxyphenyl)methyl, 3-(imidazol-1-yl)bis(4′,4′-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

In some embodiments, a hydroxyl protecting group is acetyl, t-butyl, tbutoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, benzoyl, p-phenylbenzoyl, 2,6-dichlorobenzyl, diphenylmethyl, p-nitrobenzyl, triphenylmethyl (trityl), 4,4′-dimethoxytrityl, trimethylsilyl, triethylsilyl, t-butyldimethylsilyl, t-butyldiphenylsilyl, triphenylsilyl, triisopropylsilyl, benzoylformate, chloroacetyl, trichloroacetyl, trifluoroacetyl, pivaloyl, 9-fluorenylmethyl carbonate, mesylate, tosylate, triflate, trityl, monomethoxytrityl (MMTr), 4,4′-dimethoxytrityl, (DMTr) and 4,4′,4″-trimethoxytrityl (TMTr), 2-cyanoethyl (CE or Cne), 2-(trimethylsilyl)ethyl (TSE), 2-(2-nitrophenyl)ethyl, 2-(4-cyanophenyl)ethyl 2-(4-nitrophenyl)ethyl (NPE), 2-(4-nitrophenylsulfonyl)ethyl, 3,5-dichlorophenyl, 2,4-dimethylphenyl, 2-nitrophenyl, 4-nitrophenyl, 2,4,6-trimethylphenyl, 2-(2-nitrophenyl)ethyl, butylthiocarbonyl, 4,4′,4″-tris(benzoyloxy)trityl, diphenylcarbamoyl, levulinyl, 2-(dibromomethyl)benzoyl (Dbmb), 2-(isopropylthiomethoxymethyl)benzoyl (Ptmt), 9-phenylxanthen-9-yl (pixyl) or 9-(p-methoxyphenyl)xanthine-9-yl (MOX). In some embodiments, each of the hydroxyl protecting groups is, independently selected from acetyl, benzyl, t-butyldimethylsilyl, t-butyldiphenylsilyl and 4,4′-dimethoxytrityl. In some embodiments, the hydroxyl protecting group is selected from the group consisting of trityl, monomethoxytrityl and 4,4′-dimethoxytrityl group. In some embodiments, a phosphorous linkage protecting group is a group attached to the phosphorous linkage (e.g., an internucleotidic linkage) throughout oligonucleotide synthesis. In some embodiments, a protecting group is attached to a sulfur atom of an phosphorothioate group. In some embodiments, a protecting group is attached to an oxygen atom of an internucleotide phosphorothioate linkage. In some embodiments, a protecting group is attached to an oxygen atom of the internucleotide phosphate linkage. In some embodiments a protecting group is 2-cyanoethyl (CE or Cne), 2-trimethylsilylethyl, 2-nitroethyl, 2-sulfonylethyl, methyl, benzyl, o-nitrobenzyl, 2-(p-nitrophenyl)ethyl (NPE or Npe), 2-phenylethyl, 3-(N-tert-butylcarboxamido)-1-propyl, 4-oxopentyl, 4-methylthio-1-butyl, 2-cyano-1,1-dimethylethyl, 4-N-methylaminobutyl, 3-(2-pyridyl)-1-propyl, 2-[N-methyl-N-(2-pyridyl)]aminoethyl, 2-(N-formyl,N-methyl)aminoethyl, or 4-[N-methyl-N-(2,2,2-trifluoroacetyl)amino]butyl.

Subject: As used herein, the term “subject” or “test subject” refers to any organism to which a compound (e.g., an oligonucleotide) or composition is administered in accordance with the present disclosure e.g., for experimental, diagnostic, prophylactic and/or therapeutic purposes. Typical subjects include animals (e.g., mammals such as mice, rats, rabbits, non-human primates, and humans; insects; worms; etc.) and plants. In some embodiments, a subject is a human. In some embodiments, a subject may be suffering from and/or susceptible to a disease, disorder and/or condition.

Substantially: As used herein, the term “substantially” refers to the qualitative condition of exhibiting total or near-total extent or degree of a characteristic or property of interest. A base sequence which is substantially identical or complementary to a second sequence is not fully identical or complementary to the second sequence, but is mostly or nearly identical or complementary to the second sequence. In some embodiments, an oligonucleotide with a substantially complementary sequence to another oligonucleotide or nucleic acid forms duplex with the oligonucleotide or nucleic acid in a similar fashion as an oligonucleotide with a fully complementary sequence. In addition, one of ordinary skill in the biological and/or chemical arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Sugar: The term “sugar” refers to a monosaccharide or polysaccharide in closed and/or open form. In some embodiments, sugars are monosaccharides. In some embodiments, sugars are polysaccharides. Sugars include, but are not limited to, ribose, deoxyribose, pentofuranose, pentopyranose, and hexopyranose moieties. As used herein, the term “sugar” also encompasses structural analogs used in lieu of conventional sugar molecules, such as glycol, polymer of which forms the backbone of the nucleic acid analog, glycol nucleic acid (“GNA”), etc. As used herein, the term “sugar” also encompasses structural analogs used in lieu of natural or naturally-occurring nucleotides, such as modified sugars and nucleotide sugars. In some embodiments, a sugar is a RNA or DNA sugar (ribose or deoxyribose). In some embodiments, a sugar is a modified ribose or deoxyribose sugar, e.g., 2′-modified, 5′-modified, etc. As described herein, in some embodiments, when used in oligonucleotides and/or nucleic acids, modified sugars may provide one or more desired properties, activities, etc. In some embodiments, a sugar is optionally substituted ribose or deoxyribose. In some embodiments, a “sugar” refers to a sugar unit in an oligonucleotide or a nucleic acid.

Susceptible to: An individual who is “susceptible to” a disease, disorder and/or condition is one who has a higher risk of developing the disease, disorder and/or condition than does a member of the general public. In some embodiments, an individual who is susceptible to a disease, disorder and/or condition is predisposed to have that disease, disorder and/or condition. In some embodiments, an individual who is susceptible to a disease, disorder and/or condition may not have been diagnosed with the disease, disorder and/or condition. In some embodiments, an individual who is susceptible to a disease, disorder and/or condition may exhibit symptoms of the disease, disorder and/or condition. In some embodiments, an individual who is susceptible to a disease, disorder and/or condition may not exhibit symptoms of the disease, disorder and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutic agent: As used herein, the term “therapeutic agent” in general refers to any agent that elicits a desired effect (e.g., a desired biological, clinical, or pharmacological effect) when administered to a subject. In some embodiments, an agent is considered to be a therapeutic agent if it demonstrates a statistically significant effect across an appropriate population. In some embodiments, an appropriate population is a population of subjects suffering from and/or susceptible to a disease, disorder or condition. In some embodiments, an appropriate population is a population of model organisms. In some embodiments, an appropriate population may be defined by one or more criterion such as age group, gender, genetic background, preexisting clinical conditions, prior exposure to therapy. In some embodiments, a therapeutic agent is a substance that alleviates, ameliorates, relieves, inhibits, prevents, delays onset of, reduces severity of, and/or reduces incidence of one or more symptoms or features of a disease, disorder, and/or condition in a subject when administered to the subject in an effective amount. In some embodiments, a “therapeutic agent” is an agent that has been or is required to be approved by a government agency before it can be marketed for administration to humans. In some embodiments, a “therapeutic agent” is an agent for which a medical prescription is required for administration to humans. In some embodiments, a therapeutic agent is a provided compound, e.g., a provided oligonucleotide.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Unsaturated: The term “unsaturated,” as used herein, means that a moiety has one or more units of unsaturation.

Wild-type: As used herein, the term “wild-type” has its art-understood meaning that refers to an entity having a structure and/or activity as found in nature in a “normal” (as contrasted with mutant, diseased, altered, etc.) state or context. Those of ordinary skill in the art will appreciate that wild type genes and polypeptides often exist in multiple different forms (e.g., alleles).

As those skilled in the art will appreciate, methods and compositions described herein relating to provided compounds (e.g., oligonucleotides) generally also apply to pharmaceutically acceptable salts of such compounds.

### Description of Certain Embodiments

Oligonucleotides are useful in various therapeutic, diagnostic, and research applications. Use of naturally occurring nucleic acids is limited, for example, by their susceptibility to endo- and exo-nucleases. As such, various synthetic counterparts have been developed to circumvent these shortcomings and/or to further improve various properties and activities. These include synthetic oligonucleotides that contain chemical modifications, e.g., base modifications, sugar modifications, backbone modifications, etc., which, among other things, render these molecules less susceptible to degradation and improve other properties and/or activities.

From a structural point of view, modifications to internucleotidic linkages can introduce chirality, and certain properties and activities may be affected by configurations of linkage phosphorus atoms of oligonucleotides. For example, binding affinity, sequence specific binding to complementary RNA, stability to nucleases, activities, delivery, pharmacokinetics, etc. can be affected by, inter alia, chirality of backbone linkage phosphorus atoms.

Among other things, the present disclosure utilizes technologies for controlling various structural elements, e.g., sugar modifications and patterns thereof, nucleobase modifications and patterns thereof, modified internucleotidic linkages and patterns thereof, linkage phosphorus stereochemistry and patterns thereof, additional chemical moieties (moieties that are not typically in an oligonucleotide chain) and patterns thereof, etc. With the capability to fully control structural elements of oligonucleotides, the present disclosure provides oligonucleotides with improved and/or new properties and/or activities for various applications, e.g., as therapeutic agents, probes, etc. For example, as demonstrated herein, provided oligonucleotides and compositions thereof are particularly powerful for editing target adenosine in target nucleic acids to, in some embodiments, correct a G to A mutation by converting A to I. In some embodiments, provided technologies can 1024 G>A mutation in SERPINA1.

In some embodiments, provided technologies are chirally controlled. Among other things, the present disclosure provides technologies for preparing chirally controlled (in some embodiments, stereochemically pure) oligonucleotides. In some embodiments, oligonucleotides are stereochemically pure. In some embodiments, oligonucleotides of the present disclosure are about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 15%, 20%, 25%, 30%, 35%, 40%, 45%, 50%, 55%, 60%, 65%, 70%, 75%, 80%, 85%, 90%, 95%, or 100%, or at least about 5%, 10%, 15%, 20%, 25%, 30%, 35%, 40%, 45%, 50%, 55%, 60%, 65%, 70%, 75%, 80%, 85%, 90%, 95%, or 99% stereochemically pure.

In some embodiments, an oligonucleotide composition comprises a plurality of oligonucleotides sharing a common base sequence, wherein each internucleotidic linkage comprising chiral linkage phosphorus in the oligonucleotides is independently a chirally controlled internucleotidic linkage. In some embodiments, an oligonucleotide composition comprises oligonucleotides of the same constitution, wherein one or more internucleotidic linkages are chirally controlled. In some embodiments, an oligonucleotide composition comprises oligonucleotides of the same constitution, wherein each internucleotidic linkage comprising chiral linkage phosphorus is independently a chirally controlled internucleotidic linkage. In some embodiments, at least 10%, 20%, 30%, 40%, 50%, 60%, 70%, 75%, 80%, 85%, 90% or 95% of all oligonucleotides, or all oligonucleotides of the common base sequence, are oligonucleotides of the plurality. In some embodiments, in a composition of a provided oligonucleotide or compound, each chiral phosphorus of the oligonucleotide or compound is chirally controlled.

In some embodiments, the present disclosure provides technologies for preparing, assessing and/or utilizing provided oligonucleotides and compositions thereof.

As used in the present disclosure, in some embodiments, “one or more” is 1-200, 1-150, 1-100, 1-90, 1-80, 1-70, 1-60, 1-50, 1-40, 1-30, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60. In some embodiments, “one or more” is one. In some embodiments, “one or more” is two. In some embodiments, “one or more” is three. In some embodiments, “one or more” is four. In some embodiments, “one or more” is five. In some embodiments, “one or more” is six. In some embodiments, “one or more” is seven. In some embodiments, “one or more” is eight. In some embodiments, “one or more” is nine. In some embodiments, “one or more” is ten. In some embodiments, “one or more” is at least one. In some embodiments, “one or more” is at least two. In some embodiments, “one or more” is at least three. In some embodiments, “one or more” is at least four. In some embodiments, “one or more” is at least five. In some embodiments, “one or more” is at least six. In some embodiments, “one or more” is at least seven. In some embodiments, “one or more” is at least eight. In some embodiments, “one or more” is at least nine. In some embodiments, “one or more” is at least ten.

As used in the present disclosure, in some embodiments, “at least one” is one or more.

Various embodiments are described for variables, e.g., R, R^{L}, L, etc., as examples. Embodiments described for a variable, e.g., R, are generally applicable to all variables that can be such a variable (e.g., R′, R″, R^{L}, R^{L1}, etc.).

### Oligonucleotides

Among other things, the present disclosure provides oligonucleotides of various designs, which may comprise various nucleobases and patterns thereof, sugars and patterns thereof, internucleotidic linkages and patterns thereof, and/or additional chemical moieties and patterns thereof as described in the present disclosure. In some embodiments, provided oligonucleotides can direct A to I editing in target nucleic acids. In some embodiments, oligonucleotides of the present disclosure are single-stranded oligonucleotides capable of site-directed editing of an adenosine (conversion of A into I) in a target RNA sequence. In some embodiments, provided technologies can edit 1024 G>A in SERPINA1. In some embodiments, oligonucleotides of the present disclosure contain lower levels of 2′-F modified sugars and no natural RNA sugars. In some embodiments, provided technologies provide high levels of activity (e.g., editing of 1024 G>A in SERPINA1) and stability.

In some embodiments, provided oligonucleotides are sufficiently short to facilitate delivery, reduce manufacture complexity and/or cost which maintaining desired properties and activities (e.g., editing of adenosine).

In some embodiments, a provided oligonucleotide comprises an additional chemical moiety. In some embodiments, a provided oligonucleotide comprises one or more carbohydrate moieties. In some embodiments, a provided oligonucleotide comprises one or more GalNAc moieties. In some embodiments, a provided oligonucleotide comprises one or more targeting moieties.

In some embodiments, provided oligonucleotides can direct a correction of a G to A mutation in a target sequence, or a product thereof. In some embodiments, a correction of a G to A mutation is or comprises conversion of A to I, which can be read as G during translation or other biological processes. In some embodiments, provided oligonucleotides can direct a correction of a G to A mutation in a target sequence or a product thereof via ADAR-mediated deamination. In some embodiments, provided oligonucleotides can direct a correction of a G to A mutation in a target sequence or a product thereof via ADAR-mediated deamination by recruiting an endogenous ADAR (e.g., in a target cell) and facilitating the ADAR-mediated deamination.

In some embodiments, oligonucleotide hybridizes to two or more variants of transcripts derived from a sense strand of a target site (e.g., a target sequence).

In some embodiments, provided oligonucleotides contain increased levels of one or more isotopes. In some embodiments, provided oligonucleotides are labeled, e.g., by one or more isotopes of one or more elements, e.g., hydrogen, carbon, nitrogen, etc. In some embodiments, provided oligonucleotides in provided compositions, e.g., oligonucleotides of a plurality of a composition, comprise base modifications, sugar modifications, and/or internucleotidic linkage modifications, wherein the oligonucleotides contain an enriched level of deuterium. In some embodiments, provided oligonucleotides are labeled with deuterium (replacing -¹H with -²H) at one or more positions. In some embodiments, one or more ¹H of an oligonucleotide chain or any moiety conjugated to the oligonucleotide chain (e.g., a targeting moiety, etc.) is substituted with 2H. Such oligonucleotides can be used in compositions and methods described herein.

In some embodiments, a provided oligonucleotide or composition is characterized in that, when it is contacted with a target nucleic acid comprising a target adenosine in a system (e.g., an ADAR-mediated deamination system), modification of the target adenosine (e.g., deamination of the target A) is improved relative to that observed under reference conditions (e.g., selected from the group consisting of absence of the composition, presence of a reference oligonucleotide or composition, and combinations thereof). In some embodiments, modification, e.g., ADAR-mediated deamination (e.g., endogenous ADAR-mediated deamination) is increased 10%, 20%, 30%, 40%, 50%, 60%, 70%, 80%, 90%, 100%, or 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 200, 300, 400, 500, 600, 700, 800, 900, 1000 fold or more.

In some embodiments, oligonucleotides are provided, administered or delivered as salt forms. In some embodiments, oligonucleotides are provided, administered or delivered as salts comprising negatively-charged internucleotidic linkages (e.g., phosphorothioate internucleotidic linkages, natural phosphate linkages, etc.) existing as their salt forms. In some embodiments, oligonucleotides are provided, administered or delivered as pharmaceutically acceptable salts. In some embodiments, oligonucleotides are provided, administered or delivered as metal salts. In some embodiments, oligonucleotides are provided, administered or delivered as sodium salts. In some embodiments, oligonucleotides are provided, administered or delivered as ammonium salts. In some embodiments, oligonucleotides are provided, administered or delivered as metal salts, e.g., sodium salts, wherein each negatively-charged internucleotidic linkage is independently in a salt form (e.g., for sodium salts, —O—P(O)(SNa)—O— for a phosphorothioate internucleotidic linkage, —O—P(O)(ONa)—O— for a natural phosphate linkage, etc.).

In some embodiments, oligonucleotides are chiral controlled, comprising one or more chirally controlled internucleotidic linkages. In some embodiments, each chiral phosphorus is independently chirally controlled. In some embodiments, provided oligonucleotides or compositions thereof are substantially pure of other stereoisomers with respect to chiral phosphorus. In some embodiments, provided oligonucleotides or compositions thereof are substantially pure of other stereoisomers. In some embodiments, the present disclosure provides chirally controlled oligonucleotide compositions.

As described herein, oligonucleotides of the present disclosure can be provided in high purity (e.g., about 50%-100%). In some embodiments, oligonucleotides of the present disclosure are of high stereochemical purity (e.g., about 50%-100%). In some embodiments, oligonucleotides in provided compositions are of high stereochemical purity (e.g., high percentage (e.g., 50%-100%) of a stereoisomer compared to the other stereoisomers of the same oligonucleotide). In some embodiments, a percentage is at least or about 50%. In some embodiments, a percentage is at least or about 60%. In some embodiments, a percentage is at least or about 70%. In some embodiments, a percentage is at least or about 75%. In some embodiments, a percentage is at least or about 80%. In some embodiments, a percentage is at least or about 85%. In some embodiments, a percentage is at least or about 90%. In some embodiments, a percentage is at least or about 95%.

In some embodiments, an oligonucleotide is

(SEQ ID NO: 14)
mCn001RmC*SmC*SfA*SfG*Sm5CcoAeofG*SfC*SmUmUn001RmCfA*SfGn001RfUm5Ceo*SfC*
SmCmUn001RmUTeofC*ST*Sb008U*SIn001SmUfC*SmG*SmAn001RmU,
(SEQ ID NO: 16)
mCn001RmC*SmC*SfA*SfG*Sm5CeoAeofG*SfC*STeofUn001RmCfA*SfGn001RfUm5Ceo*SfC*
SfC*SfUn001RTeoTeofC*ST*Sb008U*SIn001SmUfC*SmG*SmAn001RmU,
(SEQ ID NO: 4)
mCn001RmC*SmC*SfA*SfG*Sm5CeoAcofG*SfC*STeofUn001RmCfA*SfGn001RfUmC*SfC*SfC*S
fUn001RTeoTeofC*ST*Sb008U*SIn001SmUfC*SmG*SmAn001RmU,
(SEQ ID NO: 30)
mCn001RmC*SmC*SfA*SfG*Sm5CeoAeofG*SfC*STeofUn001RmCfA*SmGn001RfUm5Ceo*SfC*
SfC*SfUn001RTeoTeofC*ST*Sb008U*SIn001SmUfC*SmG*SmAn001RmU,
(SEQ ID NO: 29)
mCn001RmC*SmC*SfA*SfG*Sm5CcoAeofG*SfC*STeofUn001RmCfA*SmGn001RfUmC*SfC*SfC*
SfUn001RTeoTeofC*ST*Sb008U*SIn001SmUfC*SmG*SmAn001RmU,
(SEQ ID NO: 32)
mCn001RmC*SmC*SfA*SfG*Sm5CeoAeofG*Sm5CeoTeo*SmUn001Rm5CeofA*SfGn001RmUm5Ceom5Ceo*
SfC*SfU*STeoTeofC*ST*Sb008U*SIn001SmUfC*SmG*SmAn001RmU,
(SEQ ID NO: 11)
mCn001RmC*SmC*SfA*SfG*Sm5CeoAcofG*SmCTeo*SmUn001RmCfA*SfGn001RmUm5CcomC*
SfC*SfU*STeoTeofC*ST*Sb008U*SIn001SmUfC*SmG*SmAn001RmU,
(SEQ ID NO: 31)
mCn001RmC*SmC*SfA*SfG*Sm5CeoAcofG*SmCTeo*SmUn001RmCfA*SfGn001RmUmCmC*SfC*
SfU*STeoTeofC*ST*Sb008U*SIn001SmUfC*SmG*SmAn001RmU,
(SEQ ID NO: 2)
mCn001RmC*SmC*SfA*SfG*SmCmA*SfG*SfCmU*SfUn001RmCfA*SfGn001RfUmC*SfC*SfC*SfU*
SmUmUfC*ST*Sb008U*SIn001SmUfC*SmG*SmAn001RmU,
or
(SEQ ID NO: 6)
mCn001RmC*SmC*SfA*SfG*SmCmAfG*SfC*SmUfUn001RmCfA*SmGn001RfUmC*SfC*SfC*
SfUn001RmUmUfC*ST*Sb008U*SIn001SmUfC*SmG*SmAn001RmU.

In some embodiments, an oligonucleotide has a structure selected from Table 1 of WO 2022/099159 or a salt thereof, wherein the oligonucleotide targets SERPINA1 and does not contain an additional chemical moiety. In some embodiments, an oligonucleotide has a structure selected from Table 1 of WO 2022/099159 or a salt thereof, wherein the oligonucleotide targets SERPINA1 and does not contain LOO. In some embodiments, an oligonucleotide has a structure selected from Table 1 of WO 2022/099159 or a salt thereof, wherein the oligonucleotide does not contain any Mod. In some embodiments, an oligonucleotide has a structure selected from Table 1 of WO 2022/099159 or a salt thereof, wherein the oligonucleotide does not contain Mod001. In some embodiments, an oligonucleotide has a structure selected from Table 1 of WO 2022/099159 or a salt thereof, wherein the oligonucleotide does not contain Mod012. In some embodiments, an oligonucleotide has a structure selected from Table 1 of WO 2022/099159 or a salt thereof, wherein the oligonucleotide does not contain Mod001 or Mod012. In some embodiments, an oligonucleotide has a structure selected from Table 1D of WO 2022/099159 or a salt thereof. In some embodiments, an oligonucleotide has a structure selected from Table 1E of WO 2022/099159 or a salt thereof. In some embodiments, an oligonucleotide has a structure selected from Table 1F of WO 2022/099159 or a salt thereof. In some embodiments, an oligonucleotide has a structure selected from WV-42934 to WV-44247 in Table 1F of WO 2022/099159 or a salt thereof. In some embodiments, an oligonucleotide has a structure selected from WV-44248 to WV-44277 in Table 1F of WO 2022/099159 or a salt thereof. In some embodiments, an oligonucleotide has a structure selected from VVV-44349 to WV-44362 in Table 1F of WO 2022/099159 or a salt thereof. In some embodiments, an oligonucleotide has a structure selected from WV-44363 to WV-44390 in Table 1F of WO 2022/099159 or a salt thereof. In some embodiments, an oligonucleotide has a structure selected from WV-44482 to WV-44515 in Table 1F of WO 2022/099159 or a salt thereof. In some embodiments, an oligonucleotide has a structure selected from WV-46406 to WV-47042 in Table 1F of WO 2022/099159 or a salt thereof. In some embodiments, an oligonucleotide has a structure selected from WV-47339 to WV-47483 in Table 1F of WO 2022/099159 or a salt thereof. In some embodiments, an oligonucleotide has a structure selected from WV-47495 to WV-47496 in Table 1F of WO 2022/099159 or a salt thereof. In some embodiments, an oligonucleotide has a structure selected from WV-47610 to WV-47631 in Table 1F of WO 2022/099159 or a salt thereof. In some embodiments, an oligonucleotide has a structure selected from WV-48455 to WV-48459 in Table 1F of WO 2022/099159 or a salt thereof. In some embodiments, an oligonucleotide has a structure selected from WV-49094 to WV-49096 in Table 1F of WO 2022/099159 or a salt thereof. In some embodiments, an oligonucleotide has a structure selected from Table 10 of WO 2022/099159 or a salt thereof.

In some embodiments, an oligonucleotide comprises an additional chemical moiety as described herein. In some embodiments, an additional chemical moiety facilitates delivery. In some embodiments, an additional chemical moiety comprises a targeting moiety. In some embodiments, an additional chemical moiety comprises one or more carbohydrate moieties. In some embodiments, an additional chemical moiety is a carbohydrate moiety. In some embodiments, an additional chemical moiety comprises one or more lipid moieties. In some embodiments, an additional chemical moiety is a lipid moiety. In some embodiments, an additional chemical moiety comprises one or more protein ligand moieties. In some embodiments, an additional chemical moiety targets liver. In some embodiments, an additional chemical moiety comprises one or more ligands of one or more receptors expressed in liver. In some embodiments, an additional chemical moiety is a ligand of one or more receptors expressed in liver. In some embodiments, an additional chemical moiety comprises one or more ligands for one or more asialoglycoprotein receptors. In some embodiments, an additional chemical moiety is a ligand for an asialoglycoprotein receptor. In some embodiments, an additional chemical moiety comprises multiple moieties, each of which is independently a ligand for an asialoglycoprotein receptor. In some embodiments, a ligand is GalNAc or a derivative thereof. In some embodiments, a ligand is GalNAc. In some embodiments, a ligand is

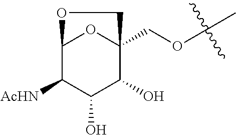 [SMILES: *OC[C@@]12CO[C@@H](O1)[C@H](NC(C)=O)[C@@H](O)[C@H]2O]

or a derivative thereof. In some embodiments, a ligand is

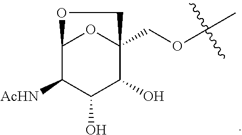 [SMILES: *OC[C@@]12CO[C@@H](O1)[C@H](NC(C)=O)[C@@H](O)[C@H]2O]

In some embodiments, an additional chemical moiety comprises GalNAc. In some embodiments, an additional chemical moiety is GalNAc. In some embodiments, an additional chemical moiety comprises multiple GalNAc. In some embodiments, an additional chemical moiety comprises three GalNAc. In some embodiments, an additional chemical moiety is or comprises

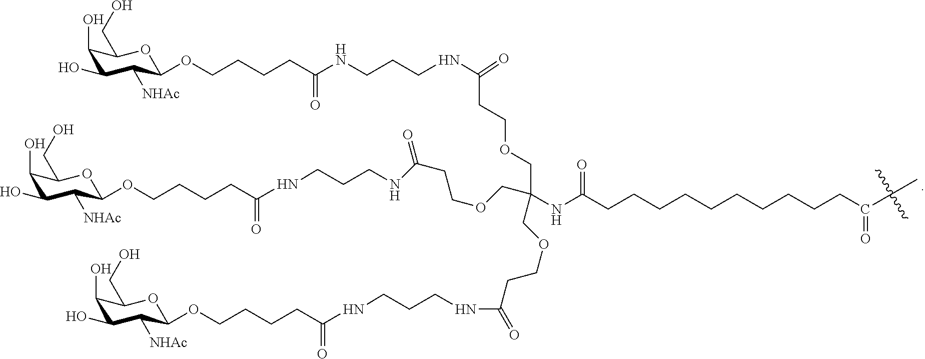

In some embodiments, an additional chemical moiety comprises

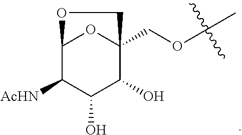 [SMILES: *OC[C@@]12CO[C@@H](O1)[C@H](NC(C)=O)[C@@H](O)[C@H]2O]

In some embodiments, an additional chemical moiety is

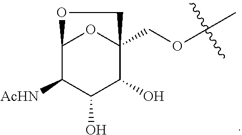 [SMILES: *OC[C@@]12CO[C@@H](O1)[C@H](NC(C)=O)[C@@H](O)[C@H]2O]

In some embodiments, an additional chemical moiety comprises multiple

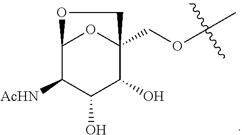 [SMILES: *OC[C@@]12CO[C@@H](O1)[C@H](NC(C)=O)[C@@H](O)[C@H]2O]

In some embodiments, an additional chemical moiety comprises three

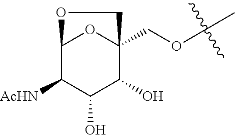 [SMILES: *OC[C@@]12CO[C@@H](O1)[C@H](NC(C)=O)[C@@H](O)[C@H]2O]

In some embodiments, an additional chemical moiety is or comprises

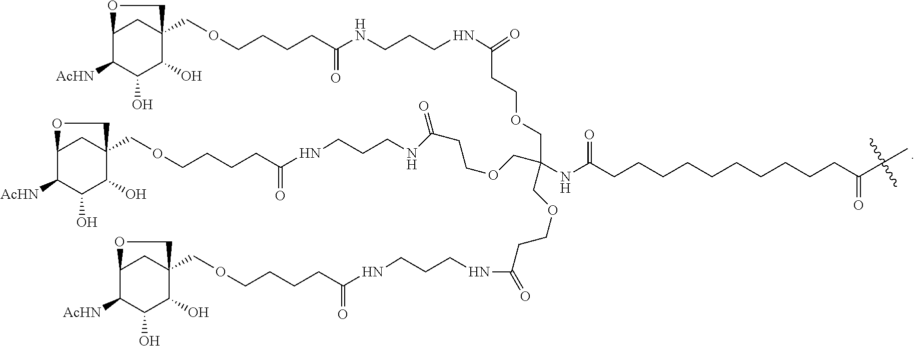 [SMILES: *C(=O)CCCCCCCCCCC(=O)NC(COCCC(=O)NCCCNC(=O)CCCCOC[C@@]12CO[C@@H](C1)[C@H](NC(C)=O)[C@@H](O)[C@H]2O)(COCCC(=O)NCCCNC(=O)CCCCOC[C@@]12CO[C@@H](C1)[C@H](NC(C)=O)[C@@H](O)[C@H]2O)COCCC(=O)NCCCNC(=O)CCCCOC[C@@]12CO[C@@H](C1)[C@H](NC(C)=O)[C@@H](O)[C@H]2O]

In some embodiments, an additional chemical moiety is directly conjugated to an oligonucleotide chain. In some embodiments, an additional chemical moiety is conjugated via a linker to an oligonucleotide chain. In some embodiments, two or more additional chemical moieties are conjugated via a linker to an oligonucleotide chain. In some embodiments, a linker is or comprises L001. In some embodiments, a linker is a polyvalent linker. In some embodiments, a polyvalent linker conjugates two or more additional chemical moieties. For example, in some embodiments, a tetravalent linker can connect three additional chemical moieties, e.g., three GalNAc, to a single point of an oligonucleotide chain. Additional chemical moieties may be independently connected to various locations of oligonucleotide chains independently and optionally through linkers. In some embodiments, an additional chemical moiety is conjugated to the 5′-end of the oligonucleotide chain. In some embodiments, an additional chemical moiety is conjugated to the 3′-end of the oligonucleotide chain. In some embodiments, an additional chemical moiety is conjugated to the middle of the oligonucleotide chain. In some embodiments, an additional chemical moiety is conjugated to a sugar. In some embodiments, an additional chemical moiety is conjugated to a nucleobase. In some embodiments, an additional chemical moiety is conjugated to an internucleotidic linkage. In some embodiments, a linker is connected to 5′-end 5′-carbon of an oligonucleotide chain. In some embodiments, a linker is connected to 3′-end 3′-carbon of an oligonucleotide chain. In some embodiments, a linker, e.g., L001, is connected to, e.g., 5′-end 5′-carbon of an oligonucleotide chain through a phosphate group. In some embodiments, it is through a phosphorothioate group.

In some embodiments, an oligonucleotide is

(SEQ ID NO: 13)
Mod001L001mCn001RmC*SmC*SfA*SfG*Sm5CcoAeofG*SfC*SmUmUn001RmCfA*SfGn001RfUm5Ceo*
SfC*SmCmUn001RmUTeofC*ST*Sb008U*SIn001SmUfC*SmG*SmAn001RmU,
(SEQ ID NO: 15)
Mod001L001mCn001RmC*SmC*SfA*SfG*Sm5CeoAcofG*SfC*STeofUn001RmCfA*SfGn001RfUm5Ceo*
SfC*SfC*SfUn001RTcoTeofC*ST*Sb008U*SIn001SmUfC*SmG*SmAn001RmU,
(SEQ ID NO: 3)
Mod001L001mCn001RmC*SmC*SfA*SfG*Sm5CeoAeofG*SfC*STeofUn001RmCfA*SfGn001RfUmC*
SfC*SfC*SfUn001RTeoTeofC*ST*Sb008U*SIn001SmUfC*SmG*SmAn001RmU,
(SEQ ID NO: 8)
Mod001L001mCh001RmC*SmC*SfA*SfG*Sm5CeoAcofG*SfC*STeofUn001RmCfA*SmGn001RfUm5Ceo*
SfC*SfC*SfUn001RTcoTeofC*ST*Sb008U*SIn001SmUfC*SmG*SmAn001RmU,
(SEQ ID NO: 7)
Mod001L001mCn001RmC*SmC*SfA*SfG*Sm5CeoAcofG*SfC*STeofUn001RmCfA*SmGn001RfUmC*
SfC*SfC*SfUn001RTeoTeofC*ST*Sb008U*SIn001SmUfC*SmG*SmAn001RmU,
(SEQ ID NO: 12)
Mod001L001mCn001RmC*SmC*SfA*SfG*Sm5CeoAeofG*Sm5CeoTeo*SmUn001Rm5CeofA*
SfGn001RmUm5Ccom5Ceo*SfC*SfU*STeoTeofC*ST*Sb008U*SIn001SmUfC*SmG*SmAn001RmU,
(SEQ ID NO: 10)
Mod001L001mCn001RmC*SmC*SfA*SfG*Sm5CeoAeofG*SmCTeo*SmUn001RmCfA*SfGn001RmUm5CeomC*
SfC*SfU*STeoTeofC*ST*Sb008U*SIn001SmUfC*SmG*SmAn001RmU,
(SEQ ID NO: 9)
Mod001L001mCn001RmC*SmC*SfA*SfG*Sm5CeoAcofG*SmCTeo*SmUn001RmCfA*SfGn001RmUmCmC*
SfC*SfU*STeoTeofC*ST*Sb008U*SIn001SmUfC*SmG*SmAn001RmU,
(SEQ ID NO: 1)
Mod001L001mCn001RmC*SmC*SfA*SfG*SmCmA*SfG*SfCmU*SfUn001RmCfA*SfGn001RfUmC*
SfC*SfC*SfU*SmUmUfC*ST*Sb008U*SIn001SmUfC*SmG*SmAn001RmU,
or
(SEQ ID NO: 5)
Mod001L001mCn001RmC*SmC*SfA*SfG*SmCmAfG*SfC*SmUfUn001RmCfA*SmGn001RfUmC*
SfC*SfC*SfUn001RmUmUfC*ST*Sb008U*SIn001SmUfC*SmG*SmAn001RmU.

In some embodiments, an oligonucleotide has a structure selected from Table 1 of WO 2022/099159, incorporated herein by reference, or a salt thereof, wherein the oligonucleotide targets SERPINA1 and comprises an additional chemical moiety, or a salt thereof. In some embodiments, an oligonucleotide has a structure selected from Table 1 of WO 2022/099159 or a salt thereof, wherein the oligonucleotide targets SERPINA11 and comprises L001. In some embodiments, an oligonucleotide has a structure selected from Table 1 of WO 2022/099159 or a salt thereof, wherein the oligonucleotide targets SERPINA1 and comprises Mod001. In some embodiments, an oligonucleotide has a structure selected from Table 1D of WO 2022/099159 or a salt thereof. In some embodiments, an oligonucleotide has a structure selected from Table 1E of WO 2022/099159 or a salt thereof. In some embodiments, an oligonucleotide has a structure selected from Table 1F of WO 2022/099159 or a salt thereof. In some embodiments, an oligonucleotide has a structure selected from WV-46312 to WV-46323 in Table 1F of WO 2022/099159 or a salt thereof. In some embodiments, an oligonucleotide has a structure selected from WV-47597 to WV-47609 in Table 1F of WO 2022/099159 or a salt thereof. In some embodiments, an oligonucleotide has a structure selected from WV-47641 to WV-48454 in Table 1F of WO 2022/099159 or a salt thereof. In some embodiments, an oligonucleotide has a structure selected from WV-47643 to WV-47648 in Table 1F of WO 2022/099159 or a salt thereof. In some embodiments, an oligonucleotide has a structure selected from WV-48453 to WV-48454 in Table 1F of WO 2022/099159 or a salt thereof. In some embodiments, an oligonucleotide has a structure selected from WV-49085 to WV-49093 in Table 1F of WO 2022/099159 or a salt thereof. In some embodiments, an oligonucleotide has a structure selected from Table 10 of WO 2022/099159 or a salt thereof.

As described herein, in some embodiments, an additional chemical moiety, e.g., Mod001, may facilitate delivery of an oligonucleotide. In some embodiments, after delivery additional chemical moiety is cleaved. In some embodiments, additional moieties are released after delivery or administration, providing oligonucleotides to be delivered. In some embodiments, linkers (e.g., LOO) for conjugating additional chemical moieties are cleaved. In some embodiments, an oligonucleotide has the structure of an oligonucleotide chain of an oligonucleotide comprising an additional chemical moiety and optionally a linker. In some embodiments, an oligonucleotide has the structure of a released oligonucleotide after an additional chemical moiety is cleaved from an oligonucleotide comprising an additional chemical moiety. In some embodiments, a linker, e.g., L001, is also cleaved from an oligonucleotide. In some embodiments, an oligonucleotide is formed by cleaving the additional chemical moiety from the oligonucleotide chain of an oligonucleotide comprising an additional chemical moiety. In some embodiments, an additional chemical moiety is cleaved after an oligonucleotide is delivered into a cell. In some embodiments, an additional chemical moiety is cleaved after an oligonucleotide is administered to a subject.

In some embodiments, the provided technology provides technologies for delivering an oligonucleotide, comprising administering a conjugate of the oligonucleotide, wherein the conjugate comprising the oligonucleotide to be delivered and an additional chemical moiety as described herein. In some embodiments, an oligonucleotide is conjugated with one or more additional chemical moieties independently and optionally through one or more linkers. In some embodiments, an oligonucleotide is conjugated with an additional chemical moiety through a linker.

Oligonucleotide, compounds, moieties, e.g., additional chemical moieties, linker moieties, etc. may contain groups that can be or comprise R′ or R as described herein. In some embodiments, R′ is R. In some embodiments, R′ is —C(O)R. In some embodiments, R′ is —C(O)OR. In some embodiments, R′ is —C(O)N(R)₂. In some embodiments, R′ is —SO₂R.

In some embodiments, R′ in various structures is a protecting group (e.g., for amino, hydroxyl, etc.), e.g., one suitable for oligonucleotide synthesis. In some embodiments, R is optionally substituted phenyl. In some embodiments, R is phenyl. In some embodiments, R is 4-nitrophenyl. In some embodiments, R is —CH₂CH₂-(4-nitrophenyl). In some embodiments, R′ is —C(O)NPh₂.

In some embodiments, each R is independently —H, or an optionally substituted group selected from C₁₋₁₀ aliphatic, C₁₋₁₀ heteroaliphatic having 1-5 heteroatoms, C₆₋₁₄ aryl, C₆₋₂₀ arylaliphatic, C₆₋₂₀ arylheteroaliphatic having 1-5 heteroatoms, 5-14 membered heteroaryl having 1-5 heteroatoms, and 3-10 membered heterocyclyl having 1-5 heteroatoms, or
two R groups are optionally and independently taken together to form a covalent bond, or:
two or more R groups on the same atom are optionally and independently taken together with the atom to form an optionally substituted, 3-15 membered, monocyclic, bicyclic or polycyclic ring having, in addition to the atom, 0-10 (e.g., 0, 1, 2, 3, 4, 5, 6, 7, 8, 9, or 10) heteroatoms; or:
two or more R groups on two or more atoms are optionally and independently taken together with their intervening atoms to form an optionally substituted, 3-15 membered, monocyclic, bicyclic or polycyclic ring having, in addition to the intervening atoms, 0-10 (e.g., 0, 1, 2, 3, 4, 5, 6, 7, 8, 9, or 10) heteroatoms.

In some embodiments, each R is independently —H, or an optionally substituted group selected from C₁₋₂₀ aliphatic, C₁₋₂₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-20 membered heteroaryl having 1-10 heteroatoms, and 3-30 membered heterocyclyl having 1-10 heteroatoms. In some embodiments, each R is independently —H, or an optionally substituted group selected from C₁₋₁₀ aliphatic, C₁₋₁₀ heteroaliphatic having 1-5 heteroatoms, C₆₋₁₄ aryl, C₆₋₂₀ arylaliphatic, C₆₋₂₀ arylheteroaliphatic having 1-5 heteroatoms, 5-14 membered heteroaryl having 1-5 heteroatoms, and 3-10 membered heterocyclyl having 1-5 heteroatoms. In some embodiments, two R groups are optionally and independently taken together to form a covalent bond. In some embodiments, two or more R groups on the same atom are optionally and independently taken together with the atom to form an optionally substituted, 3-20 membered, monocyclic, bicyclic or polycyclic ring having, in addition to the atom, 0-10 heteroatoms. In some embodiments, two groups on the same atom are optionally and independently taken together with the atom to form an optionally substituted, 3-20 membered, monocyclic, bicyclic or polycyclic ring having, in addition to the atom, 0-10 heteroatoms. In some embodiments, two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms. In some embodiments, two groups on two or more atoms are optionally and independently taken together with their intervening atoms to form an optionally substituted, 3-30 membered, monocyclic, bicyclic or polycyclic ring having, in addition to the intervening atoms, 0-10 heteroatoms. In some embodiments, a formed ring is monocyclic. In some embodiments, a formed ring is bicyclic. In some embodiments, a formed ring is polycyclic. In some embodiments, each monocyclic ring unit is independently 3-10 (e.g., 3-8, 3-7, 3-6, 5-10, 5-8, 5-7, 5-6, 3, 4, 5, 6, 7, 8, 9, or 10, etc.) membered, and is independently saturated, partially saturated, or aromatic, and independently has 0-5 heteroatom. In some embodiments, a ring is saturated. In some embodiments, a ring is partially saturated. In some embodiments, a ring is aromatic. In some embodiments, a formed ring has 1-5 heteroatom. In some embodiments, a formed ring has 1 heteroatom. In some embodiments, a formed ring has 2 heteroatoms. In some embodiments, a heteroatom is nitrogen. In some embodiments, a heteroatom is oxygen.

In some embodiments, R is —H.

In some embodiments, R is optionally substituted C₁₋₂₀, C₁₋₁₅, C₁₋₁₀, C₁₋₈, C₁₋₆, C₁₋₅, C₁₋₄, C₁₋₃, or C₁₋₂ aliphatic. In some embodiments, R is optionally substituted alkyl. In some embodiments, R is optionally substituted C₁₋₆ alkyl. In some embodiments, R is optionally substituted methyl. In some embodiments, R is optionally substituted cycloaliphatic. In some embodiments, R is optionally substituted cycloalkyl.

In some embodiments, R is optionally substituted C₁₋₂₀ heteroaliphatic having 1-10 heteroatoms.

In some embodiments, R is optionally substituted C₆₋₂₀ aryl. In some embodiments, R is optionally substituted phenyl. In some embodiments, R is phenyl.

In some embodiments, R is optionally substituted C₆₋₂₀ arylaliphatic. In some embodiments, R is optionally substituted C₆₋₂₀ arylalkyl. In some embodiments, R is benzyl. In some embodiments, R is optionally substituted C₆₋₂₀ arylheteroaliphatic having 1-10 heteroatoms.

In some embodiments, R is optionally substituted 5-20 membered heteroaryl having 1-10 heteroatoms. In some embodiments, R is optionally substituted 5-membered heteroaryl having 1-4 heteroatoms. In some embodiments, R is optionally substituted 6-membered heteroaryl having 1-4 heteroatoms. In some embodiments, R is optionally substituted 3-20 membered heterocyclyl having 1-10 heteroatoms. In some embodiments, R is optionally substituted 3-10 membered heterocyclyl having 1-5 heteroatoms. In some embodiments, R is optionally substituted 5-6 membered heterocyclyl having 1-5 heteroatoms. In some embodiments, a heterocyclyl is saturated. In some embodiments, a heterocyclyl is partially saturated.

In some embodiments, a heteroatom is selected from boron, nitrogen, oxygen, sulfur, silicon and phosphorus. In some embodiments, a heteroatom is selected from nitrogen, oxygen, sulfur, and silicon. In some embodiments, a heteroatom is selected from nitrogen, oxygen, and sulfur. In some embodiments, a heteroatom is nitrogen. In some embodiments, a heteroatom is oxygen. In some embodiments, a heteroatom is sulfur.

Certain oligonucleotides and/or compositions referred to in the present disclosure are described in WO 2021/071858 or WO 2022/099159 or a priority application, e.g., in Table 1 of WO 2021/071858 or WO 2022/099159 or a priority application. All oligonucleotides and/or compositions of WO 2021/071858 and WO 2022/099159 are incorporated herein by reference.

Certain oligonucleotides and/or compositions are described in Table 1 below.

TABLE 1
Example oligonucleotides and/or compositions that target SERPINA1.

| | | SEQ | | |
|---|---|---|---|---|
| | | ID | | Stereochemistry/ |
| ID | Description | NO | Base Sequence | Linkage |
| WV- | Mod001L001mCn001RmC*SmC*SfA* | 1 | CCCAGCAGCUUC | OnRSSSSOSSO |
| 46312 | SfG*SmCmA*SfG*SfCmU*SfUn001R | | AGUCCCUUUCTUI | SnROSnROSSS |
| | mCfA*SfGn001RfUmC*SfC*SfC*SfU | | UCGAU | SOOSSSnSOSS |
| | *SmUmUfC*ST*Sb008U*SIn001SmU | | | nR |
| | fC*SmG*SmAn001RmU | | | |
| WV- | mCn001RmC*SmC*SfA*SfG*SmCm | 2 | CCCAGCAGCUUC | nRSSSSOSSOS |
| 44515 | A*SfG*SfCmU*SfUn001RmCfA*SfG | | AGUCCCUUUCTUI | nROSnROSSSS |
| | n001RfUmC*SfC*SfC*SfU*SmUmUf | | UCGAU | OOSSSnSOSSn |
| | C*ST*Sb008U*SIn001SmUfC*SmG*S | | | R |
| | mAn001RmU | | | |
| WV- | Mod001L001mCn001RmC*SmC*SfA* | 3 | CCCAGCAGCTUC | OnRSSSSOOSS |
| 47606 | SfG*Sm5CeoAeofG*SfC*STeofUn001 | | AGUCCCUTTCTUI | OnROSnROSSS |
| | RmCfA*SfGn001RfUmC*SfC*SfC*Sf | | UCGAU | nROOSSSnSOS |
| | Un001RTeoTeofC*ST*Sb008U*SIn00 | | | SnR |
| | 1SmUfC*SmG*SmAn001RmU | | | |
| WV- | mCn001RmC*SmC*SfA*SfG*Sm5Ceo | 4 | CCCAGCAGCTUC | nRSSSSOOSSO |
| 46460 | AcofG*SfC*STeofUn001RmCfA*SfGn | | AGUCCCUTTCTUI | BROSnROSSSn |
| | 001RfUmC*SfC*SfC*SfUn001RTeoTe | | UCGAU | ROOSSSnSOSS |
| | ofC*ST*Sb008U*SIn001SmUfC*SmG | | | nR |
| | *SmAn001RmU | | | |
| WV- | Mod001L001mCn001RmC*SmC*SfA* | 5 | CCCAGCAGCUUC | OnRSSSSOOSS |
| 47608 | SfG*SmCmAfG*SfC*SmUfUn001Rm | | AGUCCCUUUCTUI | OnROSnROSSS |
| | CfA*SmGn001RfUmC*SfC*SfC*SfUn | | UCGAU | nROOSSSnSOS |
| | 001RmUmUfC*ST*Sb008U*SIn001S | | | SnR |
| | mUfC*SmG*SmAn001RmU | | | |
| WV- | mCn001RmC*SmC*SfA*SfG*SmCm | 6 | CCCAGCAGCUUC | nRSSSSOOSSO |
| 47037 | AfG*SfC*SmUfUn001RmCfA*SmGn0 | | AGUCCCUUUCTUI | nROSnROSSSn |
| | 01RfUmC*SfC*SfC*SfUn001RmUmU | | UCGAU | ROOSSSnSOSS |
| | fC*ST*Sb008U*SIn001SmUfC*SmG* | | | nR |
| | SmAn001RmU | | | |
| WV- | Mod001L001mCn001RmC*SmC*SfA* | 7 | CCCAGCAGCTUC | OnRSSSSOOSS |
| 49085 | SfG*Sm5CeoAeofG*SfC*STeofUn001 | | AGUCCCUTTCTUI | OnROSnROSSS |
| | RmCfA*SmGn001RfUmC*SfC*SfC*S | | UCGAU | nROOSSSnSOS |
| | fUn001RTeoTeofC*ST*Sb008U*SIn00 | | | SnR |
| | 1SmUfC*SmG*SmAn001RmU | | | |
| WV- | mCn001RmC*SmC*SfA*SfG*Sm5Ceo | 29 | CCCAGCAGCTUC | nRSSSSOOSSO |
| 49085b | AeofG*SfC*STeofUn001RmCfA*SmG | | AGUCCCUTTCTUI | nROSnROSSSn |
| | n001RfUmC*SfC*SfC*SfUn001RTeoT | | UCGAU | ROOSSSnSOSS |
| | cofC*ST*Sb008U*SIn001SmUfC*Sm | | | nR |
| | G*SmAn001RmU | | | |
| WV- | Mod001L001mCn001RmC*SmC*SfA* | 8 | CCCAGCAGCTUC | OnRSSSSOOSS |
| 49086 | SfG*Sm5CeoAcofG*SfC*STeofUn001 | | AGUCCCUTTCTUI | OnROSnROSSS |
| | RmCfA*SmGn001RfUm5Ceo*SfC*Sf | | UCGAU | nROOSSSnSOS |
| | C*SfUn001RTeoTeofC*ST*Sb008U*S | | | SnR |
| | In001SmUfC*SmG*SmAn001RmU | | | |
| WV- | mCn001RmC*SmC*SfA*SfG*Sm5Ceo | 30 | CCCAGCAGCTUC | nRSSSSOOSSO |
| 49086b | AcofG*SfC*STeofUn001RmCfA*SmG | | AGUCCCUTTCTUI | nROSnROSSSn |
| | n001RfUm5Ceo*SfC*SfC*SfUn001RT | | UCGAU | ROOSSSASOSS |
| | eoTeofC*ST*Sb008U*SIn001SmUfC* | | | nR |
| | SmG*SmAn001RmU | | | |
| WV- | Mod001L001mCn001RmC*SmC*SfA* | 9 | CCCAGCAGCTUC | OnRSSSSOOS |
| 49087 | SfG*Sm5CeoAeofG*SmCTeo*SmUn0 | | AGUCCCUTTCTUI | OSnROSnROO |
| | 01RmCfA*SfGn001RmUmCmC*SfC* | | UCGAU | SSSOOSSSnSO |
| | SfU*STeoTeofC*ST*Sb008U*SIn001S | | | SSnR |
| | mUfC*SmG*SmAn001RmU | | | |
| WV- | mCn001RmC*SmC*SfA*SfG*Sm5Ceo | 31 | CCCAGCAGCTUC | nRSSSSOOSOS |
| 49087b | AeofG*SmCTeo*SmUn001RmCfA*Sf | | AGUCCCUTTCTUI | nROSnROOSSS |
| | Gn001RmUmCmC*SfC*SfU*STeoTeo | | UCGAU | OOSSSnSOSSn |
| | fC*ST*Sb008U*SIn001SmUfC*SmG* | | | R |
| | SmAn001RmU | | | |
| WV- | Mod001L001mCn001RmC*SmC*SfA* | 10 | CCCAGCAGCTUC | OnRSSSSOOS |
| 49088 | SfG*Sm5CcoAeofG*SmCTeo*SmUn0 | | AGUCCCUTTCTUI | OSnROSnROO |
| | 01RmCfA*SfGn001RmUm5CeomC*Sf | | UCGAU | SSSOOSSSnSO |
| | C*SfU*STeoTeofC*ST*Sb008U*SIn0 | | | SSnR |
| | 01SmUfC*SmG*SmAn001RmU | | | |
| WV- | mCn001RmC*SmC*SfA*SfG*Sm5CeoA | 11 | CCCAGCAGCTUCA | nRSSSSOOSOSn |
| 50496 | eofG*SmCTeo*SmUn001RmCfA*SfGn0 | | GUCCCUTTCTUIUC | ROSnROOSSSO |
| | 01RmUm5CeomC*SfC*SfU*STeoTeofC | | GAU | OSSSnSOSSnR |
| | *ST*Sb008U*SIn001SmUfC*SmG*Sm | | | |
| | An001RmU | | | |
| WV- | Mod001L001mCn001RmC*SmC*SfA* | 12 | CCCAGCAGCTUC | OnRSSSSOOS |
| 49089 | SfG*Sm5CeoAcofG*Sm5CeoTeo*Sm | | AGUCCCUTTCTUI | OSnROSnROO |
| | Un001Rm5CeofA*SfGn001RmUm5Ce | | UCGAU | SSSOOSSSnSO |
| | om5Ceo*SfC*SfU*STeoTeofC*ST*Sb | | | SSnR |
| | 008U*SIn001SmUfC*SmG*SmAn001 | | | |
| | RmU | | | |
| WV- | mCn001RmC*SmC*SfA*SfG*Sm5Ceo | 32 | CCCAGCAGCTUC | nRSSSSOOSOS |
| 49089b | AcofG*Sm5CeoTeo*SmUn001Rm5Ce | | AGUCCCUTTCTUI | nROSnROOSSS |
| | ofA*SfGn001RmUm5Ceom5Ceo*SfC* | | UCGAU | OOSSSnSOSSn |
| | SfU*STeoTeofC*ST*Sb008U*SIn001S | | | R |
| | mUfC*SmG*SmAn001RmU | | | |
| WV- | Mod001L001mCn001RmC*SmC*SfA* | 13 | CCCAGCAGCUUC | OnRSSSSOOSS |
| 49090 | SfG*Sm5CeoAcofG*SfC*SmUmUn00 | | AGUCCCUUTCTUI | OnROSnROSS |
| | 1RmCfA*SfGn001RfUm5Ceo*SfC*S | | UCGAU | OnROOSSSnSO |
| | mCmUn001RmUTeofC*ST*Sb008U*S | | | SSnR |
| | In001SmUfC*SmG*SmAn001RmU | | | |
| WV- | mCn001RmC*SmC*SfA*SfG*Sm5CeoA | 14 | CCCAGCAGCUUCA | nRSSSSOOSSOn |
| 50497 | eofG*SfC*SmUmUn001RmCfA*SfGn00 | | GUCCCUUTCTUIUC | ROSnROSSOnR |
| | 1RfUmSCeo*SfC*SmCmUn001RmUTeo | | GAU | OOSSSnSOSSnR |
| | fC*ST*Sb008U*SIn001SmUfC*SmG*S | | | |
| | mAn001RmU | | | |
| WV- | Mod001L001mCn001RmC*SmC*SfA* | 15 | CCCAGCAGCTUC | OnRSSSSOOSS |
| 49092 | SfG*Sm5CeoAeofG*SfC*STeofUn001 | | AGUCCCUTTCTUI | OnROSnROSSS |
| | RmCfA*SfGn001RfUm5Ceo*SfC*SfC | | UCGAU | nROOSSSnSOS |
| | *SfUn001RTeoTeofC*ST*Sb008U*SIn | | | SnR |
| | 001SmUfC*SmG*SmAn001RmU | | | |
| WV- | mCn001RmC*SmC*SfA*SfG*Sm5CeoA | 16 | CCCAGCAGCTUCA | nRSSSSOOSSOn |
| 50498 | cofG*SfC*STeofUn001RmCfA*SfGn00 | | GUCCCUTTCTUIUC | ROSnROSSSnRO |
| | 1RfUm5Ceo*SfC*SfC*SfUn001RTeoTe | | GAU | OSSSnSOSSnR |
| | ofC*ST*Sb008U*SIn001SmUfC*SmG* | | | |
| | SmAn001RmU | | | |

### Notes:

Description, Base Sequence and Stereochemistry/Linkage, due to their length, may be divided into multiple lines in Table 1. Unless otherwise specified, all oligonucleotides in Table 1 are single-stranded. As appreciated by those skilled in the art, nucleoside units are unmodified and contain unmodified nucleobases and 2′-deoxy sugars unless otherwise indicated (e.g., with r, m, m5, eo, etc.); linkages, unless otherwise indicated, are natural phosphate linkages; and acidic/basic groups may independently exist in their salt forms. If a sugar is not specified, the sugar is a natural DNA sugar; and if an internucleotidic linkage is not specified, the internucleotidic linkage is a natural phosphate linkage. Moieties and modifications:
m: 2′-OMe;
I: nucleobase is hypoxanthine:
f: 2′-F;
eo: 2′-MOE (2′-OCH₂CH₂OCH₃);
m5Ceo: 5-methyl 2′-O-methoxyethyl C:
O, PO: phosphodiester (phosphate). It can a linkage or be an end group (or a component thereof), e.g., a linkage between a linker and an oligonucleotide chain, an internucleotidic linkage (a natural phosphate linkage), etc. Phosphodiesters are typically indicated with “O” in the Stereochemistry/Linkage column and are typically not marked in the Description column (if it is an end group, e.g., a 5′-end group, it is indicated in the Description and typically not in Stereochemistry/Linkage); if no linkage is indicated in the Description column, it is typically a phosphodiester unless otherwise indicated. Note that a phosphate linkage between a linker (e.g., L001) and an oligonucleotide chain may not be marked in the Description column, but may be indicated with “O” in the Stereochemistry/Linkage column;
*, PS: Phosphorothioate. It can be an end group (if it is an end group, e.g., a 5′-end group, it is indicated in the Description and typically not in Stereochemistry/Linkage), or a linkage, e.g., a linkage between linker (e.g., L001) and an oligonucleotide chain, an internucleotidic linkage (a phosphorothioate internucleotidic linkage), etc.;
S, Sp: Phosphorothioate in the Sp configuration. Note that * S in Description indicates a single phosphorothioate linkage in the Sp configuration;
n001:

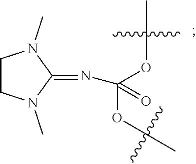

nR (when utilized for n001) or n001R: n001 in Rp configuration;
nS (when utilized for n001) or n001S: n001 in Sp configuration:
Mod001:

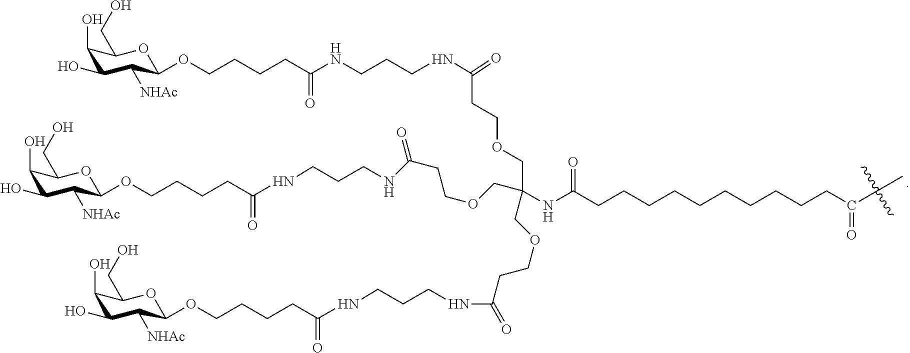

L001: —NH—(CH₂)₆— linker (C6 linker, C6 amine linker or C6 amino linker), connected to Mod (e.g., Mod001) through —NH— (e.g., forming an amide group —C(O)—NH—), and, in various cases, the 5-end of the oligonucleotide chain through a phosphate linkage (O or PO). For example, in WV-39306, L001 is connected to Mod001 through —NH— (forming an amide group —C(O)—NH—), and is connected to the oligonucleotide chain through a phosphate linkage (O). In case that Mod is not present, the —NH— is bonded to —H:
b008U: a nucleoside whose base is

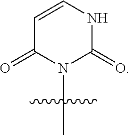 [SMILES: *n1c(=O)cc[nH]c1=O]

In some embodiments, the present disclosure provides a compound having the structure of formula A-1 or a salt thereof. In some embodiments, WV-46312 is provided, administered or delivered as one or more compounds each independently having the structure of formula A-1 or a salt thereof. In some embodiments, a composition of WV-46312 comprises one or more compounds each independently having the structure of formula A-1 or a salt thereof.

In some embodiments, the present disclosure provides a compound having the structure of formula A-2 or a salt thereof. In some embodiments, WV-49090 is provided, administered or delivered as one or more compounds each independently having the structure of formula A-2 or a salt thereof. In some embodiments, a composition of WV-49090 comprises one or more compounds each independently having the structure of formula A-2 or a salt thereof.

In some embodiments, the present disclosure provides a compound having the structure of formula A-3 or a salt thereof. In some embodiments, WV-49092 is provided, administered or delivered as one or more compounds each independently having the structure of formula A-3 or a salt thereof. In some embodiments, a composition of WV-49092 comprises one or more compounds each independently having the structure of formula A-3 or a salt thereof.

In some embodiments, the present disclosure provides a compound having the structure of formula B-1 or a salt thereof. In some embodiments, WV-44515 is provided, administered or delivered as one or more compounds each independently having the structure of formula B-1 or a salt thereof. In some embodiments, a composition of WV-44515 comprises one or more compounds each independently having the structure of formula B-1 or a salt thereof.

In some embodiments, the present disclosure provides a compound having the structure of formula B-2 or a salt thereof. In some embodiments, WV-50497 is provided, administered or delivered as one or more compounds each independently having the structure of formula B-2 or a salt thereof. In some embodiments, a composition of WV-50497 comprises one or more compounds each independently having the structure of formula B-2 or a salt thereof.

In some embodiments, the present disclosure provides a compound having the structure of formula B-3 or a salt thereof. In some embodiments, WV-50498 is provided, administered or delivered as one or more compounds each independently having the structure of formula B-3 or a salt thereof. In some embodiments, a composition of WV-50498 comprises one or more compounds each independently having the structure of formula B-3 or a salt thereof.

In some embodiments, a salt is a pharmaceutically acceptable salt. In some embodiments, each salt is independently a pharmaceutically acceptable salt.

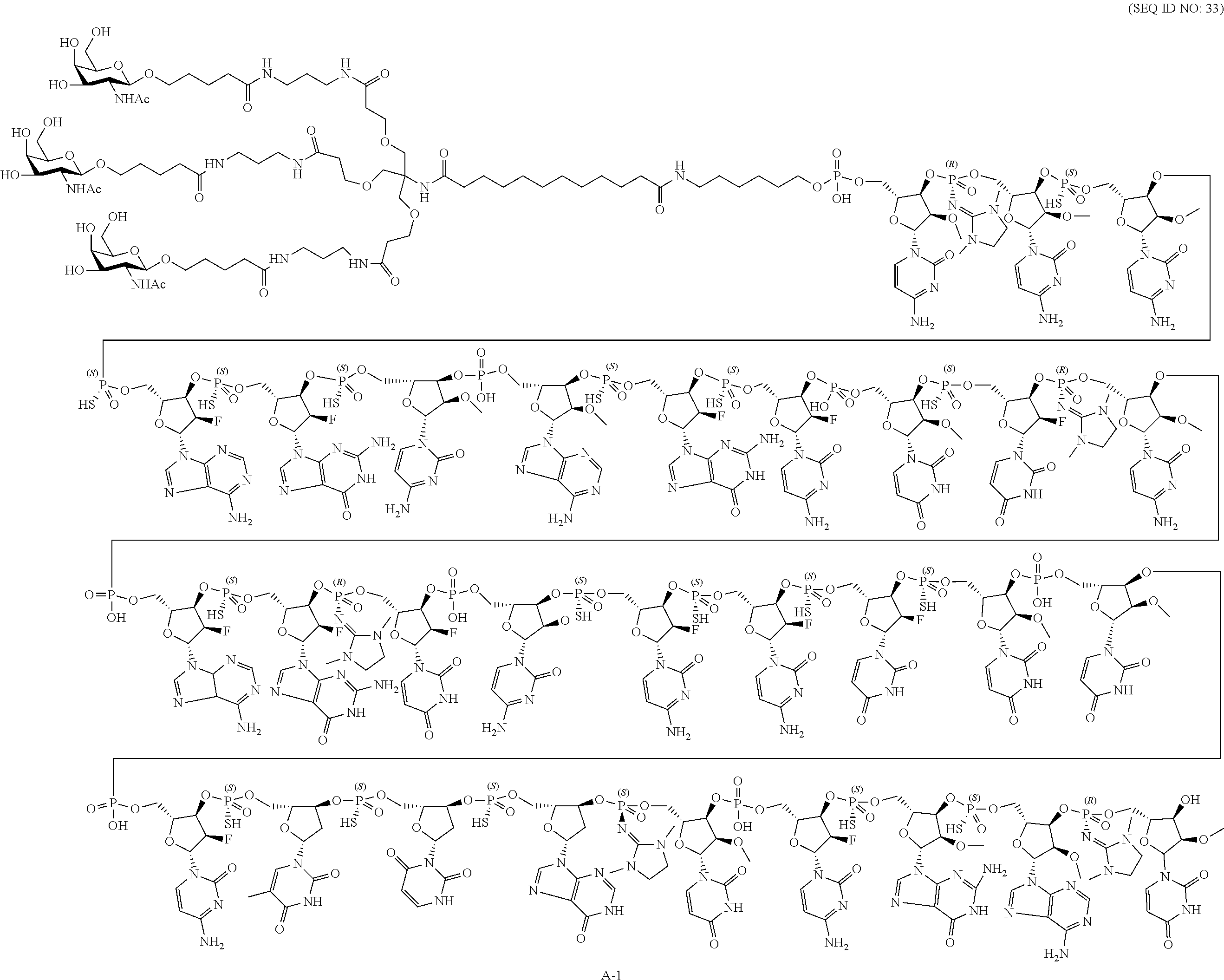

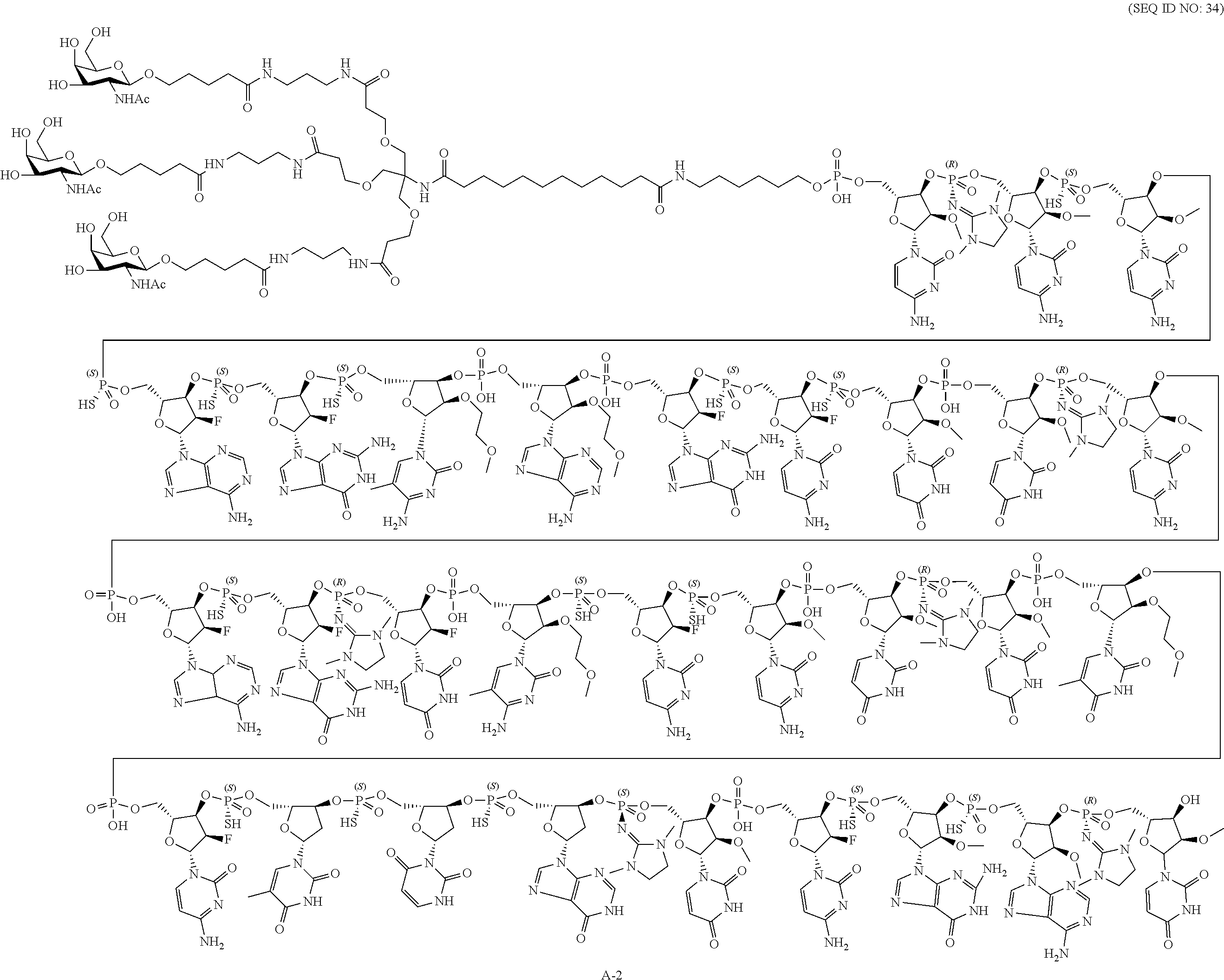

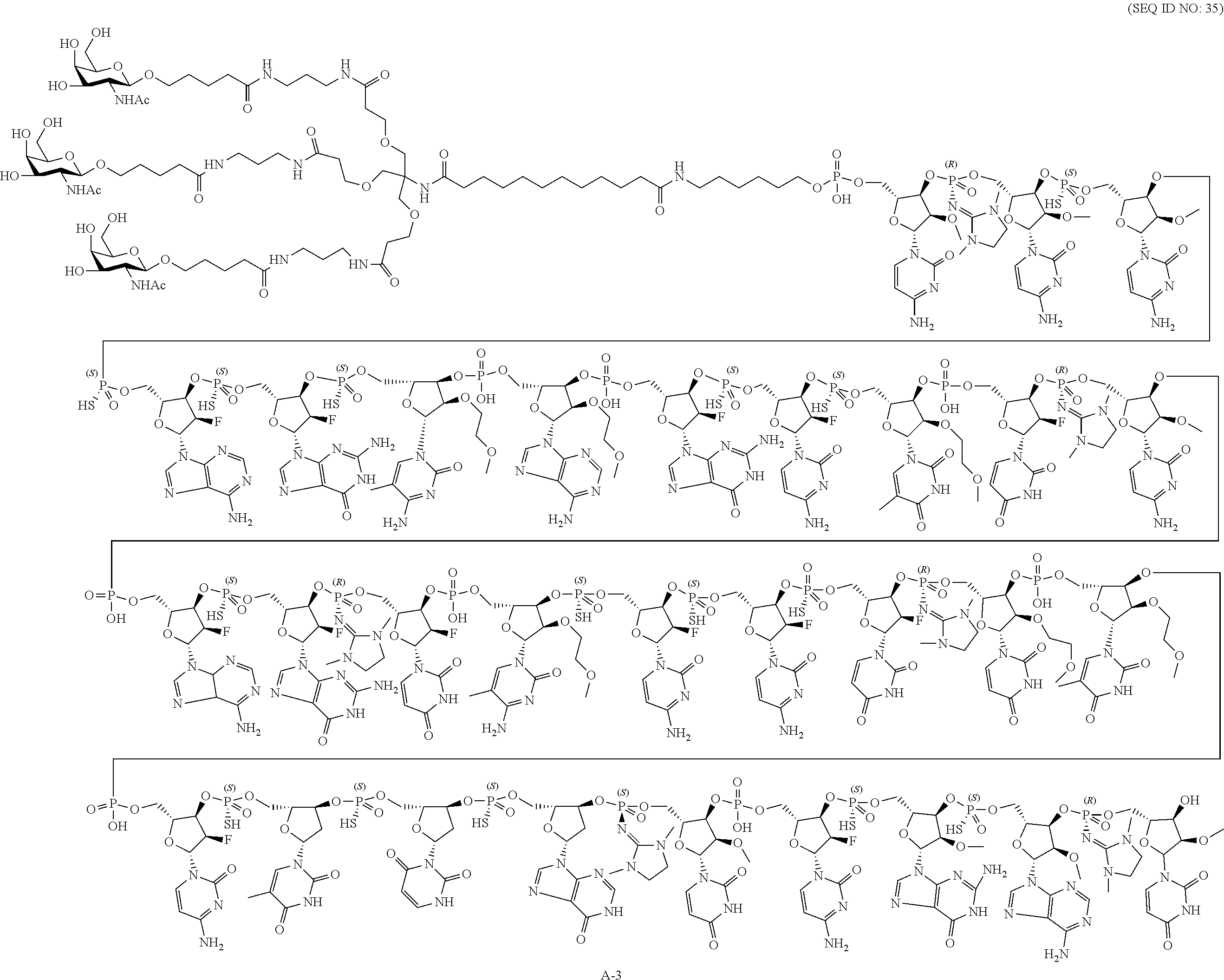

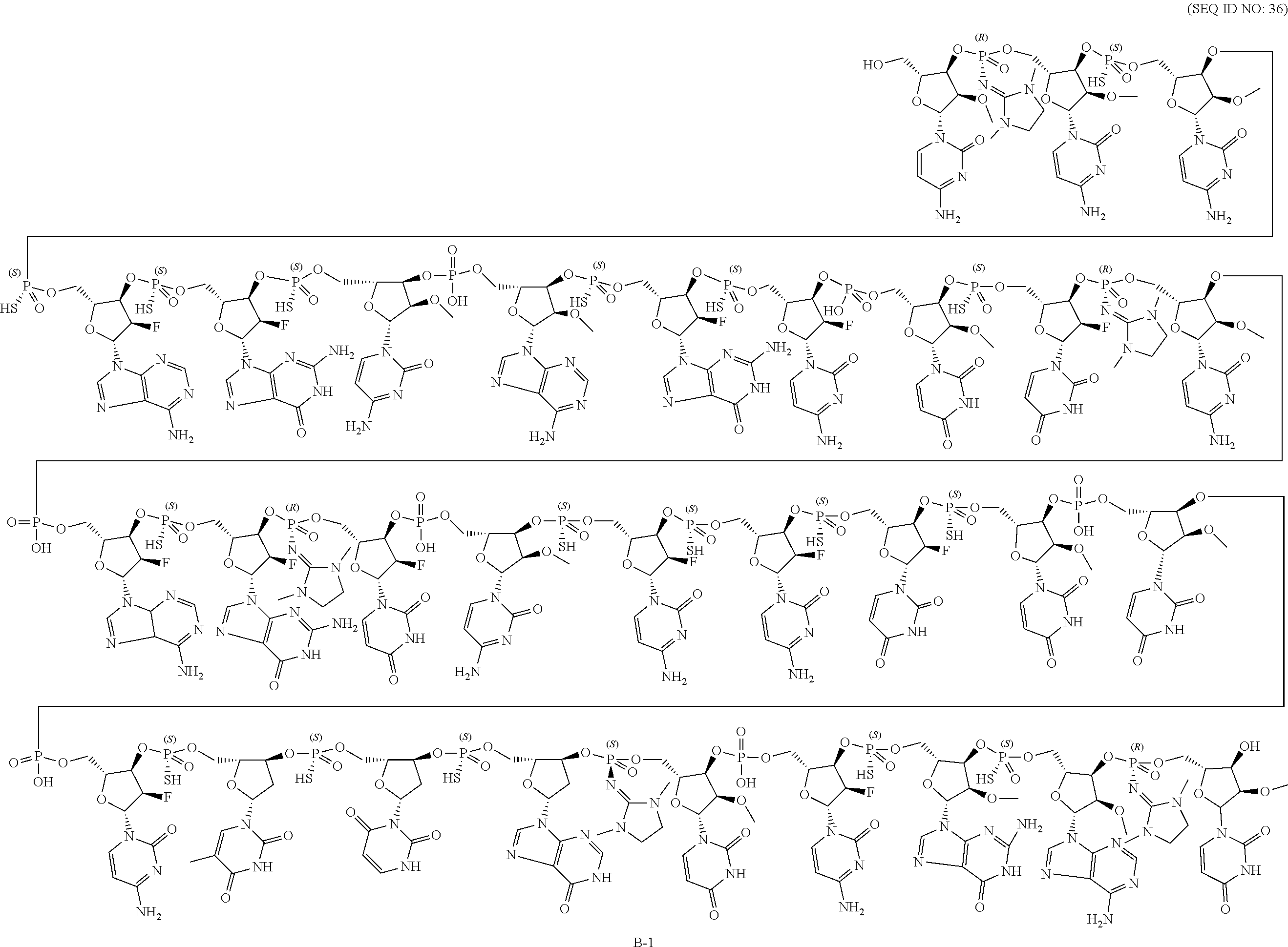

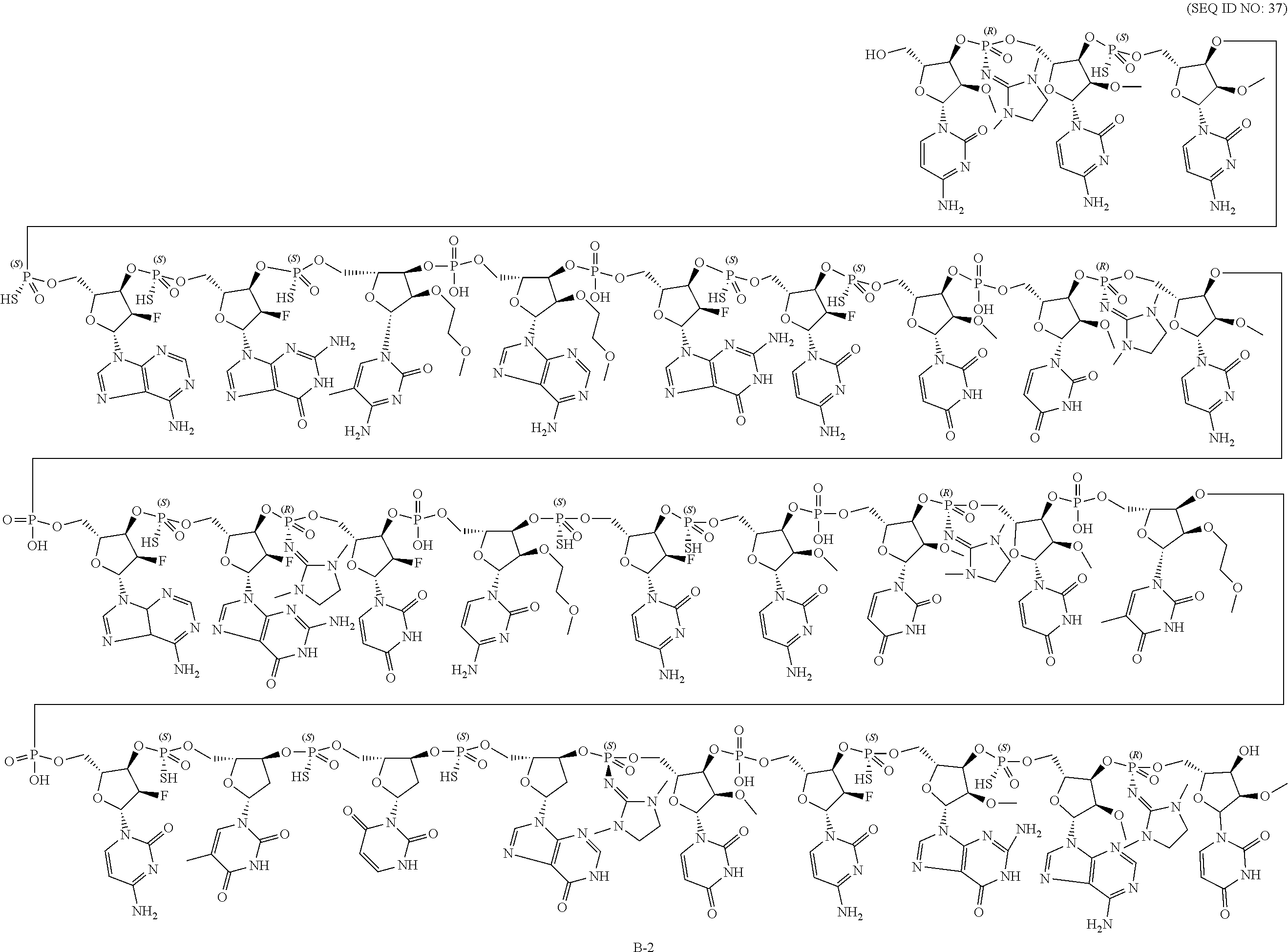

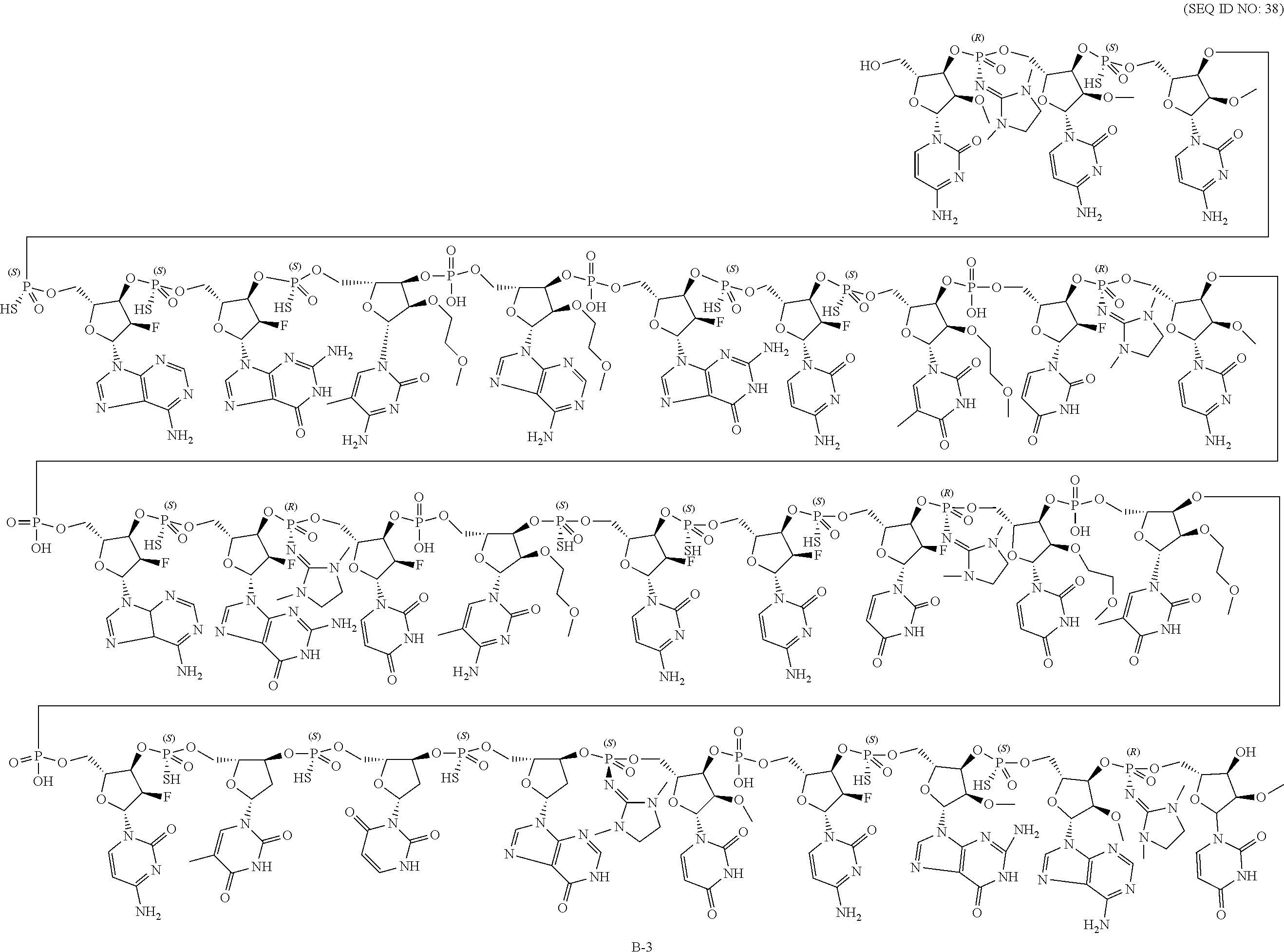

Zig-zag lines represent linkages between oxygen at 3′ to phosphorous in internucleotide linkages.

Compounds and oligonucleotides can be prepared in high purity in accordance with the present disclosure, e.g., through chirally controlled formation of chiral internucleotidic linkages such as phosphorothioate internucleotidic linkages. n001 linkages, etc. In some embodiments, diastereopurity of a compound or oligonucleotide is about or at least about (DS)^{nc}, wherein DS is about 85%-100% (e.g., about 85%, 86%, 87%, 88%, 89%, 90%, 91%, 92%, 93%, 94%, 95%, 96%, 97%, 98%, 99% or 99.5% or more) and nc is the number of chiral linkage phosphorus. In some embodiments, DS is about or at least about 90%. In some embodiments, DS is about or at least about 91%. In some embodiments, DS is about or at least about 92%. In some embodiments, DS is about or at least about 93%. In some embodiments, DS is about or at least about 94%. In some embodiments, DS is about or at least about 95%. In some embodiments, DS is about or at least about 96%. In some embodiments, DS is about or at least about 97%. In some embodiments, DS is about or at least about 98%. In some embodiments, DS is about or at least about 99%. In some embodiments, diastereopurity is determined as the product of the diastereopurity of each chiral internucleotidic linkage in the oligonucleotides. In some embodiments, diastereopurity of an internucleotidic linkage connecting two nucleosides in an oligonucleotide (or nucleic acid) is represented by the diastereopurity of an internucleotidic linkage of a dimer connecting the same two nucleosides, wherein the dimer is prepared using comparable conditions, in some instances, identical synthetic cycle conditions (e.g., for the linkage between Nx and Ny in an oligonucleotide . . . . NxNy . . . , the dimer is NxNy).

In some embodiments, diastereomeric excess of one or more (e.g., 5, 6, 7, 8, 9, 10, 11, 12, 13, 14, 15, 16, 17, 18, 19, 20 or more) chiral linkage phosphorus centers is independently about or at least about 80%, 85%, 90%, 91%, 92%, 93%, 94%, 95%, 96%, 97%, 98% or 99%. In some embodiments, diastereomeric excess of one or more (e.g., 5, 6, 7, 8, 9, 10, 11, 12, 13, 14, 15, 16, 17, 18, 19, 20 or more) chiral linkage phosphorus centers is independently about or at least about 90%, 91%, 92%, 93%, 94%, 95%, 96%, 97%, 98% or 99%. In some embodiments, diastereomeric excess of each phosphorothioate linkage phosphorus is independently about or at least about 80%, 85%, 90%, 91%, 92%, 93%, 94%, 95%, 96%, 97%, 98% or 99%. In some embodiments, diastereomeric excess of each phosphorothioate linkage phosphorus is independently about or at least about 90%, 91%, 92%, 93%, 94%, 95%, 96%, 97%, 98% or 99%. In some embodiments, diastereomeric excess of each phosphorothioate linkage phosphorus is independently about or at least about 95%. In some embodiments, diastereomeric excess of each phosphorothioate linkage phosphorus is independently about or at least about 96%. In some embodiments, diastereomeric excess of each phosphorothioate linkage phosphorus is independently about or at least about 97%. In some embodiments, diastereomeric excess of each phosphorothioate linkage phosphorus is independently about or at least about 98%. In some embodiments, diastereomeric excess of each chiral linkage phosphorus centers is independently about or at least about 80%, 85%, 90%, 91%, 92%, 93%, 94%, 95%, 96%, 97%, 98% or 99%. In some embodiments, diastereomeric excess of each chiral linkage phosphorus centers is independently about or at least about 90%, 91%, 92%, 93%, 94%, 95%, 96%, 97%, 98% or 99%. In some embodiments, diastereomeric excess of each chiral linkage phosphorus centers is independently about or at least about 95%. In some embodiments, diastereomeric excess of each chiral linkage phosphorus centers is independently about or at least about 96%. In some embodiments, diastereomeric excess of each chiral linkage phosphorus centers is independently about or at least about 97%. In some embodiments, diastereomeric excess of each chiral linkage phosphorus centers is independently about or at least about 98%.

In some embodiments, an oligonucleotide or compound has a purity of about 10%-100% (e.g., about 10%-95%, 50%-80%, 50%-85%, 50%-90%, 50%-95%, 60% A80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, or about or at least about 10%, 20%, 30%, 40%, 50%, 60%, 65%, 70%, 75%, 80%, 85%, 90%, 95%, or 100%, etc.). In some embodiments, an oligonucleotide has a purity of about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, or at least about 50%, 60%, 65%, 70%, 75%, 80%, 85%, 90%, 95%, or 100%, etc.). In some embodiments, purity is presented as area % of a UV trace of a separation technologies, e.g., HPLC, UPLC, etc. at 260 nM.

### Oligonucleotide Compositions

Among other things, the present disclosure provides various oligonucleotide compositions. In some embodiments, the present disclosure provides oligonucleotide compositions of oligonucleotides described herein. In some embodiments, an oligonucleotide composition comprises a plurality of oligonucleotides described in the present disclosure. In some embodiments, an oligonucleotide composition is chirally controlled. In some embodiments, an oligonucleotide composition is not chirally controlled (stereorandom).

Linkage phosphorus of natural phosphate linkages is achiral. Linkage phosphorus of many modified internucleotidic linkages, e.g., phosphorothioate internucleotidic linkages, are chiral. In some embodiments, during preparation of oligonucleotide compositions (e.g., in traditional phosphoramidite oligonucleotide synthesis), configurations of chiral linkage phosphorus are not purposefully designed or controlled, creating non-chirally controlled (stereorandom) oligonucleotide compositions (substantially racemic preparations) which are complex, random mixtures of various stereoisomers (diastereoisomers)—for oligonucleotides with n chiral internucleotidic linkages (linkage phosphorus being chiral), typically 2n stereoisomers (e.g., when n is 10, 210=1,032; when n is 20, 220=1,048,576). These stereoisomers have the same constitution, but differ with respect to the pattern of stereochemistry of their linkage phosphorus.

Stereoisomers within stereorandom compositions may have different properties, activities, and/or toxicities, resulting in inconsistent therapeutic effects and/or unintended side effects by stereorandom compositions, particularly compared to certain chirally controlled oligonucleotide compositions of oligonucleotides of the same constitution.

In some embodiments, the present disclosure encompasses technologies for designing and preparing chirally controlled oligonucleotide compositions. In some embodiments, the present disclosure provides chirally controlled oligonucleotide compositions, e.g., of oligonucleotides in Table 1. In some embodiments, a chirally controlled oligonucleotide composition comprises a controlled/pre-determined (not random as in stereorandom compositions) level of a plurality of oligonucleotides, wherein the oligonucleotides share the same linkage phosphorus stereochemistry at one or more chiral internucleotidic linkages (chirally controlled internucleotidic linkages). In some embodiments, the oligonucleotides share the same pattern of backbone chiral centers (stereochemistry of linkage phosphorus). In some embodiments, a pattern of backbone chiral centers is as described in the present disclosure. In some embodiments, oligonucleotides of a plurality are structural identical.

In some embodiments, the present disclosure provides a chirally controlled oligonucleotide composition comprising a plurality of oligonucleotides, wherein the oligonucleotides share:
1) a common constitution, and
2) share the same linkage phosphorus stereochemistry at one or more (e.g., 1-50, 1-40, 1-30, 1-25, 1-20, 1, 2, 3, 4, 5, 6, 7, 8, 9, 10, 11, 12, 13, 14, 15, 16, 17, 18, 19, 20, 21, 22, 23, 24, 25 or more) chiral internucleotidic linkages (chirally controlled internucleotidic linkages),
wherein the composition is enriched, relative to a substantially racemic preparation of oligonucleotides of the common constitution, for oligonucleotides of the plurality.

In some embodiments, the present disclosure provides an oligonucleotide composition comprising a plurality of oligonucleotides, wherein oligonucleotides of the plurality share:
1) a common base sequence, and
2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”);
wherein stereochemical purity of the linkage phosphorus of each chirally controlled internucleotidic linkage is independently 80%-100% (e.g., 85-100%, 90-100%, about or at least about 90%, 91%, 92%, 93%, 94%, 95%, 96%, 97%, 98%, 99% or 99.5%).

In some embodiments, the present disclosure provides a chirally controlled oligonucleotide composition comprising a plurality of oligonucleotides, wherein the oligonucleotides share:
1) a common constitution, and
2) share the same linkage phosphorus stereochemistry at one or more (e.g., 1-50, 1-40, 1-30, 1-25, 1-20, 1, 2, 3, 4, 5, 6, 7, 8, 9, 10, 11, 12, 13, 14, 15, 16, 17, 18, 19, 20, 21, 22, 23, 24, 25 or more) chiral internucleotidic linkages (chirally controlled internucleotidic linkages),
wherein stereochemical purity of the linkage phosphorus of each chirally controlled internucleotidic linkage is independently 80%-100% (e.g., 85-100%, 90-100%, about or at least about 90%, 91%, 92%, 93%, 94%, 95%, 96%, 97%, 98%, 99% or 99.5%).

In some embodiments, oligonucleotide of a plurality share the same nucleobase modifications and/or sugar modifications. In some embodiments, oligonucleotide of a plurality share the same internucleotidic linkage modifications (wherein the internucleotidic linkages may be in various acid, base, and/or salt forms). In some embodiments, oligonucleotides of a plurality share the same nucleobase modifications, sugar modifications, and internucleotidic linkage modifications, if any. In some embodiments, oligonucleotides of a plurality are of the same form, e.g., an acid form, a base form, or a particularly salt form (e.g., a pharmaceutically acceptable salt form, e.g., salt form). In some embodiments, oligonucleotides in a composition may exist as one or more forms, e.g., acid forms, base forms, and/or one or more salt forms. In some embodiments, in an aqueous solution (e.g., when dissolved in a buffer like PBS), anions and cations may dissociate. In some embodiments, oligonucleotides of a plurality are of the same constitution. In some embodiments, oligonucleotides of a plurality are structurally identical. In some embodiments, the present disclosure provides a chirally controlled oligonucleotide composition comprising a plurality of oligonucleotides, wherein the oligonucleotides are of a common constitution, and share the same linkage phosphorus stereochemistry at one or more (e.g., 1-60, 1-50, 1-40, 1-30, 1-25, 1-20, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more) chiral internucleotidic linkages (chirally controlled internucleotidic linkages), wherein the composition is enriched, relative to a substantially racemic preparation of oligonucleotides of the common constitution, for oligonucleotides of the plurality.

In some embodiments, at least one chiral internucleotidic linkage is chirally controlled. In some embodiments, at least 2 internucleotidic linkages are independently chirally controlled. In some embodiments, the number of chirally controlled internucleotidic linkages is at least 3. In some embodiments, it is at least 4. In some embodiments, it is at least 5. In some embodiments, it is at least 6. In some embodiments, it is at least 7. In some embodiments, it is at least 8. In some embodiments, it is at least 9. In some embodiments, it is at least 10. In some embodiments, it is at least 11. In some embodiments, it is at least 12. In some embodiments, it is at least 13. In some embodiments, it is at least 14. In some embodiments, it is at least 15. In some embodiments, it is at least 20. In some embodiments, it is at least 25. In some embodiments, it is at least 30. In some embodiments, each chiral internucleotidic linkage is independently a chirally controlled internucleotidic linkage.

In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of all internucleotidic linkages are chirally controlled. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%, 85%, 80%-90%, 80%, 95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.) of all chiral internucleotidic linkages are chirally controlled. In some embodiments, at least 5%-100% (e.g., about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%, 95%, 85%-100%, 90%-95%, 90%-100%, 10%, 20%, 30%, 40%, 50%, 60%, 65%, 70%, 75%, 80%, 85%, 90%, 95%, or 100%, etc.) of all phosphorothioate internucleotidic linkages are chirally controlled. In some embodiments, a percentage is at least 50%. In some embodiments, a percentage is at least 60%. In some embodiments, a percentage is at least 70%. In some embodiments, a percentage is at least 80%. In some embodiments, a percentage is at least 90%. In some embodiments, a percentage is at least 90%. In some embodiments, each chiral internucleotidic linkage is chirally controlled. In some embodiments, each phosphorothioate internucleotidic linkage is chirally controlled.

In some embodiments, the present disclosure provides a composition comprising a plurality of oligonucleotides, wherein each oligonucleotide of the plurality is independently a particular oligonucleotide or a salt thereof. In some embodiments, the present disclosure provides a composition comprising a plurality of oligonucleotides, wherein each oligonucleotide of the plurality is independently a particular oligonucleotide or a pharmaceutically acceptable salt thereof. In some embodiments, such a composition is enriched relative to a substantially racemic preparation of a particular oligonucleotide. As appreciated by those skilled in the art, oligonucleotides of the plurality share a common sequence which is the base sequence of the particular oligonucleotide. In some embodiments, at least about 5%, 100%, 10%-100%, 20-100%, 30%-100%, 40%-100%, 50%-100%, 5%-90%, 10%-90%, 20-90%, 30%-90%, 40%-90%, 50%-90%, 5%-85%, 10%-85%, 20-85%, 30%-85%, 40%-85%, 50%-85%, 5%-80%, 10%-80%, 20-80%, 30%, 80%, 40%-80%, 50%-80%, 5%-75%, 10%-75%, 20-75%, 30%-75%, 40%-75%, 50%-75%, 5%-70%, 10%-70%, 20-70%, 30%-70%, 40%-70%, 50%-70%, 5%-65%, 10%-65%, 20-65%, 30%-65%, 40%-65%, 50%-65%, 5%-60%, 10%-60%, 20-60%, 30%-60%, 40%-60%, 50%-60%, 5%, 10%, 20%, 30%, 40%, 50%, 60%, 70%, 80%, 85%, 90%, 91%, 92%, 93%, 94%, 95%, 96%, 97%, 98%, or 99% of all oligonucleotides in the composition that share the base sequence of a the particular oligonucleotide are oligonucleotide of the plurality. In some embodiments, at least about 5%-100%, 10%-100%, 20-100%, 30%-100%, 40%-100%, 50%-100%, 5%-90%, 10%-90%, 20-90%, 30%-90%, 40%-90%, 50%-90%, 5%-85%, 10%-85%, 20-85%, 30%-85%, 40%-85%, 50%-85%, 5%-80%, 10%-80%, 20-80%, 30%-80%, 40%-80%, 50%-80%, 5%-75%, 10%-75%, 20-75%, 30%-75%, 40%-75%, 50%-75%, 5%-70%, 10%-70%, 20-70%, 30%-70%, 40%-70%, 50%-70%, 5%-65%, 10%-65%, 20-65%, 30%-65%, 40%-65%, 50%-65%, 5%-60%, 10%-60%, 20-60%, 30%-60%, 40%-60%, 50%-60%, 5%, 10%, 20%, 30%, 40%, 50%, 60%, 70%, 80%, 85%, 90%, 91%, 92%, 93%, 94%, 95%, 96%, 97%, 98%, or 99% of all oligonucleotides in the composition that share the constitution of the particular oligonucleotide or a salt thereof are oligonucleotide of the plurality. In some embodiments, a percentage is at least 10%. In some embodiments, a percentage is at least 20%. In some embodiments, a percentage is at least 30%. In some embodiments, a percentage is at least 40%. In some embodiments, a percentage is at least 50%. In some embodiments, it is at least 60%. In some embodiments, it is at least 70%. In some embodiments, it is at least 80%. In some embodiments, it is at least 90%. In some embodiments, it is at least 95%. In some embodiments, it is about 5-100%. In some embodiments, it is about 10-100%. In some embodiments, it is about 20-100%. In some embodiments, it is about 30-90%. In some embodiments, it is about 30-80%. In some embodiments, it is about 30-70%. In some embodiments, it is about 40-90%. In some embodiments, it is about 40-80%. In some embodiments, it is about 40-70%. In some embodiments, a particular oligonucleotide is an oligonucleotide exemplified herein, e.g., an oligonucleotide of Table 1 or another table.

In some embodiments, an enrichment relative to a substantially racemic preparation is that at least about 5%-100%, 10%-100%, 20-100%, 30%-100%, 40%-100%, 50%-100%, 50%-90%, 10%-90%, 20-90%, 30%-90%, 40%-90%, 50%, 90%, 5%-85%, 10%-85%, 20-85%, 30%, 85%, 40%-85%, 50%-85%, 5%-80%, 10%-80%, 20-80%, 30%-80%, 40%-80%, 50%-80%, 5%-75%, 10%-75%, 20-75%, 30%-75%, 40%-75%, 50%-75%, 5%-70%, 10%-70%, 20-70%, 30%-70%, 40%-70%, 50%-70%, 5%-65%, 10%-65%, 20-65%, 30%-65%, 40%-65%, 50%-65%, 5%-60%, 10%-60%, 20-60%, 30%-60%, 40%-60%, 50%-60%, 5%, 10%, 20%, 30%, 40%, 50%, 60%, 70%, 80%, 85%, 90%, 91%, 92%, 93%, 94%, 95%, 96%, 97%, 98%, or 99% of all oligonucleotides in the composition, or all oligonucleotides in the composition that share the common base sequence of a plurality, or all oligonucleotides in the composition that share the common constitution of a plurality, are oligonucleotide of the plurality. In some embodiments, a percentage is at least 10%. In some embodiments, a percentage is at least 20%. In some embodiments, a percentage is at least 30%. In some embodiments, a percentage is at least 40%. In some embodiments, a percentage is at least 50%. In some embodiments, it is at least 60%. In some embodiments, it is at least 70%. In some embodiments, it is at least 80%. In some embodiments, it is at least 90%. In some embodiments, it is at least 95%. In some embodiments, it is about 5-100%. In some embodiments, it is about 10-100%. In some embodiments, it is about 20-100%. In some embodiments, it is about 30-90%. In some embodiments, it is about 30-80%. In some embodiments, it is about 30-7(0%. In some embodiments, it is about 40-90%. In some embodiments, it is about 40-80%. In some embodiments, it is about 40-70%.

In some embodiments, at least about 5%-100%, 10%-100%, 20-100%, 30%-100%, 40%-100%, 50%-100%, 5%-90%, 10%-90%, 20-90%, 30%-90%, 40%-90%, 50%-90%, 5%-85%, 10%-85%, 20-85%, 30%-85%, 40%-85%, 50%-85%, 5%-80%, 10%-80%, 20-80%, 30%-80%, 40%-80%, 50%-80%, 5%-75%, 10%-75%, 20-75%, 30%-75%, 40%-75%, 50%-75%, 5%-70%, 10%-70%, 20-70%, 30%-70%, 40%-70%, 50%-70%, 5%-65%, 10%-65%, 20-65%, 30%-65%, 40%-65%, 50%-65%, 5%-60%, 10%-60%, 20-60%, 30%-60%, 40%-60%, 50%-60%, 5%, 10%, 20%, 30%, 40%, 50%, 60%, 70%, 80%, 85%, 90%, 91%, 92%, 93%, 94%, 95%, 96%, 97%, 98%, or 99% of all oligonucleotides in the composition that share the common base sequence of a plurality are oligonucleotide of the plurality. In some embodiments, a percentage is at least 10%. In some embodiments, a percentage is at least 20%. In some embodiments, a percentage is at least 30%. In some embodiments, a percentage is at least 40%. In some embodiments, a percentage is at least 50%. In some embodiments, it is at least 60%. In some embodiments, it is at least 70%. In some embodiments, it is at least 80%. In some embodiments, it is at least 90%. In some embodiments, it is at least 95%. In some embodiments, it is about 5-100%. In some embodiments, it is about 10-100%. In some embodiments, it is about 20-100%. In some embodiments, it is about 30-90%. In some embodiments, it is about 30-80%. In some embodiments, it is about 30-70%. In some embodiments, it is about 40-90%. In some embodiments, it is about 40-80%. In some embodiments, it is about 40-70%.

Levels of oligonucleotides of a plurality in chirally controlled oligonucleotide compositions are controlled. In contrast, in non-chirally controlled (or stereorandom, racemic) oligonucleotide compositions (or preparations), levels of oligonucleotides are random and not controlled. In some embodiments, an enrichment relative to a substantially racemic preparation is a level described herein.

In some embodiments, a level as a percentage (e.g., a controlled level, a pre-determined level, an enrichment) is or is at least (DS)^{nc}, wherein DS (diastereopurity of an individual internucleotidic linkage) is 90%-100%, and nc is the number of chiral linkage phosphorus as described in the present disclosure (e.g., 5, 6, 7, 8, 9, 10, 11, 12, 13, 14, 15, 16, 17, 18, 19, 20 or more). In some embodiments, a level as a percentage (e.g., a controlled level, a pre-determined level, an enrichment) is or is at least (DS)^{nc}, wherein DS (diastereopurity of an individual internucleotidic linkage) is 90%-100%, and nc is the number of chirally controlled internucleotidic linkages as described in the present disclosure (e.g., 5, 6, 7, 8, 9, 10, 11, 12, 13, 14, 15, 16, 17, 18, 19, 20 or more). In some embodiments, each chiral internucleotidic linkage is chirally controlled, and nc is the number of chiral internucleotidic linkage. In some embodiments, DS is 90%, 91%, 92%, 93%, 94%, 95%, 96%, 97%, 98%, 99% or 99.5% or more. In some embodiments, DS is or is at least 90%. In some embodiments, DS is or is at least 91%. In some embodiments, DS is or is at least 92%. In some embodiments, DS is or is at least 93%. In some embodiments, DS is or is at least 94%. In some embodiments, DS is or is at least 95%. In some embodiments, DS is or is at least 96%. In some embodiments, DS is or is at least 97%. In some embodiments, DS is or is at least 98%. In some embodiments, DS is or is at least 99%. In some embodiments, a level (e.g., a controlled level, a pre-determined level, an enrichment) is a percentage of all oligonucleotides in a composition that share the same constitution, wherein the percentage is or is at least (DS)^{nc}. For example, when DS is 99% and nc is 10, the percentage is or is at least 90% ((99%)¹⁰≈0.90=90%). As appreciated by those skilled in the art, in a stereorandom preparation the percentage is typically about ½^{nc}—when nc is 10, the percentage is about ½¹⁰≈0.001=0.1%. In some embodiments, an enrichment (e.g., relative to a substantially racemic preparation), a level, etc., is that at least about (DS)^{nc} of all oligonucleotides in the composition, or all oligonucleotides in the composition that share the common base sequence of a plurality, or all oligonucleotides in the composition that share the common constitution of a plurality, are oligonucleotide of the plurality. In some embodiments, it is of all oligonucleotides in the composition. In some embodiments, it is of all oligonucleotides in the composition that share the common base sequence of a plurality. In some embodiments, it is of all oligonucleotides in the composition that share the common constitution of a plurality. In some embodiments, various forms (e.g., various salt forms) of an oligonucleotide may be properly considered to have the same constitution.

In some embodiments, oligonucleotides comprise one or more (e.g., 5, 6, 7, 8, 9, 10, 11, 12, 13, 14, 15, 16, 17, 18, 19, 20 or more) chirally controlled chiral internucleotidic linkages the diasteromeric excess (d.e.) of whose linkage phosphorus is independently about or at least about 80%, 85%, 90%, 91%, 92%, 93%, 94%, 95%, 96%, 97%, 98% or 99%. In some embodiments, about or at least about 50%, 60%, 70%, 75%, 80%, 85%, 90%, or 95% of all chiral internucleotidic linkages comprising a chiral linkage phosphorus are independently such a chirally controlled internucleotidic linkage. In some embodiments, about or at least about 50%, 60%, 70%, 75%, 80%, 85%, 90%, or 95% of phosphorothioate internucleotidic linkages are independently such a chirally controlled internucleotidic linkage. In some embodiments, each phosphorothioate internucleotidic linkage is independently such a chirally controlled internucleotidic linkage. In some embodiments, each chiral internucleotidic linkage comprising a chiral linkage phosphorus is independently such a chirally controlled internucleotidic linkage. In some embodiments, d.e. is about or at least about 80%. In some embodiments, d.e. is about or at least about 85%. In some embodiments, d.e. is about or at least about 90%. In some embodiments, d.e. is about or at least about 95%. In some embodiments, d.e. is about or at least about 96%. In some embodiments, d.e. is about or at least about 97%. In some embodiments, d.e. is about or at least about 98%.

In some embodiments, level of a diastereopurity of a plurality of oligonucleotides in a composition can be determined as the product of the diastereopurity of each chiral internucleotidic linkage in the oligonucleotides. In some embodiments, level of a diastereopurity of a plurality of oligonucleotides in a composition can be determined as the product of the diastereopurity of each chirally controlled internucleotidic linkage in the oligonucleotides. In some embodiments, diastereopurity of an internucleotidic linkage connecting two nucleosides in an oligonucleotide (or nucleic acid) is represented by the diastereopurity of an internucleotidic linkage of a dimer connecting the same two nucleosides, wherein the dimer is prepared using comparable conditions, in some instances, identical synthetic cycle conditions (e.g., for the linkage between Nx and Ny in an oligonucleotide . . . . NxNy . . . , the dimer is NxNy).

In some embodiments, a chirally controlled oligonucleotide composition comprises two or more pluralities of oligonucleotides, wherein each plurality is independently a plurality of oligonucleotides as described herein (e.g., in various chirally controlled oligonucleotide compositions). For example, in some embodiments, each plurality independently shares a common base sequence, and the same linkage phosphorus stereochemistry at one or more chiral internucleotidic linkages, and each plurality is independently enriched compared to stereorandom preparation of that plurality or each plurality is independently of a level as described herein. In some embodiments, at least two pluralities or each plurality independently targets a different adenosine. In some embodiments, at least two pluralities or each plurality independently targets a different transcript of the same or different nucleic acids. In some embodiments, at least two pluralities or each plurality independently targets transcripts of a different gene. Among other things, such compositions may be utilized to target two or more targets, in some embodiments, simultaneously and in the same system.

In some embodiments, all chiral internucleotidic linkages are chiral controlled, and the composition is a completely chirally controlled oligonucleotide composition. In some embodiments, not all chiral internucleotidic linkages are chiral controlled internucleotidic linkages, and the composition is a partially chirally controlled oligonucleotide composition.

In some embodiments, a chirally controlled oligonucleotide composition is a chirally pure (or stereopure, stereochemically pure) oligonucleotide composition, wherein the oligonucleotide composition comprises a plurality of oligonucleotides, wherein the oligonucleotides are identical [including that each chiral element of the oligonucleotides, including each chiral linkage phosphorus, is independently defined (stereodefined)], and the composition does not contain other stereoisomers. A chirally pure (or stereopure, stereochemically pure) oligonucleotide composition of an oligonucleotide stereoisomer does not contain other stereoisomers (as appreciated by those skilled in the art, one or more unintended stereoisomers may exist as impurities).

Chirally controlled oligonucleotide compositions can demonstrate a number of advantages over stereorandom oligonucleotide compositions. Among other things, chirally controlled oligonucleotide compositions are more uniform than corresponding stereorandom oligonucleotide compositions with respect to oligonucleotide structures. By controlling stereochemistry, compositions of individual stereoisomers can be prepared and assessed, so that chirally controlled oligonucleotide composition of stereoisomers with desired properties and/or activities can be developed. In some embodiments, chirally controlled oligonucleotide compositions provides better delivery, stability, clearance, activity, selectivity, and/or toxicity profiles compared to, e.g., corresponding stereorandom oligonucleotide compositions. In some embodiments, chirally controlled oligonucleotide compositions provide better efficacy, fewer side effects, and/or more convenient and effective dosage regimens. Among other things, patterns of backbone chiral centers as described herein optionally combined with other structural features described herein, e.g., modifications of nucleobases, sugars, internucleotidic linkages, etc. can be utilized to provide to provide directed adenosine editing with high efficiency.

In some embodiments, the present disclosure provides a chirally controlled oligonucleotide composition. In some embodiments, provided chirally controlled oligonucleotide compositions comprise a plurality of oligonucleotides of the same constitution, and have one or more chiral internucleotidic linkages. In some embodiments, a plurality of oligonucleotides, e.g., in a chirally controlled oligonucleotide composition, is a plurality of an oligonucleotide selected from Table 1 (and/or one or more of various salts forms thereof), wherein the oligonucleotide comprises at least one Rp or Sp linkage phosphorus in a chirally controlled internucleotidic linkage. In some embodiments, a plurality of oligonucleotides, e.g., in a chirally controlled oligonucleotide composition, is a plurality of an oligonucleotide selected from Table 1 (and/or one or more of various salts forms thereof), wherein each phosphorothioate internucleotidic linkage in the oligonucleotide is independently chirally controlled (each phosphorothioate internucleotidic linkage is independently Rp or Sp). In some embodiments, an oligonucleotide composition, e.g., an oligonucleotide composition is a substantially pure preparation of a single oligonucleotide in that oligonucleotides in the composition that are not the single oligonucleotide are impurities from the preparation process of the single oligonucleotide, in some case, after certain purification procedures.

In some embodiments, a chirally controlled oligonucleotide composition can have, relative to a corresponding stereorandom oligonucleotide composition, increased activity and/or stability, increased delivery, and/or decreased ability to elicit adverse effects such as complement, TLR9 activation, etc. In some embodiments, a stereorandom (non-chirally controlled) oligonucleotide composition differs from a chirally controlled oligonucleotide composition in that its corresponding plurality of oligonucleotides do not contain any chirally controlled internucleotidic linkages but the stereorandom oligonucleotide composition is otherwise identical to the chirally controlled oligonucleotide composition.

In some embodiments, the present disclosure pertains to a chirally controlled oligonucleotide composition which is capable of modulating level, activity or expression of a gene (e.g., SERPINA1) or a gene product thereof. In some embodiments, level, activity or expression of a gene or a gene product thereof is increased (e.g., through conversion of A to 1 (e.g., 1024 G>A in SERPINA1) to correct G to A mutations, to increase protein translation levels, to increase production of particular protein isoforms, to modulate splicing to increase levels of a particular splicing products and proteins encoded thereby, etc.), and in some embodiments, level, activity or expression of a gene or a gene product thereof is decreased (e.g., through conversion of A to I to create stop codon and/or alter codons (e.g., to correct E342K in mutant A1AT), to decrease protein translation levels, to decrease production of particular protein isoforms, to modulate splicing to decrease levels of a particular splicing products and proteins encoded thereby, etc.), as compared to a reference condition (e.g., absence of oligonucleotides and/or compositions of the present disclosure, and/or presence of a reference oligonucleotide and/or oligonucleotide composition (e.g., oligonucleotides of the same base sequence but different modifications, stereorandom compositions of oligonucleotides of comparable structures (e.g., base sequence, modifications, etc.) but lack of stereochemical control, etc.).

In some embodiments, a provided chirally controlled oligonucleotide composition is a chirally controlled oligonucleotide composition comprising a plurality of oligonucleotide. In some embodiments, a chirally controlled oligonucleotide composition is a chirally pure (or “stereochemically pure”) oligonucleotide composition. In some embodiments, the present disclosure provides a chirally pure oligonucleotide composition of an oligonucleotide in Table 1, wherein each chiral internucleotidic linkage of the oligonucleotide is independently chirally controlled (Rp or Sp, e.g., can be determined from R or S but not X in “Stereochemistry/Linkage”). As one of ordinary skill in the art will understand, chemical selectivity rarely, if ever, achieves completeness (absolute 100%). In some embodiments, a chirally pure oligonucleotide composition comprises a plurality of oligonucleotides, wherein oligonucleotides of the plurality are structurally identical and all have the same structure (the same stereoisomeric form; in the context of oligonucleotide, typically the same diastereomeric form as typically multiple chiral centers exist in an oligonucleotide), and the chirally pure oligonucleotide composition does not contain any other stereoisomers (in the context of oligonucleotide, typically diastereomers as typically multiple chiral centers exist in an oligonucleotide; to the extent, e.g., achievable by stereoselective preparation). As appreciated by those skilled in the art, stereorandom (or “racemic”, “non-chirally controlled”) oligonucleotide compositions are random mixtures of many stereoisomers (e.g., 2ⁿ diastereoisomers wherein n is the number of chiral linkage phosphorus for oligonucleotides in which other chiral centers (e.g., carbon chiral centers in sugars) are chirally controlled each independently existing in one configuration and only chiral linkage phosphorus centers are not chirally controlled).

In some embodiments, oligonucleotides are linked to a solid support. In some embodiments, a solid support is a support for oligonucleotide synthesis. In some embodiments, a solid support comprises glass. In some embodiments, a solid support is CPG (controlled pore glass). In some embodiments, a solid support is polymer. In some embodiments, a solid support is polystyrene. In some embodiments, the solid support is Highly Crosslinked Polystyrene (HCP). In some embodiments, the solid support is hybrid support of Controlled Pore Glass (CPG) and Highly Cross-linked Polystyrene (HCP). In some embodiments, a solid support is a metal foam. In some embodiments, a solid support is a resin. In some embodiments, oligonucleotides are cleaved from a solid support.

In some embodiments, purity, particularly stereochemical purity, and particularly diastereomeric purity of many oligonucleotides and compositions thereof wherein all other chiral centers in the oligonucleotides but the chiral linkage phosphorus centers have been stereodefined (e.g., carbon chiral centers in the sugars, which are defined in, e.g., phosphoramidites for oligonucleotide synthesis), can be controlled by stereoselectivity (as appreciated by those skilled in this art, diastereoselectivity in many cases of oligonucleotide synthesis wherein the oligonucleotide comprise more than one chiral centers) at chiral linkage phosphorus in coupling steps when forming chiral internucleotidic linkages. In some embodiments, a coupling step has a stereoselectivity (diastereoselectivity when there are other chiral centers) of 60% at the linkage phosphorus. After such a coupling step, the new internucleotidic linkage formed may be referred to have a 60% stereochemical purity (for oligonucleotides, typically diastereomeric purity in view of the existence of other chiral centers). In some embodiments, each coupling step independently has a stereoselectivity of at least 60%, 70%, 80%, 85%, 90%, 91%, 92%, 93%, 94%, 95%, 96%, 97%, 98%, 99%, or 99.5%. In some embodiments, a chirally controlled internucleotidic linkage is typically formed with a stereoselectivity of at least 85%, 87%, 90%, 91%, 92%, 93%, 94%, 95%, 96%, 97%, 98%, 99.5% or virtually 100% (in some embodiments, at least 85%: in some embodiments, at least 87%; in some embodiments, at least 90%; in some embodiments, at least 95%: in some embodiments, at least 96%; in some embodiments, at least 97%; in some embodiments, at least 98%: in some embodiments, at least 99%). In some embodiments, a stereoselectivity is at least 85%. In some embodiments, a stereoselectivity is at least 87%. In some embodiments, a stereoselectivity is at least 90%. In some embodiments, each coupling step independently has a stereoselectivity of virtually 100%.

In some embodiments, stereopurity of a chiral center, e.g., a chiral linkage phosphorus, in a composition is at least 60%, 70%, 80%, 85%, 87%, 90%, 91%, 92%, 93%, 94%, 95%, 96%, 97%, 98%, 99%, or 99.5%. In some embodiments, a stereopurity is at least 80%. In some embodiments, a stereopurity is at least 85%. In some embodiments, a stereopurity is at least 87%. In some embodiments, a stereopurity is at least 90%. In some embodiments, a stereopurity is virtually 100%. In some embodiments, each chirally controlled internucleotidic linkage independently has a stereochemical purity (typically diastereomeric purity for oligonucleotides with multiple chiral centers) of at least 85%, 87%, 90%, 91%, 92%, 93%, 94%, 95%, 96%, 97%, 98%, 99.5% or virtually 100% (in some embodiments, at least 85%; in some embodiments, at least 87%; in some embodiments, at least 90%; in some embodiments, at least 95%; in some embodiments, at least 96%; in some embodiments, at least 97%; in some embodiments, at least 98%; in some embodiments, at least 99%) at its chiral linkage phosphorus. In some embodiments, a chirally controlled internucleotidic linkage has a stereochemical purity of at least 90%. In some embodiments, a majority of chirally controlled internucleotidic linkages independently have a stereochemical purity of at least 90%. In some embodiments, each chirally controlled internucleotidic linkage independently has a stereochemical purity of at least 90%. In some embodiments, each phosphorothioate internucleotidic linkage is independently chirally controlled.

Stereoselectivity and stereopurity may be assessed by various technologies. In some embodiments, stereoselectivity and/or stereopurity is virtually 100% in that when a composition is analyzed by an analytical method (e.g., NMR, HPLC, etc.), virtually all detectable stereoisomers has the intended stereochemistry.

In some embodiments, at least 1, 2, 3, 4, 5, 6, 7, 8, 9, or 10 couplings of a monomer (as appreciated by those skilled in the art in many embodiments a phosphoramidite for oligonucleotide synthesis) independently have a stereoselectivity less than about 60%, 70%, 80%, 85%, or 90% [for oligonucleotide synthesis, typically diastereoselectivity with respect to formed linkage phosphorus chiral center(s)].

In some embodiments, in stereorandom (or racemic) preparations (or stereorandom/non-chirally controlled oligonucleotide compositions), at least 1, 2, 3, 4, 5, 6, 7, 8, 9, 10, 11, 12, 13, 14, 15, 16, 17, 18, 19, 20, 21, 22, 23, 24, or 25 chiral internucleotidic linkages of the oligonucleotides independently have a stereochemical purity (typically diastereomeric purity for oligonucleotides comprising multiple chiral centers) less than about 60%, 65%, 70%, 75%, 80%, or 85% with respect to chiral linkage phosphorus of the internucleotidic linkage(s). In some embodiments, a stereochemistry purity (stereopurity) is less than about 60%. In some embodiments, a stereochemistry purity (stereopurity) is less than about 65%. In some embodiments, a stereochemistry purity (stereopurity) is less than about 70%. In some embodiments, a stereochemistry purity (stereopurity) is less than about 75%. In some embodiments, a stereochemistry purity (stereopurity) is less than about 80%.

In some embodiments, compounds of the present disclosure (e.g., oligonucleotides, chiral auxiliaries, etc.) comprise multiple chiral elements (e.g., multiple carbon and/or phosphorus (e.g., linkage phosphorus of chiral internucleotidic linkages) chiral centers). In some embodiments, at least 1, 2, 3, 4, 5, 6, 7, 8, 9 or more chiral elements of a provided compound (e.g., an oligonucleotide) each independently have a diastereomeric purity as described herein. In some embodiments, a diastereomeric purity is at least 85%. In some embodiments, a diastereomeric purity is at least 86%. In some embodiments, a diastereomeric purity is at least 87%. In some embodiments, a diastereomeric purity is at least 88%. In some embodiments, a diastereomeric purity is at least 89%. In some embodiments, a diastereomeric purity is at least 90%. In some embodiments, a diastereomeric purity is at least 91%. In some embodiments, a diastereomeric purity is at least 92%. In some embodiments, a diastereomeric purity is at least 93%. In some embodiments, a diastereomeric purity is at least 94%. In some embodiments, a diastereomeric purity is at least 95%. In some embodiments, a diastereomeric purity is at least 96%. In some embodiments, a diastereomeric purity is at least 97%. In some embodiments, a diastereomeric purity is at least 98%. In some embodiments, a diastereomeric purity is at least 99%.

As understood by a person having ordinary skill in the art, in some embodiments, diastereoselectivity of a coupling or diastereomeric purity of a chiral linkage phosphorus center can be assessed through the diastereoselectivity of a dimer formation or diastereomeric purity of a dimer prepared under the same or comparable conditions, wherein the dimer has the same 5′- and 3′-nucleosides and internucleotidic linkage.

Various technologies can be utilized for identifying or confirming stereochemistry of chiral elements (e.g., configuration of chiral linkage phosphorus) and/or patterns of backbone chiral centers, and/or for assessing stereoselectivity (e.g., diastereoselectivity of couple steps in oligonucleotide synthesis) and/or stereochemical purity (e.g., diastereomeric purity of internucleotidic linkages, compounds (e.g., oligonucleotides), etc.). Example technologies include NMR [e.g., 1D (one-dimensional) and/or 2D (two-dimensional) ¹H-³¹P HETCOR (heteronuclear correlation spectroscopy)], HPLC, RP-HPLC, mass spectrometry. LC-MS, and cleavage of internucleotidic linkages by stereospecific nucleases, etc., which may be utilized individually or in combination. Example useful nucleases include benzonase, micrococcal nuclease, and svPDE (snake venom phosphodiesterase), which are specific for certain internucleotidic linkages with Rp linkage phosphorus (e.g., a Rp phosphorothioate linkage); and nuclease P1, mung bean nuclease, and nuclease SI, which are specific for internucleotidic linkages with Sp linkage phosphorus (e.g., a Sp phosphorothioate linkage). Without wishing to be bound by any particular theory, the present disclosure notes that, in at least some cases, cleavage of oligonucleotides by a particular nuclease may be impacted by structural elements, e.g., chemical modifications (e.g., 2′-modifications of a sugars), base sequences, or stereochemical contexts. For example, it is observed that in some cases, benzonase and micrococcal nuclease, which are specific for internucleotidic linkages with Rp linkage phosphorus, were unable to cleave an isolated Rp phosphorothioate internucleotidic linkage flanked by Sp phosphorothioate internucleotidic linkages.

In some embodiments, an oligonucleotide composition is a substantially pure preparation of a single oligonucleotide stereoisomer in that oligonucleotides in the composition that are of the same constitution but are not of the stereoisomer are impurities from the preparation process of said oligonucleotide stereoisomer, in some case, after certain purification procedures.

### Editing Region

In some embodiments, the present disclosure provides oligonucleotides comprising editing regions, e.g., regions comprising or consisting of 5′-N₁N₀N₋₁-3′ as described herein. In some embodiments, an editing region is or comprises a nucleoside opposite to a target adenosine (typically, when base sequences of oligonucleotides are aligned with target sequences for maximal complementarity, and/or oligonucleotides hybridize with target nucleic acids) and its neighboring nucleosides. In some embodiments, an editing region is or comprises three nucleobases, wherein the nucleobase in the middle is a nucleoside opposite to a target adenosine. In some embodiments, a nucleoside opposite to a target adenosine is N₀ as described herein.

In some embodiments, the nucleobase of a nucleoside opposite to a target adenosine (may be referred to as BA₀) is b008U. In some embodiments, sugar of N₀ is a natural DNA sugar. See, e.g., various oligonucleotides in Table 1. In some embodiments, it was observed that b008U as BA₀ can provide improved adenosine editing efficiency. In some embodiments, a reference nucleobase is U. In some embodiments, a reference nucleobase is T. In some embodiments, a reference nucleobase is C.

In some embodiments, a nucleoside opposite to a target adenosine, e.g., N₀, is b008U (which when utilized for a nucleoside refers to

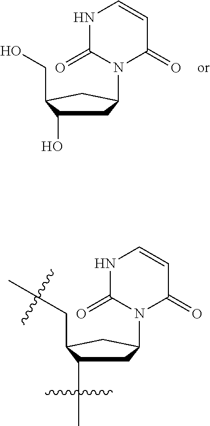

in an oligonucleotide chain unless specified otherwise). See, e.g., various oligonucleotides in Table 1. In some embodiments, it was observed that b008U can provide improved editing, e.g., when compared to dC at positions opposite to target adenosines.

In some embodiments, replacing guanine with hypoxanthine at position −1 (e.g., replacing dG with dI) can provide improved editing. In some embodiments, the sugar of each of N₁, N₀, and N₋₁ is independently a natural DNA sugar. See, e.g., various oligonucleotides in Table 1.

### Nucleobases

Various nucleobases may be utilized in oligonucleotides in accordance with the present disclosure. In some embodiments, a nucleobase is a natural nucleobase, the most commonly occurring ones being A, T, C, G and U. In some embodiments, a nucleobase is a modified nucleobase in that it is not A, T, C, G or U. In some embodiments, a nucleobase is optionally substituted A, T, C, G or U, or a substituted tautomer of A T, C, G or U. In some embodiments, a nucleobase is optionally substituted A, T, C, G or U, e.g., 5mC, 5-hydroxymethyl C, etc. In some embodiments, a nucleobase is A. In some embodiments, a nucleobase is T. In some embodiments, a nucleobase is C. In some embodiments, a nucleobase is G. In some embodiments, a nucleobase is U. In some embodiments, a nucleobase is 5mC. In some embodiments, a nucleobase is substituted A, T, C, G or U. In some embodiments, a nucleobase is a substituted tautomer of A, T, C, G or U. In some embodiments, a nucleobase is a modified base. In some embodiments, a base is b008U

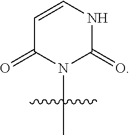 [SMILES: *n1c(=O)cc[nH]c1=O]

In some embodiments, a base is optionally substituted b008U. In some embodiments, a base is optionally protected b008U. In some embodiments, a nucleobase is hypoxanthine. In some embodiments, a nucleobase optionally substituted hypoxanthine or a tautomer thereof. In some embodiments, a nucleobase is an optionally protected hypoxanthine or a tautomer thereof. In some embodiments, substitution protects certain functional groups in nucleobases to minimize undesired reactions during oligonucleotide synthesis. Suitable technologies for nucleobase protection in oligonucleotide synthesis are widely known in the art and may be utilized in accordance with the present disclosure. In some embodiments, modified nucleobases improves properties and/or activities of oligonucleotides. For example, in many cases, 5mC may be utilized in place of C to modulate certain undesired biological effects, e.g., immune responses. In some embodiments, when determining sequence identity, a substituted nucleobase having the same hydrogen-bonding pattern is treated as the same as the unsubstituted nucleobase, e.g., 5mC may be treated the same as C [e.g., an oligonucleotide having 5mC in place of C (e.g., AT5mCG) is considered to have the same base sequence as an oligonucleotide having C at the corresponding location(s) (e.g., ATCG)].

In some embodiments, a nucleobase is a modified base.

In some embodiments, a nucleoside is b008U

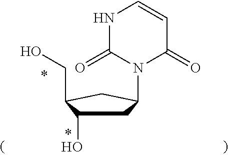

or a salt thereof, wherein “*” indicates connection to internucleotidic linkages when in various oligonucleotides.

Certain useful nucleobases, nucleosides, etc. are described in WO 2021/071858 and WO 2022/099159, the entirety of each of which is incorporated herein by reference.

### Sugars

Various sugars, including modified sugars, can be utilized in accordance with the present disclosure. In some embodiments, the present disclosure provides sugar modifications and patterns thereof optionally in combination with other structural elements (e.g., internucleotidic linkage modifications and patterns thereof, pattern of backbone chiral centers thereof, etc.) that when incorporated into oligonucleotides can provide improved properties and/or activities.

The most common naturally occurring nucleosides comprise ribose sugars (e.g., in RNA) or deoxyribose sugars (e.g., in DNA) linked to the nucleobases adenosine (A), cytosine (C), guanine (G), thymine (T) or uracil (U). In some embodiments, a sugar, e.g., various sugars in many oligonucleotides in Table 1 (unless otherwise notes), is a natural DNA sugar (in DNA nucleic acids or oligonucleotides, having the structure of

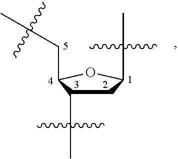

wherein a nucleobase is attached to the 1′ position, and the 3′ and 5′ positions are connected to internucleotidic linkages (as appreciated by those skilled in the art, if at the 5′-end of oligonucleotide, the 5′ position may be connected to a 5′-end group (e.g., —OH), and if at the 3′-end of an oligonucleotide, the 3′ position may be connected to a 3′-end group (e.g., —OH). In some embodiments, a sugar is a natural RNA sugar (in RNA nucleic acids or oligonucleotides, having the structure of

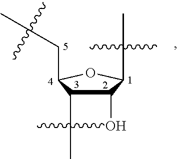

wherein a nucleobase is attached to the 1′ position, and the 3′ and 5′ positions are connected to internucleotidic linkages (as appreciated by those skilled in the art, if at the 5′-end of an oligonucleotide, the 5′ position may be connected to a 5′-end group (e.g., —OH), and if at the 3′-end of an oligonucleotide, the 3′ position may be connected to a 3′-end group (e.g., —OH). In some embodiments, a sugar is a modified sugar in that it is not a natural DNA sugar or a natural RNA sugar. Among other things, modified sugars may provide improved stability. In some embodiments, modified sugars can be utilized to alter and/or optimize one or more hybridization characteristics. In some embodiments, modified sugars can be utilized to alter and/or optimize target nucleic acid recognition. In some embodiments, modified sugars can be utilized to optimize Tm. In some embodiments, modified sugars can be utilized to improve oligonucleotide activities.

Among other things, the present disclosure demonstrates that various non-natural RNA sugars, such as natural DNA sugar, various modified sugars, etc., may be utilized in accordance with the present disclosure. For example, oligonucleotides in Table 1 comprise natural DNA sugars, 2′-F modified sugars, 2′-OMe modified sugars and in some cases, 2′-MOE modified sugars. Among other things, the present disclosure demonstrates that designed oligonucleotides such as those illustrated in Table 1 can provide high editing efficiency with short lengths and relatively low levels of 2′-F modified sugar (e.g., about or less than 50% of all sugars, about or lower than the level of 2′-OR modified sugars wherein R is optionally substituted C₁₋₆ aliphatic, and/or without a large number (e.g., about 5 or more) of consecutive 2′-F modified sugars).

In some embodiments, a sugar is an optionally substituted natural DNA or RNA sugar. In some embodiments, a sugar is optionally substituted

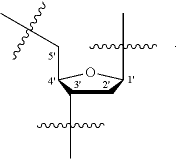

In some embodiments, the 2′ position is optionally substituted. In some embodiments, a sugar is

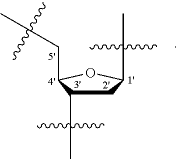

In some embodiments, a 2′-modified sugar has the structure of

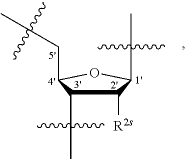

wherein R^{2s} is a 2′-modification. In some embodiments, a sugar has the structure of

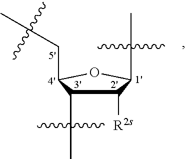

wherein R^{2s} is —H, halogen, or —OR, wherein R is optionally substituted C₁₋₆ aliphatic. In some embodiments, R^{2s} is —H. In some embodiments, R^{2s} is —F. In some embodiments, R^{2s} is —OMe. In some embodiments, a modified nucleoside is mA, mT, mC, m5mC, mG, mU, etc., in which R^{2s} is —OMe. In some embodiments, R^{2s} is —OCH₂CH₂OMe. In some embodiments, a modified nucleoside is Aeo, Teo, Ceo, m5Ceo, Geo, Ueo, etc., in which R^{2s} is —OCH₂CH₂OMe. In some embodiments, R^{2s} is —OCH₂CH₂OH. In some embodiments, an oligonucleotide comprises a 2′-F modified sugar having the structure of

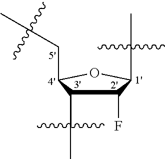

(e.g., as in fA, fT, fC, f5mC, fG, fU, etc.). In some embodiments, an oligonucleotide comprises a 2′-OMe modified sugar having the structure of

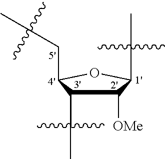

(e.g., as in mA, mT, mC, m5mC, mG, mU, etc.). In some embodiments, an oligonucleotide comprises a 2′-MOE modified sugar having the structure of

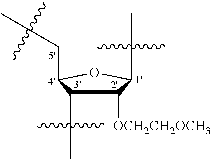

(e.g., as in Aeo, Teo, Ceo, m5Ceo, Geo, Ueo, etc.).

In some embodiments, a sugar has the structure of

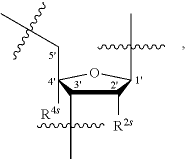

wherein R^{2s} and R^{4s} are taken together to form -L^{s}-, wherein L^{s} is a covalent bond or optionally substituted bivalent C₁₋₆ aliphatic or heteroaliphatic having 1-4 heteroatoms. In some embodiments, each heteroatom is independently selected from nitrogen, oxygen or sulfur). In some embodiments, L^{s} is optionally substituted C2-O—CH₂—C4. In some embodiments, L^{s} is C2-O—CH₂—C4. In some embodiments, L^{s} is C2-O—(R)—CH(CH₂CH₃)—C4. In some embodiments, L^{s} is C2-O—(S)—CH(CH₂CH₃)—C4.

In some embodiments, a sugar is a sugar described in WO 2018/022473, WO 2018/098264, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, WO 2021/071858, and/or WO 2022/099159, the sugars and modified sugars of each of which are independently incorporated herein by reference.

### Internucleotidic Linkages

Among other things, the present disclosure provides various internucleotidic linkages, including various modified internucleotidic linkages, that may be utilized together with other structural elements, e.g., various sugars as described herein, to provide oligonucleotides and compositions thereof.

In some embodiments, oligonucleotides comprise base modifications, sugar modifications, and/or internucleotidic linkage modifications. Various internucleotidic linkages can be utilized in accordance with the present disclosure to link units comprising nucleobases, e.g., nucleosides. In some embodiments, provided oligonucleotides comprise both one or more modified internucleotidic linkages and one or more natural phosphate linkages. As widely known by those skilled in the art, natural phosphate linkages are widely found in natural DNA and RNA molecules; they have the structure of —OP(O)(OH)O—, connect sugars in the nucleosides in DNA and RNA, and may be in various salt forms, for example, at physiological pH (about 7.4), natural phosphate linkages are predominantly exist in salt forms with the anion being —OP(O)(O⁻)O—. A modified internucleotidic linkage, or a non-natural phosphate linkage, is an internucleotidic linkage that is not natural phosphate linkage or a salt form thereof. Modified internucleotidic linkages, depending on their structures, may also be in their salt forms. For example, as appreciated by those skilled in the art, phosphorothioate internucleotidic linkages which have the structure of —OP(O)(SH)O— may be in various salt forms, e.g., at physiological pH (about 7.4) with the anion being —OP(O)(S—)O—. In some embodiments, an internucleotidic linkage may exist in neutral form, e.g., at physiological pH (about 7.4).

In some embodiments, a linkage contains a linkage phosphorus atom bonded to an oxygen atom which oxygen atom is not bonded to or is not part of a backbone sugar (“a PO linkage”, e.g., a natural phosphate linkage). In some embodiments, a linkage contains a linkage phosphorus atom bonded to a sulfur atom which sulfur atom is not bonded to or is not part of a backbone sugar (“a PS linkage”, e.g., a phosphorothioate internucleotidic linkage). In some embodiments, a linkage contains a linkage phosphorus atom bonded to a nitrogen atom which nitrogen atom is not bonded to or is not part of a backbone sugar (“a PN linkage”, e.g., n001). In some embodiments, an oligonucleotide comprises one or more PO linkages, one or more PS linkages, and one or more PN linkages. In some embodiments, an oligonucleotide comprises one or more natural phosphate linkages, one or more phosphorothioate internucleotidic linkages, and one or more n001 linkages. In some embodiments, each chiral linkage phosphorus is independently chirally controlled.

In some embodiments, an internucleotidic linkage is described in WO 2018/022473, WO 2018/098264, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, WO 2021/071858, and/or WO 2022/099159, the internucleotidic linkages of each of which are independently incorporated herein by reference.

### Additional Chemical Moieties

In some embodiments, an oligonucleotide comprises one or more additional chemical moieties. Various additional chemical moieties, e.g., targeting moieties, carbohydrate moieties, lipid moieties, etc. are known in the art and can be utilized in accordance with the present disclosure to modulate properties and/or activities of provided oligonucleotides, e.g., stability, half life, activities, delivery, pharmacodynamics properties, pharmacokinetic properties, etc. In some embodiments, certain additional chemical moieties facilitate delivery of oligonucleotides to desired cells, tissues and/or organs, including but not limited the cells of the central nervous system. In some embodiments, certain additional chemical moieties facilitate internalization of oligonucleotides. In some embodiments, certain additional chemical moieties increase oligonucleotide stability. In some embodiments, the present disclosure provides technologies for incorporating various additional chemical moieties into oligonucleotides.

In some embodiments, an additional chemical moiety is or comprises a small molecule moiety. In some embodiments, a small molecule is a ligand of a protein (e.g., receptor). In some embodiments, a small molecule binds to a polypeptide. In some embodiments, a small molecule is an inhibitor of a polypeptide. In some embodiments, an additional chemical moiety is or comprises a peptide moiety (e.g., an antibody). In some embodiments, an additional chemical moiety is or comprises a nucleic acid moiety. In some embodiments, a nucleic acid provides a new property and/or activity. In some embodiments, a nucleic acid moiety forms a duplex or other secondary structure with the original oligonucleotide chain (before conjugation) or a portion thereof. In some embodiments, a nucleic acid is or comprises an oligonucleotide targeting the same or a different target, and may perform its activity through the same or a different mechanism. In some embodiments, a nucleic acid is or comprises a RNAi agent. In some embodiments, a nucleic acid is or comprises a miRNA agent. In some embodiments, a nucleic acid is or comprises RNase H dependent. In some embodiments, a nucleic acid is or comprises a gRNA. In some embodiments, a nucleic acid is or comprises an aptamer. In some embodiments, an additional chemical moiety is or comprises a carbohydrate moiety as described herein. Many useful agents, e.g., small molecules, peptides, carbohydrates, nucleic acid agents, etc., may be conjugated with oligonucleotides herein in accordance with the present disclosure.

In some embodiments, an oligonucleotide comprises an additional chemical moiety provides increased delivery to and/or activity in an tissue compared to a reference oligonucleotide, e.g., a reference oligonucleotide which does not have the additional chemical moiety but is otherwise identical.

In some embodiments, additional chemical moieties are carbohydrate moieties, targeting moieties, etc., which, when incorporated into oligonucleotides, can improve one or more properties. In some embodiments, an additional chemical moiety is selected from: glucose, GluNAc (N-acetyl amine glucosamine) and anisamide moieties. In some embodiments, a provided oligonucleotide can comprise two or more additional chemical moieties, wherein the additional chemical moieties are identical or non-identical, or are of the same category (e.g., carbohydrate moiety, sugar moiety, targeting moiety, etc.) or not of the same category.

In some embodiments, an additional chemical moiety is a targeting moiety. In some embodiments, an additional chemical moiety is or comprises a carbohydrate moiety. In some embodiments, an additional chemical moiety is or comprises a lipid moiety. In some embodiments, an additional chemical moiety is or comprises a ligand moiety for, e.g., cell receptors such as a sigma receptor, an asialoglycoprotein receptor, etc. In some embodiments, a ligand moiety is or comprises an anisamide moiety, which may be a ligand moiety for a sigma receptor. In some embodiments, a ligand moiety is or comprises a GalNAc moiety, which may be a ligand moiety for an asialoglycoprotein receptor. In some embodiments, an additional chemical moiety facilitates delivery to liver.

In some embodiments, a provided oligonucleotide can comprise one or more linkers and additional chemical moieties (e.g., targeting moieties), and/or can be chirally controlled or not chirally controlled, and/or have a bases sequence and/or one or more modifications and/or formats as described herein.

Various linkers, carbohydrate moieties and targeting moieties, including many known in the art, can be utilized in accordance with the present disclosure. In some embodiments, a carbohydrate moiety is a targeting moiety. In some embodiments, a targeting moiety is a carbohydrate moiety.

In some embodiments, additional chemical moieties are any of ones described in the Examples, including examples of various additional chemical moieties incorporated into various oligonucleotides.

In some embodiments, an additional chemical moiety conjugated to an oligonucleotide is capable of targeting the oligonucleotide to a cell in the central nervous system.

In some embodiments, an additional chemical moiety comprises or is a cell receptor ligand. In some embodiments, an additional chemical moiety comprises or is a protein binder, e.g., one binds to a cell surface protein. Such moieties among other things can be useful for targeted delivery of oligonucleotides to cells expressing the corresponding receptors or proteins. In some embodiments, an additional chemical moiety of a provided oligonucleotide comprises anisamide or a derivative or an analog thereof and is capable of targeting the oligonucleotide to a cell expressing a particular receptor, such as the sigma 1 receptor.

In some embodiments, a provided oligonucleotide is formulated for administration to a body cell and/or tissue expressing its target. In some embodiments, an additional chemical moiety conjugated to an oligonucleotide is capable of targeting the oligonucleotide to a cell.

In some embodiments, an additional chemical moiety is or comprises an asialoglycoprotein receptor (ASGPR) ligand. Without wishing to be bound by any particular theory, the present disclosure notes that ASGPR1 has also been reported to be expressed in the hippocampus region and/or cerebellum Purkinje cell layer of the mouse.

Various other ASGPR ligands are known in the art and can be utilized in accordance with the present disclosure. In some embodiments, an ASGPR ligand is a carbohydrate. In some embodiments, an ASGPR ligand is GalNac or a derivative or an analog thereof. In some embodiments, an ASGPR ligand is one described in Sanhueza et al. J. Am. Chem. Soc., 2017, 139 (9), pp 3528-3536. In some embodiments, an ASGPR ligand is one described in Mamidyala et al. J. Am. Chem. Soc., 2012, 134, pp 1978-1981. In some embodiments, an ASGPR ligand is one described in US 20160207953. In some embodiments, an ASGPR ligand is a substituted-6,8-dioxabicyclo[3.2.1]octane-2,3-diol derivative disclosed in, e.g., US 20160207953. In some embodiments, an ASGPR ligand is one described in, e.g., US 20150329555. In some embodiments, an ASGPR ligand is a substituted-6,8-dioxabicyclo[3.2.1]octane-2,3-diol derivative disclosed e.g., in US 20150329555. In some embodiments, an ASGPR ligand is one described in U.S. Pat. No. 8,877,917, US 20160376585, U.S. Ser. No. 10/086,081, or U.S. Pat. No. 8,106,022. In some embodiments, various GalNAc derivatives and uses thereof are described in WO 2022/076922 and can be utilized in accordance with the present disclosure. ASGPR ligands described in these documents are incorporated herein by reference. Those skilled in the art will appreciate that various technologies are known in the art, including those described in these documents, for assessing binding of a chemical moiety to ASGPR and can be utilized in accordance with the present disclosure. In some embodiments, a provided oligonucleotide is conjugated to an ASGPR ligand. In some embodiments, a provided oligonucleotide comprises an ASGPR ligand. In some embodiments, an additional chemical moiety comprises an ASGPR ligand is

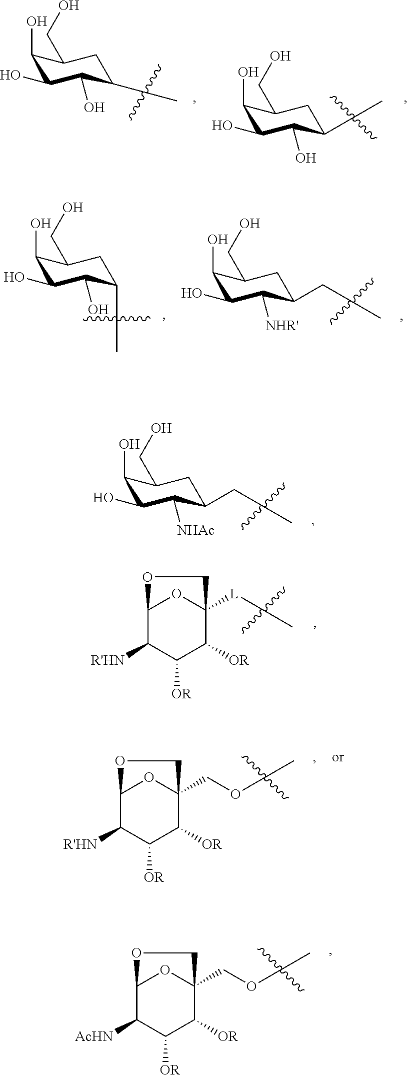

wherein each variable is independently as described in the present disclosure. In some embodiments, R is —H. In some embodiments, R′ is —C(O)R.

In some embodiments, an additional chemical moiety is or comprises

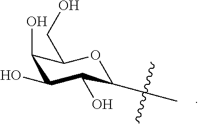

In some embodiments, an additional chemical moiety is or comprises

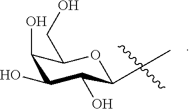

In some embodiments, an additional chemical moiety is or comprises

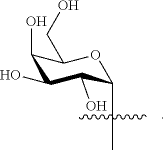

In some embodiments, an additional chemical moiety is or comprises

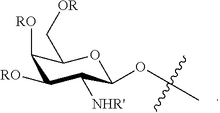

In some embodiments, an additional chemical moiety is or comprises optionally substituted

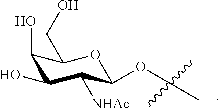

In some embodiments, an additional chemical moiety is or comprises

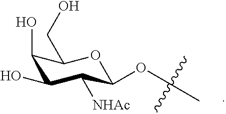

In some embodiments, an additional chemical moiety is or comprises

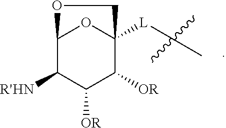

In some embodiments, an additional chemical moiety is or comprises

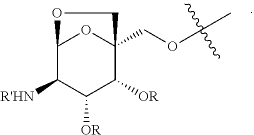

In some embodiments, an additional chemical moiety is or comprises

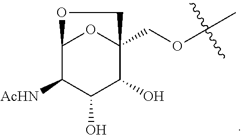 [SMILES: *OC[C@@]12CO[C@@H](O1)[C@H](NC(C)=O)[C@@H](O)[C@H]2O]

In some embodiments, an additional chemical moiety comprises one or more moieties that can bind to, e.g., oligonucleotide target cells. For example, in some embodiments, an additional chemistry moiety comprises one or more protein ligand moieties, e.g., in some embodiments, an additional chemical moiety comprises multiple moieties, each of which independently is an ASGPR ligand. In some embodiments, as in Mod 001 and Mod083, an additional chemical moiety comprises three such ligands.

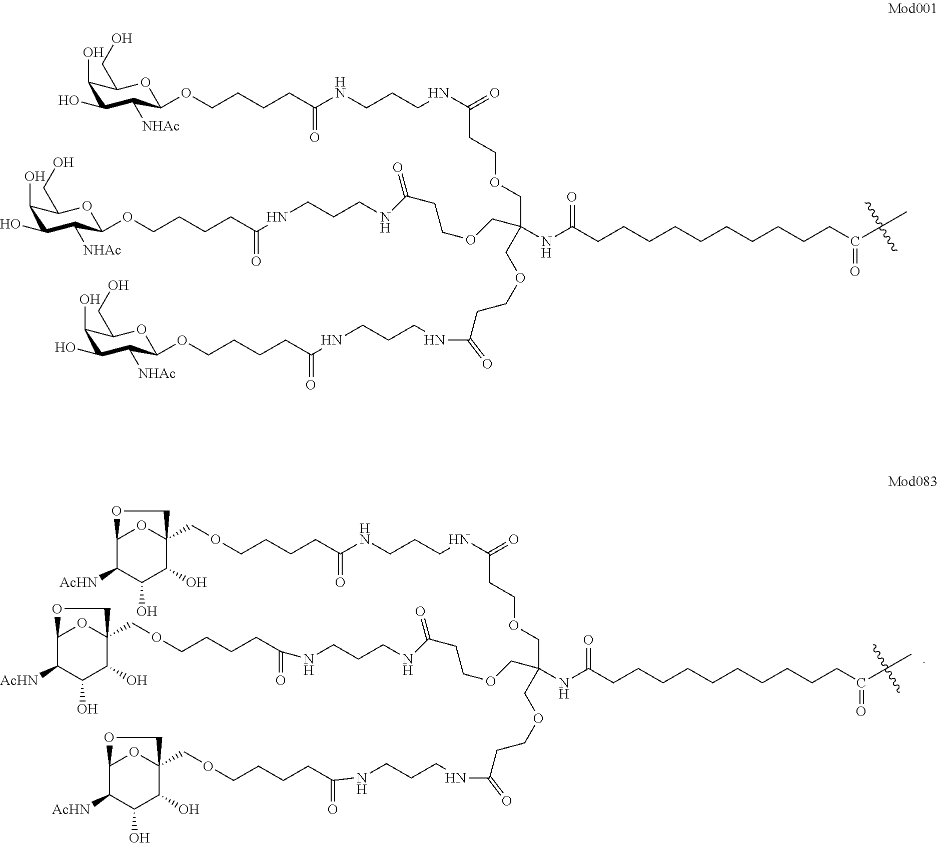

In some embodiments, an oligonucleotide comprises

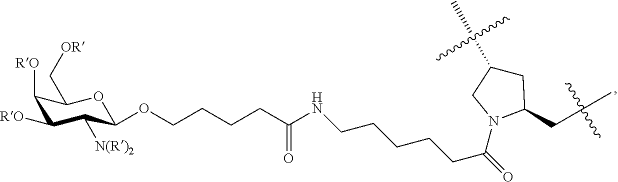

wherein each variable is independently as described herein. In some embodiments, each —OR is —OAc, and —N(R′)₂ is —NHAc. In some embodiments, an oligonucleotide comprises

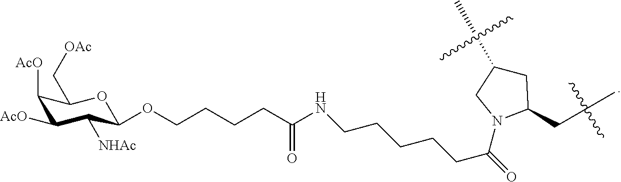

In some embodiments, each R′ is —H. In some embodiments, each —OR′ is —OH, and each —N(R′)₂ is —NHC(O)R. In some embodiments, each —OR′ is —OH, and each —N(R′)₂ is —NHAc. In some embodiments, an oligonucleotide comprises

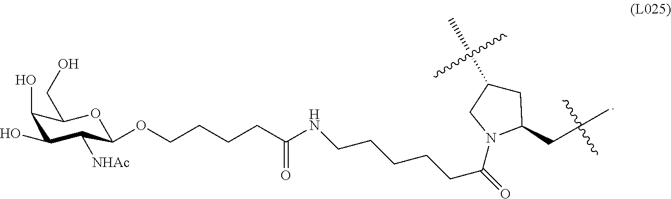

In some embodiments, the —CH₂— connection site is utilized as a C5 connection site in a sugar. In some embodiments, the connection site on the ring is utilized as a C3 connection site in a sugar. Such moieties may be introduced utilizing, e.g., phosphoramidites such as

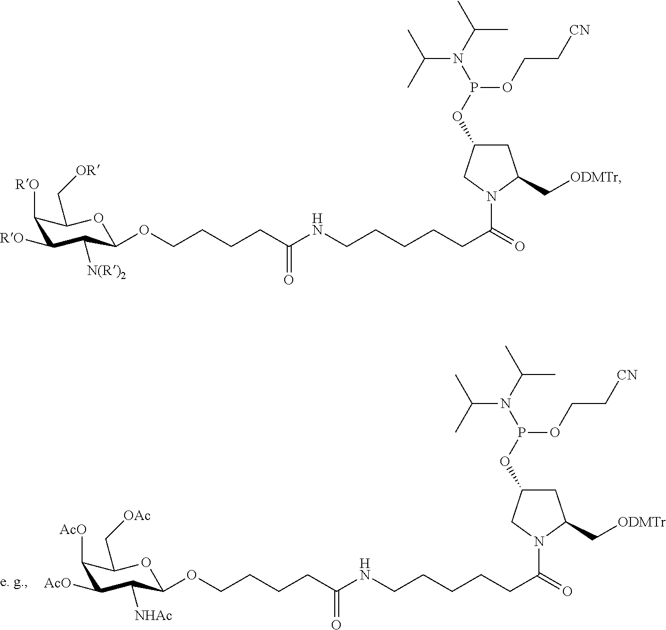

(those skilled in the art appreciate that one or more other groups, such as protection groups for —OH, —NH₂—, —N(i-Pr)₂, —OCH₂CH₂CN, etc., may be alternatively utilized, and protection groups can be removed under various suitable conditions, sometimes during oligonucleotide de-protection and/or cleavage steps). In some embodiments, an oligonucleotide comprises 2, 3 or more (e.g., 3 and no more than 3)

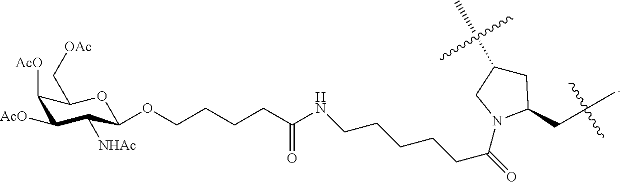

In some embodiments, an oligonucleotide comprises 2, 3 or more (e.g., 3 and no more than 3)

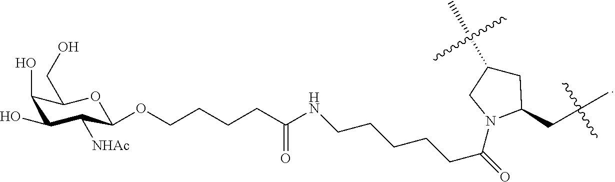

In some embodiments, copies of such moieties are linked by internucleotidic linkages, e.g., natural phosphate linkages, as described herein. In some embodiments, when at a 5′-end, a —CH₂— connection site is bonded to —OH. In some embodiments, an oligonucleotide comprises

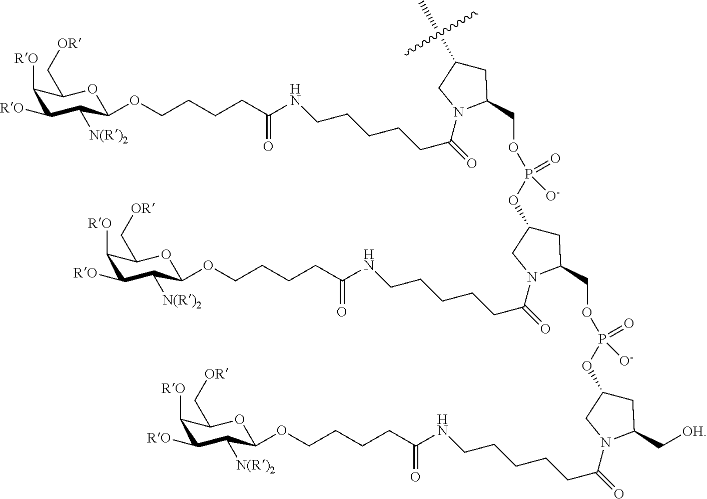

In some embodiments, an oligonucleotide comprises

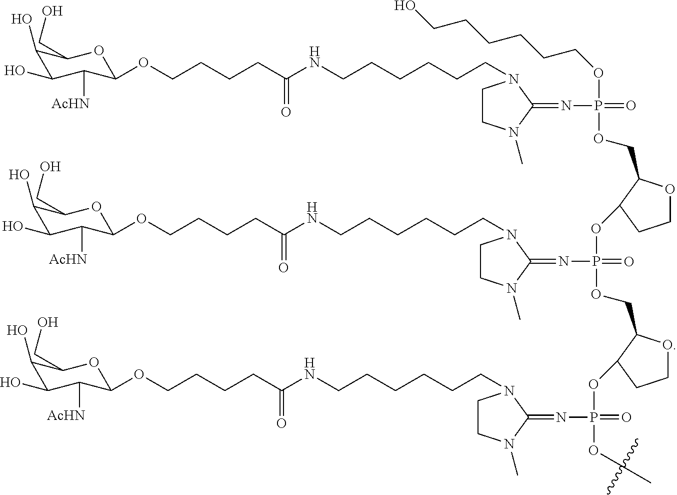 [SMILES: *OP(=O)(N=C1N(C)CCN1CCCCCCNC(=O)CCCCOC1OC(CO)C(O)C(O)C1NC(C)=O)OC1CCO[C@@H]1COP(=O)(N=C1N(C)CCN1CCCCCCNC(=O)CCCCOC1OC(CO)C(O)C(O)C1NC(C)=O)OC1CCO[C@@H]1COP(=O)(N=C1N(C)CCN1CCCCCCNC(=O)CCCCOC1OC(CO)C(O)C(O)C1NC(C)=O)OCCCCCCO]

In some embodiments, each —OR′ is —OAc, and —N(R′)₂ is —NHAc. In some embodiments, an oligonucleotide comprises

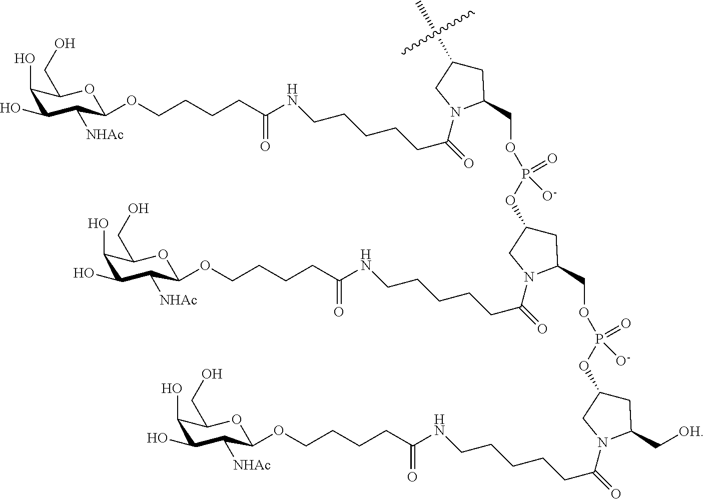

Among other things,

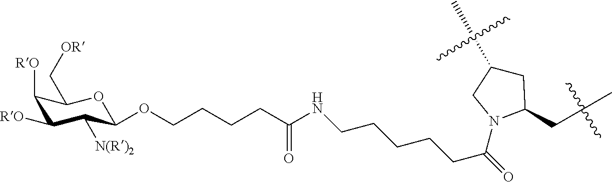

may be utilized to introduce

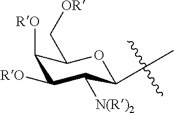

with comparable and/or better activities and/or properties. In some embodiments, it provides improved preparation efficiency and/or lower cost for the same number of

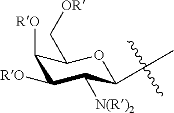

(e.g., when compared to Mod001).

In some embodiments, an additional chemical moiety is a Mod group described herein, e.g., in Table 1.

In some embodiments, an additional chemical moiety is Mod001. In some embodiments, an additional chemical moiety is Mod083. In some embodiments, an additional chemical moiety, e.g., a Mod group, is directly conjugated (e.g., without a linker) to the remainder of the oligonucleotide. In some embodiments, an additional chemical moiety is conjugated via a linker to the remainder of the oligonucleotide. In some embodiments, additional chemical moieties, e.g., Mod groups, may be directly connected, and/or via a linker, to nucleobases, sugars and/or internucleotidic linkages of oligonucleotides. In some embodiments, Mod groups are connected, either directly or via a linker, to sugars. In some embodiments, Mod groups are connected, either directly or via a linker, to 5′-end sugars. In some embodiments, Mod groups are connected, either directly or via a linker, to 5′-end sugars via 5′ carbon. For examples, see various oligonucleotides in Table 1. In some embodiments, Mod groups are connected, either directly or via a linker, to 3′-end sugars. In some embodiments, Mod groups are connected, either directly or via a linker, to 3′-end sugars via 3′ carbon. In some embodiments, Mod groups are connected, either directly or via a linker, to nucleobases. In some embodiments, Mod groups are connected, either directly or via a linker, to internucleotidic linkages. In some embodiments, provided oligonucleotides comprise Mod001 connected to 5′-end of oligonucleotide chains through L001.

As appreciated by those skilled in the art, an additional chemical moiety may be connected to an oligonucleotide chain at various locations, e.g., 5′-end, 3′-end, or a location in the middle (e.g., on a sugar, a base, an internucleotidic linkage, etc.). In some embodiments, it is connected at a 5′-end. In some embodiments, it is connected at a 3′-end. In some embodiments, it is connected at a nucleotide in the middle.

Certain additional chemical moieties (e.g., lipid moieties, targeting moieties, carbohydrate moieties), such as Mod012, Mod039, Mod062, Mod085, Mod086, and Mod094, etc., and various linkers for connecting additional chemical moieties to oligonucleotide chains, such as L001, L003, L004, L008, L009, L010, etc., and their uses, are described in WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO 2019/032612, WO 2019/055951, WO 2019/075357, WO 2019/200185, WO 2019/217784, WO 2019/032612, WO 2020/191252, WO 2021/071858, and/or WO 2022/099159, the additional chemical moieties and linkers and uses thereof of each of which are independently incorporated herein by reference, and can be utilized in accordance with the present disclosure. In some embodiments, an additional chemical moiety is digoxigenin or biotin or a derivative thereof.

In some embodiments, an additional chemical moiety (e.g., a linker, lipid, solubilizing group, conjugate group, targeting group, and/or targeting ligand) is one described in WO 2012/030683 or WO 2021/030778. In some embodiments, a provided oligonucleotide comprise a chemical structure (e.g., a linker, lipid, solubilizing group, and/or targeting ligand) described in WO 2012/030683, WO 2021/030778, WO 2019112485, US 20170362270, WO 2018156056, or WO 2018056871, WO 2021/030778, WO 2020/154344, WO 2020/154343, WO 2020/154342, WO 2020/165077, WO 2020/201406, WO 2020/216637, or WO 2020/252376.

In some embodiments, an additional chemical moiety (e.g., a targeting group, a conjugate group, etc.) and/or a modification (e.g., of nucleobase, sugar, internucleotidic linkage, etc.) are described in: U.S. Pat. Nos. 5,688,941; 6,294,664; 6,320,017; 6,576,752; 5,258,506; 5,591,584; 4,958,013; 5,082,830; 5,118,802; 5,138,045; 6,783,931; 5,254,469; 5,414,077; 5,486,603; 5,112,963; 5,599,928; 6,900,297; 5,214,136; 5,109,124; 5,512,439; 4,667,025; 5,525,465; 5,514,785; 5,565,552; 5,541,313; 5,545,730; 4,835,263; 4,876,335; 5,578,717; 5,580,731; 5,451,463; 5,510,475; 4,904,582; 5,082,830; 4,762,779; 4,789,737; 4,824,941; 4,828,979; 5,595,726; 5,214,136; 5,245,022; 5,317,098; 5,371,241; 5,391,723; 4,948,882; 5,218,105; 5,112,963; 5,567,810; 5,574,142; 5,578,718; 5,608,046; 4,587,044; 4,605,735; 5,585,481; 5,292,873; 5,552,538; 5,512,667; 5,597,696; 5,599,923; 7,037,646; 5,587,371; 5,416,203; 5,262,536; 5,272,250; or 8,106,022.

In some embodiments, an additional chemical moiety, e.g., a Mod, is connected via a linker. Various linkers are available in the art and may be utilized in accordance with the present disclosure, for example, those utilized for conjugation of various moieties with proteins (e.g., with antibodies to form antibody-drug conjugates), nucleic acids, etc. Certain useful linkers are described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, WO 2018/237194, WO 2019/032607, WO 2019/055951, WO 2019/075357, WO 2019/200185, WO 2019/217784, WO 2019/032612, WO 2020/191252, WO 2021/071858, and/or WO 2022/099159, the linker moieties of each which are independently incorporated herein by reference. In some embodiments, a linker is, as non-limiting examples, L001, L004, L009 or L010. In some embodiments, an oligonucleotide comprises a linker, but not an additional chemical moiety other than the linker. In some embodiments, an oligonucleotide comprises a linker, but not an additional chemical moiety other than the linker, wherein the linker is L001, L004, L009, or L010. In some embodiments, a linker is or comprises a moiety having the structure of an internucleotidic linkage as described herein. In some embodiments, such a moiety in a linker does not connect two nucleosides. In some embodiments, a linker has the structure of L. In some embodiments, a linker is bivalent. In some embodiments, a linker is polyvalent. In some embodiments, a linker can connect two or more additional chemical moieties to an oligonucleotide chain as described herein. For example, some embodiments, one or two or three or more additional chemical moieties, e.g., GalNAc moieties, are connected to an oligonucleotide chain (e.g., at 5′-end) through a multivalent linker moiety.

In some embodiments, an additional chemical moiety is cleaved from the remainder of an oligonucleotide, e.g., an oligonucleotide chain, e.g., after administration to a system, cell, tissue, organ, subject, etc. In some embodiments, additional chemical moieties promote, increase, and/or accelerate delivery to certain cells, and after delivery of oligonucleotides into such cells, additional chemical moieties are cleaved from oligonucleotides. In some embodiments, linker moieties comprise one or more cleavable moieties that can be cleaved at desirable locations (e.g., within certain type of cells, subcellular compartments such as lysosomes, etc.) and/or timing. In some embodiments, a cleavable moiety is selectively cleaved by a polypeptide, e.g., an enzyme such as a nuclease. Many useful cleavable moieties and cleavable linkers are reported and can be utilized in accordance with the present disclosure. In some embodiments, a cleavable moiety is or comprises one or more functional groups selected from amide, ester, ether, phosphodiester, disulfide, carbamate, etc. In some embodiments, a linker is as described in WO 2012/030683, WO 2021/030778, WO 2020/154344, WO 2020/154343, WO 2020/154342, WO 2020/165077, WO 2020/201406, WO 2020/216637, or WO 2020/252376.

As demonstrated herein, provided technologies can provide high levels of activities and/or desired properties, in some embodiments, without utilizing particular structural elements (e.g., modifications, linkage configurations and/or patterns, etc.) reported to be desired and/or necessary (e.g., those reported in WO 2019/219581), though certain such structural elements may be incorporated into oligonucleotides in combination with various other structural elements in accordance with the present disclosure.

### ADAR

Among other things, provided technologies can provide modification/editing of target adenosine by converting A to I. In some embodiments, oligonucleotides and/or duplexes formed by oligonucleotides with target nucleic acids interact with proteins, e.g., ADAR proteins. In some embodiments, such proteins comprise adenosine modifying activities and can modify target adenosine in target nucleic acids, e.g., converting them to inosine.

ADAR proteins are naturally expressed proteins in various cells, tissues, organs and/or organism. It has been reported that some ADAR proteins, e.g., ADAR1 and ADAR2, can edit adenosine through deamination, converting adenosine to inosine which can provide a number of functions including being read as or similar to G during translation. Mechanism of ADAR-mediated mRNA editing (e.g., deamination) has been reported. For example, ADAR proteins are reported to catalyze conversion of adenosine to inosine on double-stranded RNA substrates with mismatches. As appreciated by those skilled in the art, inosine can be recognized as guanosine by cellular translation and/or splicing machinery. ADAR can thus be used for functional adenosine to guanosine editing of nucleic acids, e.g., pre-mRNA and mRNA substrates.

In some embodiments, the present disclosure provides oligonucleotides and compositions thereof for ADAR-mediated editing of target adenosine in target nucleic acids, e.g. RNA, ADAR-mediated RNA-editing can offer several advantages over DNA-editing, e.g., delivery is simplified as expression of recombinant proteins like Cas9 is not required. Both ADAR1 and ADAR2 are endogenous enzymes, so cellular delivery of oligonucleotides alone can be sufficient for editing. Off-target effects, if any, are transient and changes are not made to genomic DNA. Additionally, ADAR-mediated editing can be used in post-mitotic cells and it does not require an HDR-template for repair. Three vertebrate ADAR genes have been reported with common functional domains (Nishikura Nat Rev Mol Cell Biol. 2016 February; 17(2): 83-96; Nishikura Annu Rev Biochem. 2010; 79: 321-349; Thomas and Beal Bioessays. 2017 April; 39(4)). All 3 ADARs contain a dsRNA-binding domains (dsRBD), which can contact dsRNA substrates. Some ADAR1 also contains Z-DNA-binding domains. ADAR1 has been reported to expressed significantly in brain, lung, kidney, liver, and heart, etc., and may occur in two isoforms. In some embodiments, isoform p150 can be induced by interferon while isoform p110 can be constitutively expressed. In some embodiments, it can be beneficial to utilize p110 as it is reported to be ubiquitously and constitutively expressed. ADAR2 can be highly expressed, e.g. in the brain and lungs, and is reported to be exclusively localized to the nucleus. ADAR3 is reported to be catalytically inactive and expressed only in the brain. Potential differences in tissue expression can be taken into consideration when choosing a therapeutic target.

Use of oligonucleotides for RNA editing by ADAR has been reported. Among other things, the present disclosure recognizes that previously reported technologies generally suffer one or more disadvantages, such as low stability (e.g., oligonucleotides with natural RNA sugars), low editing efficiency, low editing specificity (e.g., a number of As are edited in a portion of a target nucleic acid substantially complementary to an oligonucleotide), specific structures in oligonucleotides for ADAR recognition/recruitment, exogenous proteins (e.g., those engineered to recognize oligonucleotides with specific structures and/or duplexes thereof (e.g., with target nucleic acids) for editing), etc. Additionally, previously reported technologies typically utilize stereorandom oligonucleotide compositions when oligonucleotides comprise one or more chiral linkage phosphorus of modified internucleotidic linkages.

For example, various reported oligonucleotides contain ADAR-recruiting domains. Merkle et al., Nat Biotechnol. 2019 February; 37(2):133-138 disclosed oligonucleotides comprising an imperfect 20-bp hairpin ADAR-recruiting domain that is an intramolecular stem loop to recruit endogenous human ADAR2 to edit endogenous transcript. Oligonucleotides reported in Mali et al., Nat Methods. 2019 March:16(3):239-242 contain ADAR substrate GluR2 pre-messenger RNA sequences or MS2 hairpins in addition to specificity domains that hybridize to the target mRNA.

Certain reported editing approach utilizes exogenous or engineered proteins, e.g., those utilizing CRISPR/Cas9 system. For example, Komor et al. Nature 2016 volume533, pages420-424 disclosed deaminase coupled with CRISPR-Cas9 to create programmable DNA base editors. Since it engages in exogenous editing proteins, it requires the delivery of both the CRISPR/Cas9 system and the guide RNA.

Among other things, the present disclosure provides technologies comprising one or more features such as sugar modifications, base modifications, internucleotidic linkage modifications, control of stereochemistry, various patterns thereof, etc. to solve one or more or all disadvantaged suffered from prior adenosine editing technologies, for example, through providing chirally controlled oligonucleotide compositions of designed oligonucleotides described herein. For example, as demonstrated herein. ADAR-recruiting loops are optional and not required for provided technology.

As appreciated by those skilled in the art, one or more of such useful features may be utilized to improve oligonucleotides in prior technologies (e.g., those described in WO 2016097212, WO 2017220751, WO 2018041973, WO 2018134301, oligonucleotides and oligonucleotide compositions of each of which are independently incorporated by reference). In some embodiments, the present disclosure provides improvements of prior technologies by apply one or more useful features described herein to prior reported oligonucleotide base sequences. In some embodiments, the present disclosure provides chirally controlled oligonucleotide compositions of previously reported oligonucleotides that may be useful for adenosine editing. In some embodiments, the present disclosure provides improvements of previously reported adenosine editing using stereorandom oligonucleotide compositions by performing such editing using chirally controlled oligonucleotide compositions.

As reported, ADAR proteins may have various isoforms. For example, ADAR1 has, among others, a reported p110 isoform and a reported p150 isoform. In some embodiments, it was observed that certain chirally controlled oligonucleotide compositions can provide high levels of adenosine modification (e.g., conversion of A to I) with multiple isoforms, in some embodiments, both p110 and p150 isoforms, while stereorandom compositions provide low levels of adenosine modification for one or more isoforms (e.g., p110). In some embodiments, chirally controlled oligonucleotide composition are particularly useful for adenosine modification in systems (e.g., cells, tissues, organs, organisms, subjects, etc.) expressing or comprising the p110 isoform of ADAR1, particularly those expressing or comprising high levels of the p110 isoform of ADAR1 relative to the p150 isoform, or those expressing no or low levels of ADAR1 p150.

In some embodiments, the present disclosure provides Cis-acting (CisA) oligonucleotide that do not require stem loop in the structure. In some embodiments, a provided oligonucleotide can form a dsRNA structure with a target mRNA through base pairing. In some embodiments, formed dsRNA structures (optionally with secondary mismatches) contain bulges that promote ADAR binding and therefore, can facilitate ADAR-mediated editing (e.g., deamination of a target adenosine). In some embodiments, oligonucleotides of the present disclosure are shorter than LSL oligonucleotides or CSL oligonucleotides, e.g., no more than or about 32 nt, no more than or about 31 nt, no more than or about 30 nt, no more than or about 29 nt, no more than or about 28 nt, no more than or about 27 nt, or no more than or about 26 nt in length, and can provide high editing efficiency.

### Duplexing and Targeting Regions

In some embodiments, an oligonucleotide comprises a duplexing region and a targeting region. In some embodiments, a duplexing region forms a duplex with another nucleic acid, e.g., a duplexing oligonucleotide. Useful duplexing technologies including duplexing oligonucleotides and uses thereof with provided oligonucleotides are described in WO 2022/099159 and are incorporated herein by reference.

### Production of Oligonucleotides and Compositions

Various methods can be utilized for production of oligonucleotides and compositions and can be utilized in accordance with the present disclosure. For example, traditional phosphoramidite chemistry (e.g., phosphoramidites comprising —CH₂CH₂CN and —N(i-Pr)₂) can be utilized to prepare stereorandom oligonucleotides and compositions, and certain reagents and chirally controlled technologies can be utilized to prepare chirally controlled oligonucleotide compositions, e.g., as described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, WO 2018/237194, WO 2019/032607, WO 2019/055951, WO 2019/075357, WO 2019/200185, WO 2019/217784, WO 2019/032612, WO 2020/191252, WO 2021/071858, and/or WO 2022/099159, the reagents and methods of each of which is incorporated herein by reference.

In some embodiments, chirally controlled/stereoselective preparation of oligonucleotides and compositions thereof comprise utilization of a chiral auxiliary, e.g., as part of monomers, dimers (e.g., chirally pure dimers from separation), monomeric phosphoramidites, dimeric phosphoramidites (e.g., chirally pure dimers from separation), etc. Examples of such chiral auxiliary reagents, monomers, dimers, and phosphoramidites are described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, WO 2018/237194, WO 2019/032607, WO 2019/055951, WO 2019/075357, WO 2019/200185, WO 2019/217784, WO 2019/032612, WO 2020/191252, WO 2021/071858, and/or WO 2022/099159, the chiral auxiliary reagents, monomers, dimers, and phosphoramidites of each of which are independently incorporated herein by reference. In some embodiments, a chiral auxiliary is a chiral auxiliary described in any of: WO 2018/022473, WO 2018/098264, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, WO 2021/071858, and/or WO 2022/099159, the chiral auxiliaries of each of which are independently incorporated herein by reference.

In some embodiments, chirally controlled preparation technologies, including oligonucleotide synthesis cycles, reagents and conditions are described in U.S. Pat. No. 9,982,257. US 20170037399, US 20180216108. US 20180216107, U.S. Pat. No. 9,598,458, WO 2017/062862, WO 2018/067973, WO 2017/160741, WO 2017/192679. WO 2017/210647, and/WO 2018/098264, WO 2018/022473, WO 2018/223056, WO 2018/223073, WO 2018/223081, WO 2018/237194, WO 2019/032607, WO 2019/032612, WO 2019/055951, WO 2019/075357, WO 2019/200185, WO 2019/217784, WO 2019/032612, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, WO 2021/071858, and/or WO 2022/099159, the oligonucleotide synthesis methods, cycles, reagents and conditions of each of which are independently incorporated herein by reference.

Once synthesized, provided oligonucleotides and compositions are typically further purified. Suitable purification technologies are widely known and practiced by those skilled in the art, including but not limited to those described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, WO 2018/237194, WO 2019/032607, WO 2019/055951, WO 2019/075357, WO 2019/200185, WO 2019/217784, WO 2019/032612, WO 2020/191252, WO 2021/071858, and/or WO 2022/099159, the purification technologies of each of which are independently incorporated herein by reference.

In some embodiments, a cycle comprises or consists of coupling, capping, modification and deblocking. In some embodiments, a cycle comprises or consists of coupling, capping, modification, capping and deblocking. These steps are typically performed in the order they are listed, but in some embodiments, as appreciated by those skilled in the art, the order of certain steps, e.g., capping and modification, may be altered. If desired, one or more steps may be repeated to improve conversion, yield and/or purity as those skilled in the art often perform in syntheses. For example, in some embodiments, coupling may be repeated; in some embodiments, modification (e.g., oxidation to install ═O, sulfurization to install ═S, etc.) may be repeated; in some embodiments, coupling is repeated after modification which can convert a P(Ill) linkage to a P(V) linkage which can be more stable under certain circumstances, and coupling is routinely followed by modification to convert newly formed P(III) linkages to P(V) linkages. In some embodiments, when steps are repeated, different conditions may be employed (e.g., concentration, temperature, reagent, time, etc.).

Technologies for formulating provided oligonucleotides and/or preparing pharmaceutical compositions, e.g., for administration to subjects via various routes, are readily available in the art and can be utilized in accordance with the present disclosure, e.g., those described in U.S. Pat. No. 9,982,257, US 20170037399, US 20180216108. US 20180216107, U.S. Pat. No. 9,598,458, WO 2017/062862, WO 2018/067973, WO 2017/160741, WO 2017/192679, WO 2017/210647, WO 2018/098264, WO 2018/223056, or WO 2018/237194 and references cited therein.

Technologies for formulating provided oligonucleotides and/or preparing pharmaceutical compositions, e.g., for administration to subjects via various routes, are readily available in the art and can be utilized in accordance with the present disclosure, e.g., those described in U.S. Pat. No. 9,982,257. US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, or WO 2018/237194 and references cited therein.

In some embodiments, a useful chiral auxiliary has the structure of

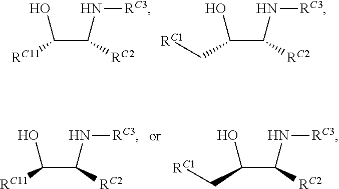

or a salt thereof, wherein R^{C11} is -L^{C1}-R^{C1}, L^{C1} is optionally substituted —CH₂—, R^{C1} is R, —Si(R)₃, —SO₂R or an electron-withdrawing group, and R^{C2} and R^{C3} are taken together with their intervening atoms to form an optionally substituted 3-10 membered saturated ring having, in addition to the nitrogen atom, 0-2 heteroatoms. In some embodiments, a useful chiral auxiliary has the structure of

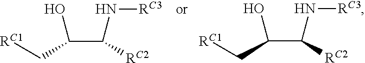

wherein R^{C1} is R, —Si(R)₃ or —SO₂R, and R^{C2} and R^{C3} are taken together with their intervening atoms to form an optionally substituted 3-7 membered saturated ring having, in addition to the nitrogen atom, 0-2 heteroatoms, is a formed ring is an optionally substituted 5-membered ring. In some embodiments, a useful chiral auxiliary has the structure of

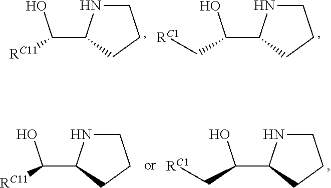

or a salt thereof. In some embodiments, a useful chiral auxiliary has the structure of

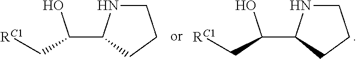

In some embodiments, a useful chiral auxiliary is a DPSE chiral auxiliary. In some embodiments, purity or stereochemical purity of a chiral auxiliary is at least 85%, 90%, 95%, 96%, 97%, 98%, or 99%. In some embodiments, it is at least 85%. In some embodiments, it is at least 90%. In some embodiments, it is at least 95%. In some embodiments, it is at least 96%. In some embodiments, it is at least 97%. In some embodiments, it is at least 98%. In some embodiments, it is at least 99%.

In some embodiments, L^{C1} is —CH₂—. In some embodiments, L^{C1} is substituted —CH₂—. In some embodiments, L^{C1} is mono-substituted —CH₂—.

In some embodiments, R^{C1} is R. In some embodiments, R^{C1} is optionally substituted phenyl. In some embodiments, R^{C1} is —SiR₃. In some embodiments, R is —SiPh₂Me. In some embodiments, R^{C1} is —SO₂R. In some embodiments, R is not hydrogen. In some embodiments, R is optionally substituted phenyl. In some embodiments, R is phenyl. In some embodiments, R is optionally substituted C₁₋₆ aliphatic. In some embodiments, R is C₁₋₆ alkyl. In some embodiments, R is methyl. In some embodiments, R is t-butyl.

In some embodiments, R^{C1} is an electron-withdrawing group, such as —C(O)R. —OP(O)(OR)₂, —OP(O)(R)₂, —P(O)(R)₂, —S(O)R, —S(O)₂R, etc. In some embodiments, chiral auxiliaries comprising electron-withdrawing group R^{C1} groups are particularly useful for preparing chirally controlled non-negatively charged internucleotidic linkages and/or chirally controlled internucleotidic linkages bonded to natural RNA sugar.

In some embodiments, R^{C2} and R^{C3} are taken together with their intervening atoms to form an optionally substituted 3-10 (e.g., 3, 4, 5, 6, 7, 8, 9, or 10) membered saturated ring having no heteroatoms in addition to the nitrogen atom. In some embodiments, R^{C2} and R^{C3} are taken together with their intervening atoms to form an optionally substituted 5-membered saturated ring having no heteroatoms in addition to the nitrogen atom.

In some embodiments, methods for preparing oligonucleotides and/or compositions comprise using a chiral auxiliary described herein, e.g., for constructing one or more chirally controlled internucleotidic linkages. In some embodiments, one or more chirally controlled internucleotidic linkages are independently constructed using a DPSE chiral auxiliary. In some embodiments, each chirally controlled phosphorothioate internucleotidic linkage is independently constructed using a DPSE chiral auxiliary. In some embodiments, one or more chirally controlled internucleotidic linkages are independently constructed using

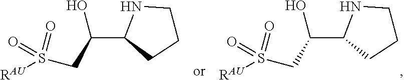

or a salt thereof, wherein R^{AU} is as described herein. In some embodiments, each chirally controlled non-negatively charged internucleotidic linkage (e.g., n001) is independently constructed using

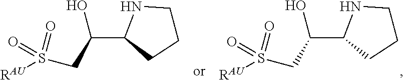

or a salt thereof. In some embodiments, each chirally controlled internucleotidic linkage is independently constructed using

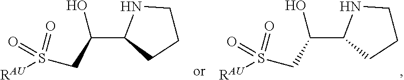

or a salt thereof. In some embodiments, R^{AU} is optionally substituted C₁₋₂₀, C₁₋₁₀, C₁₋₆, C₁₋₅, or C₁₋₄ aliphatic. In some embodiments, R^{AU} is optionally substituted C₁₋₂₀, C₁₋₁₀, C₁₋₆, C₁₋₅, or C₁₋₄ alkyl. In some embodiments, R^{AU} is optionally substituted aryl. In some embodiments, R^{AU} is phenyl. In some embodiments, one or more chirally controlled internucleotidic linkages are constructed using a PSM chiral auxiliary. In some embodiments, each chirally controlled non-negatively charged internucleotidic linkage (e.g., n001) is independently constructed using a PSM chiral auxiliary. In some embodiments, each chirally controlled internucleotidic linkages is independently constructed using a PSM chiral auxiliary. As appreciated by those skilled in the art, a chiral auxiliary is often utilized in a phosphoramidite (e.g.,

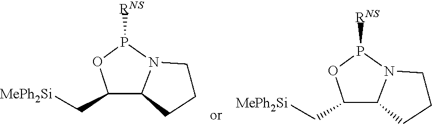

(DPSE phosphoramidites),

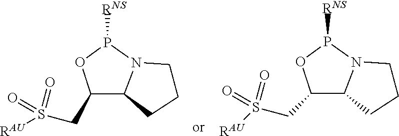

(wherein R^{AU} is independently as described herein; when R^{AU} is -Ph, PSM phosphoramidites), wherein R^{NS} is an optionally substituted/protected nucleoside (e.g., optionally protected for oligonucleotide synthesis), or a salt thereof, etc.) for oligonucleotide preparation. In some embodiments, a phosphoramidite is a compound having the structure of

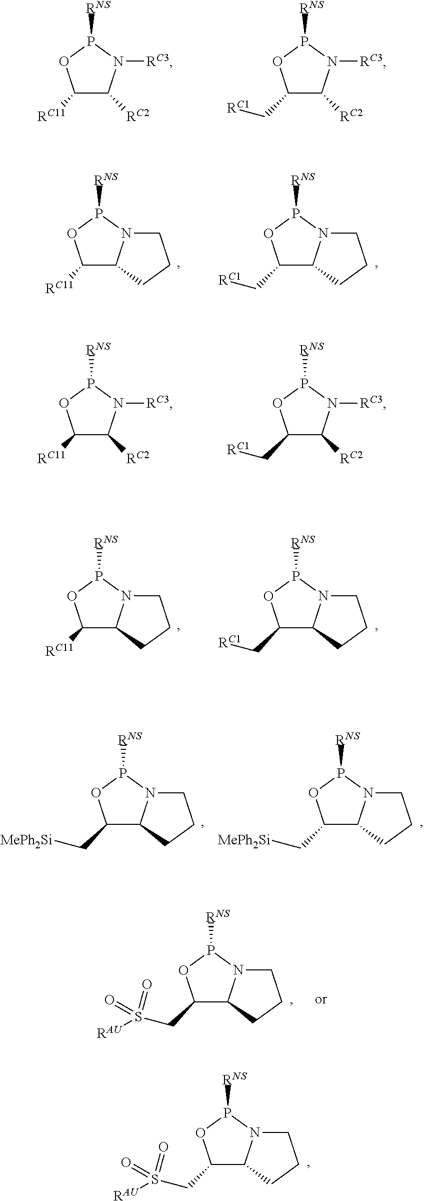

or salt thereof, wherein each variable is independently as described herein. In some embodiments, R^{UA} is optionally substituted phenyl. In some embodiments, R^{AU} is phenyl. In some embodiments, R^{NS} is an optionally substituted or protected nucleoside comprising hypoxanthine. In some embodiments, R^{NS} comprises optionally substituted or protected hypoxanthine. In some embodiments, RN is optionally substituted or protected inosine. In some embodiments, R^{NS} is optionally substituted or protected deoxyinosine. In some embodiments, R^{NS} is optionally substituted or protected 2′-F inosine (2′-OH replaced with 2′-F). In some embodiments, R^{NS} is optionally substituted or protected 2′-OR modified inosine (2′-OH replaced with a 2′-OR modification as described herein (e.g., 2′-OMe, 2′-MOE, etc.)). In some embodiments, hypoxanthine is O⁶ protected. In some embodiments, hypoxanthine is O⁶ protected with -L-Si(R)₃, wherein L is optionally substituted —CH₂—CH₂—, and each R is independently as described herein and not —H. In some embodiments, each R is independently an optionally substituted group selected from C₁₋₆ aliphatic and phenyl. In some embodiments, each R is independently optionally substituted C₁₋₆ alkyl. In some embodiments, -L-Si(R)₃ is —CH₂CH₂Si(Me)₃. In some embodiments, compounds comprising O⁶ protected hypoxanthine (e.g., with —CH₂CH₂Si(Me)₃) have higher solubility than corresponding (Y unprotected compounds and may provide various benefits and advantages when utilized for oligonucleotide synthesis in accordance with the present disclosure. In some embodiments, in a compound having the structure of

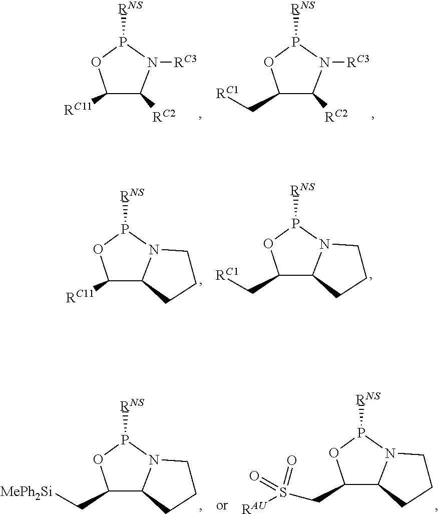

or salt thereof, R^{NS} comprises an O⁶ protected hypoxanthine (e.g., with —CH₂CH₂Si(Me)₃). In some embodiments, R^{NS} is O⁶-protected inosine. In some embodiments, R^{NS} is O⁶-protected deoxyinosine. In some embodiments, R^{NS} is O⁶-protected 2′-F inosine. In some embodiments, R^{NS} O⁶-protected 2′-OR modified inosine whose 2′-OR modification is as described herein (e.g., 2′-OMe, 2′-MOE, etc.). Among other things, the present disclosure encompasses the recognition that such a compound has sufficient solubility for oligonucleotide synthesis and can be utilized in oligonucleotide synthesis while a corresponding compound without O⁶ protection may not have sufficient solubility for efficient oligonucleotide synthesis. In some embodiments, a phosphoramidite is (1S,3S,3aS)-1-(((2R,3S,5R)-2-((bis(4-methoxyphenyl)(phenyl)methoxy)methyl)-5-(6-(2-(trimethylsilyl)ethoxy)-9H-purin-9-yl)tetrahydrofuran-3-yl)oxy)-3-((methyldiphenylsilyl)methyl)tetrahydro-1H,3H-pyrrolo[1,2-c][1,3,2]oxazaphosphole. In some embodiments, a phosphoramidite is (1S,3S,3aS)-1-(((2R,3S,5R)-2-((bis(4-methoxyphenyl)(phenyl)methoxy)methyl)-5-(6-(2-(trimethylsilyl)ethoxy)-9H-purin-9-yl)tetrahydrofuran-3-yl)oxy)-3-((phenylsulfonyl)methyl)tetrahydro-1H,3H-pyrrolo[1,2-c][1,3,2]oxazaphosphole. In some embodiments, in a compound having the structure of

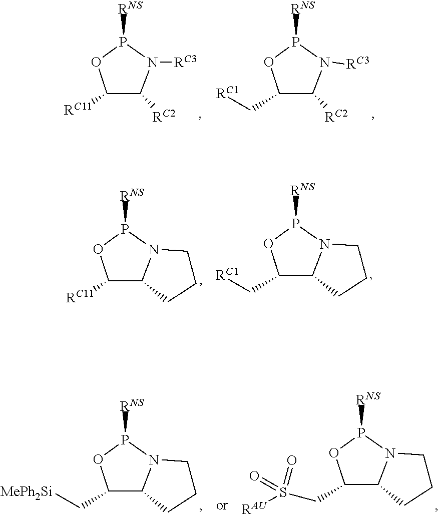

or salt thereof, R^{NS} comprises an O⁶ unprotected hypoxanthine. In some embodiments, R^{NS} is optionally substituted or protected inosine wherein the hypoxanthine is unprotected. In some embodiments, R^{NS} is optionally substituted or protected deoxyinosine wherein the hypoxanthine is unprotected. In some embodiments, R^{NS} is optionally substituted or protected 2′-F inosine wherein the hypoxanthine is unprotected. In some embodiments, R-s is optionally substituted or protected 2′-OR modified inosine wherein the hypoxanthine is unprotected and whose 2′-OR modification is as described herein (e.g., 2′-OMe, 2′-MOE, etc.). Among other things, the present disclosure encompasses the recognition that such a compound has sufficient solubility for oligonucleotide synthesis and can be utilized in oligonucleotide synthesis without 06 protection.

In some embodiments, a method comprises providing a DPSE and/or a PSM phosphoramidite or a salt thereof. In some embodiments, a provided method comprises contacting a DPSE and/or a PSM phosphoramidite or a salt thereof with —OH (e.g., 5′-OH of a nucleoside or an oligonucleotide chain). As those skilled in the art appreciate, contacting can be performed under various suitable conditions so that a phosphorus linkage is formed. In some embodiments, preparation of each chirally controlled internucleotidic linkage independently comprises contacting a DPSE or PSM phosphoramidite or a salt thereof with —OH (e.g., 5′-OH of a nucleoside or an oligonucleotide chain). In some embodiments, preparation of each chirally controlled phosphorothioate internucleotidic linkage independently comprises contacting a DPSE phosphoramidite or a salt thereof with —OH (e.g., 5′-OH of a nucleoside or an oligonucleotide chain). In some embodiments, preparation of each chirally controlled non-negatively charged internucleotidic linkage (e.g., n001) independently comprises contacting a PSM phosphoramidite or a salt thereof with —OH (e.g., 5′-OH of a nucleoside or an oligonucleotide chain). In some embodiments, preparation of each chirally controlled internucleotidic linkage independently comprises contacting a PSM phosphoramidite or a salt thereof with —OH (e.g., 5′-OH of a nucleoside or an oligonucleotide chain). In some embodiments, contacting forms a P(III) linkage comprising a phosphorus atom bonded to two sugars and a chiral auxiliary moiety (e.g.,

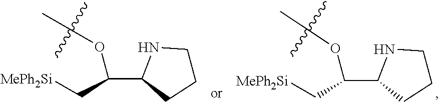

or a salt form thereof (e.g., from DPSE phosphoramidites or salts thereof),

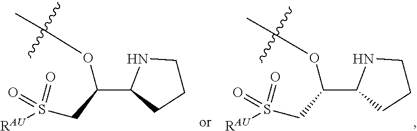

or a salt form thereof (wherein R^{AU} is independently as described herein; when R^{AU} is -Ph, e.g., from PSM phosphoramidites or salts thereof), etc.). In some embodiments, an oligonucleotide comprises a P(III) linkage comprising a chiral auxiliary moiety, e.g., from a DPSE or PSM phosphoramidite. In some embodiments, a P(III) linkage comprising a chiral auxiliary moiety is chirally controlled. In some embodiments, a chiral auxiliary moiety may be protected, e.g., before converting a P(III) linkage to a P(V) linkage (e.g., before sulfurization, reacting with azide, etc.). In some embodiments, a protected chiral auxiliary has the structure of

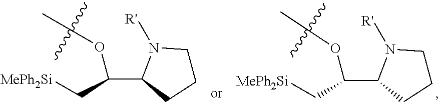

or a salt form thereof (e.g., wherein R′ is independently as described herein; e.g., from DPSE phosphoramidites or salts thereof), or

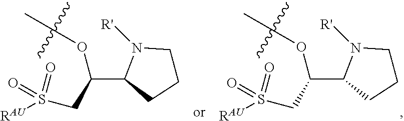

or a salt form thereof (wherein each R′ and R^{AU} is independently as described herein; when R^{AU} is -Ph, e.g., from PSM phosphoramidites or salts thereof), wherein each R′ is independently as described herein. In some embodiments, R′ is —C(O)R, wherein R is as described herein. In some embodiments, R is —CH₃. In some embodiments, an oligonucleotide comprises a protected chiral auxiliary. In some embodiments, each chirally controlled internucleotidic linkage in an oligonucleotide independently comprises

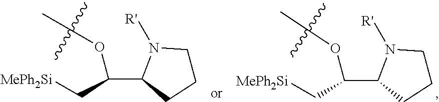

or a salt form thereof, or

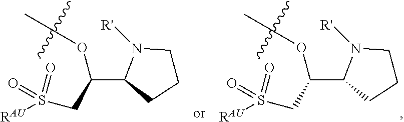

or a salt form thereof. In some embodiments, each chirally controlled internucleotidic linkage in an oligonucleotide independently comprises

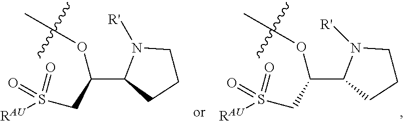

or a salt form thereof. In some embodiments, R′ is —C(O)R. In some embodiments, R′ is —C(O)CH₃. In some embodiments, R^{AU} is Ph. In some embodiments, an oligonucleotide comprises one or more

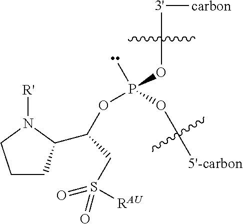

or a salt form thereof (PIII-1), wherein each variable independently as described herein. In some embodiments, an oligonucleotide comprises one or more

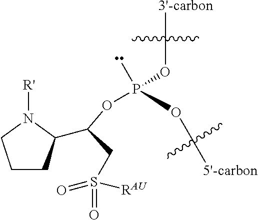

or a salt form thereof (PIII-2), wherein each variable independently as described herein. In some embodiments, an oligonucleotide comprises one or more

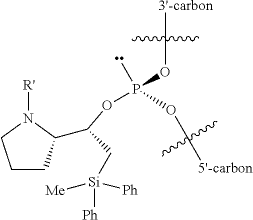

or a salt form thereof (PIII-5), wherein each variable independently as described herein. In some embodiments, an oligonucleotide comprises one or more

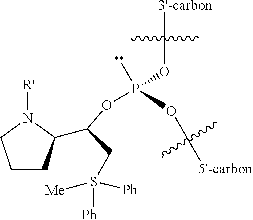

or a salt form thereof (PIII-6), wherein each variable independently as described herein. In some embodiments, a 5′-end internucleotidic linkage is PIII-1, PIII-2, PIII-5, or PIII-6. In some embodiments, a 5′-end internucleotidic linkage is PIII-1 or PIII-2. In some embodiments, R′ is —H. In some embodiments, R′ is —C(O)R. In some embodiments, R′ is —C(O)CH₃. In some embodiments, R^{AU} is -Ph. In some embodiments, a P(III) linkage is converted into a P(V) linkage. In some embodiments, a P(V) linkage comprises a phosphorus atom bonded to two sugars, a chiral auxiliary moiety (e.g.,

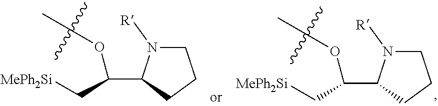

or a salt form thereof (wherein R′ is as described herein; e.g., from DPSE phosphoramidites or salts thereof),

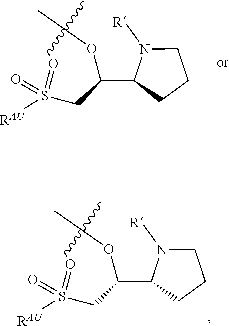

or a salt form thereof (wherein each of R′ and R^{AU} is independently as described herein; when R^{AU} is -Ph, e.g., from PSM phosphoramidites or salts thereof), etc.), and S or

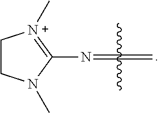 [SMILES: *=NC1=[N+](C)CCN1C]

In some embodiments, a P(V) linkage comprises a phosphorus atom bonded to two sugars,

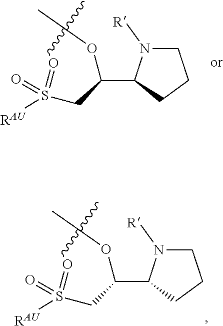

or a salt form thereof (wherein each R′ and R^{AU} is independently as described herein; when R^{AU} is -Ph, e.g., from PSM phosphoramidites or salts thereof), etc.), and S or

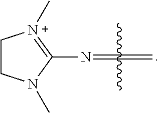 [SMILES: *=NC1=[N+](C)CCN1C]

In some embodiments, a P(V) linkage comprises a phosphorus atom bonded to two sugars,

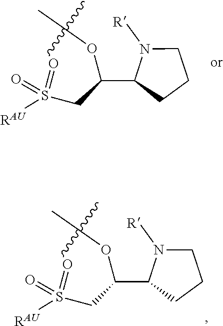

or a salt form thereof (wherein each R′ and R^{AU} is independently as described herein; when R^{UA} is -Ph, e.g., from PSM phosphoramidites or salts thereof), etc.), and S. In some embodiments, a P(V) linkage comprises a phosphorus atom bonded to two sugars

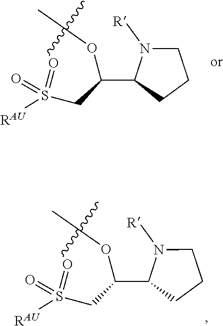

or a salt form thereof (wherein each R′ and R^{AU} is independently as described herein; when R^{AU} is -Ph, e.g., from PSM phosphoramidites or salts thereof), etc.), and

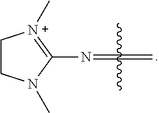 [SMILES: *=NC1=[N+](C)CCN1C]

Those skilled in the art will appreciate that

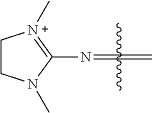 [SMILES: *=NC1=[N+](C)CCN1C]

can exist with a counterion, e.g., in some embodiments, PF₆⁻. In some embodiments, an oligonucleotide comprises one or more

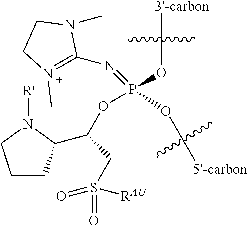

or a salt form thereof (PV-1), wherein each variable independently as described herein. In some embodiments, an oligonucleotide comprises one or more

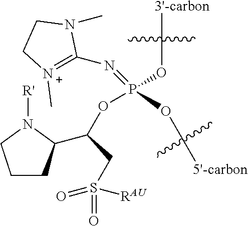

or a salt form thereof (PV-2), wherein each variable independently as described herein. In some embodiments, an oligonucleotide comprises one or more

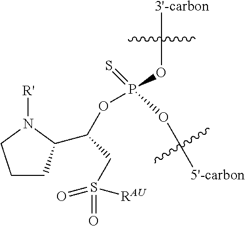

or a salt form thereof (PV-3), wherein each variable independently as described herein. In some embodiments, an oligonucleotide comprises one or more

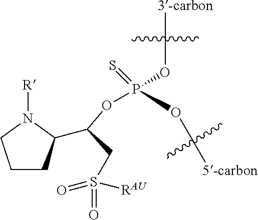

or a salt form thereof (PV-4), wherein each variable independently as described herein. In some embodiments, an oligonucleotide comprises one or more

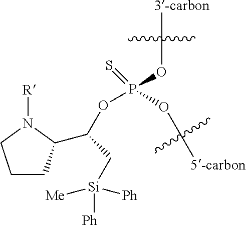

or a salt form thereof (PV-5), wherein each variable independently as described herein. In some embodiments, an oligonucleotide comprises one or more

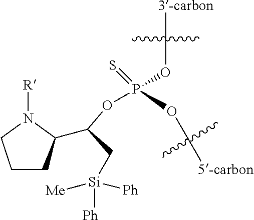

or a salt form thereof (PV-6), wherein each variable independently as described herein. In some embodiments, each chiral internucleotidic linkage, or each chirally controlled internucleotidic linkage, of an oligonucleotide is independently selected from PIII-1, PIII-2, PIII-5, PIII-6, PV-1, PV-2, PV-3, PV-4, PV-5, and PV-6. In some embodiments, each chiral internucleotidic linkage, or each chirally controlled internucleotidic linkage, of an oligonucleotide is independently selected from PIT-1, PIII-2, PV-1, PV-2, PV-3, and PV-4. In some embodiments, a linkage of PIII-1, PIII-2, PIII-5, or PIII-6 is typically the 5′-end internucleotidic linkage. In some embodiments, each chiral internucleotidic linkage, or each chirally controlled internucleotidic linkage, of an oligonucleotide is independently selected from PV-1, PV-2, PV-3, PV-4, PV-5, and PV-6. In some embodiments, each chiral internucleotidic linkage, or each chirally controlled internucleotidic linkage, of an oligonucleotide is independently selected from PV-1, PV-2, PV-3, or PV-4. In some embodiments, a provided oligonucleotide is an oligonucleotide as described herein, e.g., of Table 1, wherein each *S is independently replaced with PV-3 or PV-5, each *R is independently replaced with PV-4 or PV-6, each n001R is independently replaced with PV-1, and each n001 S is independently replaced with PV-2. In some embodiments, a provided oligonucleotide is an oligonucleotide as described herein, e.g., of Table 1, wherein each *S is independently replaced with PV-3, each *R is independently replaced with PV-4, each n001R is independently replaced with PV-1, and each n001S is independently replaced with PV-2. In some embodiments, each natural phosphate linkage is independently replaced with a precursor, e.g.,

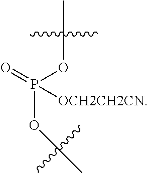

In some embodiments, R′ is —H. In some embodiments, R′ is —C(O)R. In some embodiments, R′ is —C(O)CH₃. In some embodiments, R^{AU} is -Ph. In some embodiments, a method comprises removal of one or more chiral auxiliary moieties so that phosphorothioate and/or non-negatively charged internucleotidic linkages (e.g., n001) are formed (e.g., from V-1, PV-2, PV-3, PV-4, PV-5, PV-6, etc.). In some embodiments, removal of a chiral auxiliary (e.g., PSM) comprises contacting an oligonucleotide with a base (e.g., N(R)₃ such as DEA) under anhydrous conditions.

In some embodiments, as appreciated by those skilled in the art, for preparation of a chirally controlled internucleotidic linkage, a monomer or a phosphoramidite (e.g., a DPSE or PSM phosphoramidite) is typically utilized in a chirally enriched or pure form (e.g., of a purity as described herein (e.g., about or at least about 90%, 91%, 92%, 93%, 94%, 95%, 96%, 97%, 98%, or 99%, or about 100%)).

In some embodiments, the present disclosure provides useful reagents for preparation of oligonucleotides and compositions thereof. In some embodiments, monomers and phosphoramidites comprise nucleosides, nucleobases and sugars as described herein. In some embodiments, nucleobases and sugars are properly protected for oligonucleotide synthesis as those skilled in the art will appreciate. In some embodiments, a phosphoramidite has the structure of R^{NS}—P(OR)N(R)₂, wherein R^{NS} is a optionally protected nucleoside moiety. In some embodiments, a phosphoramidite has the structure of R^{NS}—P(OCH₂CH₂CN)N(i-Pr)₂. In some embodiments, a phosphoramidite comprises a chiral auxiliary moiety, wherein the phosphorus is bonded to an oxygen and a nitrogen atom of the chiral auxiliary moiety. In some embodiments, a phosphoramidite has the structure of

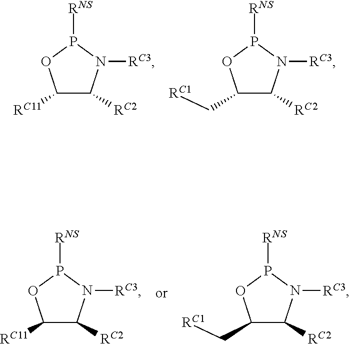

or a salt thereof, wherein R^{NS} is a protected nucleoside moiety (e.g., 5′-OH and/or nucleobases suitably protected for oligonucleotide synthesis), and each other variable is independently as described herein. In some embodiments, a phosphoramidite has the structure of

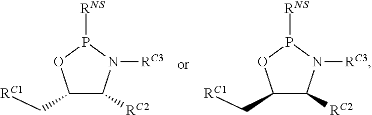

wherein R^{NS} is a protected nucleoside moiety (e.g., 5′-OH and/or nucleobases suitably protected for oligonucleotide synthesis), R^{C1} is R, —Si(R)₃ or —SO₂R, and R^{C2} and R^{C3} are taken together with their intervening atoms to form an optionally substituted 3-7 membered saturated ring having, in addition to the nitrogen atom, 0-2 heteroatoms, wherein the coupling forms an internucleotidic linkage. In some embodiments, 5′-OH of R^{NS} is protected. In some embodiments, 5′-OH of R^{NS} is protected as -ODMTr. In some embodiments, R^{NS} is bonded to phosphorus through its 3′-O—. In some embodiments, a formed ring by R^{C2} and R^{C3} is an optionally substituted 5-membered ring. In some embodiments, a phosphoramidite has the structure of

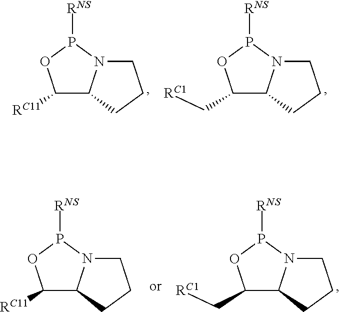

or a salt thereof. In some embodiments, a phosphoramidite has the structure of

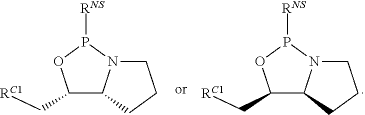

In some embodiments, as described herein R^{NS} comprises a modified nucleobase (e.g., b001A, b002A, b003A, b008U, b001C, etc.) which is optionally protected for oligonucleotide synthesis. In some embodiments, each —OH is optionally and independently substituted or protected. In some embodiments, BA^{s} is optionally substituted or protected nucleobase, and each —OH of the nucleoside is independently protected, wherein at least one —OH is protected as DMTrO-. In some embodiments, —OH for coupling, e.g., with another monomer or phosphoramidite, is protected as DMTrO-. In some embodiments, an —OH group for coupling, e.g., with another monomer or phosphoramidite, is protected different from an —OH group that is not for coupling. In some embodiments, a non-coupling —OH is protected such that the protection remains when DMTrO- is deprotected. In some embodiments, a non-coupling —OH is protected such that the protection remains during oligonucleotide synthesis cycles. In some embodiments, BA^{s} is an optionally protected nucleobase selected from A, T, C, G, U, b008U, hypoxanthine and tautomers thereof. In some embodiments, R^{NS} comprises an optionally substituted or protected nucleobase as described herein or a tautomer thereof and a sugar as described herein.

In some embodiments, purity or stereochemical purity of a monomer or a phosphoramidite is at least 85%, 90%, 95%, 96%, 97%, 98%, or 99%. In some embodiments, it is at least 85%. In some embodiments, it is at least 90%. In some embodiments, it is at least 95%.

In some embodiments, the present disclosure provides a method for preparing an oligonucleotide or composition, comprising coupling a free —OH e.g., a free 5′-OH, of an oligonucleotide or a nucleoside with a monomer as described herein. In some embodiments, the present disclosure provides a method for preparing an oligonucleotide or composition, comprising coupling a free —OH, e.g., a free 5′-OH, of an oligonucleotide or a nucleoside with a phosphoramidite as described herein.

In some embodiments, the present disclosure provides an oligonucleotide, wherein the oligonucleotide comprises one or more modified internucleotidic linkages each independently having the structure of —O⁵—P^{L}(W)(R^{CA})—O³—, wherein:
P^{L} is P, or P(═W):
W is O, S, or W^{N};
W^{N} is =N—C(—N(R¹)₂=N⁺(R¹)₂Q⁻;
Q⁻ is an anion:
R^{CA} is or comprises an optionally capped chiral auxiliary moiety,
O⁵ is an oxygen bonded to a 5′-carbon of a sugar, and
O³ is an oxygen bonded to a 3′-carbon of a sugar.

In some embodiments, a modified internucleotidic linkage is optionally chirally controlled. In some embodiments, a modified internucleotidic linkage is optionally chirally controlled.

In some embodiments, a provided methods comprising removing R^{CA} from such a modified internucleotidic linkages. In some embodiments, after removal, bonding to R^{CA} is replaced with —OH. In some embodiments, after removal, bonding to R^{CA} is replaced with ═O, and bonding to W^{N} is replaced with —N═C(N(R¹)₂)₂.

In some embodiments, P^{L} is P=S, and when R^{CA} is removed, such an internucleotidic linkage is converted into a phosphorothioate internucleotidic linkage.

In some embodiments, P^{L} is P=W^{N}, and when R^{CA} is removed, such an internucleotidic linkage is converted into an internucleotidic linkage having the structure of

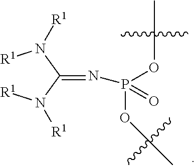

In some embodiments, an internucleotidic linkage having the structure of

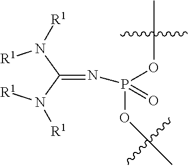

has the structure of

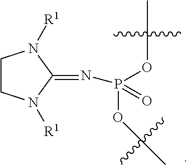

In some embodiments, an internucleotidic linkage having the structure of

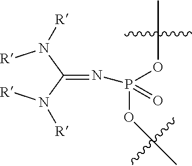

has the structure of

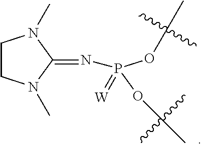

In some embodiments, P^{L} is P (e.g., in newly formed internucleotidic linkage from coupling of a phosphoramidite with a 5′-OH). In some embodiments, W is O or S. In some embodiments, W is S (e.g., after sulfurization). In some embodiments, W is O (e.g., after oxidation). In some embodiments, certain non-negatively charged internucleotidic linkages or neutral internucleotidic linkages may be prepared by reacting a P(III) phosphite triester internucleotidic linkage with azido imidazolinium salts (e.g., compounds comprising

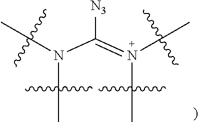

under suitable conditions. In some embodiments, an azido imidazolinium salt is a salt of PF₆⁻. In some embodiments, an azido imidazolinium salt is a salt of

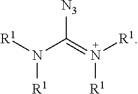

In some embodiments, an azido imidazolinium salt is 2-azido-1,3-dimethylimidazolinium hexafluorophosphate.

As appreciated by those skilled in the art, Q⁻ can be various suitable anion present in a system (e.g., in oligonucleotide synthesis), and may vary during oligonucleotide preparation processes depending on cycles, process stages, reagents, solvents, etc. In some embodiments, Q is PF₆⁻.

In some embodiments, R^{CA} is

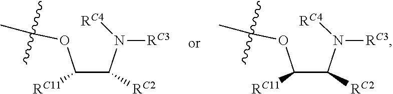

wherein R^{C4} is —H or —C(O)R′, and each other variable is independently as described herein. In some embodiments, R^{CA} is

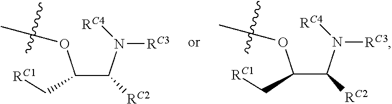

wherein R^{C1} is R, —Si(R)₃ or —SO₂R, R^{C2} and R^{C3} are taken together with their intervening atoms to form an optionally substituted 3-7 membered saturated ring having, in addition to the nitrogen atom, 0-2 heteroatoms, R^{C4} is —H or —C(O)R′. In some embodiments, R^{C4} is —H. In some embodiments, R^{C4} is —C(O)CH₃. In some embodiments, R^{C2} and R^{C3} are taken together to form an optionally substituted 5-membered ring.

In some embodiments, R^{C4} is —H (e.g., in n newly formed internucleotidic linkage from coupling of a phosphoramidite with a 5′-OH). In some embodiments, R^{C4} is —C(O)R (e.g., after capping of the amine). In some embodiments, R is methyl.

In some embodiments, each chirally controlled phosphorothioate internucleotidic linkage is independently converted from —O⁵—P^{L}—(W)(R^{CA})—O³—.

In some embodiments, linkers (e.g., L001) are installed via cycles through coupling with suitable phosphoramidites. In some embodiments, additional chemical moieties (e.g., Mod001) are coupled to linkers (e.g., L001). In some embodiments, additional chemical moieties, or additional chemical moieties and linkers, are installed via cycles through coupling with phosphoramidites comprising additional chemical moieties, or additional chemical moieties and linkers, respectively.

### Assessment/Characterization of Providing Technologies

As appreciated by those skilled in the art, various technologies may be utilized to assess/characterize provided technologies in accordance with the present disclosure. Certain useful technologies are described in the Examples; as demonstrated, among other things, the present disclosure describes various in vivo and in vitro technologies suitable for assessing and characterizing provided technologies. In some embodiments, provided technologies are assessed/characterized, e.g., in cells, with or without exogenous ADAR polypeptides; additionally or alternatively, in some embodiments, provided technologies are assessed/characterized, e.g., in animals, e.g., non-human primates and mice.

In some embodiments, cells and non-human animals are engineered to express human ADAR1 polypeptide or a characteristic portion thereof. In some embodiments, such cells and human are useful for assessing and characterizing provided technologies. In some embodiments, a human ADAR1 polypeptide or a characteristic portion thereof is or comprises human ADAR1 polypeptide or a characteristic portion thereof. In some embodiments, a human ADAR1 polypeptide or a characteristic portion thereof is or comprises human ADAR1 p110 polypeptide or a characteristic portion thereof. In some embodiments, a human ADAR1 polypeptide or a characteristic portion thereof is or comprises human ADAR1 p150 polypeptide or a characteristic portion thereof. In some embodiments, a human ADAR1 polypeptide or a characteristic portion thereof is or comprises human ADAR1. In some embodiments, a human ADAR1 polypeptide or a characteristic portion thereof is or comprises a human ADAR1 p110 peptide. In some embodiments, a human ADAR1 polypeptide or a characteristic portion thereof is or comprises a human ADAR1 p150 peptide. In some embodiments, a human ADAR1 polypeptide or a characteristic portion thereof is or comprises one or more or all of the following domains of human ADAR1: Z-DNA binding domains, dsRNA binding domains, and deaminase domain. In some embodiments, a human ADAR1 polypeptide or a characteristic portion thereof is or comprises one or both of human ADAR1 Z-DNA binding domains; alternatively or additionally. In some embodiments, a human ADAR1 polypeptide or a characteristic portion thereof is or comprises one, two or all of human ADAR1 dsRNA binding domains; alternatively or additionally, a human ADAR1 polypeptide or a characteristic portion thereof is or comprises a human deaminase domain. In some embodiments, a human ADAR1 polypeptide or a characteristic portion thereof may be expressed together with a mouse ADAR1 polypeptide or a characteristic portion thereof, e.g., one or more human dsRNA binding domains may be engineered to be expressed together with a mouse deaminase domain to form a human-mouse hybrid ADAR1 polypeptide. In some embodiments, cells and/or non-human animals are engineered to comprise and/or express a polynucleotide encoding a human ADAR1 polypeptide or a characteristic portion thereof as described herein. In some embodiments, genomes of cells and/or non-human animals are engineered to comprise a polynucleotide encoding a human ADAR1 polypeptide or a characteristic portion thereof as described herein. In some embodiments, germline genomes of cells and/or non-human animals are engineered to comprise a polynucleotide encoding a human ADAR1 polypeptide or a characteristic portion thereof as described herein. In some embodiments, cells and non-human animals are engineered to comprise, e.g., in their genomes (in some embodiments, germline genomes), one or more G to A mutations each independently associated with a condition, disorder or disease (e.g., a mutation (e.g., c. 1024G>A) in SERPINA1 gene that leads to a glutamate to lysine substitution at amino acid position 342 (E342K) of an A1AT protein). As demonstrated herein, among other things such cells and animals are useful for assessing/characterizing provided technologies, e.g., various oligonucleotides and compositions thereof, e.g., for their editing properties and/or activities, including for their uses against one or more conditions, disorders or diseases. In some embodiments, cells are rodent cells. In some embodiments, cells are mouse cells. In some embodiments, an animal is a rodent. In some embodiments, an animal is a mice.

In some embodiments, provided technologies are assessed in animals, e.g., mice, or cells thereof that do not contain or express human ADAR polypeptide or a characteristic portion thereof. In some embodiments, cells, animals, etc. are engineered to comprise and/or express a G to A mutation, e.g., 1024 G>A in SERPINA1.

Among other things, the present disclosure provides oligonucleotide designs comprising sugar modifications, base modifications, internucleotidic linkage modifications, linkage phosphorus stereochemistry, and/or patterns thereof, that can greatly improve one or more properties and/or activities of oligonucleotides compared to comparable oligonucleotides of similar or identical base sequences but of reference designs. For example, it was observed that oligonucleotides of various provided designs and compositions thereof can provide high levels of editing in mice that do not express a human ADAR protein (e.g., mice only expressing mouse ADAR proteins), in some embodiments comparable to or no lower than in mice that are engineered to express a human ADAR protein, while comparable oligonucleotides of reference designs and compositions thereof provide low levels of editing in mice that do not express a human ADAR protein (e.g., mice only expressing mouse ADAR proteins), in some embodiments significantly lower than in mice that are engineered to express a human ADAR protein. In some embodiments, a reference design is a design reported in WO 2016/097212, WO 2017/220751, WO 2018/041973, WO 2018/134301A1, WO 2019/158475, WO 2019/219581, WO 2020/157008, WO 2020/165077, WO 2020/201406 or WO 2020/252376. In some embodiments, a reference design is a design in WO 2021/071858. In some embodiments, a reference design is a design in WO 2022/099159.

Certain useful technologies for assessing provided oligonucleotides, compounds, compositions, methods, etc., are described in WO 2021/071858 and WO 2022/099159, and are incorporated herein by reference.

### Uses and Applications

As appreciated by those skilled in the art, oligonucleotides are useful for multiple purposes. In some embodiments, provided technologies (e.g., oligonucleotides, compositions, methods, etc.) can be useful for modulating levels and/or activities of various nucleic acids (e.g., RNA) and/or products encoded thereby (e.g., proteins). In some embodiments, provided technologies can reduce levels and/or activities of undesired target nucleic acids (e.g., comprising undesired adenosine, e.g., 1024 G>A in SERPINA1) and/or products thereof. In some embodiments, provided technologies can increase levels and/or activities of desired target nucleic acids (e.g., comprising I instead of undesired adenosine at one or more locations) and/or products thereof.

For example, in some embodiments, provided technologies can be utilized as single-stranded oligonucleotides for site-directed editing of target adenosine in SERPINA1 transcripts. In some embodiments, provided technologies are capable of modulating levels of expressions and activities. Among other things, the present disclosure provides improvement by provided technologies which can be improvement of various desired biological functions, including but not limited to treatment and/or prevention of various conditions, disorders or diseases (e.g., those associated with G to A mutation such as 1024 G>A in SERPINA1).

In some embodiments, provided technologies can modulate activities and/or functions of a target gene, e.g., SERPINA1. In some embodiments, provided technologies can increase levels of SERPINA1 transcripts without 1024 G>A and/or products encoded thereby, and/or reduce levels of SERPINA1 transcripts with 1024 G>A and/or products encoded thereby.

In some embodiments, provided oligonucleotides and compositions are useful for treating various conditions, disorders, or diseases, by reducing levels and/or activities of target transcripts (e.g., SERPINA1 transcripts comprising 1024 G>A) and/or products encoded thereby (e.g., E342K A1AT) that are associated with the conditions, disorders, or diseases, and optionally providing transcripts and/or products encoded thereby that are less associated or not associated with the conditions, disorders or diseases (e.g., by conversion of target adenosine to inosine to correct G to A mutations, etc.). In some embodiments, the present disclosure provides methods for preventing or treating a condition, disorder, or disease, comprising administering to a subject susceptible thereto or suffering therefrom an effective amount of a provided oligonucleotide or composition. In some embodiments, the present disclosure provides methods for preventing or treating a condition, disorder, or disease, comprising administering to a subject susceptible to or suffering from a condition, disorder or disease a provided single-stranded oligonucleotide for site-directed editing of a nucleotide (e.g. target adenosine) in a target RNA sequence, or a composition thereof. In some embodiments, a provided single-stranded oligonucleotide for site-directed editing of a nucleotide in a target RNA sequence is of a base sequence that partially or fully complementary to a portion of a transcript, which transcript is associated with a condition, disorder, or disease. In some embodiments, a base sequence is such that it preferentially binds to a transcript associated with a condition, disorder or disease over other transcripts that are not associated with said condition, disorder, or disease. In some embodiments, a condition, disorder, or disease is associated with a G to A mutation. In some embodiments, a condition, disorder, or disease is associated with a G to A mutation in SERPINA1. In some embodiments, a condition, disorder, or disease is associated with 1024 G>A (E342K) mutation in human SERPINA. In some embodiments, a condition, disorder or disease is a liver condition, disorder or disease. In some embodiments, a condition, disorder or disease is a metabolic liver condition, disorder or disease. In some embodiments, a condition, disorder or disease is alpha-1 antitrypsin deficiency. In some embodiments, provided technologies increase levels, properties, and/or activities of desired products (e.g., properly folded wild-type A1AT protein in serum) and/or decreases levels, properties, and/or activities of undesired products (e.g., mutant (e.g., E342K) A1AT protein in serum), in absolute amounts (e.g., ng/mL in serum) and/or relatively (e.g., as % of total proteins or total A1AT proteins). In some embodiments, the present disclosure provides a method for increasing levels and/or activities of an alpha-1 antitrypsin (A1AT) polypeptide in the serum or blood of a subject, comprising administering to the subject an effective amount of an oligonucleotide or composition. In some embodiments, an A1AT polypeptide provides one or more higher activities compared to a reference AAT polypeptide. In some embodiments, an A1AT polypeptide is a wild-type A1AT polypeptide. In some embodiments, method increase the amount of the A1AT polypeptide in serum. In some embodiments, a method decrease the amount of a reference A1AT polypeptide in serum. In some embodiments, a method increase the ratio of the AAT polypeptide over a reference A1AT polypeptide in serum or blood. In some embodiments, a reference A1AT polypeptide is mutated. In some embodiments, a reference A1AT polypeptide is not properly folded. In some embodiments, a reference A1AT polypeptide is an E342K A1AT polypeptide. In some embodiments, the present disclosure provides a method for decreasing levels and/or activities of a mutant alpha-1 antitrypsin (A1AT) polypeptide in the serum or blood of a subject, comprising administering to the subject an effective amount of an oligonucleotide or composition. In some embodiments, a subject is susceptible to or suffering from a condition, disorder or disease. In some embodiments, a condition, disorder or disease is alpha-1 antitrypsin deficiency. In some embodiments, a subject is a human. In some embodiments, a subject comprises a mutation in human SERPINA1. In some embodiments, a subject comprises 1024 G>A (E342K) mutation in human SERPINA1. In some embodiments, a subject is homozygous with respect to the mutation. In some embodiments, a subject is heterozygous with respect to a mutation.

In some embodiments, a condition, disorder or disease is not associated with a G to A mutation. In some embodiments, a condition, disorder or disease is associated with increased level and/or activity of a transcript (e.g., a 1024 G>A in SERPINA1 transcript) and/or an encoded product thereby, and a provided technology can reduce level and/or activity of a transcript and/or an encoded product thereby, e.g., through introducing one or more A to I to a transcript. In some embodiments, a condition, disorder or disease is associated with decreased level and/or activity of a transcript and/or an encoded product thereby, and a provided technology can increase level and/or activity of a transcript (e.g., wild-type SERPINA1 transcript) and/or an encoded product thereby, e.g., through introducing one or more A to I to a transcript. In some embodiments, a condition, disorder or disease is associated with splicing, and a provided technology provides splicing modulation through introducing one or more A to I to a transcript (e.g., pre-mRNA).

In some embodiments, oligonucleotide compositions in provided methods are chirally controlled oligonucleotide compositions. In some embodiments, a method of treating a condition, disorder or disease can include administering a composition comprising a plurality of oligonucleotides sharing a common base sequence, which base sequence is complementary to a target sequence in a target transcript. Among other things, the present disclosure provides an improvement that comprises administering as the oligonucleotide composition a chirally controlled oligonucleotide composition as described in the present disclosure, characterized in that, when it is contacted with the target transcript in a system, adenosine editing of the transcript is improved relative to that observed under a reference condition selected from the group consisting of absence of the composition, presence of a reference composition, and any combinations thereof. In some embodiments, a reference composition is a racemic preparation of oligonucleotides of the same sequence or constitution. In some embodiments, a target transcript is an oligonucleotide transcript.

In some embodiments, technologies of the present disclosure provide editing such as A to I editing in nucleic acids like mRNA and can change properties, structures, functions, etc. of amino acid residues, e.g., size, polarity, charge, etc. in polypeptides encoded thereby and can modulate properties, structures, functions, activity levels, etc. of polypeptides. For example, editing of 1024 G>A in SERPINA1 corrects E342K. Those skilled in the art appreciate that through modulating properties of amino acid residues, various properties, functions, structures, etc. of polypeptides, e.g., stability, localization, processing, folding, interactions, modifications, etc. can be independently modulated, including independently enhancing, reducing and/or maintaining at comparable levels of various properties, functions, structures, etc., and in some embodiments, new properties, functions, structures, etc. may be introduced.

In some embodiments, the present disclosure provides a method for prevent or treating a condition, disorder or disease, comprising administering to a subject susceptible thereto or suffering therefrom an effective amount of an oligonucleotide or a composition thereof as described herein. Those skilled in the art appreciate that through, e.g., editing a nucleobase such as A in a RNA, a protein encoded thereby can be edited. In some embodiments, an amino acid residue is replaced with another amino acid residue. In some embodiments, expression, level, function, stability, property and/or activity are modulated.

In some embodiments, a provided technology modifies protein function. In some embodiments, a provided technology changes one or more properties and/or functions of a nucleic acid (e.g., a transcript) and/or a protein. In some embodiments, a provided technology increases, promotes, or enhances one or more properties and/or functions of a nucleic acid (e.g., a transcript) and/or a protein. In some embodiments, a provided technology provide one or more new properties and/or activities, e.g., of a nucleic acid (e.g., a transcript) and/or a protein. In some embodiments, a provided technology decreases, inhibits, or removes one or more properties and/or functions of a nucleic acid (e.g., a transcript) and/or a protein. In some embodiments, a provided technology alter protein processing. For example, in some embodiments, protease cleavage sites are edited. In some embodiments, provided technologies edit one or more residues involved in protein-protein interactions. In some embodiments, provided technologies edit amino acid residues at protein-protein interactions domains. In some embodiments, through editing mRNAs that encode proteins, residues at various regions of polypeptides, e.g., protease cleavage sites, various domains (e.g., protein-protein interactions domains), modification sites, miRNA targeting sites, ubiquitination sites, etc. can be edited. In some embodiments, provided technologies modulate signaling pathways.

In some embodiments, provided technologies restore, increase or enhance levels of functional proteins, e.g., wild-type A1AT. In some embodiments, provided technologies reduce levels and/or activities of mutant or undesired nucleic acids (e.g., 1024 G>A in SERPINA1 transcripts) and proteins (e.g., E342K A1AT).

In some embodiments, provided technologies modulate enzymatic activities. In some embodiments, provided technologies increase an enzymatic activity, e.g., through editing a codon to a codon encoding an amino acid residue that can increase an enzymatic activity. In some embodiments, provided technologies decrease an enzymatic activity, e.g., those associated with a condition, disorder or disease, through editing a codon to a codon encoding an amino acid residue that can decrease an enzymatic activity. Various enzymatic activities, in many cases with amino acid residues involved for such activities, are reported or can be identified and characterized, and can be modulated in accordance with the present disclosure. In some embodiments, an activity is a kinase activity.

In some embodiments, editing of a protein (e.g., through editing of its encoding mRNA to change one or more amino acid residues) decreases degradation of the protein or a protein which it interacts with. In some embodiments, editing of a protein upregulate its levels. In some embodiments, editing of a protein modulate protein processing. In some embodiments, editing of a protein modulate its folding. In some embodiments, editing of a protein modulate its stability. In some embodiments, editing of a protein modulate protein modification (e.g., increasing, decreasing, removing or introducing a modification site, etc.). In some embodiments, editing of a protein modulate post-translational modification (e.g., increasing, decreasing, removing or introducing a modification site, etc.). In some embodiments, provided technologies are useful for treating associated conditions, disorders or diseases, such as dementias, familial epilepsies, neuropathic pain, neuromuscular disorders, dementias, haploinsufficient diseases, loss of function conditions, disorders or diseases, etc.

In some embodiments, technologies herein modulate activities of nucleic acids (e.g., RNA such as various transcripts). In some embodiments, technologies herein increase level of an activity of a nucleic acid (e.g., RNA such as various transcripts). In some embodiments, technologies herein decrease level of an activity of a nucleic acid (e.g., RNA such as various transcripts). In some embodiments, an activity is a new activity which is not observed prior to adenosine editing. In some embodiments, editing of target adenosines can modulate interactions of nucleic acids (e.g., RNA such as various transcripts) with other agents, e.g., nucleic acids, polypeptides, etc. In some embodiments, interactions are enhanced. In some embodiments, interactions are reduced. Those skilled in the art appreciate that in some embodiments, for a nucleic acid interactions can be independently modulated. In some embodiments, for interaction between a nucleic acid and an interacting agent, in some circumstances it may be enhanced while in other circumstances it may be reduced or maintained at comparable levels. In some embodiments, an interaction is not observed prior to adenosine editing. In some embodiments, adenosines in functional motifs are edited. Those skilled in the art appreciate that various functional motifs have been reported, and various tools have been developed and/or can be developed to identify functional motifs. In some embodiments, technologies herein are utilized to edit adenosines in various functional motifs to modulate properties, structures, functions, activity levels, etc. of various nucleic acids comprising such functional motifs.

Technologies of present disclosure can provide efficient editing in various types of cells, tissues, organs and/or organisms. In some embodiments, provided technologies can provide efficient editing in liver.

Certain applications are described, e.g., in WO 2016/097212, WO 2017/220751, WO 2018/041973, WO 2018/134301A1, WO 2020/154344, WO 2020/154343, WO 2020/154342, WO 2020/165077, WO 2020/201406, WO 2020/216637, or WO 2020/252376.

In some embodiments, when an oligonucleotide or oligonucleotide composition is contacted with a target nucleic acid comprising a target adenosine in a system, a target adenosine in a target nucleic acid is modified. In some embodiments, when an oligonucleotide or oligonucleotide composition is contacted with a target nucleic acid comprising a target adenosine in a system, level of a target nucleic acid is reduced compared to absence of the product or presence of a reference oligonucleotide. In some embodiments, when an oligonucleotide or oligonucleotide composition is contacted with a target nucleic acid comprising a target adenosine in a system, splicing of a target nucleic acid or a product thereof is altered compared to absence of the oligonucleotide or presence of a reference oligonucleotide. In some embodiments, when an oligonucleotide or oligonucleotide composition is contacted with a target nucleic acid comprising a target adenosine in a system, level of a product of a target nucleic acid is altered compared to absence of the product or presence of a reference oligonucleotide. In some embodiments, level of a product is increased, wherein the product is or is encoded by a nucleic acid which is otherwise identical to a target nucleic acid but a target adenosine is modified. In some embodiments, level of a product is increased, wherein the product is or is encoded by a nucleic acid which is otherwise identical to a target nucleic acid but a target adenosine is replaced with inosine. In some embodiments, level of a product is increased, wherein the product is or is encoded by a nucleic acid which is otherwise identical to a target nucleic acid but the adenine of a target adenosine is replaced with guanine. In some embodiments, a product is a protein. In some embodiments, a target adenosine is a mutation from guanine. In some embodiments, a target adenosine is more associated with a condition, disorder or disease than a guanine at the same position. In some embodiments, an oligonucleotide is capable of forming a double-stranded complex with a target nucleic acid. In some embodiments, a target nucleic acid or a portion thereof is or comprises RNA. In some embodiments, a target adenosine is of an RNA. In some embodiments, a target adenosine is modified, and the modification is or comprises deamination of a target adenosine. In some embodiments, a target adenosine is modified and the modification is or comprises conversion of a target adenosine to an inosine. In some embodiments, a modification is promoted by an ADAR protein. In some embodiments, a system is an in vitro or ex vivo system comprising an ADAR protein. In some embodiments, a system is or comprises a cell that comprises or expresses an ADAR protein. In some embodiments, a system is a subject comprising a cell that comprises or expresses an ADAR protein. In some embodiments, a ADAR protein is ADAR1. In some embodiments, an ADAR1 protein is or comprises p110 isoform. In some embodiments, an ADAR1 protein is or comprises p150 isoform. In some embodiments, an ADAR1 protein is or comprises p110 and p150 isoform. In some embodiments, a ADAR protein is ADAR2. As demonstrated herein, the present disclosure among other things provides technologies for recruiting enzymes to target sites (e.g., those comprising target As), comprising contacting such target sites with, or administering to systems comprising or expressing polynucleotide (e.g., RNA) comprising such target sites, provided oligonucleotides or compositions thereof. In some embodiments, an enzyme is an RNA-editing enzyme such as ADAR1, ADAR2, etc. as described herein.

In some embodiments, an oligonucleotide composition comprising a plurality of oligonucleotides provide a greater level, e.g., a target adenosine is modified at a greater level, than that is observed with a comparable reference oligonucleotide composition. In some embodiments, a reference oligonucleotide composition comprises no or a lower level of oligonucleotides of the plurality. In some embodiments, a reference composition does not contain oligonucleotides that have the same constitution as an oligonucleotide of the plurality. In some embodiments, a reference composition does not contain oligonucleotides that have the same structure as an oligonucleotide of the plurality. In some embodiments, a reference oligonucleotide composition is a composition whose oligonucleotides having the same base sequence as oligonucleotides of the plurality contain a lower level of 2′-F modifications compared to oligonucleotides of the plurality. In some embodiments, a reference oligonucleotide composition is a composition whose oligonucleotides having the same base sequence as oligonucleotides of the plurality contain a lower level of 2′-OMe modifications compared to oligonucleotides of the plurality. In some embodiments, a reference oligonucleotide composition is a composition whose oligonucleotides having the same base sequence as oligonucleotides of the plurality have a different sugar modification pattern compared to oligonucleotides of the plurality. In some embodiments, a reference oligonucleotide composition is a composition whose oligonucleotides having the same base sequence as oligonucleotides of the plurality contain a lower level of modified internucleotidic linkages compared to oligonucleotides of the plurality. In some embodiments, a reference oligonucleotide composition is a composition whose oligonucleotides having the same base sequence as oligonucleotides of the plurality contain a lower level of phosphorothioate internucleotidic linkages compared to oligonucleotides of the plurality. In some embodiments, a composition is a stereorandom oligonucleotide composition. In some embodiments, a reference composition is a stereorandom oligonucleotide composition of oligonucleotides of the same constitution as oligonucleotides of the plurality.

In some embodiments, the present disclosure provides technologies for modifying a target adenosine in a target nucleic acid, comprising contacting a target nucleic acid with an provided oligonucleotide or oligonucleotide composition as described herein. In some embodiments, the present disclosure provides a method for deaminating a target adenosine in a target nucleic acid, comprising contacting a target nucleic acid with an oligonucleotide or composition as described herein. In some embodiments, the present disclosure provides a method for producing, or restoring or increasing level of a product of a particular nucleic acid, comprising contacting a target nucleic acid with a provided oligonucleotide or composition wherein a target nucleic acid comprises a target adenosine, and the particular nucleic acid differs from a target nucleic acid in that the particular nucleic acid has an I or G instead of a target adenosine. In some embodiments, the present disclosure provides a method for reducing level of a product of a target nucleic acid, comprising contacting a target nucleic acid with an oligonucleotide or composition of the present disclosure, wherein a target nucleic acid comprises a target adenosine. In some embodiments, a product is a protein. In some embodiments, a product is a mRNA.

Among other things, oligonucleotide designs of the present disclosure, e.g., nucleobase, sugar, internucleotidic linkage modifications, control of linkage phosphorus stereochemistry, and/or patterns thereof, can be applied to improve prior technologies. In some embodiments, the present disclosure provides improvement over prior technologies by introducing one or more structural features of the present disclosure, e.g., nucleobase, sugar, internucleotidic linkage modifications, control of linkage phosphorus stereochemistry, and/or patterns thereof to oligonucleotides in prior technologies. In some embodiments, an improvement is or comprises improvement from control of linkage phosphorus stereochemistry.

In some embodiments, the present disclosure provides technologies for improving adenosine editing by a polypeptide, e.g., ADAR1, ADAR2, etc., comprising incorporating into an oligonucleotide a design (e.g., one or more modifications and/or patterns thereof) as described herein. In some embodiments, a design is or comprises a modified base as described herein, e.g., at the position opposite to a target adenosine and/or one or both of its neighboring positions. In some embodiments, a design is or comprises one or more sugar modifications and/or patterns thereof, one or more base modifications and/or patterns thereof, one or more modified internucleotidic linkages and/or patterns thereof, and/or controlled stereochemistry at one or more positions and/or patterns thereof. In some embodiments, a provided technology improves editing by ADAR1 more than ADAR2. In some embodiments, a provided technology improves editing by ADAR2 more than ADAR1. In some embodiments, a provided technology improves editing by ADAR1 p110 more than p150 (e.g., in some embodiments, Rp (e.g., of phosphorothioate internucleotidic linkages) at one or more positions). In some embodiments, a provided technology improves editing by ADAR1 p150 more than p110.

In some embodiments, a provided technology comprises increasing levels of an adenosine editing polypeptide, e.g., ADAR1 (p110 or p150) or ADAR2, or a portion thereof. In some embodiments, an increase is through expression of an exogenous of a polypeptide.

In some embodiments, a provided oligonucleotide or oligonucleotide composition does not cause significant degradation of a nucleic acid (e.g., no more than about 5%-100% (e.g., no more than about 10%-100%, 20-100%, 30%-100%, 40%-100%, 50%-80%, 50%-85%, 50%-90%, 50%-95%, 60%-80%, 60%-85%, 60%-90%, 60%-95%, 60%-100%, 65%-80%, 65%, 85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.)). In some embodiments, a composition does not cause significant undesired exon skipping or altered exon inclusion in a target nucleic acid (e.g., no more than about 5%-100% (e.g., no more than about 10%-100%, 20-100%, 30%-100%, 40%-100%, 50%-80%, 50%-85%, 50%-90%, 50%-95%, 60%, 80%, 60%-85%, 60%, 90%, 60%-95%, 60%-100%, 65%-80%, 65%-85%, 65%-90%, 65%-95%, 65%-10%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, 10%, 20%, 30%, 40%, 50%, 60%, 65%, 70%, 75%, 80%, 85%, 90%, 95%, or 100%, etc.)).

In some embodiments, provided technologies can provide high levels of adenosine editing (e.g., conversion to inosine). In some embodiments, percentage of target adenosine editing is about 10%-100%, e.g., at least about 10%, 15%, 20%, 25%, 30%, 35%, 40%, 45%, 50%, 60%, 70%, 75%, 80%, 85%, 90%, or 95%. In some embodiments, it is at least 10%. In some embodiments, it is at least 15%. In some embodiments, it is at least 20%. In some embodiments, it is at least 25%. In some embodiments, it is at least 30%. In some embodiments, it is at least 35%. In some embodiments, it is at least 40%. In some embodiments, it is at least 45%. In some embodiments, it is at least 50%. In some embodiments, it is at least 60%. In some embodiments, it is at least 70%. In some embodiments, it is at least 75%. In some embodiments, it is at least 80%. In some embodiments, it is at least 85%. In some embodiments, it is at least 90%. In some embodiments, it is at least 95%. In some embodiments, it is at least about 100%.

In some embodiments, an oligonucleotide or a composition thereof is capable of mediating a decrease in the expression or level of a target nucleic acid or a product thereof (e.g., by modifying a target adenosine into inosine). In some embodiments, an oligonucleotide or a composition thereof is capable of mediating a decrease in the expression or level of a target gene or a gene product thereof (e.g., by modifying a target adenosine into inosine) in a cell in vitro. In some embodiments, expression or level can be decreased by at least about 10%, 15%, 20%, 25%, 30%, 35%, 40%, 45%, 50%, 60%, 70%, 75%, 80%, 85%, 90%, or 95%. In some embodiments, expression or level of a target gene or a gene product thereof can be decreased by at least about 10%, 15%, 20%, 25%, 30%, 35%, 40%, 45%, 50%, 60%, 70%, 75%, 80%, 85%, 90%, or 95% by ADAR-mediated deamination directed by an oligonucleotide or a composition thereof, e.g., at a concentration of 10 uM or less in a cell(s) in vitro. In some embodiments, an oligonucleotide or a composition thereof is capable of provide suitable levels of activities at a concentration of 1 nM, 5 nM, 10 nM or less (e.g., when assayed in cells in vitro or in vivo).

In some embodiments, activity of provided oligonucleotides and compositions may be assessed by IC50, which is the inhibitory concentration to decrease level of a target nucleic acid or a product thereof by 50% in a suitable condition, e.g., cell-based in vitro assays. In some embodiments, provided oligonucleotides or compositions have an IC50 no more than 0.001, 0.01, 0.1, 0.5, 1, 2, 5, 10, 50, 100, 200, 500 or 1000 nM, e.g., when assessed in cell-based assays. In some embodiments, an IC50 is no more than about 500 nM. In some embodiments, an IC50 is no more than about 200 nM. In some embodiments, an IC50 is no more than about 100 nM. In some embodiments, an IC50 is no more than about 50 nM. In some embodiments, an IC50 is no more than about 25 nM. In some embodiments, an IC50 is no more than about 10 nM. In some embodiments, an IC50 is no more than about 5 nM. In some embodiments, an IC50 is no more than about 2 nM. In some embodiments, an IC50 is no more than about 1 nM. In some embodiments, an IC50 is no more than about 0.5 nM.

In some embodiments, provided technologies can provide selective editing of target adenosine over other adenosine residues in a target adenosine. In some embodiments, selectivity of a target adenosine over a non-target adenosine is at least 1.5, 2, 3, 4, 5, 6, 7, 8, 9, 10, 15, 20, 25, 30, 40, 50, 60, 70, 80, 90, 100 fold or more (e.g., as measured by level of editing of a target adenosine over a non-target adenosine at a suitable condition, or by oligonucleotide concentrations for a certain level of editing (e.g., 0.5%, 1%, 2%, 5%, 10%, 15%, 20%, 25%, 30%, 40%, 50%, etc.). In some embodiments, a selectivity is at least 2 fold. In some embodiments, a selectivity is at least 3 fold. In some embodiments, a selectivity is at least 4 fold. In some embodiments, a selectivity is at least 5 fold. In some embodiments, a selectivity is at least 10 fold. In some embodiments, a selectivity is at least 25 fold. In some embodiments, a selectivity is at least 50 fold. In some embodiments, a selectivity is at least 100 fold.

In some embodiments, the present disclosure provides a method for suppression of a transcript from a target nucleic acid sequence for which one or more similar nucleic acid sequences exist within a population, each of the target and similar sequences contains a specific characteristic sequence element that defines the target sequence relative to the similar sequences, the method comprising contacting a sample comprising transcripts of target nucleic acid sequence with an oligonucleotide, or a composition comprising a plurality of oligonucleotides sharing a common base sequence, wherein the base sequence of the oligonucleotide, or the common base sequence of the plurality of oligonucleotide, is or comprises a sequence that is complementary to the characteristic sequence element that defines the target nucleic acid sequence. In some embodiments, wherein when the oligonucleotide, or the oligonucleotide composition, is contacted with a system comprising transcripts of both the target nucleic acid sequence and a similar nucleic acid sequences, transcripts of the target nucleic acid sequence are suppressed at a greater level than a level of suppression observed for a similar nucleic acid sequence. In some embodiments, suppression of the transcripts of the target nucleic acid sequence can be 1.1-100, 2-100, 1.5, 2, 2.5, 3, 4, 5, 6, 7, 8, 9, or 10-fold greater than suppression observed for a similar nucleic acid sequence. In some embodiments, a target nucleic acid sequence is associated with (or more associated with compared to a similar nucleic acid sequence) a condition, disorder or disease. As those skilled in the art will appreciate, selective reduction of a transcript (and/or products thereof) associated with conditions, disorders or diseases, while maintaining transcripts that are not, or are less, associated with conditions, disorders or diseases can provide a number of advantages, for example, providing disease treatment and/or prevention while maintaining one or more desired biological functions (which may provide, among other things, fewer or less severe side effects).

In some embodiments, as demonstrated herein, selectivity is at least 10 fold, or 20, 30, 40, or 50 fold or more in a system, e.g. a reporter assay described herein. In some embodiments, an oligonucleotide or composition can effectively reduce levels of mutant protein (e. g., at least 50%, 60%, 70% or more reduction of a mutant protein) while maintaining levels of wild-type protein (e.g. at least 70%, 75%, 80%, 85%, 90%, 95%, or more wild-type protein remaining) in a system. In some embodiments, provided oligonucleotides are stable in various biological systems, e.g. in mouse brain homogenates (e.g., at least 70%, 75%, 80%, 85%, 90%, 95%, or more remaining after 1, 2, 3, 4, 5, 6, 7, or 8 days). In some embodiments, provided oligonucleotides are of low toxicity. In some embodiments, provided oligonucleotides and compositions thereof, e.g., chirally controlled oligonucleotides and compositions thereof, do not significant activate TLR9 (e.g., when compared to reference oligonucleotides and compositions thereof (e.g., corresponding stereorandom oligonucleotides and compositions thereof)). In some embodiments, provided oligonucleotides and compositions thereof, e.g., chirally controlled oligonucleotides and compositions thereof, do not significantly induce complement activation (e.g., when compared to reference oligonucleotides and compositions thereof (e.g., corresponding stereorandom oligonucleotides and compositions thereof)).

For various applications, provided oligonucleotides and/or compositions may be provided as pharmaceutical compositions. In some embodiments, the present disclosure provides a pharmaceutical composition which comprises or delivers an effective amount of an oligonucleotide or a pharmaceutically acceptable salt thereof. In some embodiments, a pharmaceutical composition may comprise various forms of an oligonucleotide, e.g., acid, base and various pharmaceutically acceptable salt forms. In some embodiments, a pharmaceutically acceptable salt is sodium salt. In some embodiments, a pharmaceutically acceptable salt is a potassium salt. In some embodiments, a pharmaceutically acceptable salt is a amine salt (e.g., of an amine having the structure of N(R)₃). In some embodiments, a pharmaceutical composition further comprises a pharmaceutically acceptable carrier. In some embodiments, a pharmaceutical composition is or comprises a liquid solution. In some embodiments, a liquid composition has a controlled pH range, e.g., around or being physiological pH. In some embodiments, a pharmaceutical composition comprises or is formulated as a solution in a physiologically compatible buffers such as Hanks's solution, Ringer's solution, cerebral spinal fluid, artificial cerebral spinal fluid (aCSF) or physiological saline buffer. In some embodiments, a pharmaceutical composition comprises or is formulated as a solution in artificial cerebral spinal fluid (aCSF). In some embodiments, a pharmaceutical composition is an injectable suspension or solution. In certain embodiments, injectable suspensions or solutions are prepared using appropriate liquid carriers, suspending agents and the like. Pharmaceutical compositions can be administered in various suitable routes. In some embodiments, pharmaceutical compositions are formulated for oral administration, for example, drenches (aqueous or non-aqueous solutions or suspensions), tablets, e.g., those targeted for buccal, sublingual, and systemic absorption, boluses, powders, granules, pastes for application to the tongue, parenteral administration, for example, by subcutaneous, intramuscular, intravenous, intrathecal, intracerebroventricular, intravitreal, subretinal, suprachoroidal or epidural injection as, for example, a sterile solution or suspension, e.g., in physiologically compatible buffers such as Hanks's solution, Ringer's solution, artificial cerebral spinal fluid (aCSF) or physiological saline buffer or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces. In some embodiments, technologies herein provide various delivery advantages, e.g., high delivery efficiency, delivery without lipid vehicles, etc. In some embodiments, oligonucleotides or compositions are administered or delivered parenterally. In some embodiments, oligonucleotides or compositions are administered or delivered intravenously. In some embodiments, oligonucleotides or compositions are administered or delivered intrathecally. In some embodiments, oligonucleotides or compositions are administered or delivered intravitreally. In some embodiments, oligonucleotides or compositions are administered or delivered subcutaneously.

Among other things, the present disclosure provides technologies for preventing or treating conditions, disorders or diseases. In some embodiments, the present disclosure provides a method for preventing or treating a condition, disorder or disease, comprising administering or delivering to a subject susceptible thereto or suffering therefrom an effective amount of an oligonucleotide or composition as described herein. In some embodiments, a condition, disorder or disease is amenable to (e.g., can benefit from) A to I conversion. In some embodiments, the present disclosure provides a method for preventing or treating a condition, disorder or disease associated with a G to A mutation, comprising administering to a subject susceptible thereto or suffering therefrom an effective amount of an oligonucleotide or composition as described herein. In some embodiments, the present disclosure provides a method for preventing or treating a condition, disorder or disease amenable to a G to A mutation, comprising administering to a subject susceptible thereto or suffering therefrom an effective amount of an oligonucleotide or composition as described herein. In some embodiments, the present disclosure provides a method for preventing or treating a condition, disorder or disease associated with a G to A mutation, comprising administering to a subject susceptible thereto or suffering therefrom an effective amount of an oligonucleotide or composition as described herein. In some embodiments, the base sequence of the oligonucleotide or oligonucleotides in the oligonucleotide composition is substantially complementary to that of the target nucleic acid comprising a target adenosine. In some embodiments, cells, tissues or organs associated with the condition, disorder or disease comprise or express an ADAR protein. In some embodiments, cells, tissues or organs associated with the condition, disorder or disease comprise or express ADAR1 (e.g., a p110 and/or a p150 forms). In some embodiments, cells, tissues or organs associated with the condition, disorder or disease comprise or express ADAR2. In some embodiments, a condition, disorder or disease is as described herein. In some embodiments, a condition, disorder or disease is alpha-1 antitrypsin deficiency. In some embodiments, a method comprises converting a target adenosine to 1.

In some embodiments, the present disclosure provides an oligonucleotide comprising a sequence complementary to a target sequence. In some embodiments, the present disclosure provides an oligonucleotide which directs site-specific (can also be referred as site directed) editing (e.g., deamination). In some embodiments, the present disclosure provides an oligonucleotide which directs site-specific adenosine editing mediated by ADAR (e.g., an endogenous ADAR). Various provided oligonucleotides can be utilized as single-stranded oligonucleotides for site-directed editing of a nucleotide in a target RNA sequence. In some embodiments, the present disclosure provides methods for preventing and/or treating conditions, disorders, or diseases associated with a G to A mutation in a target sequence using provided single-stranded oligonucleotides for site-directed editing of a nucleotide in a target RNA sequence and compositions thereof. In some embodiments, the present disclosure provides oligonucleotides and compositions thereof for use as medicaments, e.g., for conditions, disorders, or diseases associated with a G to A mutation in a target sequence. In some embodiments, the present disclosure provides oligonucleotides and compositions thereof for use in the treatment of conditions, disorders or diseases associated with a G to A mutation in a target sequence. In some embodiments, the present disclosure provides oligonucleotides and compositions thereof for the manufacture of medicaments for the treatment of a related conditions, disorders or diseases associated with a G to A mutation in a target sequence.

In some embodiments, the present disclosure provides a method for preventing, treating or ameliorating a condition, disorder or disease associated with a G to A mutation in a target sequence in a subject susceptible thereto or suffering therefrom, comprising administering to the subject a therapeutically effective amount of an oligonucleotide or a pharmaceutical composition thereof.

In some embodiments, the present disclosure provides a method for deaminating a target adenosine in a target sequence in a cell, comprising: contacting the cell with an oligonucleotide or a composition thereof. In some embodiments, the present disclosure provides a method deaminating a target adenosine in a target sequence (e.g., a transcript) in a cell, comprising: contacting the cell with an oligonucleotide or a composition thereof. In some embodiments, the present disclosure provides a method for reducing the level of a protein associated with a G to A mutation in a cell, comprising: contacting the cell with an oligonucleotide or a composition thereof. In some embodiments, provided methods can selectively reduce levels of a transcripts and/or products encoded thereby that are related to conditions, disorders or diseases associated with a G to A mutation. In some embodiments, provided methods can selectively edit target nucleic acids, e.g., transcripts comprising an undesired A (e.g., a G to A mutation) over otherwise identical nucleic acids which have G at positions of target A.

In some embodiments, the present disclosure provides a method for decreasing a mutated gene (e.g., a G to A mutation) expression in a mammal in need thereof, comprising administering to the mammal a nucleic acid-lipid particle comprising a provided single-stranded oligonucleotide for site-directed editing of a nucleotide in a target RNA sequence or a composition thereof.

In some embodiments, the present disclosure provides a method for in vivo delivery of an oligonucleotide, comprising administering to a mammal an oligonucleotide or a composition thereof.

In some embodiments, a subject or patient suitable for treatment of a condition, disorder, or disease associated with a G to A mutation, can be identified or diagnosed by a health care professional.

In some embodiments, a symptom of a condition, disorder or disease associated with a G to A mutation can be any condition, disorder or disease that can benefit from an A to I conversion.

In some embodiments, a provided single-stranded oligonucleotide for site-directed editing of a nucleotide in a target RNA sequence or a composition thereof can prevent, treat, ameliorate, or slow progression of a condition, disorder or disease associated with a G to A mutation, or at least one symptom of a condition, disorder or disease associated with a G to A mutation.

In some embodiments, a method of the present disclosure can be for the treatment of a condition, disorder or disease associated with a G to A mutation in a subject wherein the method comprises administering to a subject a therapeutically effective amount of an oligonucleotide or a pharmaceutical composition thereof.

In some embodiments, a provided method can reduce at least one symptom of a condition, disorder or disease associated with a G to A mutation wherein the method comprises administering to a subject a therapeutically effective amount of an oligonucleotide or a pharmaceutical composition thereof.

In some embodiments, administration of an oligonucleotide to a patient or subject can be capable of mediating any one or more of: slowing the progression of a condition, disorder or disease associated with a G to A mutation; delaying the onset of a condition, disorder or disease associated with a G to A mutation or at least one symptom thereof; improving one or more indicators of a condition, disorder or disease associated with a G to A mutation; and/or increasing the survival time or lifespan of the patient or subject.

In some embodiments, slowing disease progression can relate to the prevention of, or delay in, a clinically undesirable change in one or more clinical parameters in an individual susceptible to or suffering from a condition, disorder, or disease associated with a G to A mutation, such as those described herein. It is well within the abilities of a physician to identify a slowing of disease progression in an individual susceptible to or suffering a condition, disorder, or disease associated with a G to A mutation, using one or more of the disease assessment tests described herein. Additionally, it is understood that a physician may administer to the individual diagnostic tests other than those described herein to assess the rate of disease progression in an individual susceptible to or suffering from a condition, disorder, or disease associated with a G to A mutation.

A physician may use family history of a condition, disorder, or disease associated with a G to A mutation or comparisons to other patients with similar genetic profile.

In some embodiments, indicators of a condition, disorder, or disease associated with a G to A mutation include parameters employed by a medical professional, such as a physician, to diagnose or measure the progression of the condition, disorder, or disease.

In some embodiments, a subject is administered an oligonucleotide or a composition thereof and an additional agent and/or method, e.g., an additional therapeutic agent and/or method. In some embodiments, an oligonucleotide or composition thereof can be administered alone or in combination with one or more additional therapeutic agents and/or treatment. When administered in combination each component may be administered at the same time or sequentially in any order at different points in time. In some embodiments, each component may be administered separately but sufficiently closely in time so as to provide the desired therapeutic effect. In some embodiments, provided oligonucleotides and additional therapeutic components are administered concurrently. In some embodiments, provided oligonucleotides and additional therapeutic components can be administered as one composition. In some embodiments, at a time point a subject being administered can be exposed to both provided oligonucleotides and additional components at the same time.

In some embodiments, oligonucleotides or compounds are delivered, e.g., to cells, tissues, organs, etc. through administering conjugates comprising oligonucleotides or compounds to be delivered and additional chemical moieties. In some embodiments, oligonucleotides or compounds to be delivered and additional chemical moieties are conjugated optionally through linkers. In some embodiments, oligonucleotides or compounds to be delivered are released in cells, tissues, organs, etc. after additional chemical moieties and/or linkers are cleaved.

In some embodiments, an additional agent can be physically conjugated to an oligonucleotide. In some embodiments, an additional agent is GalNAc. In some embodiments, a provided single-stranded oligonucleotide for site-directed editing of a nucleotide in a target RNA sequence can be physically conjugated with an additional agent. In some embodiments, additional agent oligonucleotides can have base sequences, sugars, nucleobases, internucleotidic linkages, patterns of sugar, nucleobase, and/or internucleotidic linkage modifications, patterns of backbone chiral centers, etc., or any combinations thereof, as described in the present disclosure, wherein each T may be independently replaced with U and vice versa. In some embodiments, an oligonucleotide can be physically conjugated to a second oligonucleotide which can decrease (directly or indirectly) the expression, activity, and/or level of a target sequence, or which is useful for treating a condition, disorder, or disease associated with a G to A mutation.

In some embodiments, a provided single-stranded oligonucleotide for site-directed editing of a nucleotide in a target RNA sequence may be administered with one or more additional (or second) therapeutic agent for a condition, disorder or disease associated with a G to A mutation.

In some embodiments, a subject can be administered an oligonucleotide and an additional therapeutic agent, wherein the additional therapeutic agent is an agent described herein or known in the art which is useful for treatment of a condition, disorder or disease to be treated.

In some embodiments, provided single-stranded oligonucleotide for site-directed editing of a nucleotide in a target RNA sequence can be co-administered or be used as part of a treatment regimen along with one or more treatment for a condition, disorder or disease or a symptom thereof, including but not limited to: aptamers, lncRNAs, lncRNA inhibitors, antibodies, peptides, small molecules, other oligonucleotides to a target other targets.

In some embodiments, an additional therapeutic treatment is a method of editing a gene

In some embodiments, an additional therapeutic agent is an oligonucleotide.

In some embodiments, a second or additional therapeutic agent can be administered to a subject prior, simultaneously with, or after an oligonucleotide. In some embodiments, a second or additional therapeutic agent can be administered multiple times to a subject, and an oligonucleotide is also administered multiple times to a subject, and the administrations are in any order.

In some embodiments, an improvement may include decreasing the expression, activity and/or level of a gene or gene product which is too high in a disease state; increasing the expression, activity and/or level of a gene or gene product which is too low in the disease state; and/or decreasing the expression, activity and/or level of a mutant and/or disease-associated variant of a gene or gene product.

In some embodiments, an oligonucleotide or composition useful for treating, ameliorating and/or preventing a condition, disorder or disease associated with a G to A mutation can be administered (e.g., to a subject) via various suitable available technologies.

In some embodiments, provided oligonucleotides, e.g., single-stranded oligonucleotide for site-directed editing of a nucleotide in a target RNA sequences, can be administered as a pharmaceutical composition, e.g., for treating, ameliorating and/or preventing conditions, disorders or diseases. In some embodiments, provided oligonucleotides comprise at least one chirally controlled internucleotidic linkage. In some embodiments, provided oligonucleotide compositions are chirally controlled.

Among other things, technologies, e.g., oligonucleotides and compositions thereof, of the present disclosure can provide various improvements and advantages compared to reference technologies (e.g., absence or low levels of chiral control (e.g., stereorandom oligonucleotide compositions (e.g., of oligonucleotides of the same base sequence, or the same constitution, etc.)), and/or absence or low levels of certain modifications and patterns thereof (e.g., 2′-F, non-negatively charged internucleotidic linkages, etc.), such as improved stability, delivery, editing efficiency, pharmacokinetics, and/or pharmacodynamics. In some embodiments, a reference oligonucleotide composition is a stereorandom oligonucleotide composition of oligonucleotides with the same base sequence. In some embodiments, a reference oligonucleotide composition is a stereorandom oligonucleotide composition of oligonucleotides with the same constitution (as appreciated by those skilled in the art, in some embodiments, various salt forms may be properly considered to be of the same constitution). In some embodiments, a reference oligonucleotide is an oligonucleotide comprising no non-negatively charged internucleotidic linkages. In some embodiments, a reference oligonucleotide comprises no n001. In some embodiments, a reference oligonucleotide composition is a composition of oligonucleotides comprising no non-negatively charged internucleotidic linkages. In some embodiments, a reference oligonucleotide composition is a composition of oligonucleotides comprising no n001. In some embodiments, provided technologies may be utilized at lower unit or total doses, and/or may be administered with fewer doses and/or longer dose intervals (e.g., to achieve comparable or better effects) compared to reference technologies. In some embodiments, provided technologies can provide long durability of editing. In some embodiments, provided technologies once administered can provide activities, e.g., target editing, at or above certain levels (e.g., levels useful and/or sufficient to provide certain biological and/or therapeutic effects) for a period of time, e.g., about or at least about 2, 3, 4, 5, 6, 7, 8, 9, 10, 15, 20, 25, 30, 35, 40, 45, 50, 60 or more days, or 1, 2, 3, 4, 5, 6, 7, 8, 9, or 10 months, after a last dose. In some embodiments, provided technologies provide low toxicity. In some embodiments, provided technologies may be utilized at higher unit or total doses, and/or may be administered with more doses and/or shorter dose intervals (e.g., to achieve better effects) compared to reference technologies. In some embodiments, a total dose may be administered as a single dose. In some embodiments, a total dose may be administered as two or more single doses. In some embodiments, a total dose administered as a single dose may provide higher maximum editing levels compared to when administered as two or more single doses.

In some cases, patients who have been administered an oligonucleotide as a medicament may experience certain side effects or adverse effects, including: thrombocytopenia, renal toxicity, glomerulonephritis, and/or coagulation abnormalities; genotoxicity, repeat-dose toxicity of target organs and pathologic effects, dose response and exposure relationships: chronic toxicity; juvenile toxicity: reproductive and developmental toxicity; cardiovascular safety; injection site reactions; cytokine response complement effects: immunogenicity; and/or carcinogenicity. In some embodiments, an additional therapeutic agent is administered to counter-act a side effect or adverse effect of administration of an oligonucleotide. In some embodiments, a particular single-stranded oligonucleotide for site-directed editing of a nucleotide in a target RNA sequence can have a reduced capability of eliciting a side effect or adverse effect, compared to a different single-stranded oligonucleotide for site-directed editing of a nucleotide in a target RNA sequence.

In some embodiments, an additional therapeutic agent can be administered to the patient in order to control or alleviate one or more side effects or adverse effects associated with administration of an oligonucleotide.

In some embodiments, an oligonucleotide and one or more additional therapeutic agent can be administered to a patient (in any order), wherein the additional therapeutic agent can be administered to the patient in order to control or alleviate one or more side effects or adverse effects associated with administration of the oligonucleotide.

In some embodiments, an oligonucleotide and one or more additional therapeutic agent can be administered to a patient (in any order), wherein the additional therapeutic agent can be administered to the patient in order to control or alleviate one or more side effects or adverse effects associated with administration of the oligonucleotide.

In some embodiments, an oligonucleotide and one or more additional therapeutic agent can be administered to a patient (in any order), wherein the additional therapeutic agent can be administered to the patient in order to control or alleviate one or more side effects or adverse effects associated with administration of the oligonucleotide, and wherein the oligonucleotide operates via any biochemical mechanism, including but not limited to: decreasing the level, expression and/or activity of a target gene or a gene product thereof, increasing or decreasing skipping of one or more exons in a target gene mRNA, an ADAR-mediated deamination, a RNase H-mediated mechanism, a steric hindrance-mediated mechanism, and/or a RNA interference-mediated mechanism, wherein the oligonucleotide is single- or double-stranded.

In some embodiments, an oligonucleotide composition and one or more additional therapeutic agent can be administered to a patient (in any order), wherein the additional therapeutic agent can be administered to the patient in order to control or alleviate one or more side effects or adverse effects associated with administration of the oligonucleotide composition, and wherein the oligonucleotide composition can be chirally controlled or comprises at least one chirally controlled internucleotidic linkage (including but not limited to a chirally controlled phosphorothioate).

In some embodiments, a condition, disorder or disease is Alpha-1 antitrypsin (A1AT) deficiency (AATD).

Alpha-1 antitrypsin (A1AT) deficiency (AATD) is a genetic disease reportedly caused by defects in the SERPINA1 gene (also known as PI; AIA; AAT; PII; A1AT; PR02275; and alpha1AT). Severe A1AT deficiency is associated with various phenotypes including lung and liver phenotypes.

A1AT deficiency is reportedly one of the most common genetic diseases in subjects of Northern European descent. Prevalence of severe A1AT deficiency in the U.S. alone is 80,000-100,000. Similar numbers are estimated to be found in the EU. The worldwide estimate for severe A1AT deficiency has been pegged at 3 million people. A1AT deficiency causes emphysema, with subjects developing emphysema in their third or fourth decade. A1AT deficiency can also cause liver failure and hepatocellular carcinoma, with up to 30% of subjects with severe A1AT deficiency developing significant liver disease, including cirrhosis, fulminant liver failure, and hepatocellular carcinoma.

A mutation (i.e., c. 1024G>A) in SERPINA1 gene leads to a glutamate to lysine substitution at amino acid position 342 (E342K, “Z mutation”) of the mature A1AT protein. This missense mutation affect protein conformation and secretion leading to reduced circulating levels of A1AT. Alleles carrying the Z mutation are identified as PiZ alleles. Subjects homozygous for the PiZ allele are termed PiZZ carriers, and express 10-15% of normal levels of serum A1AT. Approximately 95% of subjects who are symptomatic for A1AT deficiency have the PiZZ genotype. Subjects heterozygous for the Z mutation are termed PiMZ mutants, and express 60% of normal levels of serum A1AT. Of those diagnosed, 90% of patients with severe A1AT deficiency have the ZZ mutation. About between 30,000 and 50,000 individuals in the United States have the PiZZ genotype.

The pathophysiology of A1AT deficiency can vary by the organ affected. Liver disease is reported to be due to a gain-of-function mechanism. Abnormally folded A1AT, especially Z-type A1AT (Z-AT), aggregates and polymerizes within hepatocytes. A1AT inclusions are found in PiZZ subjects and are thought to cause cirrhosis and, in some cases, hepatocellular carcinoma. Evidence for the gain-of-function mechanism in liver disease is supported by null homozygotes. These subjects produce no A1AT and do not develop hepatocyte inclusions or liver disease.

It is reported that A1AT deficiency leads to liver disease in up to about 50% of A1AT subjects and leads to severe liver disease in up to about 30% of subjects. Liver disease may manifest as: (a) cirrhosis during childhood that is self-limiting, (b) severe cirrhosis during childhood or adulthood that requires liver transplantation or leads to death and (c) hepatocellular carcinoma that is often deadly. The onset of liver disease is reported to be bi-modal, predominantly affecting children or adults. Childhood disease is self-limiting in many cases but may be led to end-stage, deadly cirrhosis. It is reported that up to about 18% of subjects with the PiZZ genotype may develop clinically significant liver abnormalities during childhood. Approximately 2% of PiZZ subjects are reported to develop severe liver cirrhosis leading to death during childhood (Sveger 1988; Volpert 2000). Adult-onset liver disease may affect subjects with all genotypes, but presents earlier in subjects with the PiZZ genotype. Approximately 2-10% of A1AT deficient subjects are reported to develop adult-onset liver disease.

Lung disease associated with A1AT deficiency is currently treated with intravenous administration of human-derived replacement A1AT protein, but in addition to being costly and requiring frequent injections over a subject's entire lifetime, this approach is only partially effective. A1AT-deficient subjects with hepatocellular carcinoma are currently treated with chemotherapy and surgery, but there is no satisfactory approach for preventing the potentially deadly liver manifestations of A1AT deficiency.

Among other things, the present disclosure recognizes a need for improved treatment of A1AT deficiency, e.g., including liver and lung manifestations thereof. In some embodiments, the present disclosure provides technologies for preventing or treating conditions, disorders or diseases associated Alpha-1 antitrypsin (A1AT) deficiency, e.g., by providing oligonucleotides and/or compositions that can convert the A mutation to I which can be read as G during protein translation and thus correcting the G to A mutation for protein translation. Among other things, alteration of SERPINA1 in one or more of hepatocytes can prevent the progression of liver disease in subjects with A1AT deficiency by reducing or eliminating production of the toxic Z protein (Z-AAT). In certain embodiments, Z protein production is eliminated or reduced by utilizing provided technologies. In certain embodiments, the disease is cured, does not progress, or has delayed progression compared to a subject who has not received the therapy.

In some embodiments, AATD dual pathologies have been reported in liver and lung. In some embodiments, inability to secrete polymerized Z-ATT has been reported to lead to, e.g., liver damage/cirrhosis. In some embodiments, one or both lungs are open to unchecked proteases, which in some embodiments lead to inflammation and lung damage. Many patients (e.g., reportedly ˜200,000 in the US and EU) are with homozygous ZZ genotype which is reported to be associated with the most common form of sever AATD. It has been reported that approved therapies modestly increase circulating levels of wide-type AAT in those with lung pathology, and no therapies address liver pathology. In some embodiments, provided technologies increase or restore expression, levels, properties and/or activities of wild-type AAT in liver. In some embodiments, provided technologies target liver, e.g., through incorporating moieties targeting liver (e.g., ligands such as GalNAc targeting receptors expressed in liver) into oligonucleotides. In some embodiments, provided technologies restore, increase or enhance wild-type AAT physiological regulation in liver. In some embodiments, provided technologies reduce Z-AAT protein aggregation. In some embodiments, provided technologies restore, increase or enhance wild-type AAT physiological regulation in liver and reduce Z-AAT protein aggregation. In some embodiments, provided technologies increase secretion into bloodstream. In some embodiments, provided technologies increase circulating wild-type AAT. In some embodiments, provided technologies increase circulating, lung-bond wild-type AAT. In some embodiments, provided technologies increase or restore expression, levels, properties and/or activities of wild-type AAT in lung. In some embodiments, provided technologies protect lungs from undesired proteases. In some embodiments, provided technologies reduce or prevent inflammation and/or lung damage. In some embodiments, provided technologies provide benefits at both livers and lungs. In some embodiments, provided technology reduces or prevents liver damage or cirrhosis, and reduces or prevents inflammation and/or lung damage. In some embodiments, provided oligonucleotides, e.g., those comprising certain moieties such ligands (e.g., GalNAc) targeting receptors expressed in livers, provide benefits at livers and lungs. In some embodiments, provided technologies simultaneously provide benefits at livers and lungs. In some embodiments, provided technologies address lung and/or liver manifestation of AATD. In some embodiments, provided technologies simultaneously address lung and liver manifestation of AATD. In some embodiments, provided technologies comprise using GalNAc conjugated oligonucleotides and compositions thereof to correct RNA base mutation in mRNA coded by SERPINA1 Z allele that triggers AATD. In some embodiments, provided technologies simultaneously reduce aggregation of mutated, misfolded alpha-1 protein and increase circulating levels of wild-type alpha-1 antitrypsin protein, and in some embodiments address both liver and lung manifestations of AATD. In some embodiments, provided technologies avoid risk of permanent off-target changes to DNA. In some embodiments, oligonucleotides or compositions, e.g., for preventing or treating AATD, are administered or delivered subcutaneously.

In certain embodiments, technologies as described herein can provide a selective advantage to survival of one or more of treated hepatocytes. In certain embodiments, a target cell is modified. In some embodiments, cells treated with technologies herein may not produce toxic Z protein. In some embodiments, diseased cells that are not modified produce toxic Z proteins and may undergo apoptosis secondary to endoplasmic reticulum (ER) stress induced by Z protein misfolding. In certain embodiments, after treatment using the provided technologies, treated cells will survive and untreated cells will die. This selective advantage can drive eventual colonization of hepatocytes with the majority being SERPINA1 corrected cells.

In some embodiments, provided technologies alleviate aggregation of Z-AAT in liver. In some embodiments, provided technologies alleviate liver inflammation. In some embodiments, provided technologies correct or reduce levels of gain-of-function phenotypes of Z-AAT, e.g., progressive liver disease associated with Z-AAT aggregation. In some embodiments, provided technologies prevent, reduce severity of, delay onset of, and/or slow progression of various conditions, disorders or diseases, e.g., those associated with Z-AAT such as liver fibrosis, cirrhosis and hepatocellular carcinoma. In some embodiments, the present disclosure provides methods for reducing Z-AAT aggregation, e.g., in liver. In some embodiments, the present disclosure provides methods for increasing AAT serum concentration, e.g., to about or above about 11 uM. In some embodiments, the present disclosure provides methods for increasing M-AAT serum concentration. In some embodiments, the present disclosure provides methods for increasing M-AAT serum percentage of total AAT, e.g., to about or above about 60%, 65%, 70%, or 75%. In some embodiments, the present disclosure provides methods for inhibiting elastase. In some embodiments, the present disclosure provides methods for inhibiting neutrophil elastase. In some embodiments, the present disclosure provides methods for increasing elastase inhibition. In some embodiments, the present disclosure provides methods for increasing neutrophil elastase inhibition. In some embodiments, inhibition comprises inhibition in a lung. In some embodiments, the present disclosure provides methods for reducing liver inflammation. In some embodiments, the present disclosure provides methods for reducing lobular inflammation. In some embodiments, the present disclosure provides methods for reducing liver PAS-D positive area (e.g., by percentage). In some embodiments, the present disclosure provides methods for reducing liver globular diameter. In some embodiments, the present disclosure provides methods for preventing liver fibrosis. In some embodiments, the present disclosure provides methods for preventing liver cirrhosis. In some embodiments, the present disclosure provides methods for preventing hepatocellular carcinoma. In some embodiments, the present disclosure provides methods for treating liver fibrosis. In some embodiments, the present disclosure provides methods for treating liver cirrhosis. In some embodiments, the present disclosure provides methods for treating hepatocellular carcinoma. In some embodiments, provided methods comprise administering or delivering to a subject an effective amount of an oligonucleotide or oligonucleotide composition. In some embodiments, a subject comprises 1024 G>A (E342K) mutation in SERPINA1. In some embodiments, a subject is homozygous for 1024 G>A (E342K) mutation in SERPINA1. In some embodiments, a subject is heterozygous for 1024 G>A (E342K) mutation in SERPINA1. In some embodiments, a subject comprises a PiZ allele. In some embodiments, a subject is a PiZZ carrier. In some embodiments, an oligonucleotide is capable of editing 1024 G>A mutation in SERPINA1 to 1. In some embodiments, an oligonucleotide is capable of correcting E342K mutation in SERPINA1. In some embodiments, after administration or delivery, adenosine editing, production of edited AAT (e.g., M-AAT), reduction of Z-AAT, increase of scrum AAT, increase of serum edited AAT (e.g., M-AAT; absolute concentration and/or %), reduction of Z-AAT aggregation, increased neutrophil elastase inhibition, reduced liver inflammation, reduced liver PAS-D positive area, reduced liver globular diameter, reduced liver fibrosis, and/or reduced liver cirrhosis are achieved compared to absence of such administration or delivery, or administration or delivery of a reference agent (e.g., an otherwise comparable or identical composition without oligonucleotides or with oligonucleotides that are not designed for editing the same adenosine).

In some embodiments, an oligonucleotide, when administered to a patient suffering from or susceptible to a condition, disorder or disease that is associated with a G to A mutation is capable of reducing at least one symptom of the condition, disorder or disease and/or capable of delaying or preventing the onset, worsening, and/or reducing the rate and/or degree of worsening of at least one symptom of the condition, disorder or disease that's due to a G to A mutation in a gene or gene product.

In some embodiments, provided technologies can provide editing of two or more sites in a system (e.g., a cell, tissue, organ, animal, etc.) (“multiplex editing”). In some embodiments, provided technologies can target and provide editing of two or more sites of the same transcripts. In some embodiments, provided technologies can target and provide editing of two or more different transcripts, either from the same nucleic acid or different nucleic acids. In some embodiments, provided technologies can target and provide editing of transcripts from two or more different nucleic acids. In some embodiments, provided technologies can target and provide editing of transcripts from two or more different genes. In some embodiments, of the targets simultaneously edited, each is independently at a biologically and/or therapeutically relevant level. In some embodiments, in multiplex editing one or more or all targets are independently edited at a comparable level as editing conducted individually under comparable conditions. In some embodiments, multiplex editing are performed utilizing two or more separate compositions, each of which independently target one or more targets. In some embodiments, compositions are administered concurrently. In some embodiments, compositions are administered with suitable intervals. In some embodiments, one or more compositions are administered prior or subsequently to one or more other compositions. In some embodiments, multiplex editing are performed utilizing a single composition, e.g., a composition comprising two or more pluralities of oligonucleotides, wherein the pluralities target different targets. In some embodiments, each plurality independently targets a different adenosine. In some embodiments, each plurality independently targets a different transcript. In some embodiments, each plurality independently targets a different gene. In some embodiments, two or more pluralities may target the same target, but the pluralities together target the desired targets.

As described herein, provided technologies can provide a number of advantages. For example, in some embodiments, provided technologies are safer than technologies that act on DNA, as provided technologies can provide RNA edits that are both reversible and tunable (e.g., through adjusting of doses). Additionally and alternatively, as demonstrated herein, provided technologies can provide high levels of editing in systems expressing endogenous ADAR proteins thus avoiding the requirement of introduction of exogenous proteins in various instances. Still further, provided technologies do not require complex oligonucleotides that depend on ancillary delivery vehicles, such as viral vectors or lipid nanoparticles, as utilized in many other technologies, particularly for application beyond cell culture. In some embodiments, provided technologies can provide sequence-specific A-to-I RNA editing with high efficiency using endogenous ADAR enzymes and can be delivered to various systems, e.g., cells, in the absence of artificial delivery agents.

Those skilled in the art reading the present disclosure will understand that provided oligonucleotides and compositions thereof may be delivered using a number of technologies in accordance with the present disclosure. In some embodiments, provided oligonucleotides and compositions may be delivered via transfection or lipofection. In some embodiments, provided oligonucleotides and compositions thereof may be delivered in the absence of delivery aids, such as those utilized in transfection or lipofection. In some embodiments, provided oligonucleotides and compositions thereof are delivered with gymnotic delivery. In some embodiments, provided oligonucleotides comprise additional chemical moieties that can facilitate delivery. For example, in some embodiments, additional chemical moieties are or comprise ligand moieties (e.g., N-acetylgalactosamine (GalNAc)) for receptors (e.g., asialoglycoprotein receptors). In some embodiments, provided oligonucleotides and compositions thereof can be delivered through GalNAc-mediated delivery. In some embodiments, provided technologies are delivered selectively to target cell populations, locations, tissues, organs, etc. In some embodiments, oligonucleotides or compositions are delivered through targeted delivery, e.g., using ligand moieties like GalNAc. In some embodiments, delivery is systemic delivery. In some embodiments, delivery is local delivery (e.g., via IT, IVT, etc.). In some embodiments, provided technologies provide advantages including delivery and editing without complex delivery vehicles. For example, in some embodiments, substantial delivery and RNA editing without lipids or ligand moieties (e.g., GalNAc) was observed in multiple tissues following a single subcutaneous dose to mice, including in heart, kidney, lung, spleen, white adipose tissue (WAT), brown adipose tissue (BAT), liver, and various liver associated cells such as CD3+ cells (T-cells and subset of NK cells), EpCAM+ cells (e.g., cholangiocytes in liver), liver sinosoidal endothelial cells (LSEC), macrophages (e.g., Kupfer cells), etc.

In some embodiments, provided technologies, e.g., methods, dosage regimens, etc., may comprise one or more loading doses. In some embodiments, technologies comprising loading doses may provide one or more desired effects or results faster than without such loading doses, for example, in some embodiments, editing levels may be increased or achieved faster than without loading doses. In some embodiments, a loading dose is the same as a non-loading dose (e.g., a maintenance dose, a dose administered in regimens without loading doses, etc.). In some embodiments, a loading dose contains about the same amount of agents, e.g., oligonucleotides, as a non-loading dose. In some embodiments, a loading dose is different from a non-loading dose. In some embodiments, a loading dose contains a reduced amount of agents, e.g., oligonucleotides, compared to a non-loading dose. In some embodiments, a loading dose contains an increased amount of agents, e.g., oligonucleotides, compared to a non-loading dose. In some embodiments, two or more loading doses arc utilized, each of which independently contains about the same amount of, less or more agents compared to a non-loading dose. In some embodiments, each loading dose is about the same. In some embodiments, each loading dose contains about the same amount of agents, e.g., oligonucleotides, as a non-loading dose. In some embodiments, one or more loading doses are different from one or more other loading doses. In some embodiments, one or more or all loading doses independently contains more agents, e.g., oligonucleotides, compared to a non-loading dose. In some embodiments, one or more or all loading doses independently contains less agents, e.g., oligonucleotides, compared to a non-loading dose. In some embodiments, each non-loading dose is about the same. In some embodiments, technologies without loading doses can provide comparable or about the same effects or results after a period of time, e.g., 1, 2, 3, 4, 5, 6, 7, 8, 9, 10 or more weeks, or 1, 2, 3, 4, 5, 6, 7, 8, 9, 10 or more months.

Among other things, the present disclosure provides the following Example Embodiments:

1. An oligonucleotide having the structure of:
Mod001L001mCn001RmC*SmC*SfA*SfG*SmCmA*SfG*SfCmU*SfUn001RmCfA*SfGn001RfUmC*SfC*SfC*SfU*SmUmUfC*ST*Sb008U*SIn001SmUfC*SmG*SmAn001RmU (SEQ ID NO: 1), or a salt thereof, wherein:
Mod001 represents

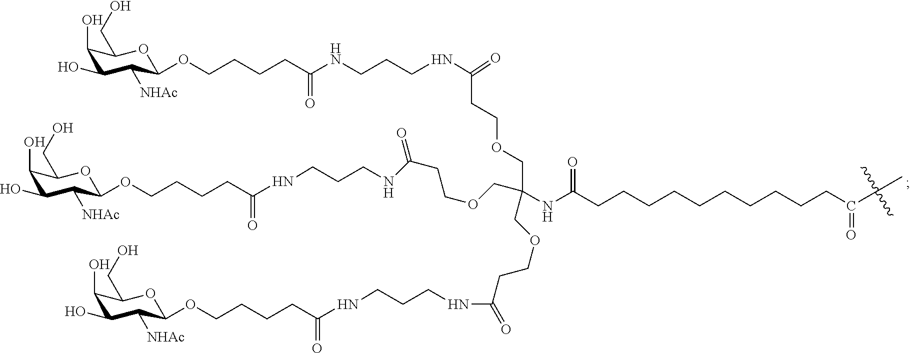

L001: —NH—(CH₂)₆—, connected to Mod001 through —NH— and the 5′-end of the oligonucleotide chain through a phosphate linkage;
f represents a 2′-F modification to a nucleoside;
m represents a 2′-OMe modification to a nucleoside:
n001R represents a Rp n001 linkage;
n001S represents a Sp n001 linkage;
a n001 linkage has the structure of

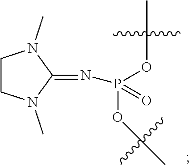 [SMILES: *OP(=O)(N=C1N(C)CCN1C)O*]

*S represents a Sp phosphorothioate linkage;
I represents the nucleobase is hypoxanthine; and
b008U represents a nucleoside whose base is

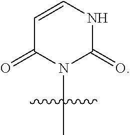 [SMILES: *n1c(=O)cc[nH]c1=O]

2. An oligonucleotide having the structure of:

(SEQ ID NO: 3)
Mod001L001mCn001RmC*SmC*SfA*SfG*Sm5CeoAcofG*SfC*
STeofUn001RmCfA*SfGn001RfUmC*SfC*SfC*
SfUn001RTeoTeofC*ST*Sb008U*SIn001SmUfC*SmG*
SmAn001RmU,

or a salt thereof, wherein:
Mod001 represents

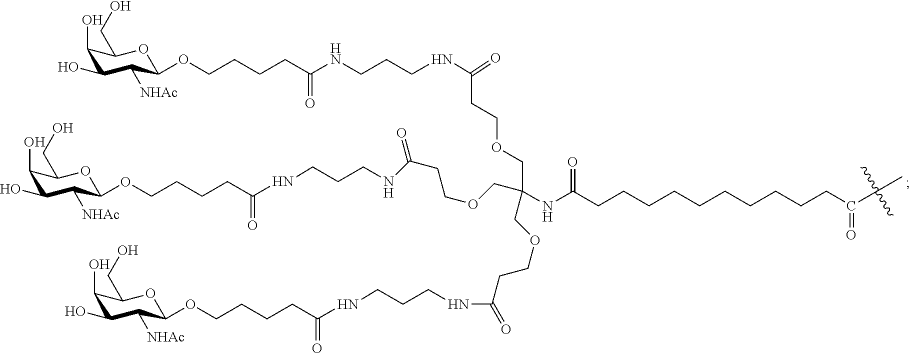

L001: —NH—(CH₂)₆—, connected to Mod001 through —NH—and the 5′-end of the oligonucleotide chain through a phosphate linkage;
f represents a 2′-F modification to a nucleoside;
m represents a 2′-OMe modification to a nucleoside;
eo represents a 2′-OCH₂CH₂OCH₃ modification to a nucleoside;
m5Ceo represents 5-methyl 2′-O-methoxyethyl C;
n001R represents a Rp n001 linkage;
n001S represents a Sp n001 linkage:
a n001 linkage has the structure of

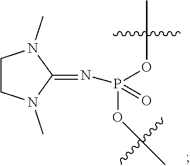 [SMILES: *OP(=O)(N=C1N(C)CCN1C)O*]

*S represents a Sp phosphorothioate linkage;
I represents the nucleobase is hypoxanthine; and
b008U represents a nucleoside whose base is

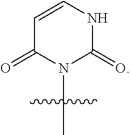 [SMILES: *n1c(=O)cc[nH]c1=O]

3. An oligonucleotide having the structure of:
Mod001L001 mCn001RmC*nSmC*SfA*SfG*SmCmAfG*SfC*SmUfUn001RmCfA*SmGn001RfUmC*SfC*SfC*SfJn001RmUmUfC*ST*Sb008U*SIn001SmUfC*SmG*SmAn001RmU (SEQ ID NO: 5), or
   a salt thereof, wherein:
Mod001 represents

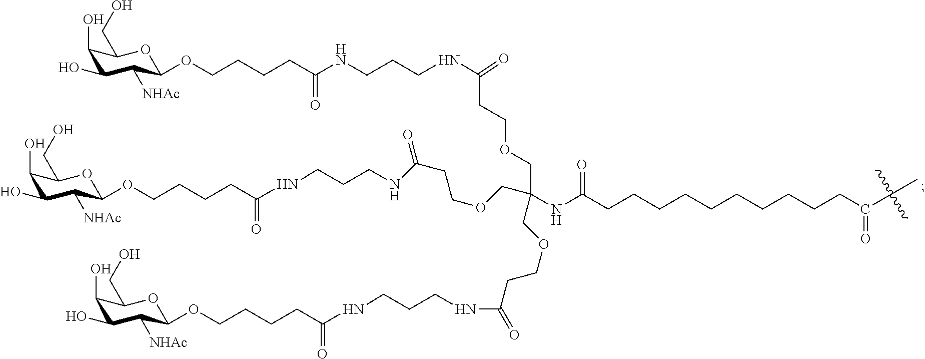

L001: —NH—(CH₂)₆—, connected to Mod001 through —NH— and the 5-end of the oligonucleotide chain through a phosphate linkage;
f represents a 2′-F modification to a nucleoside;
m represents a 2′-OMe modification to a nucleoside:
n001R represents a Rp n001 linkage;
n001S represents a Sp n001 linkage;
a n001 linkage has the structure of

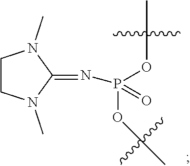 [SMILES: *OP(=O)(N=C1N(C)CCN1C)O*]

* S represents a Sp phosphorothioate linkage.
I represents the nucleobase is hypoxanthine; and
b008U represents a nucleoside whose base is

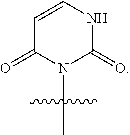 [SMILES: *n1c(=O)cc[nH]c1=O]

4. An oligonucleotide having the structure of:
Mod001L001mCn001RmC*SmC*SfA*SfG*Sm5CeoAeofG*SfC*STeofUn001RmCfA*SmGn001RfUmC*SfC*SfC*SfUn001RTeoTeofC*ST*Sb008U*SIn001SmUfC*SmG*SmAn001RmU (SEQ ID NO: 7),
   or a salt thereof, wherein:

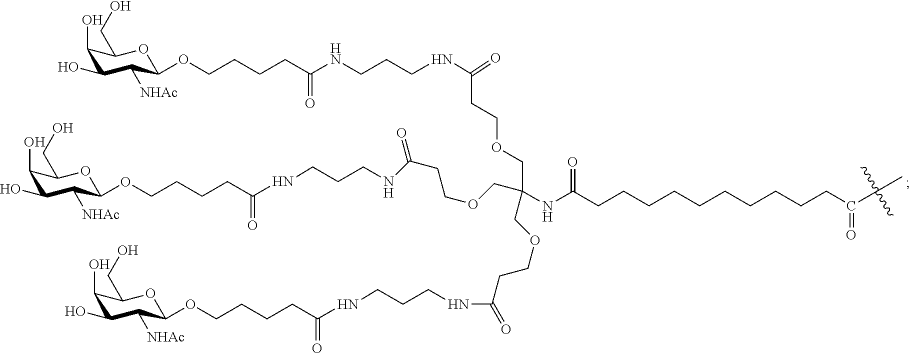

L001: —NH—(CH₂)₆—, connected to Mod001 through —NH— and the 5′-end of the oligonucleotide chain through a phosphate linkage;
f represents a 2′-F modification to a nucleoside;
in represents a 2′-OMe modification to a nucleoside:
eo represents a 2′-OCH₂CH₂OCH₃ modification to a nucleoside:
m5Ceo represents 5-methyl 2′-O-methoxyethyl C;
n001R represents a Rp n001 linkage:
n001S represents a Sp n001 linkage:
a n001 linkage has the structure of

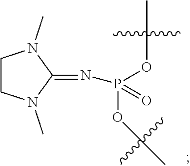 [SMILES: *OP(=O)(N=C1N(C)CCN1C)O*]

*S represents a Sp phosphorothioate linkage:
I represents the nucleobase is hypoxanthine; and
b008U represents a nucleoside whose base is

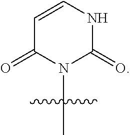 [SMILES: *n1c(=O)cc[nH]c1=O]

5. An oligonucleotide having the structure of
Mod001L001mCn001RmC*SmC*SfA*SfG*Sm5CeoAeofG*SfC*STeofUn001RmCfA*SmGn001R fUm5Ceo*SfC*SfC*SfUn001RTeoTeofC*ST*Sb008U*SIn001SmUfC*SmG*SmAn001RmU (SEQ ID NO: 8), or a salt thereof, wherein:
Mod001 represents

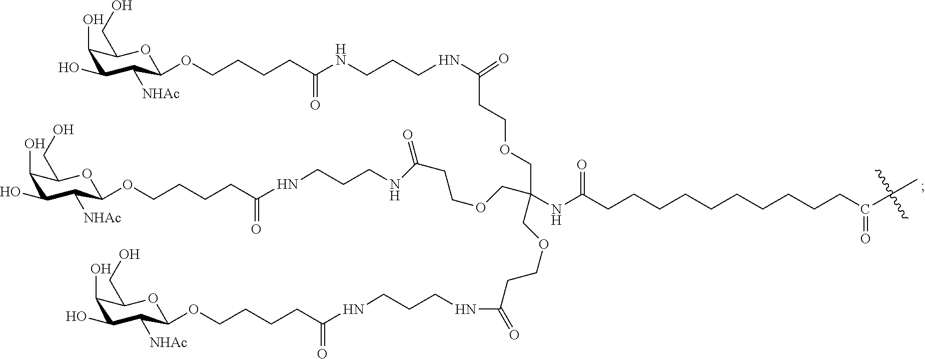

L001: —NH—(CH₂)₆—, connected to Mod001 through —NH— and the 5′-end of the oligonucleotide chain through a phosphate linkage:
f represents a 2′-F modification to a nucleoside;
m represents a 2′-OMe modification to a nucleoside;
eo represents a 2′-OCH₂CH₂OCH₃ modification to a nucleoside;
m5Ceo represents 5-methyl 2′-O-methoxyethyl C;
n001R represents a Rp n001 linkage;
n001S represents a Sp n001 linkage;
a n001 linkage has the structure of

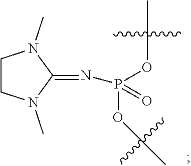 [SMILES: *OP(=O)(N=C1N(C)CCN1C)O*]

*S represents a Sp phosphorothioate linkage.
I represents the nucleobase is hypoxanthine; and
b008U represents a nucleoside whose base is

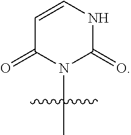 [SMILES: *n1c(=O)cc[nH]c1=O]

6. An oligonucleotide having the structure of:
Mod001L001 mCn001RmC*SmC*SfA*SfG*Sm5CeoAeofG*SmCTeo*SmUn001RmCfA*SfGn001 RmUmCmC*SfC*SfU*STeoTeofC*ST*Sb008U*SIn001SmUfC*SmG*SmAn001RmU (SEQ ID NO: 9), or a salt thereof, wherein:
Mod001 represents

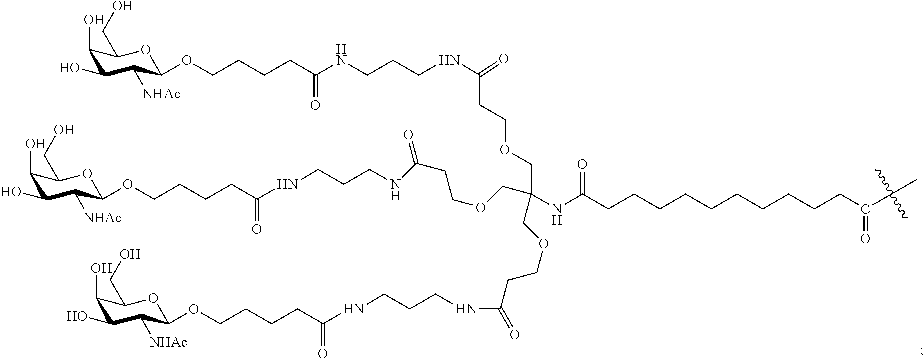

L001: —NH—(CH₂)₆—, connected to Mod001 through —NH— and the 5′-end of the oligonucleotide chain through a phosphate linkage:
f represents a 2′-F modification to a nucleoside;
m represents a 2′-OMe modification to a nucleoside;
eo represents a 2′-OCH₂CH₂OCH₃ modification to a nucleoside;
m5Ceo represents 5-methyl 2′-O-methoxyethyl C;
n001R represents a Rp n001 linkage;
n001S represents a Sp n001 linkage;
a n001 linkage has the structure of

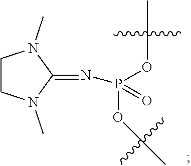 [SMILES: *OP(=O)(N=C1N(C)CCN1C)O*]

*S represents a Sp phosphorothioate linkage;
I represents the nucleobase is hypoxanthine; and
b008U represents a nucleoside whose base is

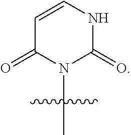 [SMILES: *n1c(=O)cc[nH]c1=O]

7. An oligonucleotide having the structure of:
Mod001L001 mCn001RmC*SmC*SfA*SfG*Sm5CeoAeofG*SmCTeo*SmUn001RmCfA*SfGn001 RmUm5CeomC*SfC*SfU*STeoTeofC*ST*Sb008U*SIn001SmUfC*SmG*SmAn001RmU (SEQ ID NO: 10), or a salt thereof, wherein:
Mod001 represents

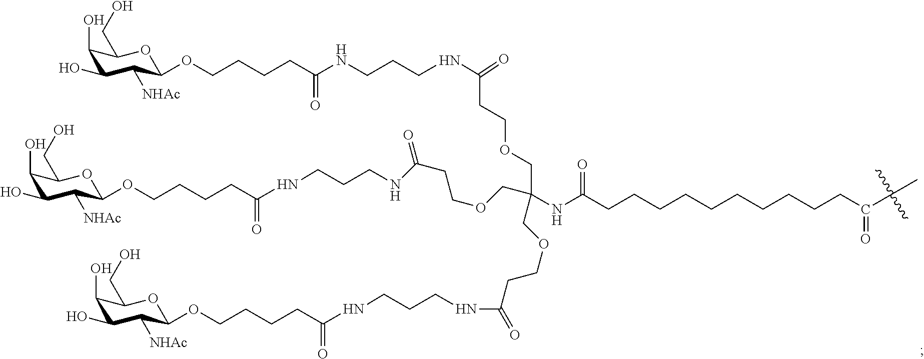

L001: —NH—(CH₂)₆—, connected to Mod001 through —NH— and the 5′-end of the oligonucleotide chain through a phosphate linkage:
f represents a 2′-F modification to a nucleoside;
m represents a 2′-OMe modification to a nucleoside;
eo represents a 2′-OCH₂CH₂OCH₃ modification to a nucleoside;
m5Ceo represents 5-methyl 2′-O-methoxyethyl C;
n001R represents a Rp n001 linkage;
n001S represents a Sp n001 linkage;
a n001 linkage has the structure of

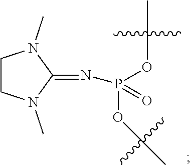 [SMILES: *OP(=O)(N=C1N(C)CCN1C)O*]

*S represents a Sp phosphorothioate linkage;
I represents the nucleobase is hypoxanthine; and
b008U represents a nucleoside whose base is

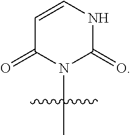 [SMILES: *n1c(=O)cc[nH]c1=O]

8. An oligonucleotide having the structure of:
Mod001L001 mCn001RmC*SmC*SfA*SfG*Sm5CeoAeofG*Sm5CeoTeo*SmUn001Rm5CeofA*Sf Gn001RmUm5Ceom5Ceo*SfC*SfU*STeoTeofC*ST*Sb008U*SIn001SmUfC*SmG*SmAn001RmU (SEQ ID NO: 12), or a salt thereof, wherein:
Mod001 represents

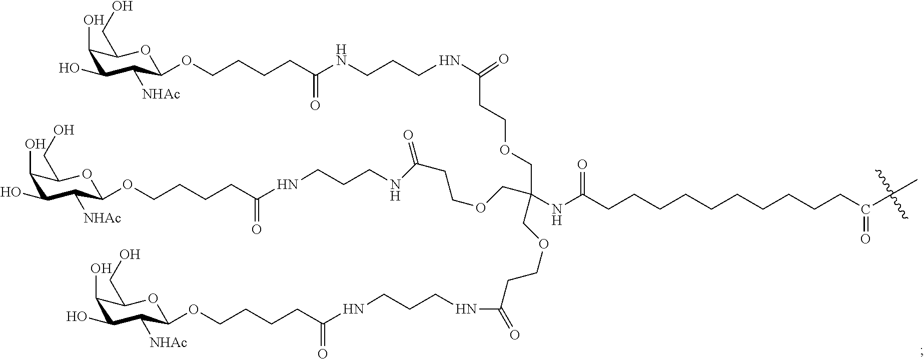

L001: —NH—(CH₂)₆—, connected to Mod001 through —NH— and the 5′-end of the oligonucleotide chain through a phosphate linkage:
f represents a 2′-F modification to a nucleoside;
m represents a 2′-OMe modification to a nucleoside;
eo represents a 2′-OCH₂CH₂OCH₃ modification to a nucleoside;
m5Ceo represents 5-methyl 2′-O-methoxyethyl C;
n001R represents a Rp n001 linkage;
n001S represents a Sp n001 linkage;
n001 linkage has the structure of

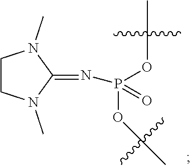 [SMILES: *OP(=O)(N=C1N(C)CCN1C)O*]

*S represents a Sp phosphorothioate linkage.
I represents the nucleobase is hypoxanthine; and
b008U represents a nucleoside whose base is

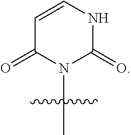 [SMILES: *n1c(=O)cc[nH]c1=O]

9. An oligonucleotide having the structure of:
Mod001L001mCn001RmC*SmC*SfA*Sf G*Sm5CeoAeofG*SfC*SmUmUn001RmCfA*SfGn001RfUm5Ceo*SfC*SmCmUn001RmUTeofC*ST*Sb008U*SIn001SmUfC*SmG*SmAn001RmU (SEQ ID NO: 13), or a salt thereof, wherein:
Mod001 represents

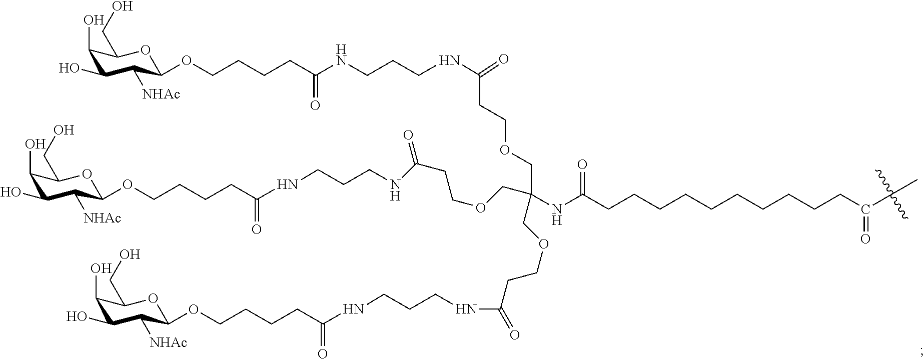

L001: —NH—(CH₂)₆—, connected to Mod001 through —NH— and the 5′-end of the oligonucleotide chain through a phosphate linkage:
f represents a 2′-F modification to a nucleoside;
m represents a 2′-OMe modification to a nucleoside;
eo represents a 2′-OCH₂CH₂OCH₃ modification to a nucleoside;
m5Ceo represents 5-methyl 2′-O-methoxyethyl C;
n001R represents a Rp n001 linkage;
n001S represents a Sp n001 linkage;
a n001 linkage has the structure of

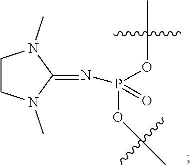 [SMILES: *OP(=O)(N=C1N(C)CCN1C)O*]

*S represents a Sp phosphorothioate linkage;
I represents the nucleobase is hypoxanthine; and
b008U represents a nucleoside whose base is

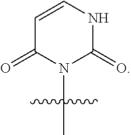 [SMILES: *n1c(=O)cc[nH]c1=O]

10. An oligonucleotide having the structure of:
Mod001L001 mCn001RmC*SmC*SfA*SfG*Sm5CeoAeofU*SfC*STeofUn001RmCfA*SfGn001RfUm5Ceo*SfC*SfC*SfUn001RTeoTeofC*ST*Sb008U*SIn001SmUfC*SmG*SmAn001RmU (SEQ ID NO: 15), or a salt thereof, wherein:
Mod001 represents

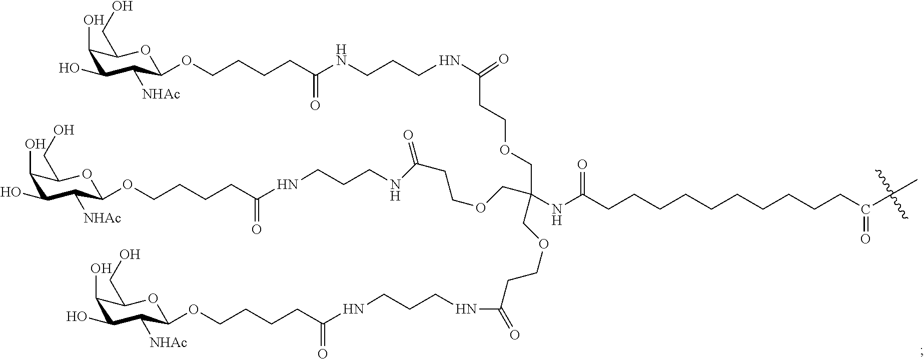

L001: —NH—(CH₂)₆—, connected to Mod001 through —NH— and the 5′-end of the oligonucleotide chain through a phosphate linkage:
f represents a 2′-F modification to a nucleoside;
m represents a 2′-OMe modification to a nucleoside;
eo represents a 2′-OCH₂CH₂OCH₃ modification to a nucleoside;
m5Ceo represents 5-methyl 2′-O-methoxyethyl C;
n001R represents a Rp n001 linkage;
n001S represents a Sp n001 linkage;
a n001 linkage has the structure of

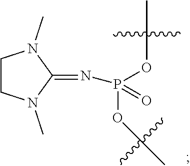 [SMILES: *OP(=O)(N=C1N(C)CCN1C)O*]

*S represents a Sp phosphorothioate linkage;
I represents the nucleobase is hypoxanthine; and
b008U represents a nucleoside whose base is

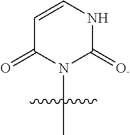 [SMILES: *n1c(=O)cc[nH]c1=O]

11. An oligonucleotide, having a structure selected from Table 1 of WO 2022/099159 or a salt thereof, wherein the oligonucleotide targets SERPINA1 and comprises an additional chemical moiety, or a salt thereof.

12. An oligonucleotide, having a structure selected from Table 1 of WO 2022/099159 or a salt thereof, wherein the oligonucleotide targets SERPINA1 and comprises L001.

13. An oligonucleotide, having a structure selected from Table 1 of WO 2022/099159 or a salt thereof, wherein the oligonucleotide targets SERPINA1 and comprises Mod001.

14. The oligonucleotide of any one of Embodiments 11-13, wherein the oligonucleotide has a structure selected from Table 1D of WO 2022/099159 or a salt thereof.

15. The oligonucleotide of any one of Embodiments 11-13, wherein the oligonucleotide has a structure selected from Table 1E of WO 2022/099159 or a salt thereof.

16. The oligonucleotide of any one of Embodiments 11-13, wherein the oligonucleotide has a structure selected from Table 1F of WO 2022/099159 or a salt thereof.

17. The oligonucleotide of any one of Embodiments 11-13, wherein the oligonucleotide has a structure selected from WV-46312 to WV-46323 in Table 1F of WO 2022/099159 or a salt thereof.

18. The oligonucleotide of any one of Embodiments 11-13, wherein the oligonucleotide has a structure selected from WV-47597 to WV-47609 in Table 1F of WO 2022/099159 or a salt thereof.

19. The oligonucleotide of any one of Embodiments 11-13, wherein the oligonucleotide has a structure selected from WV-47641 to WV-48454 in Table 1F of WO 2022/099159 or a salt thereof.

20. The oligonucleotide of any one of Embodiments 11-13, wherein the oligonucleotide has a structure selected from WV-47643 to WV-47648 in Table 1F of WO 2022/099159 or a salt thereof.

21. The oligonucleotide of any one of Embodiments 11-13, wherein the oligonucleotide has a structure selected from WV-48453 to WV-48454 in Table 1F of WO 2022/099159 or a salt thereof.

22. The oligonucleotide of any one of Embodiments 11-13, wherein the oligonucleotide has a structure selected from WV-49085 to WV-49093 in Table 1F of WO 2022/099159 or a salt thereof.

23. The oligonucleotide of any one of Embodiments 11-13, wherein the oligonucleotide has a structure selected from Table 10 of WO 2022/099159 or a salt thereof.

24. An oligonucleotide, having the structure of the oligonucleotide chain of the oligonucleotide of any one of the preceding Embodiments.

25. An oligonucleotide, formed by cleaving the additional chemical moiety from the oligonucleotide chain of an oligonucleotide of any one of Embodiments 1-23.

26. The oligonucleotide of Embodiment 25, wherein the additional chemical moiety is cleaved after the oligonucleotide of any one of Embodiments 1-23 is delivered into a cell.

27. The oligonucleotide of any one of Embodiments 25-26, wherein the additional chemical moiety is cleaved after the oligonucleotide of any one of Embodiments 1-23 is administered to a subject.

28. An oligonucleotide having the structure of:
mCn001RmC*SmC*SfA*SfG*SmCmA*SfG*SfCmU*SfUn001RmCfA*SfGn001RfUmC*SfC*Sf C*SfU*SmUmUfC*ST*Sb008U*SIn001SmUfC*SmG*SmAn001RmU (SEQ ID NO: 2), or a salt thereof, wherein:
Mod001 represents
f represents a 2′-F modification to a nucleoside;
m represents a 2′-OMe modification to a nucleoside;
n001R represents a Rp n001 linkage;
n001S represents a Sp n001 linkage;
a n001 linkage has the structure of

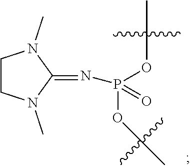 [SMILES: *OP(=O)(N=C1N(C)CCN1C)O*]

*S represents a Sp phosphorothioate linkage;
I represents the nucleobase is hypoxanthine; and
b008U represents a nucleoside whose base is

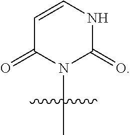 [SMILES: *n1c(=O)cc[nH]c1=O]

29. An oligonucleotide having the structure of:
mCn001RmC*SmC*SfA*SfG*Sm5CeoAeofG*SfC*STeofUn001RmCfA*SfGn001RfUmC*SfC*SfC*SfUn001RTeoTeofC*ST*Sb008U*SIn001SmUfC*SmG*SmAn001RmU (SEQ ID NO: 4), or a salt thereof, wherein:
f represents a 2′-F modification to a nucleoside;
m represents a 2′-OMe modification to a nucleoside;
eo represents a 2′-OCH₂CH₂OCH₃ modification to a nucleoside;
m5Ceo represents 5-methyl 2′-O-methoxyethyl C;
n001R represents a Rp n001 linkage;
n001S represents a Sp n001 linkage;
a n001 linkage has the structure of

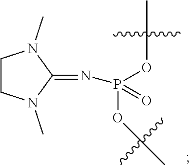 [SMILES: *OP(=O)(N=C1N(C)CCN1C)O*]

*S represents a Sp phosphorothioate linkage.
I represents the nucleobase is hypoxanthine; and
b008U represents a nucleoside whose base is

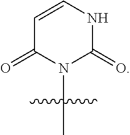 [SMILES: *n1c(=O)cc[nH]c1=O]

30. An oligonucleotide having the structure of:
mCn001RmC*SmC*SfA*SfG*SmCmAfG*SfC*SmUfUn001RmCfA*SmGn001RfUmC*SfC*SfC*SfUn001RmUmUfC*ST*Sb008U*SIn001SmUfC*SmG*SmAn001RmU (SEQ ID NO: 6), or a salt thereof, wherein:
f represents a 2′-F modification to a nucleoside:
m represents a 2′-OMe modification to a nucleoside:
n001R represents a Rp n001 linkage;
n001S represents a Sp n001 linkage:
a n001 linkage has the structure of

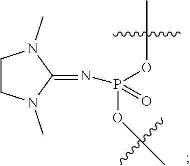 [SMILES: *OP(=O)(N=C1N(C)CCN1C)O*]

*S represents a Sp phosphorothioate linkage;
I represents the nucleobase is hypoxanthine; and
b008U represents a nucleoside whose base is

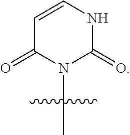 [SMILES: *n1c(=O)cc[nH]c1=O]

31. An oligonucleotide having the structure of:
mCn001RmC*SmC*SfA*SfG*Sm5CeoAeofG*SfC*STeofUn001RmCfA*SmGn001RfUmC*SfC*SfC*SfUn001RTeoTeofC*ST*Sb008U*SIn001SmUfC*SmG*SmAn001RmU (SEQ ID NO: 29), or a salt thereof, wherein:
f represents a 2′-F modification to a nucleoside;
m represents a 2′-OMe modification to a nucleoside;
eo represents a 2′-OCH₂CH₂OCH₃ modification to a nucleoside:
m5Ceo represents 5-methyl 2′-O-methoxyethyl C;
n001R represents a Rp n001 linkage;
n001S represents a Sp n001 linkage;
a n001 linkage has the structure of

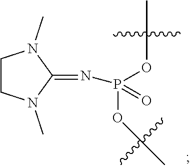 [SMILES: *OP(=O)(N=C1N(C)CCN1C)O*]

*S represents a Sp phosphorothioate linkage.
I represents the nucleobase is hypoxanthine; and
b008U represents a nucleoside whose base is

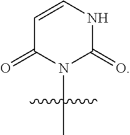 [SMILES: *n1c(=O)cc[nH]c1=O]

32. An oligonucleotide having the structure of:
mCn001RmC*SmC*SfA*SfG*Sm5CeoAeofG*SfC*STeofUn001RmCfA*SmGn001RfUm5Ceo*Sf C*SfC*SfUn001RTeoTeofC*ST*Sb008U*SIn001SmUfC*SmG*SmAn001RmU (SEQ ID NO: 30), or a salt thereof, wherein:
f represents a 2′-F modification to a nucleoside;
m represents a 2′-OMe modification to a nucleoside:
eo represents a 2′-OCH₂CH₂OCH₃ modification to a nucleoside;
m5Ceo represents 5-methyl 2′-O-methoxyethyl C;
n001R represents a Rp n001 linkage,
n001S represents a Sp n001 linkage:
a n001 linkage has the structure of

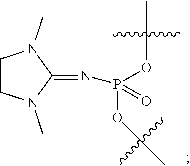 [SMILES: *OP(=O)(N=C1N(C)CCN1C)O*]

*S represents a Sp phosphorothioate linkage;
I represents the nucleobase is hypoxanthine; and
b008U represents a nucleoside whose base is

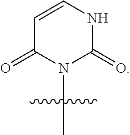 [SMILES: *n1c(=O)cc[nH]c1=O]

33. An oligonucleotide having the structure of:
mCn001RmC*SmC*SfA*SfG*Sm5CeoAeofG*SmCTeo*SmUn001RmCfA*SfGn001RmUmCmC*SfC*SfU*STeoTeofC*ST*Sb008U*SIn001SmUfC*SmG*SmAn001RmU (SEQ ID NO: 31), or a salt thereof, wherein:
f represents a 2′-F modification to a nucleoside;
m represents a 2′-OMe modification to a nucleoside;
eo represents a 2′-OCH₂CH₂OCH, modification to a nucleoside;
m5Ceo represents 5-methyl 2′-O-methoxyethyl C;
n001R represents a Rp n001 linkage;
n001S represents a Sp n001 linkage;
a n001 linkage has the structure of

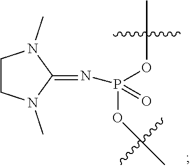 [SMILES: *OP(=O)(N=C1N(C)CCN1C)O*]

*S represents a Sp phosphorothioate linkage;
I represents the nucleobase is hypoxanthine; and
b008U represents a nucleoside whose base is

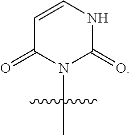 [SMILES: *n1c(=O)cc[nH]c1=O]

34. An oligonucleotide having the structure of:
mCn001RmC*SmC*SfA*SfG*Sm5CeoAeofG*SmCTeo*SmUn001RmCfA*SfGn001RmUm5Ceo mC*SfC*SfU*STeoTeofC*ST*Sb008U*SIn001SmUfC*SmG*SmAn001RmU (SEQ ID NO: 11), or a salt thereof, wherein:
f represents a 2′-F modification to a nucleoside;
m represents a 2′-OMe modification to a nucleoside;
eo represents a 2′-OCH₂CH₂OCH₃ modification to a nucleoside;
m5Ceo represents 5-methyl 2′-O-methoxyethyl C;
n001R represents a Rp n001 linkage;
n001S represents a Sp n001 linkage;
a n001 linkage has the structure of

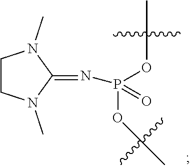 [SMILES: *OP(=O)(N=C1N(C)CCN1C)O*]

* S represents a Sp phosphorothioate linkage;
I represents the nucleobase is hypoxanthine; and
b008U represents a nucleoside whose base is

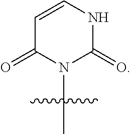 [SMILES: *n1c(=O)cc[nH]c1=O]

35. An oligonucleotide having the structure of:
mCn001RmC*SmC*SfA*SfG*Sm5CeoAeofG*Sm5CeoTeo*SmUn001Rm5CeofA*SfGn001RmU m5Ceom5Ceo*SfC*SfU*STeoTeofC*ST*Sb008U*SIn001SmUfC*SmG*SmAn001RmU (SEQ ID NO: 32), or a salt thereof, wherein:
f represents a 2′-F modification to a nucleoside;
m represents a 2′-OMe modification to a nucleoside;
eo represents a 2′-OCH₂CH₂OCH₃ modification to a nucleoside;
m5Ceo represents 5-methyl 2′-O-methoxyethyl C;
n001R represents a Rp n001 linkage;
n001S represents a Sp n001 linkage;
a n001 linkage has the structure of

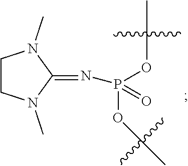 [SMILES: *OP(=O)(N=C1N(C)CCN1C)O*]

*S represents a Sp phosphorothioate linkage;
I represents the nucleobase is hypoxanthine; and
b008U represents a nucleoside whose base is

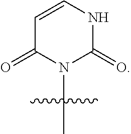 [SMILES: *n1c(=O)cc[nH]c1=O]

36. An oligonucleotide having the structure of:
mCn001RmC*SmC*SfA*SfG*Sm5CeoAeofG*SfC*SmUmUn001RmCfA*SfGn001RfUm5Ceo*SfC*SmCmUn001RmUTeofC*ST*Sb008U*SIn001SmUfC*SmG*SmAn001RmU (SEQ ID NO: 14), or a salt thereof, wherein:
f represents a 2′-F modification to a nucleoside;
m represents a 2′-OMe modification to a nucleoside;
eo represents a 2′-OCH₂CH₂OCH₃ modification to a nucleoside;
m5Ceo represents 5-methyl 2′-O-methoxyethyl C;
n001R represents a Rp n001 linkage;
n001S represents a Sp n001 linkage;
a n001 linkage has the structure of

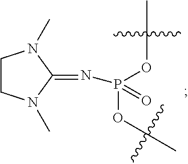 [SMILES: *OP(=O)(N=C1N(C)CCN1C)O*]

*S represents a Sp phosphorothioate linkage;
I represents the nucleobase is hypoxanthine; and
b008U represents a nucleoside whose base is

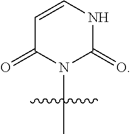 [SMILES: *n1c(=O)cc[nH]c1=O]

37. An oligonucleotide having the structure of:
mCn001RmC*SmC*SfA*SfG*Sm5CeoAeofG*SfC*STeofUn001RmCfA*SfGn001 RfUm5Ceo*Sf C*SfC*SfUn001RTeoTeofC*ST*Sb008U*SIn001SmUfC*SmG*SmAn001RmU (SEQ ID NO: 16), or a salt thereof, wherein:
f represents a 2′-F modification to a nucleoside;
m represents a 2′-OMe modification to a nucleoside:
eo represents a 2′-OCH₂CH₂OCH₃ modification to a nucleoside;
m5Ceo represents 5-methyl 2′-O-methoxyethyl C;
n001R represents a Rp n001 linkage,
n001S represents a Sp n001 linkage;
a n001 linkage has the structure of

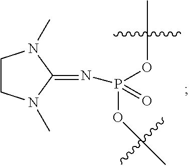 [SMILES: *OP(=O)(N=C1N(C)CCN1C)O*]

*S represents a Sp phosphorothioate linkage:
I represents the nucleobase is hypoxanthine; and
b008U represents a nucleoside whose base is

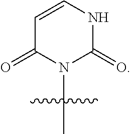 [SMILES: *n1c(=O)cc[nH]c1=O]

38. An oligonucleotide, having a structure selected from Table 1 of WO 2022/099159 or a salt thereof, wherein the oligonucleotide targets SERPINA1 and does not contain an additional chemical moiety.

39. An oligonucleotide, having a structure selected from Table 1 of WO 2022/099159 or a salt thereof, wherein the oligonucleotide targets SERPINA1 and does not contain L001.

40. An oligonucleotide, having a structure selected from Table 1 of WO 2022/099159 or a salt thereof, wherein the oligonucleotide does not contain Mod001 or Mod012.

41. The oligonucleotide of any one of Embodiments 38-40, wherein the oligonucleotide has a structure selected from Table 1D of WO 2022/099159 or a salt thereof.

42. The oligonucleotide of any one of Embodiments 38-40, wherein the oligonucleotide has a structure selected from Table 1E of WO 2022/099159 or a salt thereof.

43. The oligonucleotide of any one of Embodiments 38-40, wherein the oligonucleotide has a structure selected from Table 1F of WO 2022/099159 or a salt thereof.

44. The oligonucleotide of any one of Embodiments 38-40, wherein the oligonucleotide has a structure selected from WV-42934 to WV-44247 in Table 1F of WO 2022/099159 or a salt thereof.

45. The oligonucleotide of any one of Embodiments 38-40, wherein the oligonucleotide has a structure selected from WV-44248 to WV-44277 in Table 1F of WO 2022/099159 or a salt thereof.

46. The oligonucleotide of any one of Embodiments 38-40, wherein the oligonucleotide has a structure selected from WV-44349 to WV-44362 in Table 1F of WO 2022/099159 or a salt thereof.

47. The oligonucleotide of any one of Embodiments 38-40, wherein the oligonucleotide has a structure selected from WV-44363 to WV-44390 in Table 1F of WO 2022/099159 or a salt thereof.

48. The oligonucleotide of any one of Embodiments 38-40, wherein the oligonucleotide has a structure selected from WV-44482 to WV-44515 in Table 1F of WO 2022/099159 or a salt thereof.

49. The oligonucleotide of any one of Embodiments 38-40, wherein the oligonucleotide has a structure selected from WV-46406 to WV-47042 in Table 1F of WO 2022/099159 or a salt thereof.

50. The oligonucleotide of any one of Embodiments 38-40, wherein the oligonucleotide has a structure selected from WV-47339 to WV-47483 in Table 1F of WO 2022/099159 or a salt thereof.

51. The oligonucleotide of any one of Embodiments 38-40, wherein the oligonucleotide has a structure selected from WV-47495 to WV-47496 in Table 1F of WO 2022/099159 or a salt thereof.

52. The oligonucleotide of any one of Embodiments 38-40, wherein the oligonucleotide has a structure selected from WV-47610 to WV-47631 in Table 1F of WO 2022/099159 or a salt thereof.

53. The oligonucleotide of any one of Embodiments 38-40, wherein the oligonucleotide has a structure selected from WV-48455 to WV-48459 in Table 1F of WO 2022/099159 or a salt thereof.

54. The oligonucleotide of any one of Embodiments 38-40, wherein the oligonucleotide has a structure selected from WV-49094 to WV-49096 in Table 1F of WO 2022/099159 or a salt thereof.

55. The oligonucleotide of any one of Embodiments 38-40, wherein the oligonucleotide has a structure selected from Table 10 of WO 2022/099159 or a salt thereof.

56. An oligonucleotide which is a conjugate of an oligonucleotide of any one of Embodiments 24-55 with an additional chemical moiety or a salt thereof.

57. The oligonucleotide of Embodiment 56, wherein the additional chemical moiety is or comprises a targeting moiety.

58. The oligonucleotide of Embodiment 56, wherein the additional chemical moiety is or comprises a carbohydrate moiety.

59. The oligonucleotide of Embodiment 56, wherein the additional chemical moiety is or comprises a lipid moiety.

60. The oligonucleotide of Embodiment 56, wherein the additional chemical moiety is or comprises one or more protein ligand moieties.

61. The oligonucleotide of Embodiment 56, wherein the additional chemical moiety is or comprises two or more protein ligand moieties.

62. The oligonucleotide of Embodiment 56, wherein the additional chemical moiety targets liver.

63. The oligonucleotide of Embodiment 56, wherein the additional chemical moiety is or comprises a ligand of a receptors expressed in liver.

64. The oligonucleotide of Embodiment 56, wherein the additional chemical moiety is or comprises a ligand for an asialoglycoprotein receptor.

65. The oligonucleotide of Embodiment 56, wherein the additional chemical moiety comprises multiple moieties, each of which is independently a ligand for an asialoglycoprotein receptor.

66. The oligonucleotide of Embodiment 56, wherein the additional chemical moiety is or comprises GalNAc.

67. The oligonucleotide of Embodiment 56, wherein the additional chemical moiety comprises three GalNAc.

68. The oligonucleotide of Embodiment 56, wherein the additional chemical moiety is or comprises

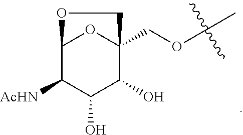 [SMILES: *OC[C@@]12CO[C@@H](O1)[C@H](NC(C)=O)[C@@H](O)[C@H]2O]

69. The oligonucleotide of Embodiment 56, wherein the additional chemical moiety is

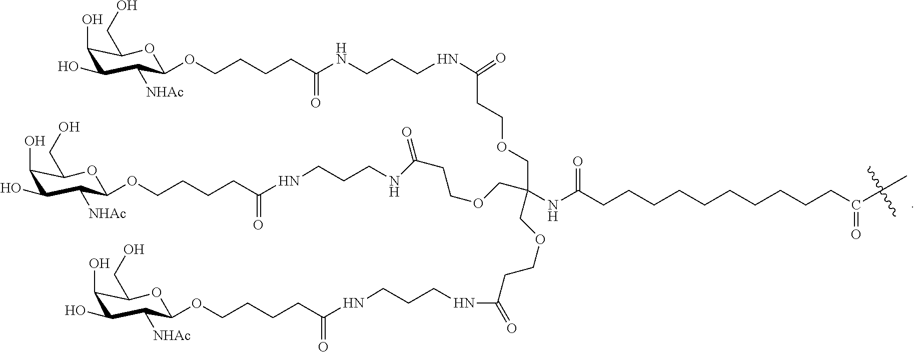

170. The oligonucleotide of Embodiment 56, wherein the additional chemical moiety is or comprises

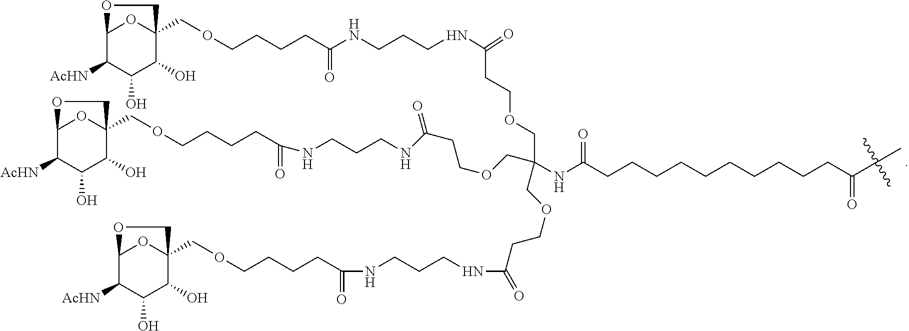 [SMILES: *C(=O)CCCCCCCCCCC(=O)NC(COCCC(=O)NCCCNC(=O)CCCCOC[C@@]12CO[C@@H](O1)[C@H](NC(C)=O)[C@@H](O)[C@H]2O)(COCCC(=O)NCCCNC(=O)CCCCOC[C@@]12CO[C@@H](O1)[C@H](NC(C)=O)[C@@H](O)[C@H]2O)COCCC(=O)NCCCNC(=O)CCCCOC[C@@]12CO[C@@H](O1)[C@H](NC(C)=O)[C@@H](O)[C@H]2O]

71. The oligonucleotide of any one of Embodiments 56-70, wherein the additional chemical moiety is directly conjugated to the remainder of the oligonucleotide.

72. The oligonucleotide of any one of Embodiments 56-70, wherein the additional chemical moiety is conjugated via a linker to the remainder of the oligonucleotide.

73. The oligonucleotide of Embodiment 72, wherein a linker is or comprises L001.

74. The oligonucleotide of any one of Embodiments 56-73, wherein the additional chemical moiety is conjugated to the 5′-end of the oligonucleotide chain.

75. The oligonucleotide of any one of Embodiments 56-73, wherein the additional chemical moiety is conjugated to the 3′-end of the oligonucleotide chain.

76. The oligonucleotide of any one of Embodiments 56-73, wherein the additional chemical moiety is conjugated to the middle of the oligonucleotide chain.

77. The oligonucleotide of any one of Embodiments 56-76, wherein the additional chemical moiety is conjugated to a sugar.

78. The oligonucleotide of any one of Embodiments 56-76, wherein the additional chemical moiety is conjugated to a nucleobase.

79. The oligonucleotide of any one of Embodiments 56-76, wherein the additional chemical moiety is conjugated to an internucleotidic linkage.

80. The oligonucleotide of Embodiment 73, wherein L001 is connected to 5′-end 5′-carbon of the oligonucleotide chain through the phosphate group.

81. The oligonucleotide of any one of the preceding Embodiments, wherein the oligonucleotide is in a salt form.

82. The oligonucleotide of any one of the preceding Embodiments, wherein the oligonucleotide is in a pharmaceutically acceptable salt form.

83. The oligonucleotide of any one of the preceding Embodiments, wherein the oligonucleotide is in a sodium salt form.

84. The oligonucleotide of any one of the preceding Embodiments, wherein the diastereopurity of the oligonucleotide is about or at least about (DS)^{nc}, wherein DS is about 85%-100% (e.g., about 85%, 86%, 87%, 88%, 89%, 90%, 91%, 92%, 93%, 94%, 95%, 96%, 97%, 98%, 99% or 99.5% or more) and nc is the number of chiral linkage phosphorus.

85. The oligonucleotide of any one of the preceding Embodiments, wherein the diastereopurity of the oligonucleotide is about or at least about (DS)^{nc}, wherein DS is about DS is about 90%-100% (e.g., about 90%, 91%, 92%, 93%, 94%, 95%, 96%, 97%, 98%, 99% or 99.5% or more) and nc is the number of chiral linkage phosphorus.

86. The oligonucleotide of any one of the preceding Embodiments, wherein the diastereopurity of the oligonucleotide is about or at least about (DS)^{nc}, wherein DS is about or at least about 95% and nc is the number of chiral linkage phosphorus.

87. The oligonucleotide of any one of the preceding Embodiments, wherein the diastereopurity of the oligonucleotide is about or at least about (DS)^{nc}, wherein DS is about or at least about 97% and nc is the number of chiral linkage phosphorus.

88. The oligonucleotide of any one of the preceding Embodiments, wherein the diastereopurity of the oligonucleotide is about or at least about (DS)^{nc}, wherein DS is about or at least about 98% and nc is the number of chiral linkage phosphorus.

89. The oligonucleotide of any one of the preceding Embodiments, wherein the diastereopurity of the oligonucleotide is about or at least about (DS)^{nc}, wherein DS is about or at least about 99% and nc is the number of chiral linkage phosphorus.

90. The oligonucleotide of any one of the preceding Embodiments, wherein diastereomeric excess of one or more (e.g., 5, 6, 7, 8, 9, 10, 11, 12, 13, 14, 15, 16, 17, 18, 19, 20 or more) chiral linkage phosphorus centers is independently about or at least about 80%, 85%, 90%, 91%, 92%, 93%, 94%, 95%, 96%, 97%, 98% or 99%.

91. The oligonucleotide of any one of the preceding Embodiments, wherein diastereomeric excess of one or more (e.g., 5, 6, 7, 8, 9, 10, 11, 12, 13, 14, 15, 16, 17, 18, 19, 20 or more) chiral linkage phosphorus centers is independently about or at least about 90%, 91%, 92%, 93%, 94%, 95%, 96%, 97%, 98% or 99%.

92. The oligonucleotide of any one of the preceding Embodiments, wherein diastereomeric excess of each phosphorothioate linkage phosphorus is independently about or at least about 80%, 85%, 90%, 91%, 92%, 93%, 94%, 95%, 96%, 97%, 98% or 99%.

93. The oligonucleotide of any one of the preceding Embodiments, wherein diastereomeric excess of each phosphorothioate linkage phosphorus is independently about or at least about 90%, 91%, 92%, 93%, 94%, 95%, 96%, 97%, 98% or 99%.

94. The oligonucleotide of any one of the preceding Embodiments, wherein diastereomeric excess of each phosphorothioate linkage phosphorus is independently about or at least about 95%.

95. The oligonucleotide of any one of the preceding Embodiments, wherein diastereomeric excess of each phosphorothioate linkage phosphorus is independently about or at least about 96%.

96. The oligonucleotide of any one of the preceding Embodiments, wherein diastereomeric excess of each phosphorothioate linkage phosphorus is independently about or at least about 97%.

97. The oligonucleotide of any one of the preceding Embodiments, wherein diastereomeric excess of each phosphorothioate linkage phosphorus is independently about or at least about 98%.

98. The oligonucleotide of any one of the preceding Embodiments, wherein diastereomeric excess of each chiral linkage phosphorus centers is independently about or at least about 80%, 85%, 90%, 91%, 92%, 93%, 94%, 95%, 96%, 97%, 98% or 99%.

99. The oligonucleotide of any one of the preceding Embodiments, wherein diastereomeric excess of each chiral linkage phosphorus centers is independently about or at least about 90%, 91%, 92%, 93%, 94%, 95%, 96%, 97%, 98% or 99%.

100. The oligonucleotide of any one of the preceding Embodiments, wherein diastereomeric excess of each chiral linkage phosphorus centers is independently about or at least about 95%.

101. The oligonucleotide of any one of the preceding Embodiments, wherein diastereomeric excess of each chiral linkage phosphorus centers is independently about or at least about 96%.

102. The oligonucleotide of any one of the preceding Embodiments, wherein diastereomeric excess of each chiral linkage phosphorus centers is independently about or at least about 97%.

103. The oligonucleotide of any one of the preceding Embodiments, wherein diastereomeric excess of each chiral linkage phosphorus centers is independently about or at least about 98%.

104. The oligonucleotide of any one of the preceding Embodiments, wherein the oligonucleotide has a purity of about 10%-100% (e.g., about 10%-95%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, or about or at least about 10%, 20%, 30%, 40%, 50%, 60%, 65%, 70%, 75%, 80%, 85%, 90%, 95%, or 100%, etc.).

105. The oligonucleotide of any one of the preceding Embodiments, wherein the oligonucleotide has a purity of about 50%-100% (e.g., about 50%-80%, 50%-85%, 50%-90%, 50%-95%, 60%-80%, 60%-85%, 60%-90%, 60%, 95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, or at least about 50%, 60%, 65%, 70%, 75%, 80%, 85%, 90%, 95%, or 100%, etc.).

106. A pharmaceutical composition comprising an oligonucleotide of any one of the preceding Embodiments and a pharmaceutically acceptable carrier.

107. The composition of Embodiment 106, wherein the oligonucleotide is in a pharmaceutically acceptable salt form.

108. The composition of Embodiment 106, wherein the oligonucleotide is in a salt form.

109. The composition of Embodiment 106, wherein the composition comprises two or more salt forms of an oligonucleotide.

110. The composition of Embodiment 106, wherein the composition comprises two or more pharmaceutically acceptable salt forms of an oligonucleotide.

111. The composition of any one of Embodiments 106-110, wherein the composition is a liquid.

112. The composition of any one of Embodiments 106-110, wherein the composition is or comprises the oligonucleotide dissolved in water.

113. The composition of any one of Embodiments 106-110, wherein the composition is or comprises the oligonucleotide dissolved in a buffer.

114. The composition of any one of Embodiments 106-113, wherein the composition delivers an effective amount of an oligonucleotide of any one of the preceding Embodiments.

115. A composition comprising a plurality of oligonucleotides, wherein each oligonucleotides of the plurality is independently a particular oligonucleotide or a salt thereof, wherein the particular oligonucleotide is an oligonucleotide of any one of Embodiments 1-105.

116. A composition comprising a plurality of oligonucleotides, wherein each oligonucleotides of the plurality is independently a particular oligonucleotide or a salt thereof, wherein the particular oligonucleotide is an oligonucleotide of any one of Embodiments 1-105, wherein at least about 51%-100%, 10%-100%, 20-100%, 30%-100%, 40%-100%, 50%-100%, 5%-90%, 10%-90%, 20-90%, 30%-90%, 40%-90%, 50%-90%, 5%-85%, 10%-85%, 20-85%, 30%-85%, 40%-85%, 50%-85%, 5%-80%, 10%-80%, 20-80%, 30%-80%, 40%-80%, 50%-80%, 5%-75%, 10%-75%, 20-75%, 30%-75%, 40%-75%, 50%-75%, 5%-70%, 10%-70%, 20-70%, 30%-70%, 40%-70%, 50%-70%, 5%-65%, 10%-65%, 20-65%, 30%-65%, 40%-65%, 50%-65%, 5%-60%, 10%-60%, 20-60%, 30%-60%, 40%-60%, 50%-60%, 5%, 10%, 20%, 30%, 40%, 50%, 60%, 70%, 80%, 85%, 90%, 91%, 92%, 93%, 94%, 95%, 96%, 97%, 98%, or 99% of all oligonucleotides in the composition that share the base sequence of the particular oligonucleotide are oligonucleotide of the plurality.

117. A composition comprising a plurality of oligonucleotides, wherein each oligonucleotides of the plurality is independently a particular oligonucleotide or a salt thereof, wherein the particular oligonucleotide is an oligonucleotide of any one of Embodiments 1-105, wherein at least about 5%-100%, 10-100%, 20-100%, 30%-100%, 40%-100%, 50%-100%, 5%-90%, 10%-90%, 20-90%, 30%-90%, 40%-90%, 50%-90%. 5%-85%, 10%-85%, 20-85%, 30%-85%, 40%-85%, 50%-85%, 5%-80%, 10%-80%, 20-80%, 30%-80%, 40%-80%, 50%-80%, 5%-75%, 10%-75%, 20-75%, 30%-75%, 40%-75%, 50%-75%, 5%-70%, 10%-70%, 20-70%, 30%-70%, 40%-70%, 50%-70%, 5%-65%, 10%-65%, 20-65%, 30%-65%, 40%-65%, 50%-65%, 5%-60%, 10%-60%, 20-60%, 30%-60%, 40%-60%, 50%-60%, 5%, 10%, 20%, 30%, 40%, 50%, 60%, 70%, 80%, 85%, 90%, 91%, 92%, 93%, 94%, 95%, 96%, 97%, 98%, or 99% of all oligonucleotides in the composition that share the constitution of the particular oligonucleotide or a salt thereof are oligonucleotide of the plurality.

118. The composition of any one of Embodiments 115-117, wherein the level of oligonucleotides of a plurality in oligonucleotides in the composition that share the constitution of the plurality is about or at least about (DS)^{nc}, wherein DS is about 85%-100% (e.g., about 85%, 86%, 87%, 88%, 89%, 90%, 91%, 92%, 93%, 94%, 95%, 96%, 97%, 98%, 99% or 99.5% or more) and nc is the number of chiral internucleotidic linkages.

119. The composition of any one of Embodiments 115-117, wherein for each plurality of oligonucleotides, the level of oligonucleotides of the plurality in oligonucleotides in the composition that share the constitution of the plurality is independently about or at least about (DS)^{nc}, wherein DS is about 85%-100% (e.g., about 85%, 86%, 87%, 88%, 89%, 90%, 91%, 92%, 93%, 94%, 95%, 96%, 97%, 98%, 99% or 99.5% or more) and nc is the number of chiral internucleotidic linkages.

120. The composition of any one of Embodiments 118-119, wherein DS is about 90%-100% (e.g., about 90%, 91%, 92%, 93%, 94%, 95%, 96%, 97%, 98%, 99% or 99.5% or more).

121. The composition of any one of Embodiments 118-119, wherein DS is about 95%-100%.

122. The composition of any one of Embodiments 118-119, wherein DS is about 97%-100%.

123. The composition of any one of Embodiments 115-117, wherein the level is at least about 10%-100%, or at least about 10%, 20%, 30%, 40%, 50%, 60%, 65%, 70%, 75%, 80%, 85%, 90%, or 95%.

124. The composition of any one of Embodiments 115-117, wherein the level is at least about 50%-100%, or at least about 50%, 60%, 65%, 70%, 75%, 80%, 85%, 90%, or 95%.

125. The composition of any one of Embodiments 115-117, wherein diastereomeric excess of each phosphorothioate linkage phosphorus in the oligonucleotides of the plurality is independently about or at least about 80%, 85%, 90%, 91%, 92%, 93%, 94%, 95%, 96%, 97%, 98% or 99%.

126. The composition of any one of Embodiments 115-117, wherein diastereomeric excess of each phosphorothioate linkage phosphorus in the oligonucleotides of the plurality is independently about or at least about 90%, 91%, 92%, 93%, 94%, 95%, 96%, 97%, 98% or 99%.

127. The composition of any one of Embodiments 115-117, wherein diastereomeric excess of each phosphorothioate linkage phosphorus in the oligonucleotides of the plurality is independently about or at least about 95%.

128. The composition of any one of Embodiments 115-117, wherein diastereomeric excess of each phosphorothioate linkage phosphorus in the oligonucleotides of the plurality is independently about or at least about 96%.

129. The composition of any one of Embodiments 115-117, wherein diastereomeric excess of each phosphorothioate linkage phosphorus in the oligonucleotides of the plurality is independently about or at least about 97%.

130. The composition of any one of Embodiments 115-117, wherein diastereomeric excess of each phosphorothioate linkage phosphorus in the oligonucleotides of the plurality is independently about or at least about 98%.

131. The composition of any one of Embodiments 115-117, wherein diastereomeric excess of each chiral linkage phosphorus centers in the oligonucleotides of the plurality is independently about or at least about 80%, 85%, 90%, 91%, 92%, 93%, 94%, 95%, 96%, 97%, 98% or 99%.

132. The composition of any one of Embodiments 115-117, wherein diastereomeric excess of each chiral linkage phosphorus centers in the oligonucleotides of the plurality is independently about or at least about 90%, 91%, 92%, 93%, 94%, 95%, 96%, 97%, 98% or 99%.

133. The composition of any one of Embodiments 115-117, wherein diastereomeric excess of each chiral linkage phosphorus centers in the oligonucleotides of the plurality is independently about or at least about 95%.

134. The composition of any one of Embodiments 115-117, wherein diastereomeric excess of each chiral linkage phosphorus centers in the oligonucleotides of the plurality is independently about or at least about 96%.

135. The composition of any one of Embodiments 115-117, wherein diastereomeric excess of each chiral linkage phosphorus centers in the oligonucleotides of the plurality is independently about or at least about 97%.

136. The composition of any one of Embodiments 115-117, wherein diastereomeric excess of each chiral linkage phosphorus centers in the oligonucleotides of the plurality is independently about or at least about 98%.

137. A compound having the structure of formula A-1 or a pharmaceutically acceptable salt thereof:

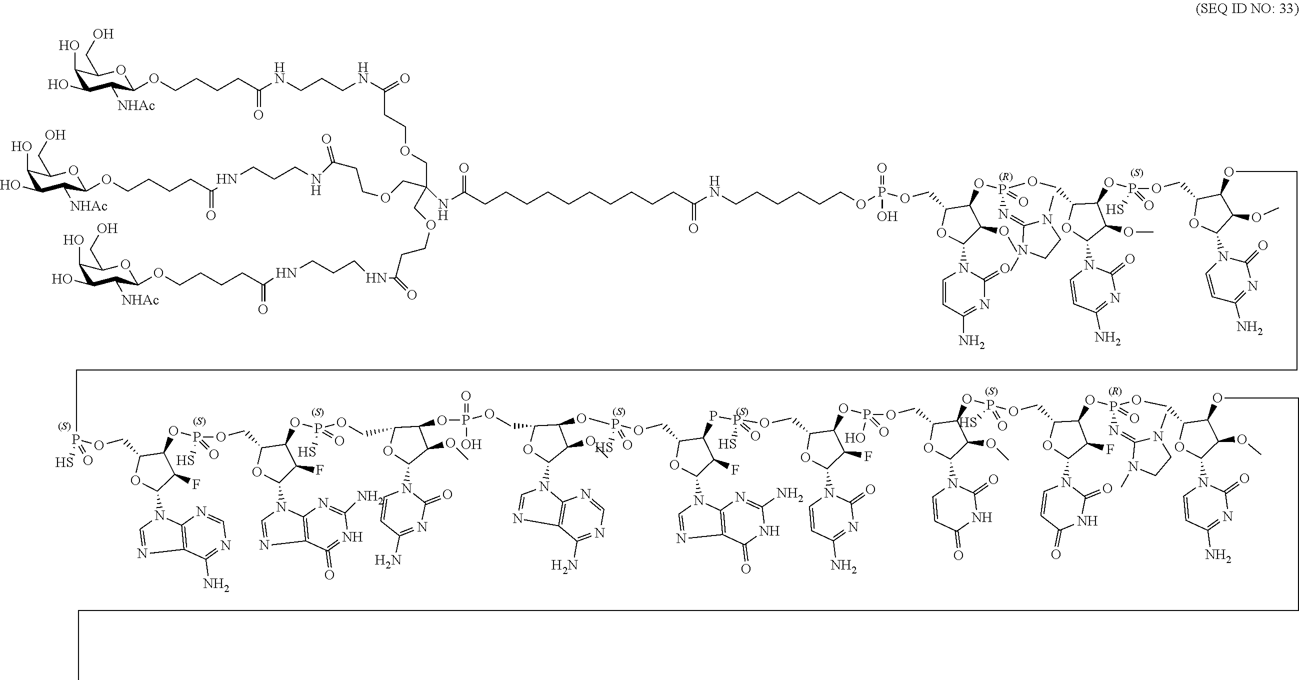

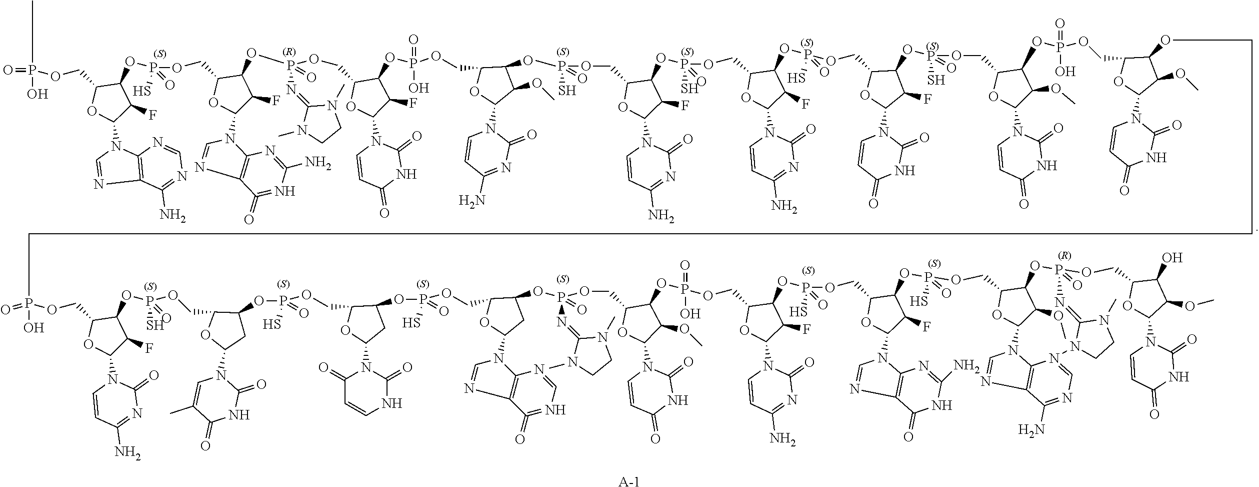

138. A compound having the structure of formula A-2 or a pharmaceutically acceptable salt thereof:

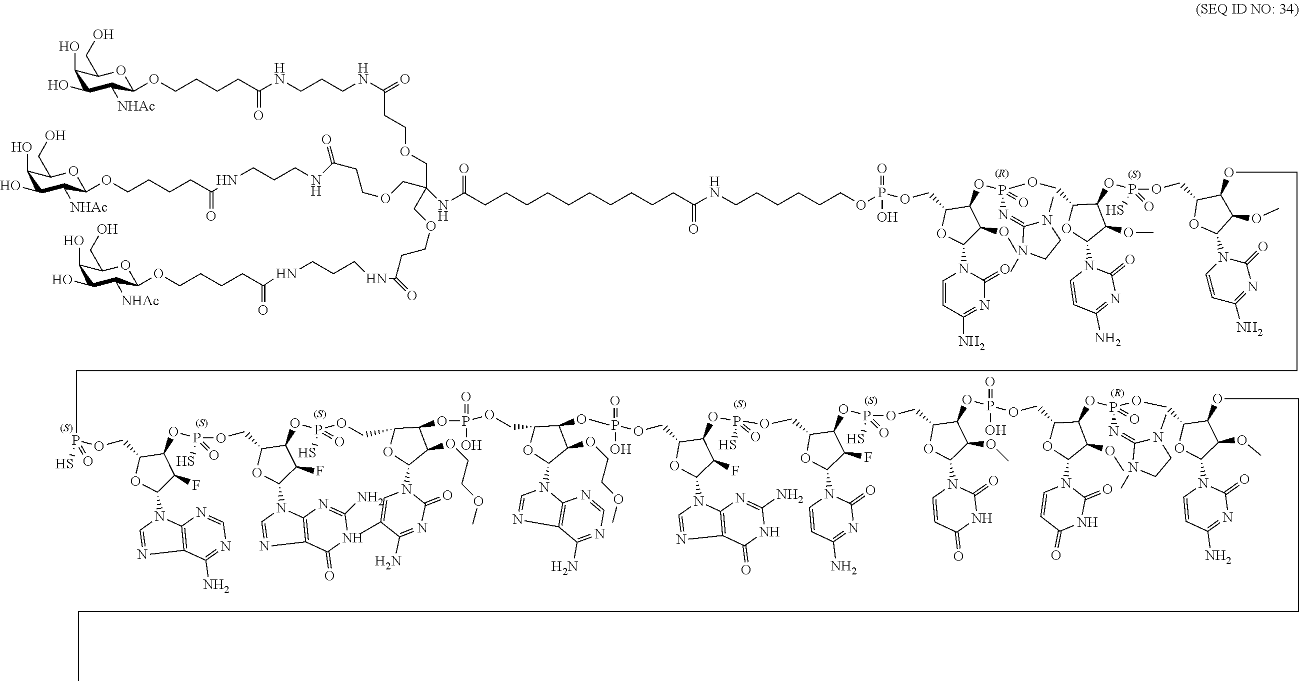

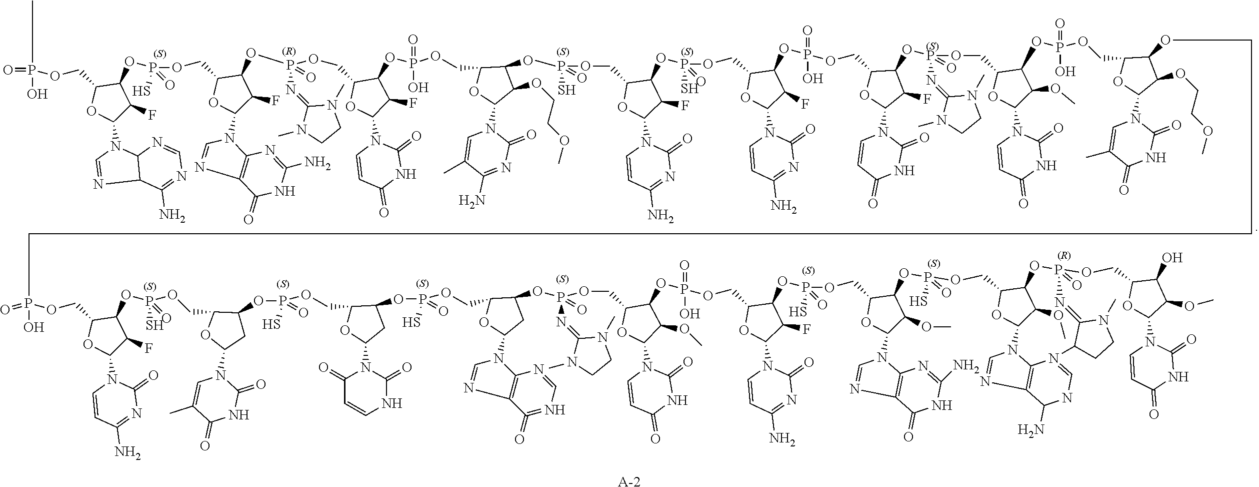

139. A compound having the structure of formula A-3 or a pharmaceutically acceptable salt thereof:

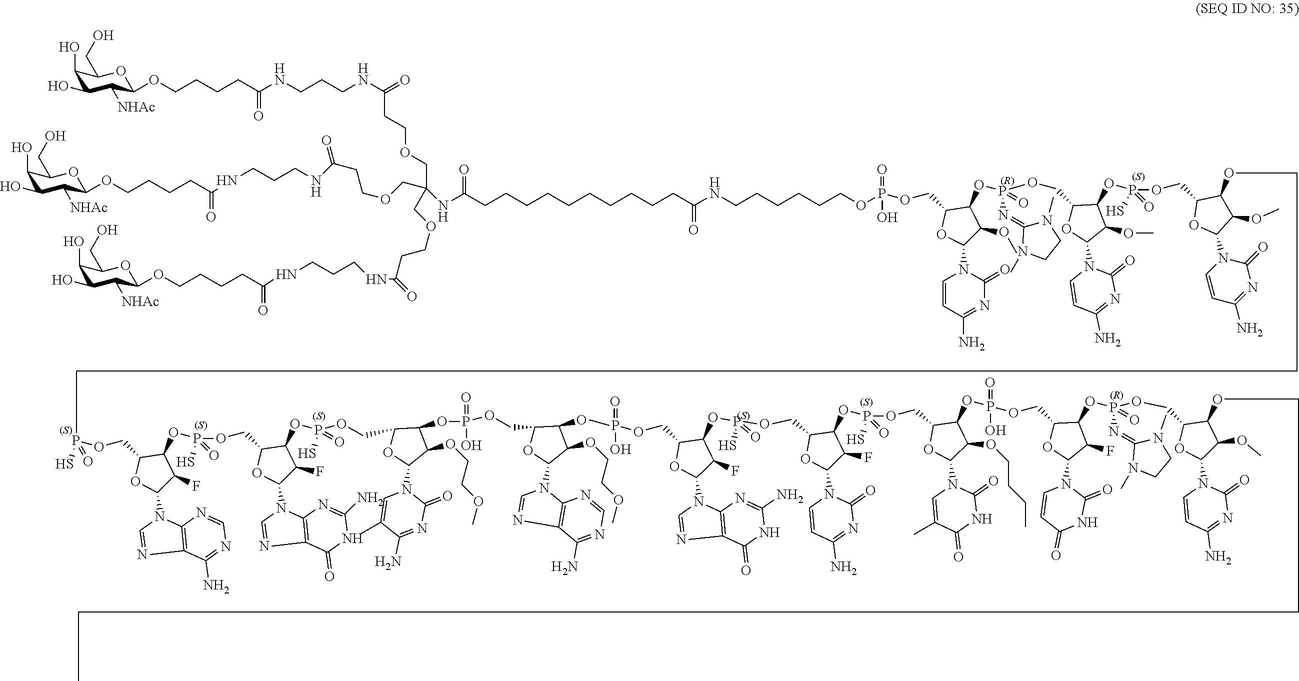

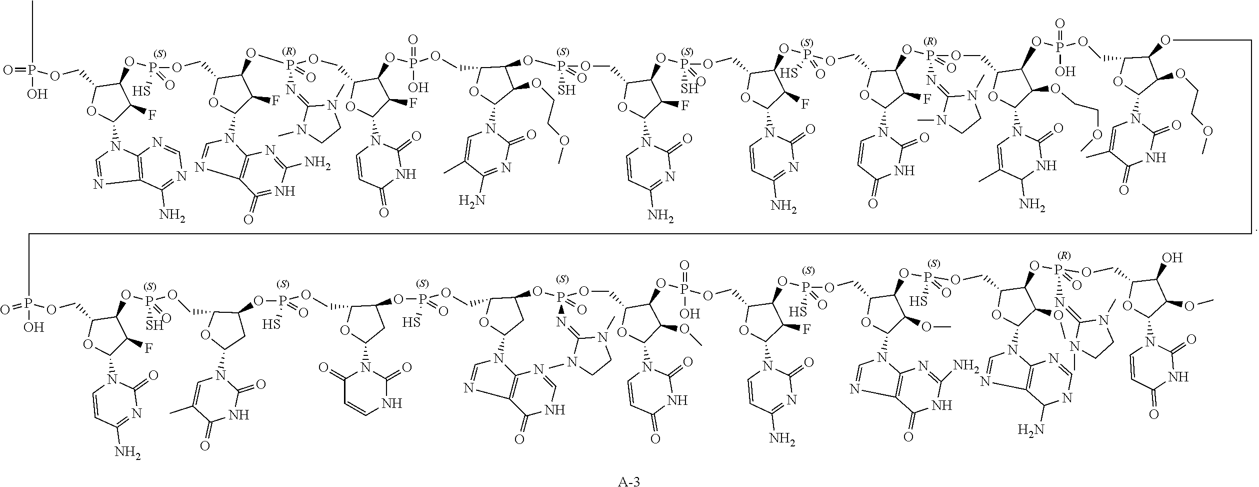

140. A compound having the structure of formula B-1 Or a pharmaceutically acceptable salt thereof:

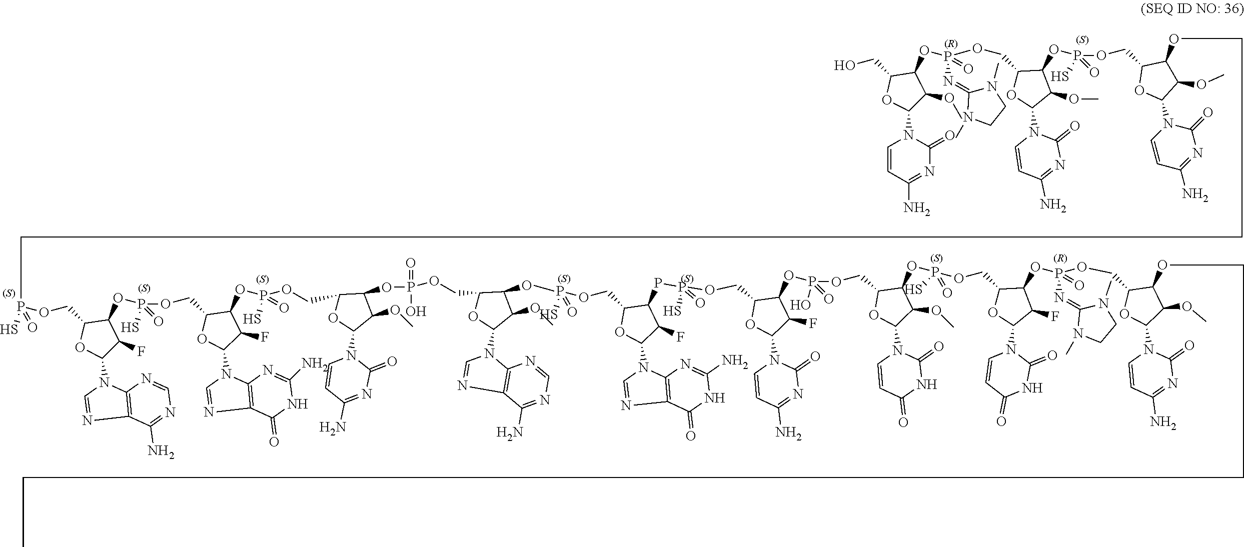

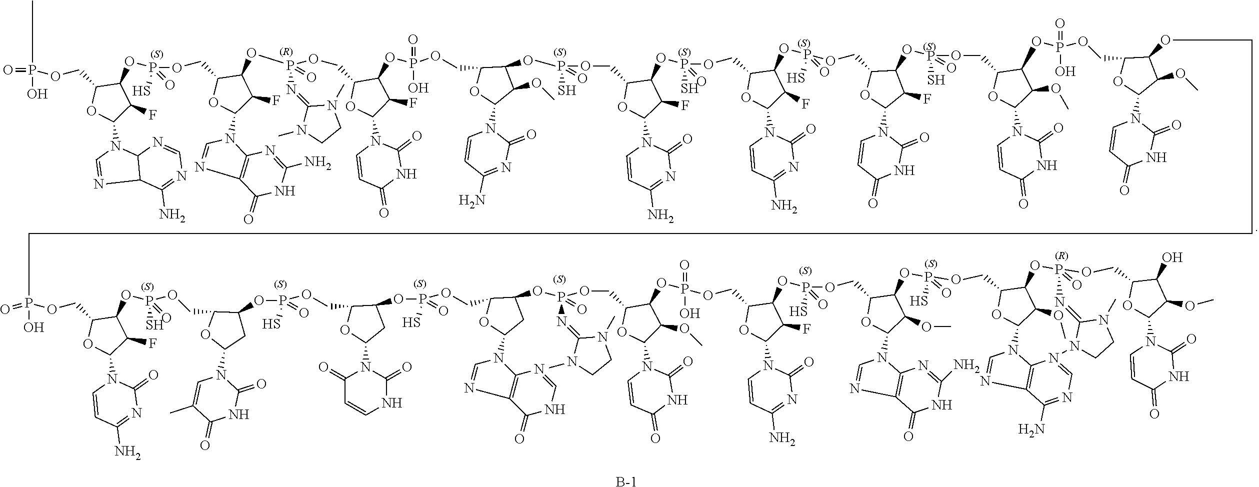

141. A compound having the structure of formula B-2 or a pharmaceutically acceptable salt thereof:

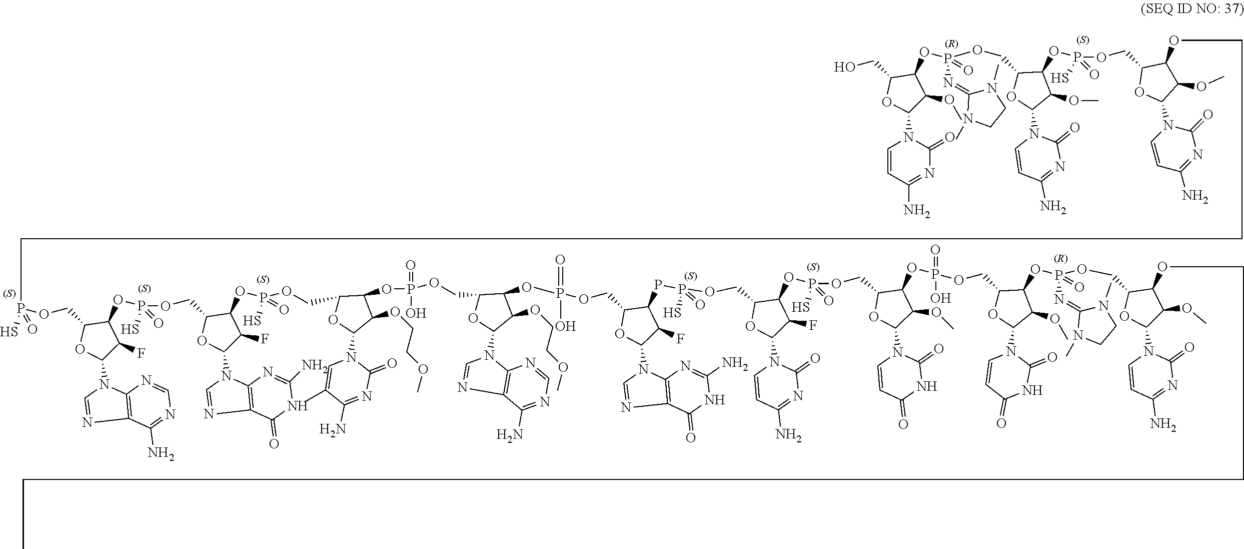

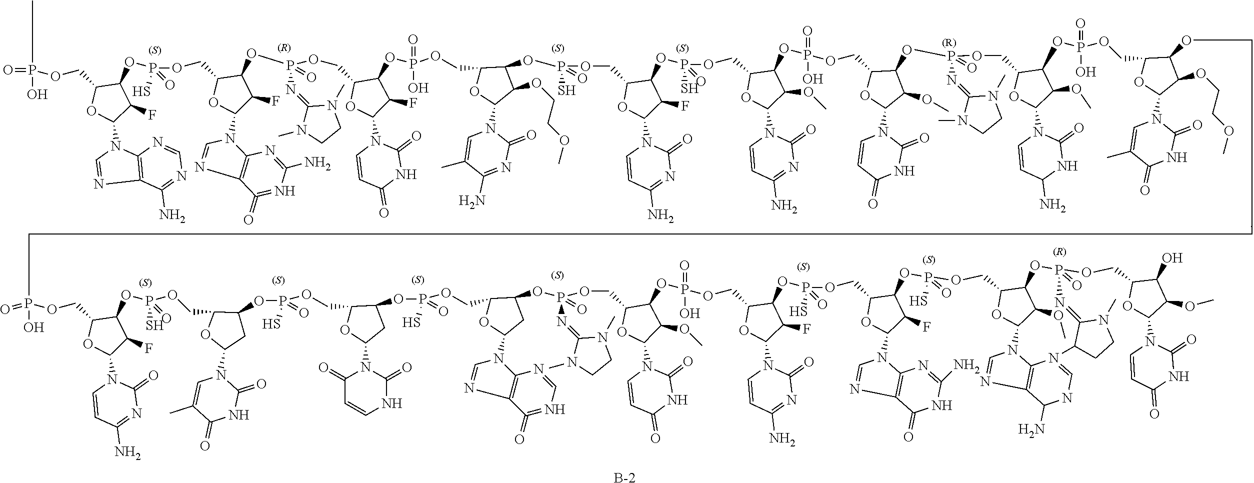

142. A compound having the structure of formula B-3 or a pharmaceutically acceptable salt thereof:

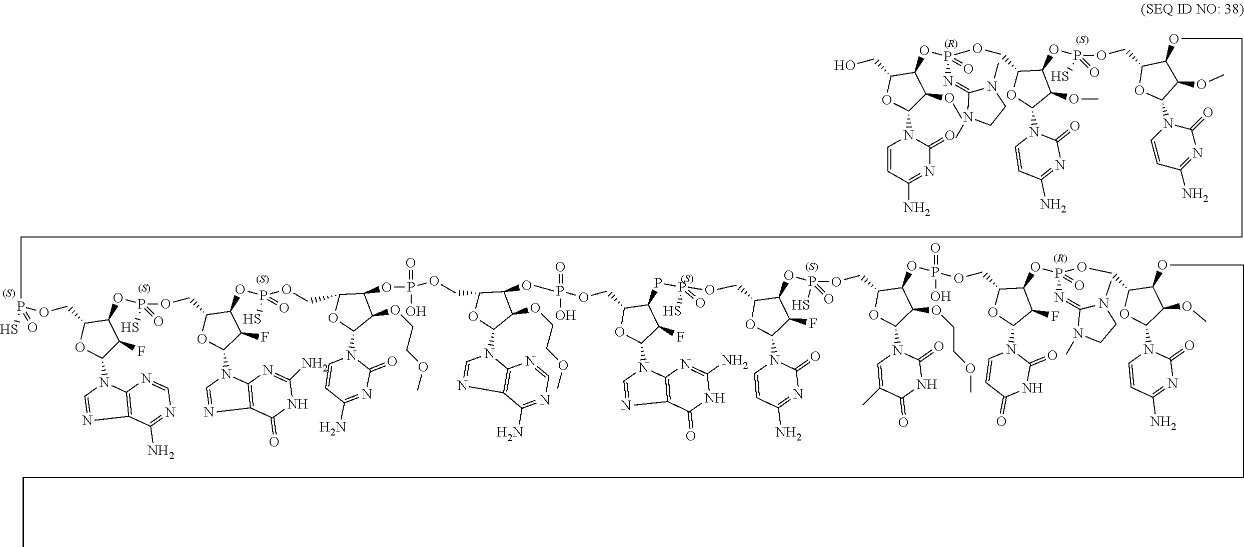

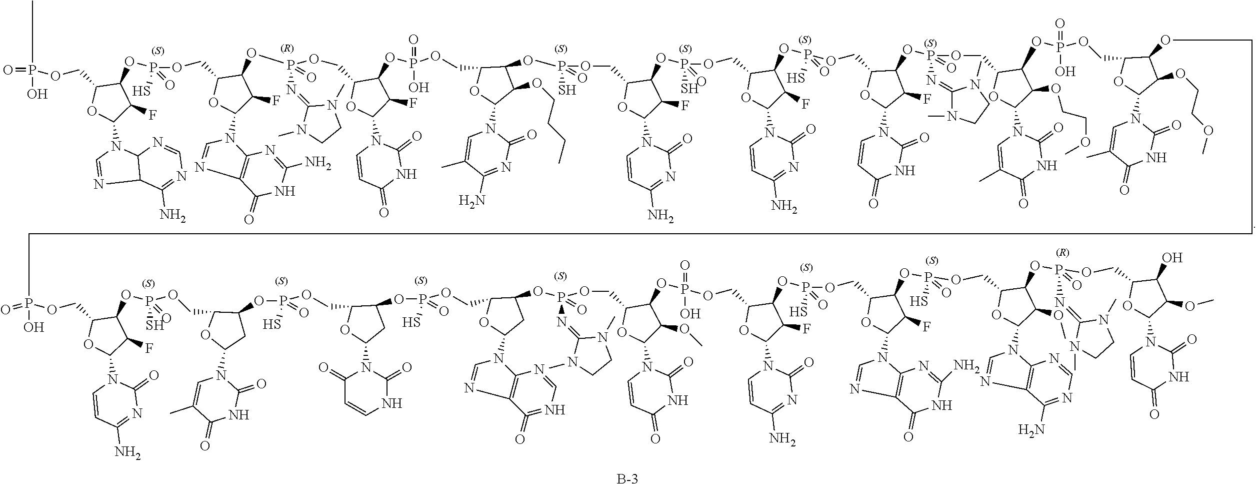

143. A compound which is a conjugate of a compound of any one of Embodiments 140-142 with an additional chemical moiety or a salt thereof.

144. The compound of Embodiment 143, wherein the additional chemical moiety is or comprises a targeting moiety.

145. The compound of Embodiment 143, wherein the additional chemical moiety is or comprises a carbohydrate moiety.

146. The compound of Embodiment 143, wherein the additional chemical moiety is or comprises a lipid moiety.

147. The compound of Embodiment 143, wherein the additional chemical moiety is or comprises one or more protein ligand moieties.

148. The compound of Embodiment 143, wherein the additional chemical moiety is or comprises two or more protein ligand moieties.

149. The oligonucleotide of Embodiment 143, wherein the additional chemical moiety targets liver.

150. The oligonucleotide of Embodiment 143, wherein the additional chemical moiety is or comprises a ligand of a receptors expressed in liver.

151. The compound of Embodiment 143, wherein the additional chemical moiety is or comprises a ligand for an asialoglycoprotein receptor.

152. The compound of Embodiment 143, wherein the additional chemical moiety comprises multiple moieties, each of which is independently a ligand for an asialoglycoprotein receptor.

153. The compound of Embodiment 143, wherein the additional chemical moiety is or comprises GalNAc.

154. The compound of Embodiment 143, wherein the additional chemical moiety comprises three GalNAc.

155. The compound of Embodiment 143, wherein the additional chemical moiety is or comprises

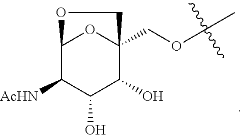 [SMILES: *OC[C@@]12CO[C@@H](O1)[C@H](NC(C)=O)[C@@H](O)[C@H]2O]

156. The compound of Embodiment 143, wherein the additional chemical moiety is

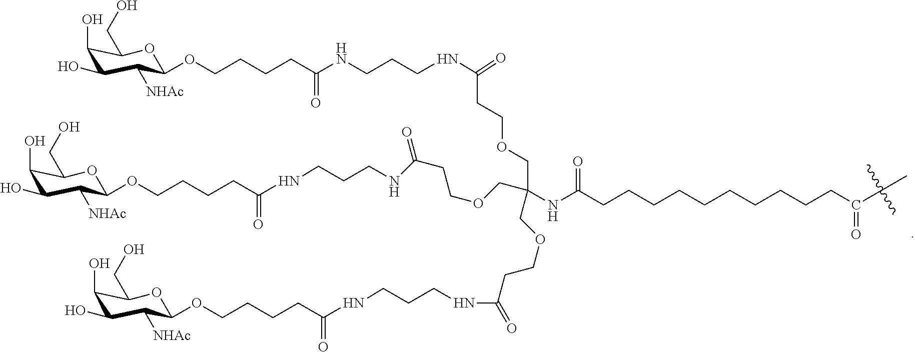

157. The compound of Embodiment 143, wherein the additional chemical moiety is or comprises

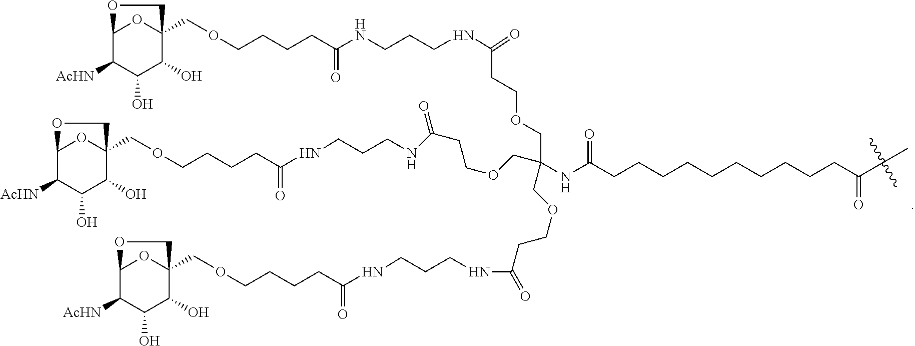 [SMILES: *C(=O)CCCCCCCCCCC(=O)NC(COCCC(=O)NCCCNC(=O)CCCCOC[C@@]12CO[C@@H](O1)[C@H](NC(C)=O)[C@@H](O)[C@H]2O)(COCCC(=O)NCCCNC(=O)CCCCOC[C@@]12CO[C@@H](O1)[C@H](NC(C)=O)[C@@H](O)[C@H]2O)COCCC(=O)NCCCNC(=O)CCCCOC[C@@]12CO[C@@H](O1)[C@H](NC(C)=O)[C@@H](O)[C@H]2O]

158. The compound of any one of Embodiments 143-157, wherein the additional chemical moiety is directly conjugated to the remainder of the compound.

159. The compound of any one of Embodiments 143-157, wherein the additional chemical moiety is conjugated via a linker to the remainder of the compound.

160. The compound of Embodiment 159, wherein a linker is or comprises L001.

161. The compound of any one of Embodiments 143-160, wherein the additional chemical moiety is conjugated to the 5′-end of the oligonucleotide chain.

162. The compound of any one of Embodiments 143-160, wherein the additional chemical moiety is conjugated to the 3′-end of the oligonucleotide chain.

163. The compound of any one of Embodiments 143-160, wherein the additional chemical moiety is conjugated to the middle of the oligonucleotide chain.

164. The compound of any one of Embodiments 143-163, wherein the additional chemical moiety is conjugated to a sugar.

165. The compound of any one of Embodiments 143-163, wherein the additional chemical moiety is conjugated to a nucleobase.

166. The compound of any one of Embodiments 143-163, wherein the additional chemical moiety is conjugated to an internucleotidic linkage.

167. The compound of Embodiment 160, wherein L001 is connected to 5′-end 5′-carbon of the oligonucleotide chain through the phosphate group.

168. The compound of any one of the preceding Embodiments, wherein the compound is in a salt form.

169. The compound of any one of the preceding Embodiments, wherein the compound is in a pharmaceutically acceptable salt form.

170. The compound of any one of the preceding Embodiments, wherein the compound is in a sodium salt form.

171. The compound of any one of the preceding Embodiments, wherein the diastereopurity of the compound is about or at least about (DS)^{nc}, wherein DS is about 85%-100% (e.g., about 85%, 86%, 87%, 88%, 89%, 90%, 91%, 92%, 93%, 94%, 95%, 96%, 97%, 98%, 99% or 99.5% or more) and nc is the number of chiral linkage phosphorus.

172. The compound of any one of the preceding Embodiments, wherein the diastereopurity of the compound is about or at least about (DS)^{nc}, wherein DS is about 90%-100% (e.g., about 90%, 91%, 92%, 93%, 94%, 95%, 96%, 97%, 98%, 99% or 99.5% or more) and nc is the number of chiral linkage phosphorus.

173. The compound of any one of the preceding Embodiments, wherein the diastereopurity of the compound is about or at least about (DS)^{nc}, wherein DS is about or at least about 95% and nc is the number of chiral linkage phosphorus.

174. The compound of any one of the preceding Embodiments, wherein the diastereopurity of the compound is about or at least about (DS)^{nc}, wherein DS is about or at least about 97% and nc is the number of chiral linkage phosphorus.

175. The compound of any one of the preceding Embodiments, wherein the diastereopurity of the compound is about or at least about (DS)^{nc}, wherein DS is about or at least about 98% and nc is the number of chiral linkage phosphorus.

176. The compound of any one of the preceding Embodiments, wherein the diastereopurity of the compound is about or at least about (DS)^{nc}, wherein DS is about or at least about 99% and nc is the number of chiral linkage phosphorus.

177. The compound of any one of the preceding Embodiments, wherein diastereomeric excess of one or more (e.g., 5, 6, 7, 8, 9, 10, 11, 12, 13, 14, 15, 16, 17, 18, 19, 20 or more) chiral linkage phosphorus centers is independently about or at least about 80%, 85%, 90%, 91%, 92%, 93%, 94%, 95%, 96%, 97%, 98% or 99%.

178. The compound of any one of the preceding Embodiments, wherein diastereomeric excess of one or more (e.g., 5, 6, 7, 8, 9, 10, 11, 12, 13, 14, 15, 16, 17, 18, 19, 20 or more) chiral linkage phosphorus centers is independently about or at least about 90%, 91%, 92%, 93%, 94%, 95%, 96%, 97%, 98% or 99%.

179. The compound of any one of the preceding Embodiments, wherein diastereomeric excess of each phosphorothioate linkage phosphorus is independently about or at least about 80%, 85%, 90%, 91%, 92%, 93%, 94%, 95%, 96%, 97%, 98% or 99%.

180. The compound of any one of the preceding Embodiments, wherein diastereomeric excess of each phosphorothioate linkage phosphorus is independently about or at least about 90%, 91%, 92%, 93%, 94%, 95%, 96%, 97%, 98% or 99%.

181. The compound of any one of the preceding Embodiments, wherein diastereomeric excess of each phosphorothioate linkage phosphorus is independently about or at least about 95%.

182. The compound of any one of the preceding Embodiments, wherein diastereomeric excess of each phosphorothioate linkage phosphorus is independently about or at least about 96%.

183. The compound of any one of the preceding Embodiments, wherein diastereomeric excess of each phosphorothioate linkage phosphorus is independently about or at least about 97%.

184. The compound of any one of the preceding Embodiments, wherein diastereomeric excess of each phosphorothioate linkage phosphorus is independently about or at least about 98%.

185. The compound of any one of the preceding Embodiments, wherein diastereomeric excess of each chiral linkage phosphorus centers is independently about or at least about 80%, 85%, 90%, 91%, 92%, 93%, 94%, 95%, 96%, 97%, 98% or 99%.

186. The compound of any one of the preceding Embodiments, wherein diastereomeric excess of each chiral linkage phosphorus centers is independently about or at least about 90%, 91%, 92%, 93%, 94%, 95%, 96%, 97%, 98% or 99%.

187. The compound of any one of the preceding Embodiments, wherein diastereomeric excess of each chiral linkage phosphorus centers is independently about or at least about 95%.

188. The compound of any one of the preceding Embodiments, wherein diastereomeric excess of each chiral linkage phosphorus centers is independently about or at least about 96%.

189. The compound of any one of the preceding Embodiments, wherein diastereomeric excess of each chiral linkage phosphorus centers is independently about or at least about 97%.

190. The compound of any one of the preceding Embodiments, wherein diastereomeric excess of each chiral linkage phosphorus centers is independently about or at least about 98%.

191. The compound of any one of the preceding Embodiments, wherein the compound has a purity of about 10%-100% (e.g., about 10%-95%, 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%, 100%, 80%-85%, 80%-90%, 80%-95%, 80%-100%, 85%-90%, 85%-95%, 85%-100%, 90%-95%, 90%-100%, or about or at least about 10%, 20%, 30%, 40%, 50%, 60%, 65%, 70%, 75%, 80%, 85%, 90%, 95%, or 100%, etc.).

192. The compound of any one of the preceding Embodiments, wherein the compound has a purity of about 50%-100% (e.g., about 50%-80%, 50%-85%, 50%-90%, 50%-95%, 60%-80%, 60%-85%, 60%-90%, 60%-95%, 60%-100%, 65%-80%, 65%-85%, 65%-90%, 65%-95%, 65%-100%, 70%-80%, 70%-85%, 70%-90%, 70%-95%, 70%-100%, 75%-80%, 75%-85%, 75%-90%, 75%-95%, 75%-100%, 80%-85%, 80%-90%, 80%-95%, 80%-100%, 85%-90%, 85%-95%, 85%-100%, 90%-95%, 90%-100%, or at least about 50%, 60%, 65%, 70%, 75%, 80%, 85%, 90%, 95%, or 100%, etc.).

193. A pharmaceutical composition comprising a compound of any one of the preceding Embodiments and a pharmaceutically acceptable carrier.

194. The composition of Embodiment 193, wherein the compound is in a pharmaceutically acceptable salt form.

195. The composition of Embodiment 193, wherein the composition comprises two or more pharmaceutically acceptable salt forms of a compound.

196. The composition of any one of Embodiments 193-195, wherein the composition is a liquid.

197. The composition of any one of Embodiments 193-196, wherein the composition is or comprises the compound dissolved in water.

198. The composition of any one of Embodiments 193-196, wherein the composition is or comprises the compound dissolved in a buffer.

199. The composition of any one of Embodiments 193-198, wherein the composition delivers an effective amount of a compound of any one of the preceding Embodiments.

200. The composition of any one of Embodiments 193-199, wherein diastereomeric excess of each phosphorothioate linkage phosphorus in the compound is independently about or at least about 80%, 85%, 90%, 91%, 92%, 93%, 94%, 95%, 96%, 97%, 98% or 99%, 201. The composition of any one of Embodiments 193-199, wherein diasteromeric excess of each phosphorothioate linkage phosphorus in the compound is independently about or at least about 90%, 91%, 92%, 93%, 94%, 95%, 96%, 97%, 98% or 99%.

202. The composition of any one of Embodiments 193-199, wherein diastereomeric excess of each phosphorothioate linkage phosphorus in the compound is independently about or at least about 95%.

203. The composition of any one of Embodiments 193-199, wherein diastereomeric excess of each phosphorothioate linkage phosphorus in the compound is independently about or at least about 96%.

204. The composition of any one of Embodiments 193-199, wherein diastereomeric excess of each phosphorothioate linkage phosphorus in the compound is independently about or at least about 97%.

205. The composition of any one of Embodiments 193-199, wherein diastereomeric excess of each phosphorothioate linkage phosphorus in the compound is independently about or at least about 98%.

206. The composition of any one of Embodiments 193-199, wherein diastereomeric excess of each chiral linkage phosphorus centers in the compound is independently about or at least about 80%, 85%, 90%, 91%, 92%, 93%, 94%, 95%, 96%, 97%, 98% or 99%.

207. The composition of any one of Embodiments 193-199, wherein diastereomeric excess of each chiral linkage phosphorus centers in the compound is independently about or at least about 90%, 91%, 92%, 93%, 94%, 95%, 96%, 97%, 98% or 99%.

208. The composition of any one of Embodiments 193-199, wherein diastereomeric excess of each chiral linkage phosphorus centers in the compound is independently about or at least about 95%.

209. The composition of any one of Embodiments 193-199, wherein diastereomeric excess of each chiral linkage phosphorus centers in the compound is independently about or at least about 96%.

210. The composition of any one of Embodiments 193-199, wherein diastereomeric excess of each chiral linkage phosphorus centers in the compound is independently about or at least about 97%.

211. The composition of any one of Embodiments 193-199, wherein diastereomeric excess of each chiral linkage phosphorus centers in the compound is independently about or at least about 98%.

212. A method for modifying a target adenosine in a target nucleic acid, comprising contacting the target nucleic acid with an oligonucleotide, compound or composition of any one of the preceding Embodiments, wherein the target adenosine is 1024 G>A in human SERPINA1.

213. A method for deaminating a target adenosine in a target nucleic acid, comprising contacting the target nucleic acid with an oligonucleotide, compound or composition of any one of the preceding Embodiments, wherein the target adenosine is 1024 G>A in human SERPINA1.

214. A method for producing, or restoring or increasing level of a product of a particular nucleic acid, comprising contacting a target nucleic acid with an oligonucleotide, compound or composition of any one of the preceding Embodiments, wherein the target nucleic acid comprises a target adenosine, and the particular nucleic acid differs from the target nucleic acid in that the particular nucleic acid has an I or G instead of the target adenosine, wherein the target nucleic acid is a human SERPINA1 transcript with a 1024 G>A mutation, and the target adenosine is 1024 G>A in human SERPINA1.

215. A method for reducing level of a product of a target nucleic acid, comprising contacting a target nucleic acid with an oligonucleotide, compound or composition of any one of the preceding Embodiments, wherein the target nucleic acid comprises a target adenosine, wherein the target nucleic acid is a human SERPINA1 transcript with a 1024 G>A mutation, and the target adenosine is 1024 G>A in human SERPINA1.

216. The method of Embodiment 214 or 215, wherein the product is a protein.

217. The method of Embodiment 214 or 215, wherein the product is a mRNA.

218. The method of any one of Embodiments 212-217, wherein the target nucleic acid is in a sample.

219. A method, comprising:
contacting an oligonucleotide, compound or composition of any one of the preceding Embodiments with a sample comprising a target nucleic acid and an adenosine deaminase, the target nucleic acid comprises a target adenosine; wherein:
the target nucleic acid is a human SERPINA1 transcript with a 1024 G>A mutation, and the target adenosine is 1024 G>A in human SERPINA1; and
the target adenosine is modified.

220. The method of Embodiment 219, wherein the deaminase is an ADAR enzyme.

221. The method of Embodiment 219, wherein the deaminase is ADAR1.

222. The method of Embodiment 219, wherein the deaminase is ADAR2A.

223. The method of any one of Embodiments 212-222, wherein a sample is a cell.

224. A method for preventing a condition, disorder or disease, comprising administering to a subject susceptible thereto or suffering therefrom an effective amount of an oligonucleotide, compound or composition of any one of the preceding Embodiments, wherein the subject comprises 1024 G>A (E342K) mutation in human SERPINA1.

225. A method for preventing a condition, disorder or disease, comprising delivering to a subject susceptible thereto or suffering therefrom an effective amount of an oligonucleotide, compound or composition of any one of the preceding Embodiments, wherein the subject comprises 1024 G>A (E342K) mutation in human SERPINA1.

226. A method for treating a condition, disorder or disease, comprising administering to a subject susceptible thereto or suffering therefrom an effective amount of an oligonucleotide, compound or composition of any one of the preceding Embodiments, wherein the subject comprises 1024 G>A (E342K) mutation in human SERPINA1.

227. A method for treating a condition, disorder or disease, comprising delivering to a subject susceptible thereto or suffering therefrom an effective amount of an oligonucleotide, compound or composition of any one of the preceding Embodiments, wherein the subject comprises 1024 G>A (E342K) mutation in human SERPINA1.

228. A method for reducing Z-AAT in liver of a subject, comprising administering to the subject an effective amount of an oligonucleotide, compound or composition of any one of the preceding Embodiments, wherein the subject comprises 1024 G>A (E342K) mutation in human SERPINA1.

229. A method for reducing Z-AAT in liver of a subject, comprising delivering to the subject an effective amount of an oligonucleotide, compound or composition of any one of the preceding Embodiments, wherein the subject comprises 1024 G>A (E342K) mutation in human SERPINA1.

230. A method for reducing liver inflammation in a subject, comprising administering to the subject an effective amount of an oligonucleotide, compound or composition of any one of the preceding Embodiments, wherein the subject comprises 1024 G>A (E342K) mutation in human SERPINA1.

231. A method for reducing liver inflammation in a subject, comprising delivering to the subject an effective amount of an oligonucleotide, compound or composition of any one of the preceding Embodiments, wherein the subject comprises 1024 G>A (E342K) mutation in human SERPINA1.

232. A method for inhibiting elastase in a subject, comprising administering to the subject an effective amount of an oligonucleotide, compound or composition of any one of the preceding Embodiments, wherein the subject comprises 1024 G>A (E342K) mutation in human SERPINA1.

233. A method for inhibiting elastase in a subject, comprising delivering to the subject an effective amount of an oligonucleotide, compound or composition of any one of the preceding Embodiments, wherein the subject comprises 1024 G>A (E342K) mutation in human SERPINA1.

234. A method for increasing levels and/or activities of an alpha-1 antitrypsin (A1AT) polypeptide in the serum or blood of a subject, comprising administering to the subject an effective amount of an oligonucleotide, compound or composition of any one of the preceding Embodiments, wherein the subject comprises 1024 G>A (E342K) mutation in human SERPINA1.

235. A method for increasing levels and/or activities of an alpha-1 antitrypsin (A1AT) polypeptide in the serum or blood of a subject, comprising delivering to the subject an effective amount of an oligonucleotide, compound or composition of any one of the preceding Embodiments, wherein the subject comprises 1024 G>A (E342K) mutation in human SERPINA1.

236. The method of Embodiment 234 or 235, wherein the A1AT polypeptide provides one or more higher activities compared to a reference A1AT polypeptide.

237. The method of Embodiment 234 or 235, wherein the A1AT polypeptide provides one or more higher activities compared to E342 A1AT polypeptide.

238. The method of any one of Embodiments 234-237, wherein the A1AT polypeptide is a wild-type A1AT polypeptide.

239. The method of any one of Embodiments 234-238, wherein the method increase the amount of the A1AT polypeptide in serum.

240. The method of any one of Embodiments 234-238, wherein the method decrease the amount of a reference A1AT polypeptide in serum.

241. The method of any one of Embodiments 234-240, wherein the method increase the ratio of the A1AT polypeptide over a reference A1AT polypeptide in serum or blood.

242. The method of any one of Embodiments 234-241, wherein the reference A1AT polypeptide is mutated.

243. The method of any one of Embodiments 234-242, wherein the reference A1AT polypeptide is an E342K A1AT polypeptide.

244. A method for decreasing levels and/or activities of a mutant alpha-1 antitrypsin (A1AT) polypeptide in the serum or blood of a subject, comprising administering to the subject an effective amount of an oligonucleotide, compound or composition of any one of the preceding Embodiments, wherein the subject comprises 1024 G>A (E342K) mutation in human SERPINA1.

245. A method for decreasing levels and/or activities of a mutant alpha-1 antitrypsin (A1AT) polypeptide in the serum or blood of a subject, comprising delivering to the subject an effective amount of an oligonucleotide, compound or composition of any one of the preceding Embodiments, wherein the subject comprises 1024 G>A (E342K) mutation in human SERPINA1.

246. The method of Embodiment 244 or 245, wherein the mutant A1AT polypeptide is an E342K A1AT polypeptide.

247. The method of any one of Embodiments 234-246, wherein the subject is susceptible to or suffering from a condition, disorder or disease.

248. The method of any one of Embodiments 224-247, wherein the condition, disorder or disease is alpha-1 antitrypsin deficiency.

249. The method of any one of Embodiments 224-248, wherein the subject is homozygous with respect to 1024 G>A (E342K) mutation in human SERPINA1.

250. The method of any one of Embodiments 224-248, wherein the subject is heterozygous with respect to 1024 G>A (E342K) mutation in human SERPINA1.

251. The method of any one of Embodiments 224-248, wherein the subject is heterozygous with respect to 1024 G>A (E342K) mutation in human SERPINA1, and one allele is wild type.

252. The method of any one of Embodiments 224-248, wherein the subject has a heterozygous ZZ genotype.

253. The method of any one of Embodiments 224-248, wherein the subject has a homozygous ZZ genotype.

254. The method of any one of Embodiments 224-253, wherein the method increase or restores level or activity of wild-type A1AT at liver.

255. The method of any one of Embodiments 224-254, wherein the method reduces Z-AAT aggregation.

256. The method of any one of Embodiments 224-255, wherein the method reduces or prevents liver damage.

257. The method of any one of Embodiments 224-256, wherein the method reduces or prevents cirrhosis.

258. The method of any one of Embodiments 224-257, wherein the method increases level of wild-type AAT in blood.

259. The method of any one of Embodiments 224-258, wherein the method increases level of circulating, lung-bound wild-type AAT in blood.

260. The method of any one of Embodiments 224-259, wherein the method reduces or prevents lung damage.

261. The method of any one of Embodiments 224-260, wherein the method reduces or prevents lung damage from protease.

262. The method of any one of Embodiments 224-261, wherein the method reduces or prevents lung inflammation.

263. The method of any one of Embodiments 224-253, wherein the condition, disorder or disease is a liver condition, disorder or disease.

264. The method of any one of Embodiments 224-253, wherein the condition, disorder or disease is a metabolic liver condition, disorder or disease.

265. The method of any one of Embodiments 224-264, wherein the oligonucleotide, compound or composition administered to the subject comprise a targeting moiety.

266. The method of any one of Embodiments 224-265, comprising administering to the subject an effective amount of an oligonucleotide of any one of Embodiments 1-23.

267. The method of any one of Embodiments 224-265, comprising administering to the subject an effective amount of an oligonucleotide of Embodiment 1.

268. The method of any one of Embodiments 224-265, comprising administering to the subject an effective amount of an oligonucleotide of Embodiment 2.

269. The method of any one of Embodiments 224-265, comprising administering to the subject an effective amount of an oligonucleotide of Embodiment 3.

270. The method of any one of Embodiments 224-265, comprising administering to the subject an effective amount of an oligonucleotide of Embodiment 4.

271. The method of any one of Embodiments 224-265, comprising administering to the subject an effective amount of an oligonucleotide of Embodiment 5.

272. The method of any one of Embodiments 224-265, comprising administering to the subject an effective amount of an oligonucleotide of Embodiment 6.

273. The method of any one of Embodiments 224-265, comprising administering to the subject an effective amount of an oligonucleotide of Embodiment 7.

274. The method of any one of Embodiments 224-265, comprising administering to the subject an effective amount of an oligonucleotide of Embodiment 8.

275. The method of any one of Embodiments 224-265, comprising administering to the subject an effective amount of an oligonucleotide of Embodiment 9.

276. The method of any one of Embodiments 224-265, comprising administering to the subject an effective amount of an oligonucleotide of Embodiment 10.

277. The method of any one of Embodiments 224-265, comprising administering to the subject an effective amount of an oligonucleotide of Embodiment 28.

278. The method of any one of Embodiments 224-265, comprising administering to the subject an effective amount of an oligonucleotide of Embodiment 29.

279. The method of any one of Embodiments 224-265, comprising administering to the subject an effective amount of an oligonucleotide of Embodiment 30.

280. The method of any one of Embodiments 224-265, comprising administering to the subject an effective amount of an oligonucleotide of Embodiment 31.

281. The method of any one of Embodiments 224-265, comprising administering to the subject an effective amount of an oligonucleotide of Embodiment 32.

282. The method of any one of Embodiments 224-265, comprising administering to the subject an effective amount of an oligonucleotide of Embodiment 33.

283. The method of any one of Embodiments 224-265, comprising administering to the subject an effective amount of an oligonucleotide of Embodiment 34.

284. The method of any one of Embodiments 224-265, comprising administering to the subject an effective amount of an oligonucleotide of Embodiment 35.

285. The method of any one of Embodiments 224-265, comprising administering to the subject an effective amount of an oligonucleotide of Embodiment 36.

286. The method of any one of Embodiments 224-265, comprising administering to the subject an effective amount of an oligonucleotide of Embodiment 37.

287. The method of any one of Embodiments 224-265, comprising administering to the subject an effective amount of a compound of Embodiment 137.

288. The method of any one of Embodiments 224-265, comprising administering to the subject an effective amount of a compound of Embodiment 138.

289. The method of any one of Embodiments 224-265, comprising administering to the subject an effective amount of a compound of Embodiment 139.

290. The method of any one of Embodiments 224-265, comprising administering to the subject an effective amount of a compound of Embodiment 140.

291. The method of any one of Embodiments 224-265, comprising administering to the subject an effective amount of a compound of Embodiment 141.

292. The method of any one of Embodiments 224-265, comprising administering to the subject an effective amount of a compound of Embodiment 142.

293. The method of any one of Embodiments 224-265, comprising delivering to the subject an effective amount of an oligonucleotide of any one of Embodiments 24-55.

294. The method of any one of Embodiments 224-265, comprising delivering to the subject an effective amount of an oligonucleotide of any one of Embodiments 28-37.

295. The method of any one of Embodiments 224-265, comprising delivering to the subject an effective amount of an oligonucleotide of any one of Embodiments 28-37.

296. The method of any one of Embodiments 224-265, comprising delivering to the subject an effective amount of an oligonucleotide of Embodiment 28.

297. The method of any one of Embodiments 224-265, comprising delivering to the subject an effective amount of an oligonucleotide of Embodiment 29.

298. The method of any one of Embodiments 224-265, comprising delivering to the subject an effective amount of an oligonucleotide of Embodiment 30.

299. The method of any one of Embodiments 224-265, comprising delivering to the subject an effective amount of an oligonucleotide of Embodiment 31.

300. The method of any one of Embodiments 224-265, comprising delivering to the subject an effective amount of an oligonucleotide of Embodiment 32.

301. The method of any one of Embodiments 224-265, comprising delivering to the subject an effective amount of an oligonucleotide of Embodiment 33.

302. The method of any one of Embodiments 224-265, comprising delivering to the subject an effective amount of an oligonucleotide of Embodiment 34.

303. The method of any one of Embodiments 224-265, comprising delivering to the subject an effective amount of an oligonucleotide of Embodiment 35.

304. The method of any one of Embodiments 224-265, comprising delivering to the subject an effective amount of an oligonucleotide of Embodiment 36.

305. The method of any one of Embodiments 224-265, comprising delivering to the subject an effective amount of an oligonucleotide of Embodiment 37.

306. The method of any one of Embodiments 224-265, comprising delivering to the subject an effective amount of a compound of Embodiment 140.

307. The method of any one of Embodiments 224-265, comprising delivering to the subject an effective amount of a compound of Embodiment 141.

308. The method of any one of Embodiments 224-265, comprising delivering to the subject an effective amount of a compound of Embodiment 142.

309. The method of any one of Embodiments 293-308, wherein the oligonucleotide or compound is delivered by administering to the subject an effective amount of a conjugate of the oligonucleotide or compound, respectively, with an additional chemical moiety or a salt thereof.

310. The method of Embodiment 309, wherein the additional chemical moiety is or comprises a targeting moiety.

311. The method of Embodiment 309, wherein the additional chemical moiety is or comprises a carbohydrate moiety.

312. The method of Embodiment 309, wherein the additional chemical moiety is or comprises a lipid moiety.

313. The method of Embodiment 309, wherein the additional chemical moiety is or comprises one or more protein ligand moieties.

314. The method of Embodiment 309, wherein the additional chemical moiety is or comprises two or more protein ligand moieties.

315. The oligonucleotide of Embodiment 309, wherein the additional chemical moiety targets liver.

316. The oligonucleotide of Embodiment 309, wherein the additional chemical moiety is or comprises a ligand of a receptors expressed in liver.

317. The method of Embodiment 309, wherein the additional chemical moiety is or comprises a ligand for an asialoglycoprotein receptor.

318. The method of Embodiment 309, wherein the additional chemical moiety comprises multiple moieties, each of which is independently a ligand for an asialoglycoprotein receptor.

319. The method of Embodiment 309, wherein the additional chemical moiety is or comprises GalNAc.

320. The method of Embodiment 309, wherein the additional chemical moiety comprises three GalNAc.

321. The method of Embodiment 309, wherein the additional chemical moiety is or comprises

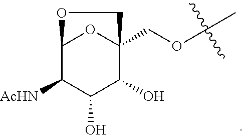 [SMILES: *OC[C@@]12CO[C@@H](O1)[C@H](NC(C)=O)[C@@H](O)[C@H]2O]

322. The method of Embodiment 309, wherein the additional chemical moiety is

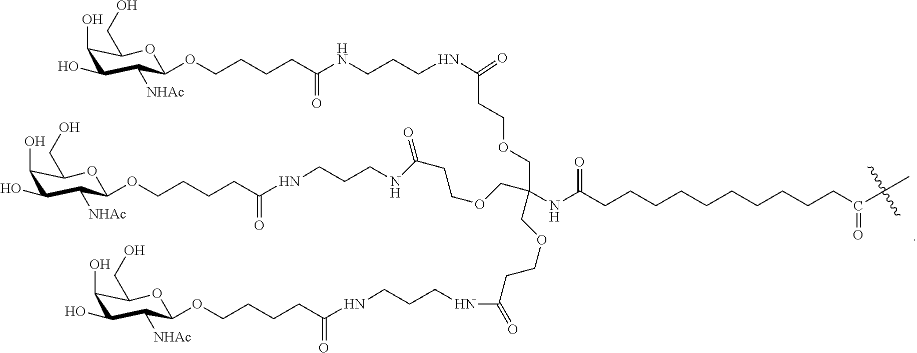

323. The method of Embodiment 309, wherein the additional chemical moiety is or comprises

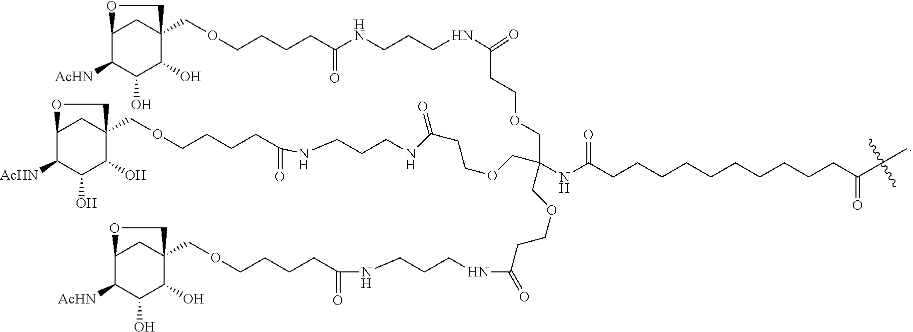 [SMILES: *C(=O)CCCCCCCCCCC(=O)NC(COCCC(=O)NCCCNC(=O)CCCCOC[C@@]12CO[C@@H](C1)[C@H](NC(C)=O)[C@@H](O)[C@H]2O)(COCCC(=O)NCCCNC(=O)CCCCOC[C@@]12CO[C@@H](C1)[C@H](NC(C)=O)[C@@H](O)[C@H]2O)COCCC(=O)NCCCNC(=O)CCCCOC[C@@]12CO[C@@H](C1)[C@H](NC(C)=O)[C@@H](O)[C@H]2O]

324. The method of any one of Embodiments 309-323, wherein the additional chemical moiety is directly conjugated to the remainder of the compound.

325. The method of any one of Embodiments 309-323, wherein the additional chemical moiety is conjugated via a linker to the remainder of the compound.

326. The method of Embodiment 325, wherein a linker is or comprises L001.

327. The method of any one of Embodiments 309-326, wherein the additional chemical moiety is conjugated to the 5′-end of the oligonucleotide chain.

328. The method of any one of Embodiments 309-326, wherein the additional chemical moiety is conjugated to the 3′-end of the oligonucleotide chain.

329. The method of any one of Embodiments 309-326, wherein the additional chemical moiety is conjugated to the middle of the oligonucleotide chain.

330. The method of any one of Embodiments 309-329, wherein the additional chemical moiety is conjugated to a sugar.

331. The method of any one of Embodiments 309-329, wherein the additional chemical moiety is conjugated to a nucleobase.

332. The method of any one of Embodiments 309-329, wherein the additional chemical moiety is conjugated to an internucleotidic linkage.

333. The method of Embodiment 326, wherein L001 is connected to 5′-end 5′-carbon of the oligonucleotide chain through the phosphate group.

334. A method for delivering to a system an oligonucleotide of any one of Embodiments 24-55, comprising administering to the system a conjugate of the oligonucleotide with an additional chemical moiety or a salt thereof.

335. A method for delivering to a system a compound of any one of Embodiments 140-142, comprising administering to the system a conjugate of the compound with an additional chemical moiety or a salt thereof.

336. The method of any one of Embodiments 334-335, wherein the additional chemical moiety is or comprises a targeting moiety.

337. The method of any one of Embodiments 334-335, wherein the additional chemical moiety is or comprises a carbohydrate moiety.

338. The method of any one of Embodiments 334-335, wherein the additional chemical moiety is or comprises a lipid moiety.

339. The method of any one of Embodiments 334-335, wherein the additional chemical moiety is or comprises one or more protein ligand moieties.

340. The method of any one of Embodiments 334-335, wherein the additional chemical moiety is or comprises two or more protein ligand moieties.

341. The method of any one of Embodiments 334-335, wherein the additional chemical moiety targets liver.

342. The method of any one of Embodiments 334-335, wherein the additional chemical moiety is or comprises a ligand of a receptors expressed in liver.

343. The method of any one of Embodiments 334-335, wherein the additional chemical moiety is or comprises a ligand for an asialoglycoprotein receptor.

344. The method of any one of Embodiments 334-335, wherein the additional chemical moiety comprises multiple moieties, each of which is independently a ligand for an asialoglycoprotein receptor.

345. The method of any one of Embodiments 334-335, wherein the additional chemical moiety is or comprises GalNAc.

346. The method of any one of Embodiments 334-335, wherein the additional chemical moiety comprises three GalNAc.

347. The method of any one of Embodiments 334-335, wherein the additional chemical moiety is or

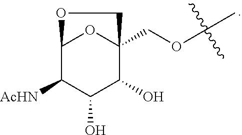 [SMILES: *OC[C@@]12CO[C@@H](O1)[C@H](NC(C)=O)[C@@H](O)[C@H]2O]

348. The method of any one of Embodiments 334-335, wherein the additional chemical moiety is

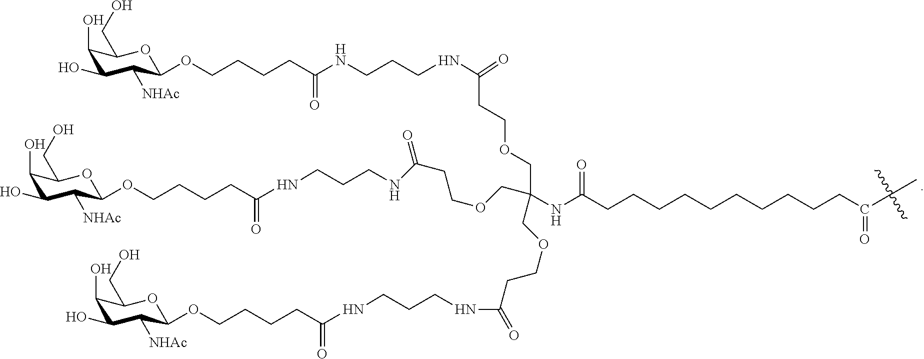

349. The method of any one of Embodiments 334-335, wherein the additional chemical moiety is or comprise

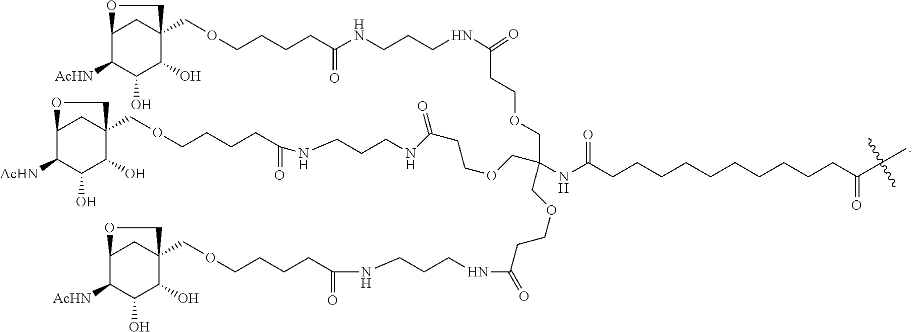 [SMILES: *C(=O)CCCCCCCCCCC(=O)NC(COCCC(=O)NCCCNC(=O)CCCCOC[C@@]12CO[C@@H](C1)[C@H](NC(C)=O)[C@@H](O)[C@H]2O)(COCCC(=O)NCCCNC(=O)CCCCOC[C@@]12CO[C@@H](C1)[C@H](NC(C)=O)[C@@H](O)[C@H]2O)COCCC(=O)NCCCNC(=O)CCCCOC[C@@]12CO[C@@H](C1)[C@H](NC(C)=O)[C@@H](O)[C@H]2O]

350. The method of any one of Embodiments 334-349, wherein the additional chemical moiety is directly conjugated to the remainder of the compound.

351. The method of any one of Embodiments 334-349, wherein the additional chemical moiety is conjugated via a linker to the remainder of the compound.

352. The method of Embodiment 351, wherein a linker is or comprises L001.

353. The method of any one of Embodiments 334-352, wherein the additional chemical moiety is conjugated to the 5′-end of the oligonucleotide chain.

354. The method of any one of Embodiments 334-352, wherein the additional chemical moiety is conjugated to the 3′-end of the oligonucleotide chain.

355. The method of any one of Embodiments 334-352, wherein the additional chemical moiety is conjugated to the middle of the oligonucleotide chain.

356. The method of any one of Embodiments 334-355, wherein the additional chemical moiety is conjugated to a sugar.

357. The method of any one of Embodiments 334-355, wherein the additional chemical moiety is conjugated to a nucleobase.

358. The method of any one of Embodiments 334-355, wherein the additional chemical moiety is conjugated to an internucleotidic linkage.

359. The method of Embodiment 352, wherein L001 is connected to 5′-end 5′-carbon of the oligonucleotide chain through the phosphate group.

360. A method for delivering to a system an oligonucleotide of any one of Embodiments 28-37, comprising administering to the system the corresponding oligonucleotide described in Embodiments 1-10.

361. A method for delivering to a system an oligonucleotide of Embodiment 28, comprising administering to the system the oligonucleotide of Embodiment 1.

362. A method for delivering to a system an oligonucleotide of Embodiment 29, comprising administering to the system the oligonucleotide of Embodiment 2.

363. A method for delivering to a system an oligonucleotide of Embodiment 30, comprising administering to the system the oligonucleotide of Embodiment 3.

364. A method for delivering to a system an oligonucleotide of Embodiment 31, comprising administering to the system the oligonucleotide of Embodiment 4.

365. A method for delivering to a system an oligonucleotide of Embodiment 32, comprising administering to the system the oligonucleotide of Embodiment 5.

366. A method for delivering to a system an oligonucleotide of Embodiment 33, comprising administering to the system the oligonucleotide of Embodiment 6.

367. A method for delivering to a system an oligonucleotide of Embodiment 34, comprising administering to the system the oligonucleotide of Embodiment 7.

368. A method for delivering to a system an oligonucleotide of Embodiment 35, comprising administering to the system the oligonucleotide of Embodiment 8.

369. A method for delivering to a system an oligonucleotide of Embodiment 36, comprising administering to the system the oligonucleotide of Embodiment 9.

370. A method for delivering to a system an oligonucleotide of Embodiment 37, comprising administering to the system the oligonucleotide of Embodiment 10.

371. A method for delivering to a system a compound of any one of Embodiments 140-142, comprising administering to the system the corresponding compound described in Embodiments 137-139.

372. A method for delivering to a system a compound of Embodiment 140, comprising administering to the system the compound of Embodiment 137.

373. A method for delivering to a system a compound of Embodiment 141, comprising administering to the system the compound of Embodiment 138.

374. A method for delivering to a system a compound of Embodiment 142, comprising administering to the system the compound of Embodiment 139.

375. The method of any one of Embodiments 334-374, wherein the system is or comprises a cell.

376. The method of any one of Embodiments 334-374, wherein the system is or comprises a tissue.

377. The method of any one of Embodiments 334-374, wherein the system is or comprises an organ.

378. The method of any one of Embodiments 334-374, wherein the system is a subject.

379. The method of any one of Embodiments 334-374, wherein the system is a human.

380. The method of any one of Embodiments 334-379, wherein the system comprises 1024 G>A (E342K) mutation in human SERPINA1.

381. The method of any one of Embodiments 334-380, wherein the system is homozygous with respect to 1024 G>A (E342K) mutation in human SERPINA1.

382. The method of any one of Embodiments 334-380, wherein the system is heterozygous with respect to 1024 G>A (E342K) mutation in human SERPINA1.

383. The method of any one of Embodiments 334-380, wherein the system is heterozygous with respect to 1024 G>A (E342K) mutation in human SERPINA1, and one allele is wild type.

384. The method of any one of Embodiments 334-383, wherein the 1024 G>A (E342K) in a transcript is edited.

385. The method of any one of the preceding Embodiments, wherein the oligonucleotide or compound is a pharmaceutically acceptable salt form.

386. The method of any one of the preceding Embodiments, wherein the oligonucleotide or compound is administered in one or more pharmaceutically acceptable salt forms.

387. The method of any one of the preceding Embodiments, wherein the oligonucleotide or compound is delivered in one or more pharmaceutically acceptable salt forms.

388. The method of any one of the preceding Embodiments, wherein 1024 G>A (E342K) mutation in a SERPINA1 transcript is edited.

389. The method of any one of the preceding Embodiments, wherein E342K is corrected in an A1AT polypeptide.

390. The method of any one of the preceding Embodiments, wherein serum A1AT level is increased compared to a level prior to the administration or delivery.

391. The method of any one of the preceding Embodiments, wherein serum E342 A1AT level is increased compared to a level prior to the administration or delivery.

392. The method of any one of the preceding Embodiments, wherein serum E342K A1AT level is decreased compared to a level prior to the administration or delivery.

393. The method of any one of the preceding Embodiments, wherein the oligonucleotide, compound or composition is administered without loading doses.

394. The method of any one of Embodiments 212-393, wherein the oligonucleotide, compound or composition is administered with one or more loading doses.

395. An oligonucleotide, compound or composition of any one of the preceding Embodiments, for use in a method of any one of the preceding Embodiments.

396. An oligonucleotide, compound or composition of any one of the preceding Embodiments, for treating a condition, disorder or disease described in any one of the preceding Embodiments.

397. An oligonucleotide, compound or composition of any one of the preceding Embodiments, for treating a condition, disorder or disease associated with 1024 G>A (E342K) mutation in human SERPINA1.

398. An oligonucleotide, compound or composition of any one of the preceding Embodiments, for treating AATD associated with 1024 G>A (E342K) mutation in human SERPINA1.

399. An oligonucleotide, compound or composition of any one of the preceding Embodiments, for manufacturing a medicament for a method of any one of the preceding Embodiments.

400. An oligonucleotide, compound or composition of any one of the preceding Embodiments, for manufacturing a medicament for treating a condition, disorder or disease associated with 1024 G>A (E342K) mutation in human SERPINA1.

401. An oligonucleotide, compound or composition of any one of the preceding Embodiments, for manufacturing a medicament for treating AATD associated with 1024 G>A (E342K) mutation in human SERPINA1.

402. A method of preparing an oligonucleotide or compound of any one of the preceding Embodiments, comprising coupling a phosphoramidite comprising a chiral auxiliary with a hydroxyl group.

403. A method of preparing an oligonucleotide or compound of any one of the preceding Embodiments, comprising coupling a phosphoramidite with a hydroxyl group, wherein the phosphoramidite has the structure of having the structure of

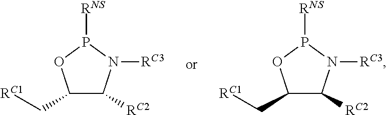

or a salt thereof,
R^{NS} is a optionally protected nucleoside moiety;
R^{C1} is R, —Si(R)₃ or —SO₂R;
each of R^{C2} and R^{C3} is independently R; and
each R is independently —H, or an optionally substituted group selected from C₁₋₂₀ aliphatic, C₁₋₂₀ heteroaliphatic having 1-10 heteroatoms, C₆₋₁₀ aryl, C₆₋₂₀ arylaliphatic, C₆₋₁₀ arylheteroaliphatic having 1-10 heteroatoms, 5-20 membered heteroaryl having 1-10 heteroatoms, and 3-20 membered heterocyclyl having 1-10 heteroatoms, or:
two R groups are optionally and independently taken together to form a covalent bond, or:
two or more R groups on the same atom are optionally and independently taken together with the atom to form an optionally substituted, 3-20 membered, monocyclic, bicyclic or polycyclic ring having, in addition to the atom, 0-10 heteroatoms; or:
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

404. The method of any one of Embodiments 402-403, comprising coupling a phosphoramidite comprising a chiral auxiliary with a free hydroxyl group of an optionally substituted oligonucleotide or nucleoside.

405. The method of any one of Embodiments 402-403, comprising coupling a phosphoramidite comprising a chiral auxiliary with a free 5′—OH group of an optionally substituted oligonucleotide or nucleoside.

406. The method of any one of Embodiments 403-405, wherein R^{C2} and R^{C3} are taken together with their intervening atoms to form an optionally substituted 3-7 membered saturated or partially unsaturated ring having, in addition to the nitrogen atom, 0-2 heteroatoms.

407. The method of any one of Embodiments 403-406, wherein R^{C2} and R^{C3} are taken together with their intervening atoms to form an optionally substituted 5-membered saturated ring having no heteroatoms in addition to the nitrogen atom.

408. The method of any one of Embodiments 402-407, wherein for an occurrence of phosphoramidite, R^{C1} is —Si(R)₃ wherein each R is independently an optionally substituted group selected from C₁₋₂₀ aliphatic and C₁₋₂₀ aryl.

409. The method of any one of Embodiments 402-407, wherein for an occurrence of phosphoramidite. R^{C1} is —SiPh₂Me.

410. The method of any one of Embodiments 402-409, wherein for an occurrence of phosphoramidite. R^{C1} is —SO₂R wherein R is not —H.

411. The method of any one of Embodiments 402-409, wherein for an occurrence of phosphoramidite. R^{C1} is —SO₂R, wherein R is optionally substituted C₁₋₁₀ aliphatic.

412. The method of any one of Embodiments 402-409, wherein for an occurrence of phosphoramidite, R^{C1} is —SO₂R, wherein R is optionally substituted phenyl.

413. The method of any one of Embodiments 402-409, wherein for an occurrence of phosphoramidite, R^{C1} is —SO₂R, wherein R is phenyl.

414. The method of any one of Embodiments 402-413, wherein a hydroxyl group of R^{NS} is protected.

415. The method of any one of Embodiments 402-413, wherein a hydroxyl group of R^{NS} is protected as ODMTr.

416. The method of any one of Embodiments 402-413, wherein the 5′-OH of R^{NS} is protected.

417. The method of any one of Embodiments 402-413, wherein the 5′-OH of R^{NS} is protected as ODMTr.

418. The method of any one of Embodiments 402-417, wherein each heteroatom is independently selected from nitrogen, oxygen, silicon, phosphorus and sulfur.

419. The method of any one of Embodiments 402-418, wherein the method comprises coupling with C-6 amino linker.

420. The method of any one of Embodiments 402-419, wherein the method comprises conjugation with an GalNAc-containing acid.

421. The method of any one of Embodiments 402-418, wherein the method comprises coupling with a phosphoramidite comprising an optionally substituted additional chemical moiety.

422. The method of any one of Embodiments 402-418, wherein the method comprises coupling with a phosphoramidite comprising an optionally substituted additional chemical moiety and linker.

423. The method of Embodiment 420, wherein the GalNAc-containing acid has the structure of

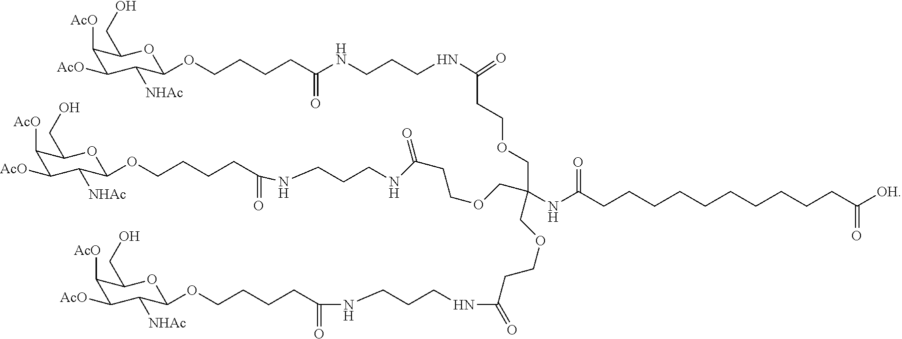 [SMILES: CC(=O)NC1C(OCCCCC(=O)NCCCNC(=O)CCOCC(COCCC(=O)NCCCNC(=O)CCCCOC2OC(CO)C(OC(C)=O)C(OC(C)=O)C2NC(C)=O)(COCCC(=O)NCCCNC(=O)CCCCOC2OC(CO)C(OC(C)=O)C(OC(C)=O)C2NC(C)=O)NC(=O)CCCCCCCCCCC(=O)O)OC(CO)C(OC(C)=O)C1OC(C)=O]

424. The method of any one of Embodiments 421-422, wherein the phosphoramidite has the structure of

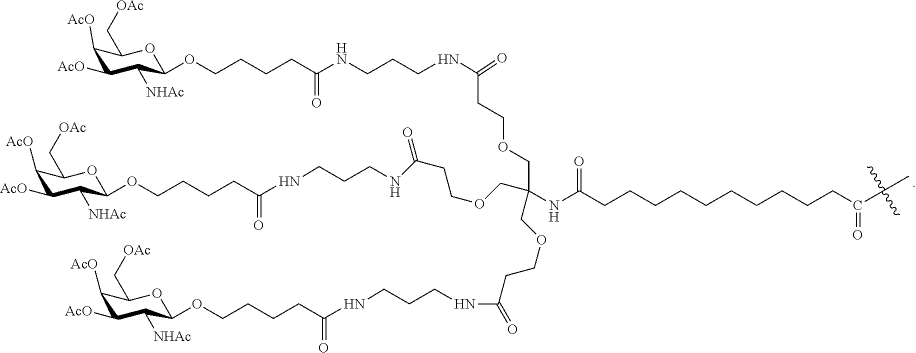

425. The method of any one of Embodiments 423-424, comprising de-protecting protected hydroxy groups in GalNAc (OAc) to —OH.

### EXEMPLIFICATION

Certain examples of provided technologies (compounds (oligonucleotides, reagents, etc.), compositions, methods (methods of preparation, use, assessment, etc.), etc.) were presented herein. Those skilled in the art appreciate that many technologies, e.g., those described in U.S. Pat. No. 9,982,257. US 20170037399. US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, WO 2018/237194, WO 2019/032607, WO 2019/055951, WO 2019/075357, WO 2019/200185, WO 2019/217784, WO 2019/032612, WO 2020/191252, WO 2021/071858, WO 2022/099159, etc., can be utilized to prepare and/or assess properties and/or activities of provided technologies in accordance with the present disclosure.

### Example 1. Useful Technologies for Assessing Provided Technologies

Various technologies can be utilized for assessing provided technologies. e.g., for adenosine editing, in accordance with the present disclosure. In some embodiments, report assays can be utilized. In some embodiments, oligonucleotides and compositions were accessed and confirmed to provide editing in various cells, e.g., mouse or human primary hepatocytes, cell lines, etc. In some embodiments, oligonucleotide and compositions were assessed and confirmed to provide editing in subjects. In some embodiments, oligonucleotides and compositions were assessed and confirmed to provide editing in animals, e.g., mice, non-human primates (e.g., cynomolgus macaques), etc. In some embodiments, cells, subjects, etc. comprise relevant target adenosine, e.g., 1024 G>A in SERPINA1.

Certain useful technologies are described in the present disclosure and the priority applications, WO 2021/071858, WO 2022/046667, or WO 2022/099159, the entirety of each of which is independently incorporated by reference.

In some embodiments, provided technologies, e.g., oligonucleotides and compositions thereof, are assessed in animal models. In some embodiments, levels, properties, and/or activities of desired products (e.g., properly folded wild-type A1AT protein in serum) are increased, and/or levels, properties, and/or activities of undesired products (e.g., mutant (e.g., E342K) A1AT protein in serum) are decreased, in observed amounts (e.g., ng/mL in serum) and/or relatively (e.g., as % of total proteins or total A1AT proteins).

Oligonucleotides and compositions can be delivered utilizing many technologies in accordance with the present disclosure. For example, in some embodiments, they were delivered by transfection. In some embodiments, they were delivered by gymnotic uptake. In some embodiments, oligonucleotides comprise moieties that can facilitate delivery. For example, in some embodiments, a moiety is a ligand for a polypeptide, e.g., a receptor, in many instances, on cell surface. In some embodiments, a polypeptide is expressed at a higher level by a type or population of cells, a tissue, etc. so that it may be utilized for delivery. In some embodiments, a ligand is an ASGPR ligand. In some embodiments, a ligand is or comprises GalNAc or a derivative thereof. In some embodiments, an oligonucleotide may comprise two or more ligand moieties, each of which is independently a ligand of a polypeptide. In some embodiments, an oligonucleotide comprises two or more copies of a ligand moiety. In some embodiments, a moiety targets one or more characteristics (e.g., pH, redox, etc.) of a location or environment.

In some embodiments, technologies of the provided technology can provide increased stability, high levels of editing, etc.

In some embodiments, provided technologies can provide high levels of selectivity. In some embodiments, about or at least about 80%, 85%, 90%, 91%, 92%, 93%, 94%, 95%, 95%, 96%, 97%, 98%, 99%, 99.5%, or 99.9% observed adenosine editing are at target adenosines. In some embodiments, about or at least about 80%, 85%, 90%, 95%, 96%, 97%, 98%, or 99% observed adenosine editing in coding regions are at target adenosines. In some embodiments, about or at least about 80%, 85%, 90%, 95%, 96%, 97%, 98%, or 99% observed adenosine editing in target nucleic acids (e.g., transcripts of target genes) are at target adenosines. In some embodiments, about or at least about 80%, 85%, 90%, 95%, 96%, 97%, 98%, or 99% observed adenosine editing in coding regions of target nucleic acids (e.g., transcripts of target genes) are at target adenosines. Various technologies, e.g., RNA-Seq, are available to those skilled in the art to assess selectivity; certain such technologies are described herein or in the priority applications, WO 2021/071858, WO 2022/046667, or WO 2022/099159, the entirety of each of which is independently incorporated herein by reference. In some embodiments, a percentage for a selectivity described herein is at least about 80%. In some embodiments, it is at least about 85%. In some embodiments, it is at least about 90%. In some embodiments, it is at least about 95%. In some embodiments, it is at least about 96%. In some embodiments, it is at least about 97%. In some embodiments, it is at least about 98%. In some embodiments, it is at least about 99%. In some embodiments, it is at least about 99.5%. In some embodiments, it is at least about 99.9%. In some embodiments, it is about 100%. In some embodiments, no off-target editing is observed. In some embodiments, provided technology provides high selectivity in vivo.

Various results are presented in, e.g., Figures and Tables herein, as examples illustrating various benefits and advantages provided technologies can provide.

Provided technologies can provide robust editing in the presence of ADAR1 and/or ADAR2. Provided technologies can provide robust editing in the presence of ADAR1-p110 and/or ADAR1-p150.

### Example 2. Technologies for Preparing Oligonucleotide and Compositions

Various technologies (e.g., phosphoramidites, nucleobases, nucleosides, etc.) for preparing provided technologies (e.g., oligonucleotides, compositions (e.g., oligonucleotide compositions, pharmaceutical compositions, etc.), etc.) can be utilized in accordance with the present disclosure, including, for example, methods and reagents described in U.S. Pat. No. 9,982,257, US 20170037399, US 20180216108. US 20180216107, U.S. Pat. No. 9,598,458, WO 2017/062862, WO 2018/067973, WO 2017/160741, WO 2017/192679, WO 2017/210647, WO 2018/098264, WO 2018/223056, WO 2018/237194, WO 2019/032607, WO 2019/055951. WO 2019/075357, WO 2019/200185, WO 2019/217784, WO 2019/032612, WO 2020/191252, WO 2021/071858, and/or WO 2022/099159, the methods and reagents of each of which are incorporated herein by reference. In some embodiments, the present disclosure provides useful technologies for preparing oligonucleotides and compositions thereof. Many oligonucleotides and compositions thereof, e.g., various oligonucleotides and compositions thereof in Table 1, were prepared and assessed and were confirmed to provide various activities, e.g., adenosine editing.

For example, the following reagents may be utilized to introduce additional chemical moieties comprising GalNAc:

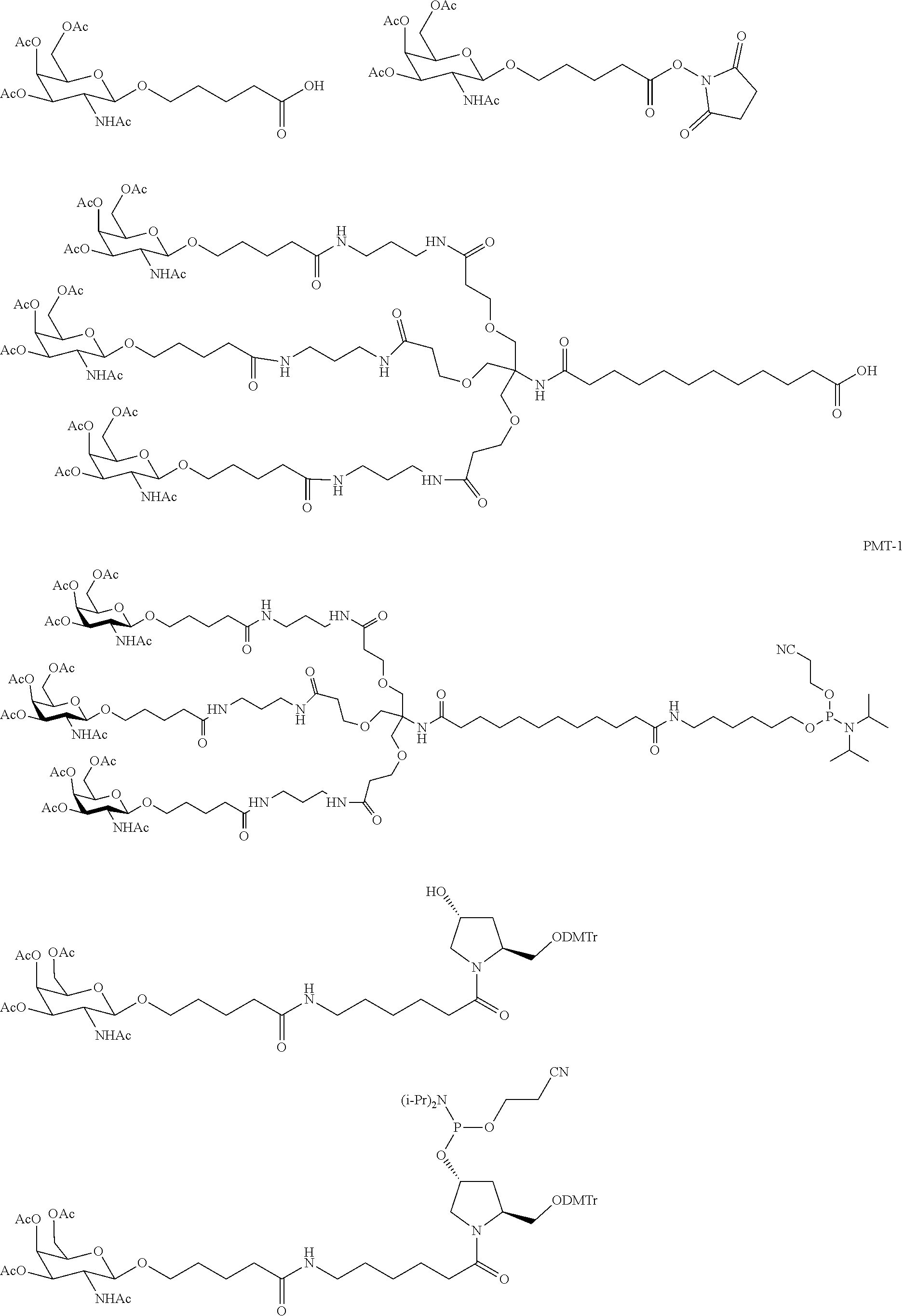

In some embodiments, preparations include one or more DPSE and/or PSM cycles.

Various chirally controlled oligonucleotide compositions were prepared. Certain useful procedures were described below as examples. In some embodiments, oligonucleotides comprises mixed PS (phosphorothioate)/PO(natural phosphate linkage)/PN (e.g., phosphoryl guanidine internucleotidic linkages such as n001) backbone. For example, in some embodiments, phosphodiester (PO) linkage were formed using cyanoethyl amidites, phosphorothioate (PS) linkages (Sp and Rp; in some embodiments, all Sp) were formed using DPSE chiral amidites, phosphoroamidate linkages (PN; e.g., n001) (Sp and Rp) linkages were formed using PSM amidites. In some embodiments, oligonucleotides comprise additional moieties such as triantennary GalNAc moiety at, e.g., 5′-end. For introduction of GalNAc moiety at 5′-end, in some embodiments oligonucleotides were synthesized by coupling with C-6 amino modifier as the last coupling cycle and after purification and desalting were conjugated with tri-antennary GalNAc to make conjugates.

### Example Procedure for Preparation of Oligonucleotide Compositions (25 μMol Scale)

For chirally controlled PS linkages. DPSE amidites were used and for chirally controlled PN linkages such as n001. PSM amidites were used. Automated solid-phase synthesis of oligonucleotides was performed according to cycles shown below: Regular amidite cycle for P0 linkages. DPSE amidite cycle for chirally controlled PS linkages, and PSM amidite cycles for chirally controlled PN linkages such as n001.

Regular Amidite Synthetic Cycle

| step | operation | reagents and solvent | volume | | waiting time | |
|---|---|---|---|---|---|---|
| 1 | detritylation | 3% TCA/DCM | 10 | mL | 65 | s |
| 2 | coupling | 0.2M monomer/20% IBN—MeCN | 0.5 | mL | 8 | min |
| | | 0.5M CMIMT/MeCN | 1.0 | mL | | |
| 3 | oxidation | 50 mM I₂/pyridine-H₂O (9:1, v/v) | 2.0 | mL | 1 | min |
| 4 | cap-2 | 20% Ac₂O, 30% 2,6-lutidine/MeCN | 1.0 | mL | 45 | s |
| | | 20% MeIm/MeCN | 1.0 | mL | | |

DPSE Amidite Synthetic Cycle

| step | operation | reagents and solvent | volume | | waiting time | |
|---|---|---|---|---|---|---|
| 1 | detritylation | 3% TCA/DCM | 10 | mL | 65 | s |
| 2 | coupling | 0.2M monomer/20% IBN—MeCN | 0.5 | mL | 8 | min |
| | | 0.5M CMIMT/MeCN | 1.0 | mL | | |
| 3 | cap-1 | 20% Ac₂O, 30% 2,6-lutidine/MeCN | 2.0 | mL | 2 | min |
| 4 | sulfurization | 0.2M XH/pyridine | 2.0 | mL | 6 | min |
| 5 | cap-2 | 20% Ac₂O, 30% 2,6-lutidine/MeCN | 1.0 | mL | 45 | s |
| | | 20% MeIm/MeCN | 1.0 | mL | | |

PSM Amidite Synthetic Cycle

| step | operation | reagents and solvent | volume | | waiting time | |
|---|---|---|---|---|---|---|
| 1 | detritylation | 3% TCA/DCM | 10 | mL | 65 | s |
| 2 | coupling | 0.2M monomer/20% IBN—MeCN | 0.5 | mL | 8 | min |
| | | 0.5M CMIMT/MeCN | 1.0 | mL | | |
| 3 | cap-1 | 20% Ac₂O, 30% 2,6-lutidine/MeCN | 2.0 | mL | 2 | min |
| 4 | imidation | 0.5M ADIH reagent/MeCN | 2.0 | mL | 6 | min |
| 5 | cap-2 | 20% Ac₂O, 30% 2,6-lutidine/MeCN | 1.0 | mL | 45 | s |
| | | 20% MeIm/MeCN | 1.0 | mL | | |

In some embodiments, for introduction of GalNAc moiety at 5′-end, oligonucleotides were synthesized by coupling with C-6 amino linker as the last coupling cycle.

### Example Procedure for Cleavage & De-Protection (25 μMol Scale)

After completion of cycles, the CPG support was treated with 20% diethylamine/acetonitrile wash step for 5 column volume/15 mins followed by ACN wash cycle. The CPG solid support was dried and transferred into 50 mL plastic tube, and was treated with 1× desilylation reagent (2.5 mL; 100 μL/umol) for 3 h at 28° C., then added conc. NH₃ (5.0 mL; 200 μL/umol) for 24 b at 37′° C. The reaction mixture was cooled to room temperature and the CPG was separated by membrane filtration and washed with 15 mL of H₂O. The crude material (filtrate) was analyzed by LTQ and RP-UPLC. For certain oligonucleotides to be conjugated with other additional chemical moieties such as GalNAc, oligonucleotides comprising suitable reactive groups such as amino groups were purified by ion exchange chromatography on AKTA pure system using a sodium chloride gradient. Desired product was desalted and further conjugated with GalNAc-containing acid. After conjugation reaction was found to be completed, the material was further purified by ion exchange chromatography and desalted using tangential flow filtration (TFF) to obtain desired products. Additional chemical moieties can also be installed by coupling with phosphoramidites comprising such additional chemical moieties (and optional linkers), e.g., PMT-1.

For example, in some embodiments, WV-47595 (L001mCn001RmC*SmC*SfA*SfG*SmCmA*SfG*SfCmU*SfUn001RmCfA*SfGn001RfUmC*SfC*SfC*SfU*SmUmUfC*ST*Sb008U*SIn001SmUfC*SmG*SmAn001RmU) was prepared and then conjugated to prepare WV-46312. A useful synthesis process is described below as an example.

In a preparation, synthesis of WV-47595 was performed on AKTA OP100 synthesizer (GE healthcare) using a 3.5 cm diameter fineline column on a 1200 μmol scale using a CPG support (loading 72 μmol/g). Certain synthetic cycles contained five steps: detritylation, coupling, capping 1 (cap-1), oxidation/sulfurization/imidation and capping 2 (cap-2).

Detritylation: Detritylation was performed using 3% DCA in toluene with a UV watch command set at 436 nm. Following detritylation, the CPG support was subjected to wash cycle using acetonitrile for 2CV.

Coupling: DPSE and PSM chiral amidites were prepared at 0.2M cone. (in ACN or 20% IBN in ACN). The amidites were mixed in-line with CMIMT activator (0.5M in acetonitrile) at a ratio of 5.83 prior to addition to the column. The coupling mixture was recycled for 10 minutes to maximize the coupling efficiency followed by column wash with 2CV of ACN. Cyanoethyl amidites were prepared at 0.2M conc. (in ACN or 20% IBN in ACN). The amidites were mixed in-line with ETT activator (0.5M in acetonitrile) at a ratio of 4.07 prior to addition to the column. The coupling mixture was recycled for 10 minutes to maximize the coupling efficiency followed by column wash with 2CV of ACN.

Capping 1: For stereodefined couplings, the column was then treated with Capping 1 solution (acetic anhydride, lutidine, ACN) for 1 CV in 2 minutes which can acetylate the chiral auxiliary amine. Following this step, the column was washed with 1.5 CV of acetonitrile. For stereorandom coupling Capping 1 was not performed.

Sulfurization/Imidation/Oxidation step: Sulfurization was performed with 0.1 M xanthane hydride in pyridine/acetonitrile (1.2 equivalent) with a contact time of 6 minute followed by 2CV wash step. Imidation was performed with 0.3 M ADIH reagent in acetonitrile with 18 equivalent and 15 min contact time followed by 2CV wash step. Oxidation step was performed using oxidation reagent (50 mM I₂/pyridine-H₂O (9:1, v/v)) 3.5 eq. 2.5 minute followed by 2CV acetonitrile wash.

Capping 2: Capping 2 step was performed using Capping A and Capping B reagents mixed inline (1:1) (e.g., see cap-2) followed by a 2 CV ACN wash.

After completion of the synthesis, the CPG support was finally treated with 20% diethylamine/acetonitrile wash step for 5 column volume/15 mins followed by ACN wash cycle. The CPG solid support was dried and transferred into pressure vessel. DPSE were removed by treating the support with desilylation reagent at a ratio of per μmole support/100 μL desilylation reagent. The desilylation reagent was made by mixing DMSO:water:TEA:TEA.3HF in ratio of 7.33:1.47:0.7:0.5. The CPG support was incubated in presence with desilylation reagent for 3 hours at 27° C. in an incubator shaker. After that cone, ammonia was added at a ratio of per μmole support/200 μL of conc. ammonia. The mixture was incubated and shaken for 24 hours at 37° C. The mixture was cooled and filtered using 0.2-0.45 micron filter and the CPG support was rinsed three times to collect all the desired material as filtrate. The filtrate containing crude oligonucleotides was analyzed by RP-UPLC and quantitation was done using a Nanodrop One Spectrophotometer (Thermo Scientific) and a yield of 110,000 OD/μmole was obtained.

Purification and desalting: Crude oligonucleotides were loaded on to Waters AP-2 glass column (2.0 cm×20 cm) packed with Source 15Q (Cytiva). Purification was performed on an AKTA150 Pure (GE healthcare) using the following buffers: (Buffer A: 20 mM NaOH, 20% Acetonitrile v/v) (Buffer B: 20 mM NaOH, 2.5M NaCl, 20% Acetonitrile v/v). Desired fractions with full length products in the range of 70-80% were pooled together. The pooled material was then desalted on a 2KD re-generated cellulose membrane followed by lyophilization to obtain oligonucleotides as a fluffy white cake ready for conjugation.

Preparation of WV-46312: Various technologies can be utilized to conjugate oligonucleotides with other moieties in accordance with the present disclosure. A useful protocol for GalNAc conjugation is described below as an example. Pre-conjugation material: WV-47595.01 (0.01 denoting the batch number).

Product material: WV-46312.01.

| | Mol. Wt. for | | Volume |
|---|---|---|---|
| Reagent | present protocol | Equivalent | (mL) |
| WV-47595 | 10050.80 | 3 | — |
| Tri-antennary GalNAc acid | 2006 | 1.8 | — |
| HATU | 382 | 1.4 | — |
| DIEA | 129 | 10 | — |
| Acetonitrile | | — | 4 |

Aqueous oligonucleotide solution

| | Conc. | Total volume | Total |
|---|---|---|---|
| Oligonucleotide solution | (mg/mL) | (mL) | (mg) |
| WV-47595 in WFI water | 50 | 8 | 400 |

The tri-antennary GalNAc acid (hydroxyl groups protected as —OAc) and HATU are weighed out in a 50 mL plastic tube and dissolved in anhydrous acetonitrile then DIEA was added into the tube. The resulting mixture was stirred for 10 min at 37° C. Lyophilized WV-47595 was reconstituted in water in a separate tube and the GalNAc mixture was added to the oligonucleotide solution and stirred for 60 min at 37° C. The reaction was monitored by RP-UPLC. Reaction was found to be complete in 1 h. The reaction mixture was concentrated under vacuum to remove the acetonitrile and the resultant GalNAc-conjugated oligonucleotides was treated with cone, ammonia for 2 h at 37° C. The formation of final product was confirmed by mass spectrometry and RP-UPLC. The conjugated material was purified by anion exchange chromatography and desalted using tangential flow filtration (TFF) to obtain the final product (Target mass: 12110.65; Observed mass: 12112.3). Using similar procedures various oligonucleotides and compositions were manufactured.

Additional technologies for preparing oligonucleotides are illustrated below as examples.

### Example Procedure for Preparation of Oligonucleotide Compositions (50 μMol Scale)

Certain stereopure oligonucleotides were synthesized at 50 umol scale using a MerMade12 synthesizer and standard CPG. In some embodiments, an amidite approach was used to incorporate GalNAc on the 5′ end. Generally, cyanoethyl amidites were used to prepare the PO linkages, DPSE amidites for the PS linkages and PSM amidites for the PN linkages.

A typical MerMade12, 50 umol cycle is outlined in the table below:

| | | | | | Approx. | |
|---|---|---|---|---|---|---|
| Step | Operation | Reagents and Solvent | Volume | | Total Time | |
| 1 | Detritylation | 3% dichloroacetic acid in toluene | 4 × 4.5 | mL | 5 | min |
| 2 | Single | 0.15M phosphoramidite in combinations of | 2.25 mL/1.5 mL | | 5.5 | min |
| | Coupling | ACN/IBN and 0.5M CMIMT in ACN | | | | |
| 3 | Cap 1 | 80% THF/10% 2,6-lutidine/10% acetic anhydride | 5 | mL | 4 | min |
| 4 | Oxidation | 0.02M iodine in 70% THF/20% pyridine/10% water | 6 | mL | 2 | min |
| | (PO) | | | | | |
| | Sulfurization | 0.1M xanthane hydride in 50% pyridine/50% ACN | 5-6 | mL | 6.5 | min |
| | (PS) | | | | | |
| | PN | 0.3M ADIH in ACN | 6 | mL | 6.5 | min |

| | | | | | |
|---|---|---|---|---|---|
| 5 | Cap 2 | 80% THF/10% 2,6-lutidine/10% acetic anhydride | 2.5 mL/2.5 mL | 1.5 | min |

and 16% n-methylimidazole in THF

CMIMT: N-cyanomethylimidazolium triflate;
ACN: acetonitrile;
IBN: isobutyronitrile;
ADIH: 2-azido-4,5-dihydro-1,3-dimethyl-1H-imidazolium hexafluorophosphate;
THF: tetrahydrofuran.
The cycles were performed multiple times until the desired length was achieved.

The GalNAc amidite was coupled either as a single 10-15 min or a two ×10 min procedure. For each coupling, 2.20-2.25 mL of 0.1M GalNAc amidite and 1.5 mL of CMIMT in ACN were added.

The first step of deprotection was performed on the synthesizer. 6 mL of 20% diethylamine in ACN was added to the column for 15 min followed by washing with ACN and drying. CPG was transferred to a tube and 5 mL of fluoride solution was added. The fluoride solution consisted of dimethylsulfoxide, water, triethylamine trihydrofluoride, and triethylamine (15.5/3.1/1.0/1.8 volume ratio). After about 1 hour at room temperature, approximately 10 mL of 30% ammonium hydroxide was added, and the reaction incubated at 37° C. overnight. The CPG was filtered off and washed with water and the filtrate collected.

The oligonucleotides were purified by anion exchange purification at room temperature. The oligonucleotide was loaded onto a column packed with Source Q15 resin after equilibration with a 20 mM sodium hydroxide-based mobile phase. The purified oligonucleotide was eluted as fractions by gradient elution with a mobile phase of 20 mM sodium hydroxide and 2.5 M sodium chloride. Fractions were analyzed, pooled to the desired purity and desalted using a G-25 Sephadex column against water for injection. Desalted samples were dried, reconstituted and sterile filtered prior to final analysis including UPLC, LC-MS and UV-Vis.

A number of oligonucleotide compositions were synthesized and assessed. For example, observed MS data and calculated MS data of oligonucleotides in certain prepared oligonucleotide compositions are illustrated blow; many other oligonucleotide compositions were prepared in accordance with the present disclosure. Oligonucleotides can be further purified.

| ID | Calculated MW | Observed MW |
|---|---|---|
| WV-44515 | 10321.65 | 10322.4 |
| WV-46460 | 10661.04 | 10662.3 |
| WV-50496 | 10672.11 | 10673.0 |
| WV-50497 | 10623.00 | 10623.8 |
| WV-50498 | 10719.12 | 10719.4 |
| WV-46312 | 12110.65 | 12109.8 |
| WV-47606 | 12450.04 | 12447.7 |
| WV-47608 | 12185.70 | 12184.5 |
| WV-49085 | 12462.07 | 12459.7 |
| WV-49086 | 12520.15 | 12517.4 |
| WV-49087 | 12403.03 | 12401.1 |
| WV-49088 | 12461.11 | 12460.2 |
| WV-49089 | 12635.35 | 12635.1 |
| WV-49090 | 12411.10 | 12410.5 |
| WV-49092 | 12508.12 | 12506.2 |

### Example 3. Provided Technologies can Provide Durable Editing In Vivo

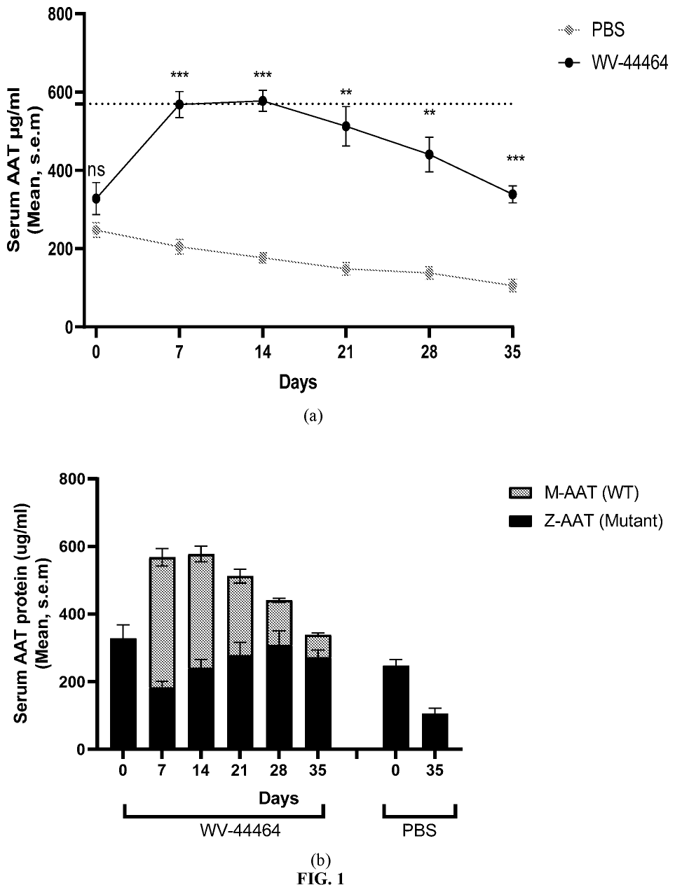

Among other things, provided technologies can provide durable editing in vivo. Certain data are presented in FIG. 48, confirming that provided technologies can provide durable editing in a mouse model. Wild-type and transgenic hADAR mice were treated with PBS or 10 mg/kg of WV-44464 oligonucleotide composition at days 0, 2, and 4. Serum was collected through weekly blood draws and levels of total human AAT protein (total, wild-type (M-AAT), and mutant (Z-AAT)) were quantified by ELISA and mass spectrometry. As shown in FIG. 1, provided technologies can increase total human AAT serum concentration, and can generate or increase wild-type AAT protein (M-AAT). In some embodiments, it was observed AAT serum concentrations were ≥3-fold higher over 30 days post last dose (FIG. 1, (a)). In some embodiments, restored wild type M-AAT was detected over 30 days post last dose (FIG. 1, (b)).

### Example 4. Provided Technologies can Provide Editing

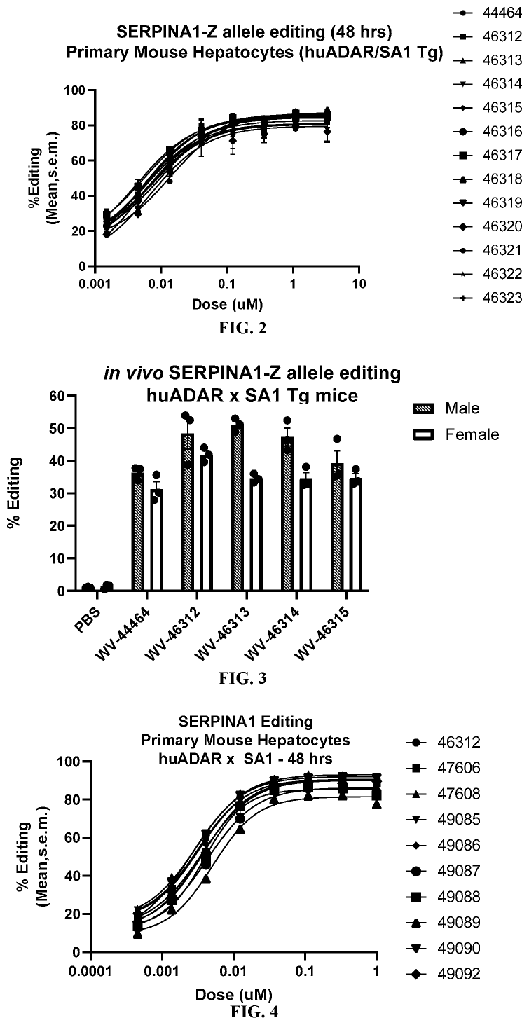

Oligonucleotides comprising various types of sugars, nucleobases, internucleotidic linkages, and stereochemistry and patterns therefor were designed and assessed, confirming that oligonucleotides of various designs can provide efficient editing, including those comprising alternating blocks comprising 2′-F and blocks comprising 2′-OR wherein R is C₁₋₆, aliphatic (2′-OMe and/or 2′-MOE) blocks, natural phosphate linkages, phosphorothioate internucleotidic linkage internucleotidic linkages, non-negatively charged internucleotidic linkages (e.g., phosphoryl guanidine internucleotidic linkages such as n001s), controlled stereochemistry, patterns thereof, etc. as described herein. As shown in FIG. 2 and FIG. 4, oligonucleotides comprising various types of sugars (e.g., DNA sugars, 2′-F modified sugars, 2′-OR modified sugars wherein R is not hydrogen, and patterns thereof), nucleobases (modified and unmodified bases and patterns thereof), internucleotidic linkages (e.g., natural phosphate linkages, non-negatively charged internucleotidic linkages, phosphorothioate internucleotidic linkages, and patterns thereof), and stereochemistry (e.g., Rp, Sp, and patterns thereof) and patterns thereof can provide robust editing activities. Primary mouse hepatocytes transgenic for hADAR p110 and SERPINA1-Z allele were treated with GalNAc-conjugated oligonucleotides via gymnotic uptake. RNA was harvested 48 hours post-treatment and RNA editing was measured by Sanger sequencing (n=2 biological replicates). Certain EC50 (nM) data were provided below (FIG. 2 and FIG. 4):

| | | 95% CI | | | 95% CI |
|---|---|---|---|---|---|
| ID | EC50 | (nM) | ID | EC50 | (nM) |
| WV-44464 | 13.67 | 8.574-21.50 | WV-46323 | 4.951 | 2.790-8.281 |
| WV-46312 | 5.139 | 3.347-7.647 | WV-46312 | 3.52 | 2.98-4.04 |
| WV-46313 | 4.304 | 2.59-6.778 | WV-47606 | 3.38 | 2.78-3.96 |
| WV-46314 | 6.179 | 2.798-12.54 | WV-47608 | 2.44 | 1.91-2.96 |
| WV-46315 | 7.121 | 3.471-13.75 | WV-49085 | 2.70 | 2.13-3.26 |
| WV-46316 | 6.957 | 4.929-9.661 | WV-49086 | 2.81 | 2.28-3.34 |
| WV-46317 | 5.34 | 3.743-7.471 | WV-49087 | 4.53 | 3.61-5.43 |
| WV-46318 | 9.433 | 6.306-13.89 | WV-49088 | 4.03 | 3.23-4.83 |
| WV-46319 | 6.068 | 2.971-11.57 | WV-49089 | 6.14 | 5.06-7.21 |
| WV-46320 | 7.058 | 3.923-12.17 | WV-49090 | 2.46 | 2.14-2.76 |
| WV-46321 | 9.851 | 5.987-15.84 | WV-49092 | 3.47 | 2.97-3.96 |
| WV-46322 | 6.574 | 4.944-8.657 | | | |

### Example 5. Provided Technologies can Provide Editing In Vivo

Oligonucleotides comprising various types of sugars, nucleobases, internucleotidic linkages, and stereochemistry and patterns therefor were designed and assessed, including those comprising alternating blocks comprising 2′-F and blocks comprising 2′-OR wherein R is C₁₋₆ aliphatic (2′-OMe and/or 2′-MOE) blocks, natural phosphate linkages, phosphorothioate internucleotidic linkage internucleotidic linkages, non-negatively charged internucleotidic linkages (e.g., phosphoryl guanidine internucleotidic linkages such as n001s), controlled stereochemistry, patterns thereof, etc. as described herein. Certain data are presented in FIG. 3, confirming that provided technologies can provide robust editing in a mouse model. Male and female transgenic hADAR mice were treated with indicated oligonucleotides at 5 mg/kg via subcutaneous administration at days 0, 2, and 4. Liver biopsies were collected at day 7 post-treatment and RNA editing was measured by Sanger sequencing (n=3 animals per gender). As shown in FIG. 3, provided oligonucleotide compositions can provide high editing levels. In some embodiments, certain oligonucleotide compositions may provide higher editing levels in male mice as compared to female mice.

### Example 6. Provided Technologies can Provide Edited Polypeptides with Desired Properties and Functions In Vivo

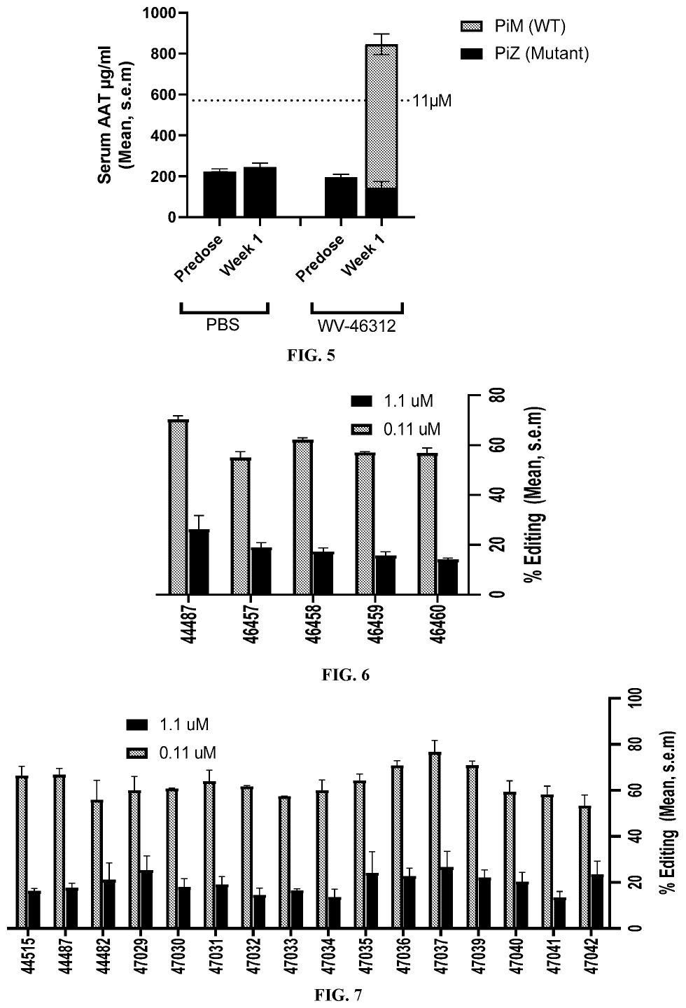

In some embodiments, the present disclosure provides oligonucleotide compositions that can, among other things, provide editing activities in various systems, e.g., in various cells, tissues, and/or organs in vivo and generate polypeptides with desired properties and activities, e.g., in some embodiments, wild-type proteins. Certain data are presented in FIG. 5, confirming that provided technologies in some embodiments can provide editing in a mouse model, and/or can produce increased levels of circulating proteins including wild-type proteins in serum. Wild-type and transgenic hADAR mice were treated with PBS or 10 mg/kg of WV-46312 oligonucleotide composition at days 0, 2, and 4. Serum was collected through weekly blood draws and levels of total human AAT protein (wild-type (PiM), and mutant (PiZ) were quantified by ELISA and mass spectrometry. As shown in FIG. 5, provided technologies can increase AAT serum concentration by about 4-fold or more, and can generate high levels of wild-type AAT in serum, relative to a reference (e.g., pre-dose levels).

### Example 7. Provided Technologies can Provide Editing In Vitro and In Vivo

Among other things, the present example provides data further confirming that provided technologies can provide editing.

FIG. 6 and FIG. 7 provide data confirming that sugar modifications. e.g., 2-OR modifications wherein R is not —H (such as 2′-OMe, 2′-MOE, etc.), 2′-F, etc., can be utilized with various other structural elements in accordance with the present disclosure to provide editing. Primary mouse hepatocytes (huADAR/SA1 Tg) were treated with indicated oligonucleotide compositions targeting SERPINA1-Z allele for 48 hrs (gymnotic). Oligonucleotides comprising various types of linkages (e.g., PS (phosphorothioate), PO (natural phosphate linkage) and/or PN (e.g., phosphoryl guanidine linkages such as n001) internucleotidic linkages) and various types of sugars (e.g., 2′-OMe modified sugars, 2′-MOE modified sugars, 2′-F modified sugars, natural DNA sugars, etc.) were assessed and confirmed to provide editing of target adenosines. In some embodiments, oligonucleotides comprising increased levels of 2′-OMe and/or 2′-MOE modified sugars and PO linkages provide comparable or increased editing of target adenosines relative to a reference at certain conditions. RNA editing was quantified by Sanger sequencing.

Dose response for various oligonucleotide compositions were assessed. Certain results for certain compositions are presented below as examples. Primary mouse (transgenic for humanADARp110 and SERPINA1-Z allele) hepatocytes were treated with indicated oligonucleotide compositions targeting SERPINA1-Z allele for 48 hrs. RNA editing was quantified by Sanger sequencing. Oligonucleotides comprising various modifications were assessed and confirmed to provide editing of target adenosines. Serial dilution concentrations from about 1000 nM to about 0.5 nM. About 15%-40% editing observed at the lowest concentration and about 85% editing observed at the highest concentrations.

| ID | Absolute EC50 (nM) | 95% CI (nM) |
|---|---|---|
| WV-46312 | 7.74 | 1.09-14.39 |
| WV-46313 | 4.19 | 1.88-6.51 |
| WV-47597 | 6.74 | 4.45-9.03 |
| WV-47598 | 7.02 | 3.53-10.52 |
| WV-47599 | 6.73 | 4.44-9.02 |
| WV-47600 | 8.24 | 6.18-10.29 |
| WV-47601 | 5.03 | 3.61-6.45 |
| WV-47602 | 3.76 | 1.32-6.2 |
| WV-47603 | 6.93 | 5.21-8.66 |
| WV-47604 | 8.01 | 6.17-9.85 |
| WV-47605 | 6.98 | 4.01-9.95 |
| WV-47606 | 4.32 | 3.19-5.46 |
| WV-47607 | 4.89 | 1.3-8.48 |
| WV-47608 | 3.26 | 0.41-6.11 |
| WV-47609 | 10.71 | 7.38-14.04 |
| WV-44464 | 10.70 | 6.11-15.29 |

As described herein, oligonucleotides may comprise duplex regions or may be utilized as duplexes. In some embodiments, a duplexing oligonucleotide forms a duplex with an oligonucleotide that can target and edit a target adenosine. Certain examples are presented below as examples. Serial dilution concentrations from about 1000 nM to about 0.5 nM. About 5%-20% editing observed at the lowest concentration and about 70%-90% editing observed at the highest concentrations. Primary mouse hepatocytes (huADAR/SA 1 Tg) were treated with indicated oligonucleotide compositions targeting SERPINA1-Z allele for 48 hrs (gymnotic). Oligonucleotides comprising various types of nucleobases, linkages (e.g., PS (phosphorothioate), P0 (natural phosphate linkage) and/or PN (e.g., phosphoryl guanidine link-ages such as n001) internucleotidic linkages) and sugars (e.g., 2′-Me modified sugars, 2′-F modified sugars, natural DNA sugars, 2′-MOE modified sugars, etc.) can form duplexes with corresponding duplexing oligonucleotides. Certain duplexes were assessed as examples and were confirmed to provide editing of target adenosines. RNA editing was quantified by Sanger sequencing. In some embodiments, certain duplexes provided comparable or increased editing activity relative to a reference. In some embodiments, duplexing oligonucleotide comprise 2′-OR modified sugars (wherein R is not —H, e.g., 2′-Me modified sugars, 2′-MOE modified sugars, etc.) and/or modified internucleotidic linkages (e.g., phosphorothioate internucleotidic linkage) at both ends. In some embodiments, duplexing oligonucleotides comprise 2′-F modified sugars, 2′-OR modified sugars (wherein R is not —H, e.g., 2′-Me modified sugars, 2′-MOE modified sugars, etc.) and/or natural RNA sugars. In some embodiments, it was observed that duplexing oligonucleotide comprising internal natural RNA sugars may provide higher editing efficiency when duplexed with targeting oligonucleotides (e.g., WV-46312).

| Oligonucleotides | Absolute EC50 | 95% CI |
|---|---|---|
| (WV-) | (nM) | (nM) |
| 46312 | 3.90 | 2.25-5.54 |
| 46312/48444 | 8.92 | 7.72-10.1 |
| 46312/48445 | 5.71 | 4.89-6.52 |
| 46312/48446 | 2.91 | 2.32-3.50 |
| 46312/48447 | 9.68 | 8.36-10.9 |
| 46312/48448 | 6.58 | 5.83-7.33 |
| 46312/48449 | 3.08 | 2.47-3.68 |
| 46312/48450 | 16.66 | 14.4-18.8 |
| 46312/48451 | 8.83 | 7.92-9.73 |
| 46312/48452 | 3.09 | 2.64-3.53 |

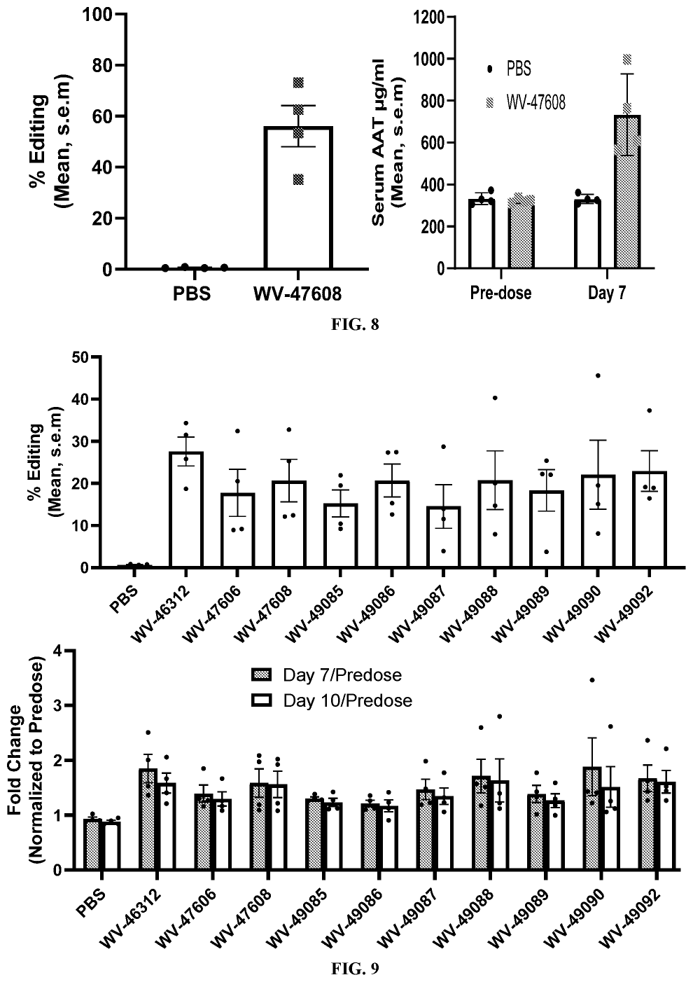

As described herein, provided technologies provide editing in vivo and can provide products, e.g., polypeptides, encoded by edited nucleic acids. For example, FIG. 8 confirms in vivo editing of SERPINA1 and increase of serum AAT levels. Mice transgenic for human ADAR and SERPINA1-Z allele were subcutaneously dosed with PBS or 10 mg/kg oligonucleotide on days 0, 2, and 4. Liver biopsies were collected at day 7 and serum AAT was collected pre-dose and day 7. As confirmed in FIG. 8, provided oligonucleotide compositions delivered significant editing activity and increased levels of serum AAT relative to reference (e.g., PBS control, pre-dose levels). Serum AAT was quantified using ELISA. Certain additional results are presented in FIG. 9, which confirms that various modifications can be utilized in accordance with the present disclosure to provide oligonucleotides that are active in vivo. Mice transgenic for human ADAR and SERPINA1-Z allele were subcutaneously dosed with PBS or 10 mg/kg oligonucleotides on day 0. Liver biopsies were collected at day 10. Serum was collected pre-dose, day 7, and day 10. Various oligonucleotide compositions were assessed and confirmed to provide editing of target adenosines and increased levels of serum AAT. RNA editing was quantified by Sanger sequencing. Serum AAT was quantified using ELISA.

### Example 8. Provided Technologies can Provide In Vivo Editing and Increase AAT Protein Levels

Among other things, provided technologies can provide in vivo editing. In some embodiments, provided technologies can edit transcripts from SERPINA1 PiZ allele to correct 1024 G>A (E342K) mutation. As confirmed herein, in some embodiments, provided technologies can increase serum AAT levels including to levels that can be therapeutically useful.

For example, in some embodiments, oligonucleotides WV-46312, WV-49090 and WV-49092 were assessed in NSG-PiZ mice (in some mice, PBS as reference). Efficient editing and production of M-AAT protein were confirmed. Oligonucleotides were administered at 10 mg/kg subcutaneously every two weeks in 7-week old NSG-PiZ mice (n=5 per group). For some mice, 3×10 mg loading doses (days 0, 2, 4 at week 0) were administered. Data confirmed that at week 13, about 50% or more editing was observed with or without loading doses in liver biopsies (SERPINA1 editing was quantified by Sanger sequencing; one-way ANOVA with adjustment for multiple comparisons (Tukey)). Total serum AAT protein was quantified by ELISA weekly. Compared to PBS, serum AAT protein levels were increased at each time point when serum AAT protein levels were assessed (up to at least about 13 weeks). In some embodiments, it was observed that serum AAT levels assessed on odd-numbered weeks (1 week after a dose, e.g., week 1, week 3, week 5, etc.) were higher than those assessed on the following even-numbered weeks (two weeks after a dose, e.g., week 2, week 4, week 6, etc.). With loading doses, serum AAT levels were increased to about 600 μg/mL or more (e.g., in some embodiments, about 600, 800, 1000, 1200, 1400, or 1600 μg/mL or more) at each time point (total serum AAT protein quantified by ELISA; Two-Way ANOVA with adjustment for multiple comparisons (Tukey)). In some embodiments, about 4-7 fold or more increases in serum AAT protein levels were observed at, e.g., week 13. In some embodiments, provided technologies can provide effective editing with or without loading doses. In some embodiments, it was observed that loading doses provided higher levels of editing at early time points (e.g., weeks 1-3), and after a few weeks, e.g., week 4, no significant difference in serum AAT levels was observed with or without loading doses, confirming that provided technologies can be utilized either with or without loading doses, e.g., according to desired effects.

Certain oligonucleotides were assessed in another animal model, mice transgenic for hADAR and SERPINA1-Z allele. Among other things, editing and increased serum AAT protein levels were confirmed. For example, WV-46312 was administered subcutaneously to mice 8-10 weeks old 10 mg/kg every two weeks (and also on days 0, 2, and 4 as loading doses). Total serum AAT protein was quantified by ELISA weekly or every two weeks. Compared to mice that were administered PBS, serum AAT protein levels were increased at all assessed time points in mice that were administered WV-46312. In some embodiments, it was observed that serum AAT levels assessed on odd-numbered weeks (1 week after a dose, e.g., week 1, week 3, week 5, etc.) were higher than those assessed on the following even-numbered weeks (two week after a dose, e.g., week 2, week 4, week 6, etc.). In a study, on several weeks, and consistently after week 9 and up to week 19, the last week in an analysis (one week after the dose at week 18), serum AAT levels were increased to about 600 μg/mL or more (in some embodiments, about 800 μg/mL or more). In some embodiments, about 5 fold or more increase in serum AAT protein level was observed at week 19. In some embodiments, about 60% RNA editing was observed at week 19 (assessed by Sanger sequeneing of samples from liver biopsies; no editing in PBS treatment). As demonstrated previously and confirmed again. M-AAT was produced: in some embodiments, it was observed about 70% of serum AAT was M-AAT (assessed by mass spectrometry of samples from liver biopsies). A significant increase in neutrophil elastase inhibition was also confirmed as compared to PBS (e.g., for PBS, average % relative elastase inhibition pre-dose was about 25% and at week 19 was under 20%; for WV-46312, average % relative elastase inhibition pre-dose was about 25% and at week 19 was over 60%; mixed-effects analysis, p<0.001 at week 19) using an available neutrophil elastase inhibition assay and mouse serum samples. It was confirmed that % PAS-D positive areas can be significantly reduced when compared to PBS (e.g., for PBS, average % PAS-D positive area over 5% at weeks 4, 8 and 19; for WV-46312, all below 5% at these time points: 2-way ANOVA, p<0.01). Data confirmed reduction of lobular inflammation; for example, at week 19, lobular inflammation grade was about 1 or lower for WV-46312 compared to about 3 or higher for PBS (Grade based on the number of inflammatory foci in lobules: Grade 0: 0; Grade 1: 1-5; Grade 2: 6-10; Grade 3: 11-15; and G4≥16; with HALO Image Analysis software; Wilcox rank-sum test, p=0.03) and reduction of mean globular diameter (PAS-D globule size, 40 largest globules per animal; with HALO Image Analysis software; Wilcox rank-sum test, p<0.0001). Similar serum AAT protein increases and patterns were observed in another study using WV-46312. WV-49090 and WV-49092, in which significant increases from week 10 to week 11 to about 600 μg/mL serum AAT protein were observed.

Toxicity of various oligonucleotides were assessed including in mice and NHP. For example. WV-49090 were assessed in various assays including in mice and NHP and was well tolerated and demonstrated good safety profile. PK data confirmed that GalNAc conjugation can deliver oligonucleotides. Certain data are presented below as examples.

| ID | Mass (observed) | Mass (Tgt) |
|---|---|---|
| WV-46312 | 10315.4 | 10315.42 |
| WV-49088 | 10665.77 | 10665.78 |
| WV-49090 | 10616.76 | 10616.78 |
| WV-49092 | 10712.77 | 10712.78 |

### Example 9. Provided Technologies can Provide Editing in Human Hepatocytes Comprising PiZ Allele

Among other things, it was confirmed the provided technologies can effectively edit transcripts from a PiZ allele in primary human hepatocytes. In a study, WV-46312, WV-49090, and WV-49092 were assessed at multiple concentrations: 0.33, 0.11, 0.03667, 0.01222, 0.00407, 0.00136, 0.00045, and 0.00015 uM, and editing was confirmed at each concentration, including at 0.00015 uM (about 55% or more % M-AAT RNA for all three oligonucleotides, compared to about 47% observed for an oligonucleotide targeting 1024 G>A (E342K) mutation in SERPINA1 but without GalNAc which oligonucleotide provided about 47% to about 74% % M-AAT RNA across the concentrations). For all three oligonucleotides, about 80% or more % M-AAT RNA was observed at about 0.01222 uM or higher concentrations. EC75 from certain assessments are presented below as examples:

| ID | Absolute EC75 (nM) | 95% CI (nM) |
|---|---|---|
| WV-46312 | 3.75 | 1.41-6.08 |
| WV-49090 | 2.47 | 1.05-3.89 |
| WV-49092 | 2.58 | 1.11-4.04 |

As confirmed herein, provided technologies can provide effective editing of RNA comprising 1024 G>A (E342K) mutation in SERPINA1.

### Example 10. Provided Technologies can Provide Editing of Target Transcripts

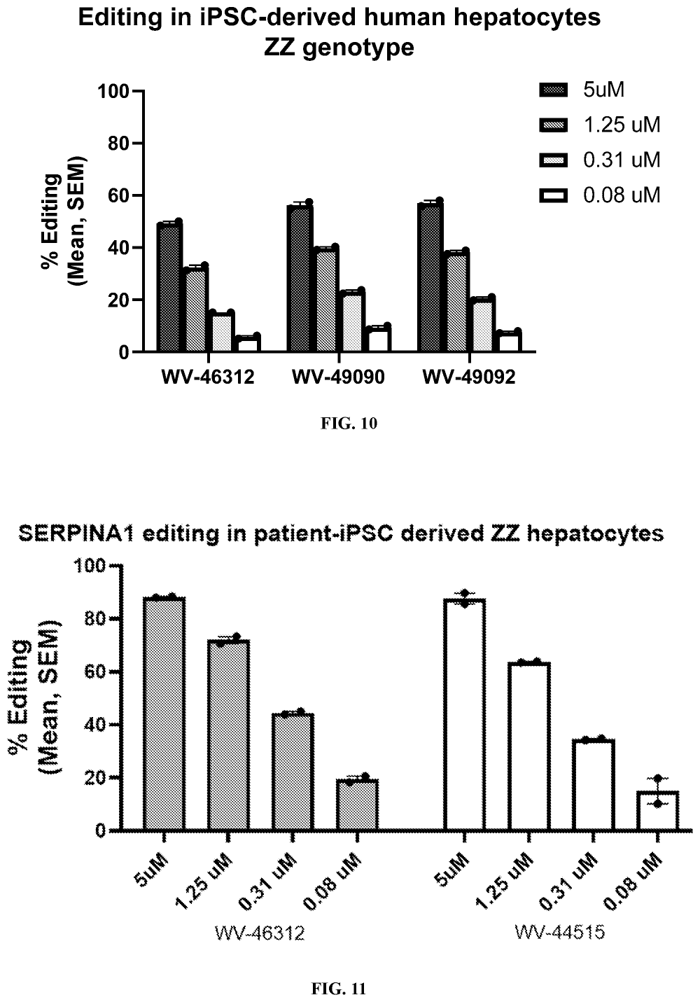

Among other things, it was confirmed the provided technologies can effectively edit transcripts from a SERPINA1 PiZ allele in human patient induced pluripotent stem cell (iPSC)-derived hepatocytes. Human patient iPSC-derived hepatocytes with the ZZ genotype were plated on day 0 and treated on day 2 with the indicated oligonucleotides (e.g., WV-46312, WV-49090, WV-49092) at various concentrations (e.g., 5, 1.25, 0.31, 0.08 uM). Media was refreshed every 2 days (e.g., on days 2, 4, 6, 8). RNA was collected on day 10 and RNA editing was quantified by Sanger sequeneing. As confirmed in FIG. 10, various oligonucleotides (e.g., WV-46312, WV-49090, WV-49092) provided editing of SERPINA1 transcripts (e.g., about 50%-60% % editing in cells treated with 5 uM of indicated oligonucleotides). In some embodiments, certain oligonucleotide compositions provide higher editing than others at specific concentrations. In some embodiments, certain oligonucleotide compositions provide dose-dependent editing of target transcripts.

### Example 11. Provided Technologies can Provide Editing of Target Transcripts

Among other things, it was confirmed the provided technologies can effectively edit transcripts from a SERPINA1 PiZ allele in human patient induced pluripotent stem cell (iPSC)-derived hepatocytes. Human patient iPSC-derived hepatocytes with the ZZ genotype were plated on day 0 and treated on day 2 with the indicated oligonucleotides (e.g., WV-46312, WV-44515) at various concentrations (e.g., 5, 1.25, 0.31, 0.08 uM). Media was changed every 2 days (e.g., on days 4, 6, 8) and oligonucleotides were redosed every 2 days (e.g., on days 4, 6, 8). RNA was collected on day 10 and RNA editing was quantified by Sanger sequeneing. As confirmed in FIG. 11, various oligonucleotides (e.g., WV-46312, WV-44515) provided editing of SERPINA1 transcripts (e.g., about 80-85% or more % editing in cells treated with 5 uM of indicated oligonucleotides). Oligonucleotide compositions with and without GalNAc conjugation provided editing of SERPINA1 transcripts. Patient iPSC-derived hepatocytes reportedly have low expression of asialoglycoprotein receptor (ASGPR), which, without the intention to be limited by any particular theory, may impact effects of GalNAc conjugation in this system. In some embodiments, provided technologies provide editing with or without GalNAc conjugation. In some embodiments, certain oligonucleotide compositions provide higher editing than others at specific concentrations. In some embodiments, certain oligonucleotide compositions provide dose-dependent editing of target transcripts.

### Example 12. Provided Technologies can Provide In Vivo Editing and Increase AAT Protein Levels

Among other things, provided technologies can provide in vivo editing. In some embodiments, provided technologies can edit transcripts from SERPINA1 PiZ allele to correct a 1024 G>A (E342K) mutation. As confirmed herein, in some embodiments, provided technologies can increase serum M-AAT levels including to levels that can be therapeutically useful.

For example, in some embodiments, oligonucleotide WV-49090 was assessed in NSG-PiZ mice. In some mice, PBS was used as a reference. Efficient editing, production of M-AAT, and functioning of wild-type (M) AAT protein were confirmed. Oligonucleotides were administered at 10 mg/kg subcutaneously every two weeks in 7-week old NSG-PiZ mice (JAX stock #028842; N=5 per treatment group). One group of mice received loading doses during week 1 (on days 0, 2, 4), while the other group received a single dose during week 1 (on day 0). The groups subsequently received additional doses every 2 weeks (e.g., during week 2, 4, 6, 8, 10, 12). A control group of mice received PBS. Serum was collected from mice prior to dosing and at week 13 following treatment. Mouse liver biopsies were collected on week 0 for baseline measurements or on week 13 following treatment. RNA was collected from the liver biopsies. Relative level of SERPINA1 mRNA was determined by qPCR. Editing of SERPINA1 was quantified by Sanger sequeneing. Relative abundance of Z (mutant) vs. M (wild-type) AAT isoforms was determined by liquid chromatography-mass spectrometry (LC-MS). Relative elastase inhibition activity in serum was determined in an in vitro reaction using a commercially available kit.

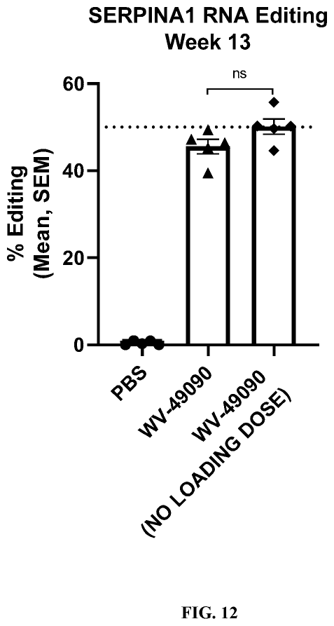

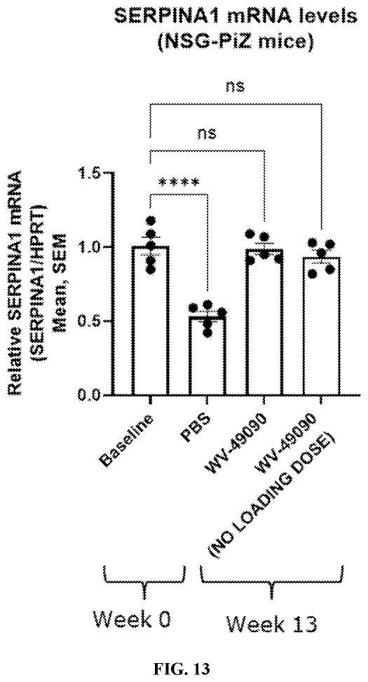

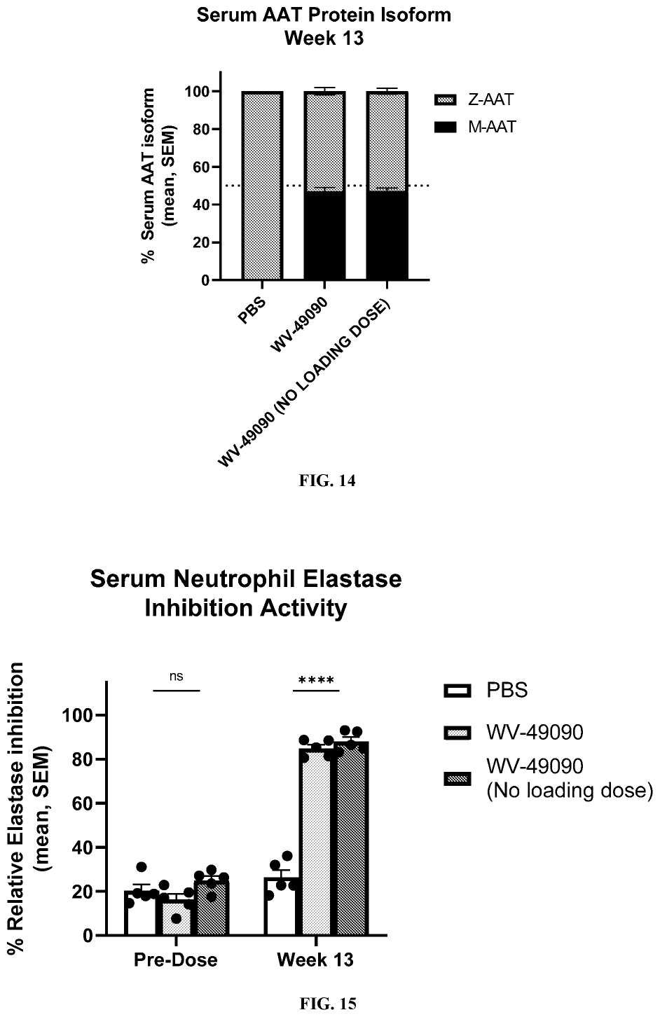

As confirmed in FIG. 12, at week 13, editing of SERPINA1 transcripts was confirmed in mice administered WV-49090 (with or without loading doses), e.g., about 45 to 50% editing, compared to about 0% editing observed for PBS. Further, as shown in FIG. 13, relative SERPINA1 mRNA levels were observed to be maintained at week 13 in mice administered WV-49090 (loading dose and no loading dose) as compared to baseline relative SERPINA1 mRNA levels at week 0. In contrast, a significant decrease (e.g., about 50%) in relative SERPINA1 mRNA levels was observed in mice administered PBS. As confirmed in FIG. 14, at week 13, about 50% of total serum AAT protein was the M-AAT isoform in mice administered WV-49090 (loading dose and no loading dose) as compared to 100% of total serum AAT protein being the Z-AAT isoform in mice administered PBS. Additionally, as displayed in FIG. 15, at week 13, a significant increase in neutrophil elastase inhibition in mice administered WV-49090 (loading dose and no loading dose) was confirmed as compared to PBS. For example, for PBS, % relative elastase inhibition was about 20% pre-dose and about 25% at week 13, while for WV-49090 (loading dose and no loading dose), % relative elastase inhibition was about 20% pre-dose and about 80% at week 13. In some embodiments, provided technologies can provide editing of target transcripts, e.g., SERPINA1 RNA, with or without loading doses. In some embodiments, provided technologies can increase SERPINA1 mRNA levels, with or without loading doses. In some embodiments, provided technologies can increase serum M-AAT levels with or without loading doses. In some embodiments, provided technologies can increase serum neutrophil elastase inhibition activity with or without loading doses.

### Example 13. Provided Technologies can Provide Editing In Vivo

Among other things, provided technologies can provide in vivo editing and increases in serum AAT levels. For example, in some embodiments, oligonucleotides (e.g., WV-46312, WV-49090, WV-49092) were confirmed to provide editing of transcripts from the SERPINA1 PiZ allele and increase serum AAT protein levels in human ADAR (huADAR) transgenic mice. Oligonucleotides were subcutaneously administered as a single dose (e.g., 10 mg/kg, 30 mg/kg, or 100 mg/kg) on day 0 in huADAR/SA-1 Tg mice, and subsequent collection of serum occurred on days 0 (pre-dose), 7, 14, 21, and 28 and liver biopsies were collected on days 7 (for 30 mg/kg) and 30 (for 10 mg/kg, 30 mg/kg, 100 mg/kg) (N=8 per group). In some mice, PBS was administered as a reference. RNA was collected from the liver biopsies. Liver biopsies were also examined for presence of oligonucleotide to determine ug of oligo/g of tissue. Editing of SERPINA1 was quantified by Sanger sequeneing. Serum AAT levels were quantified by ELISA. For all three oligonucleotides, about 45% or more editing was observed at day 7 when dosed with 30 mg/kg of the oligonucleotide composition. Further, for all three oligonucleotides, about 2.1 or higher fold change in serum AAT protein levels was observed at day 7 when dosed with 100 mg/kg of the oligonucleotide composition.

Mean % RNA editing single dose on day 0 is shown in the table below as examples:

| Dose | Day Assessed | WV-46312 | WV-49090 | WV-49092 |
|---|---|---|---|---|
| 10 mg/kg | 28 | 2.5% (sem: 0.5) | 4.0% (sem: 0.9) | 7.0% (sem: 1.2) |
| 30 mg/kg | 7 | 60.1% (sem: 4.8) | 46.3% (sem: 6.8) | 61.6% (sem: 3.9) |
| 30 mg/kg | 28 | 3.8% (sem: 0.7) | 7.1% (sem: 1.2) | 11.8% (sem: 3.1) |
| 100 mg/kg | 28 | 10.0% (sem: 1.5) | 16.5% (sem: 4.1) | 18.0% (sem: 2.7) |

sem: standard error of the mean.

Fold change in serum AAT after single dose on day 0, relative to pre-dose levels, is shown in the table below as examples:

| Dose | Day Assessed | WV-46312 | WV-49090 | WV-49092 |
|---|---|---|---|---|
| 10 mg/kg | 7 | 1.6 (sem: 0.1) | 1.5 (sem: 0.1) | 1.8 (sem: 0.1) |
| 30 mg/kg | 7 | 1.8 (sem: 0.1) | 2.0 (sem: 0.2) | 2.4 (sem: 0.1) |
| 100 mg/kg | 7 | 2.3 (sem: 0.2) | 2.7 (sem: 0.3) | 2.1 (sem: 0.2) |

sem: standard error of the mean.

Certain pharmacokinetic (PK)/pharmacodynamic (PD) data is shown in the table below as examples:

| | t_{1/2} | EC50 | EC50^{a} | V | |
|---|---|---|---|---|---|
| Compound | (Days) | (μg/g) | (μM) | (mL/kg) | a^{b} |
| WV-46312 | 4.00 | 7.04 | 0.682 | 143 | 1.01 |
| WV-49090 | 7.10 | 19.6 | 1.85 | 229 | −0.083 |
| WV-49092 | 7.54 | 7.96 | 0.743 | 248 | 0.249 |

t_{1/2}: half-life in liver tissue.
^{a}Average base weight used for conversion to μM.
^{b}Exponent for the power function between dose and bioavailability.

As confirmed herein, provided technologies can provide effective editing of transcripts from SERPINA1 PiZ allele. As confirmed herein, provided technologies can provide increases in ser um AAT protein.

While various embodiments have been described and illustrated herein, those of ordinary skill in the art will readily envision a variety of other means and/or structures for performing the functions and/or obtaining the results and/or one or more of the advantages described in the present disclosure, and each of such variations and/or modifications is deemed to be included. More generally, those skilled in the art will readily appreciate that all parameters, dimensions, materials, and configurations described herein arc meant to be example and that the actual parameters, dimensions, materials, and/or configurations may depend upon the specific application or applications for which the teachings of the present disclosure is/are used. Those skilled in the art will recognize, or be able to ascertain using no more than routine experimentation, many equivalents to the embodiments of the present disclosure. It is, therefore, to be understood that the foregoing embodiments are presented by way of example only and that, within the scope of the appended claims and equivalents thereto, claimed technologies may be practiced otherwise than as specifically described and claimed. In addition, any combination of two or more features, systems, articles, materials, kits, and/or methods, if such features, systems, articles, materials, kits, and/or methods are not mutually inconsistent, is included within the scope of the present disclosure.

1-88. (canceled)

89. An oligonucleotide, wherein the oligonucleotide is mCn001RmC*SmC*SfA*SfG*Sm5CeoAeofG*SfC*SmUmUn001RmCfA*SfGn001RfUm5Ceo*SfC*SmCmUn001RmUTeofC*ST*Sb008U*SIn001SmUfC*SmG*SmAn001RmU (SEQ ID NO: 14) or a salt thereof, wherein:
f represents a 2′-F modification to a nucleoside;
m represents a 2′-OMe modification to a nucleoside;
eo represents a 2′-OCH₂CH₂OCH₃ modification to a nucleoside;
m5Ceo represents 5-methyl 2′-O-methoxyethyl C;
n001R represents a Rp n001 linkage;
n001S represents a Sp n001 linkage;
a n001 linkage is 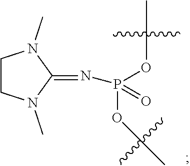 [SMILES: *OP(=O)(N=C1N(C)CCN1C)O*]
*S represents a Sp phosphorothioate linkage;
I represents the nucleobase is hypoxanthine; and
b008U represents a nucleoside whose base is

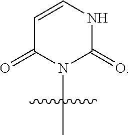 [SMILES: *n1c(=O)cc[nH]c1=O]

90. The oligonucleotide of claim 89, wherein the diastereopurity of the oligonucleotide is about or at least about (DS)^{nc}, wherein DS is about 90% and nc is the number of chiral linkage phosphorus.

91. The oligonucleotide of claim 90, wherein the oligonucleotide is a pharmaceutically acceptable salt.

92. A pharmaceutical composition, comprising an oligonucleotide of claim 89 and a pharmaceutically acceptable carrier.

93. A method selected from:
(1) a method for modifying a target adenosine in a target nucleic acid, comprising contacting the target nucleic acid with an oligonucleotide, wherein the target adenosine is 1024 G>A in human SERPINA1;
(2) a method for producing, or restoring or increasing level of a product of a particular nucleic acid, comprising contacting a target nucleic acid with an oligonucleotide, wherein the target nucleic acid comprises a target adenosine, and the particular nucleic acid differs from the target nucleic acid in that the particular nucleic acid has an I or G instead of the target adenosine, wherein the target nucleic acid is a human SERPINA1 transcript with a 1024 G>A mutation, and the target adenosine is 1024 G>A in human SERPINA1;
(3) a method for reducing level of a product of a target nucleic acid, comprising contacting a target nucleic acid with an oligonucleotide, wherein the target nucleic acid comprises a target adenosine, wherein the target nucleic acid is a human SERPINA1 transcript with a 1024 G>A mutation, and the target adenosine is 1024 G>A in human SERPINA1;
(4) a method for reducing Z-AAT in liver of a subject, comprising administering or delivering to the subject an effective amount of an oligonucleotide, wherein the subject comprises 1024 G>A (E342K) mutation in human SERPINA1;
(5) a method for reducing liver inflammation in a subject, comprising administering or delivering to the subject an effective amount of an oligonucleotide, wherein the subject comprises 1024 G>A (E342K) mutation in human SERPINA1;
(6) a method for inhibiting elastase in a subject, comprising administering or delivering to the subject an effective amount of an oligonucleotide, wherein the subject comprises 1024 G>A (E342K) mutation in human SERPINA1;
(7) a method for increasing levels and/or activities of an alpha-1 antitrypsin (A1AT) polypeptide in the serum or blood of a subject, comprising administering or delivering to the subject an effective amount of an oligonucleotide, wherein the subject comprises 1024 G>A (E342K) mutation in human SERPINA1; and
(8) a method for decreasing levels and/or activities of a mutant alpha-1 antitrypsin (A1AT); and polypeptide in the serum or blood of a subject, comprising administering or delivering to the subject an effective amount of an oligonucleotide, wherein the subject comprises 1024 G>A (E342K) mutation in human SERPINA1;
wherein the oligonucleotide is an oligonucleotide of claim 89.

94. A method for preventing or treating a condition, disorder or disease, comprising administering or delivering to a subject susceptible thereto or suffering therefrom an effective amount of an oligonucleotide of claim 89, wherein the subject comprises 1024 G>A (E342K) mutation in human SERPINA1.

95. A method for preparing an oligonucleotide of claim 89, comprising coupling a phosphoramidite comprising a chiral auxiliary with a hydroxyl group.

96. A conjugate, which is a conjugate of an oligonucleotide with an additional chemical moiety or a salt thereof, wherein:
the oligonucleotide is mCn001RmC*SmC*SfA*SfG*Sm5CeoAeofG*SfC*SmUmUn001RmCfA*SfGn001RfUm5Ceo*SfC*SmCmUn001RmUTeofC*ST*Sb008U*SIn001SmUfC*SmG*SmAn001RmU (SEQ ID NO: 14) or a salt thereof, wherein:
f represents a 2′-F modification to a nucleoside;
m represents a 2′-OMe modification to a nucleoside;
eo represents a 2′-OCH₂CH₂OCH₃ modification to a nucleoside;
m5Ceo represents 5-methyl 2′-O-methoxyethyl C;
n001R represents a Rp n001 linkage;
n001S represents a Sp n001 linkage;
a n001 linkage is 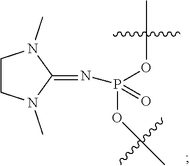 [SMILES: *OP(=O)(N=C1N(C)CCN1C)O*]
*S represents a Sp phosphorothioate linkage;
I represents the nucleobase is hypoxanthine; and
b008U represents a nucleoside whose base is 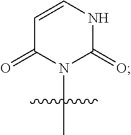 [SMILES: *n1c(=O)cc[nH]c1=O]
and
the additional chemical moiety comprises a ligand for an asialoglycoprotein receptor.

97. The conjugate of claim 96, wherein the additional chemical moiety comprises GalNAc.

98. The conjugate of claim 96, wherein the additional chemical moiety comprises three GalNAc.

99. The conjugate of claim 98, wherein the additional chemical moiety is

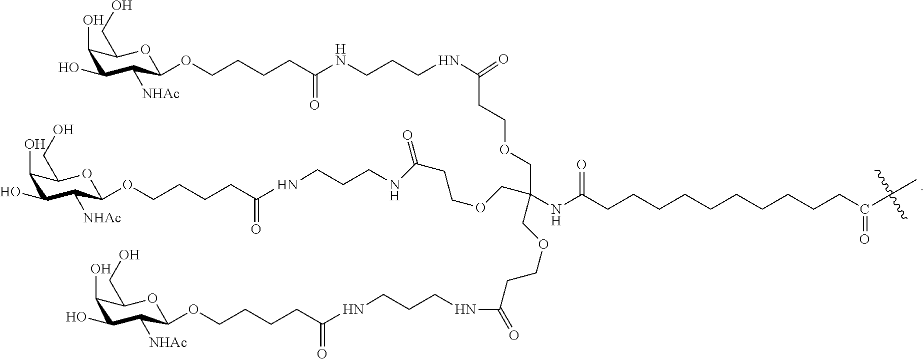

100. The conjugate of claim 96, wherein the diastereopurity of the conjugate is about or at least about (DS)^{nc}, wherein DS is about 90% and nc is the number of chiral linkage phosphorus in the oligonucleotide.

101. The conjugate of claim 96, wherein the conjugate is in a pharmaceutically acceptable salt form.

102. A pharmaceutical composition, comprising a conjugate of claim 96 and a pharmaceutically acceptable carrier.

103. A method selected from:
(1) a method for modifying a target adenosine in a target nucleic acid, comprising contacting the target nucleic acid with a conjugate, wherein the target adenosine is 1024 G>A in human SERPINA1;
(2) a method for producing, or restoring or increasing level of a product of a particular nucleic acid, comprising contacting a target nucleic acid with a conjugate, wherein the target nucleic acid comprises a target adenosine, and the particular nucleic acid differs from the target nucleic acid in that the particular nucleic acid has an I or G instead of the target adenosine, wherein the target nucleic acid is a human SERPINA1 transcript with a 1024 G>A mutation, and the target adenosine is 1024 G>A in human SERPINA1;
(3) a method for reducing level of a product of a target nucleic acid, comprising contacting a target nucleic acid with a conjugate, wherein the target nucleic acid comprises a target adenosine, wherein the target nucleic acid is a human SERPINA1 transcript with a 1024 G>A mutation, and the target adenosine is 1024 G>A in human SERPINA1;
(4) a method for reducing Z-AAT in liver of a subject, comprising administering or delivering to the subject an effective amount of a conjugate, wherein the subject comprises 1024 G>A (E342K) mutation in human SERPINA1;
(5) a method for reducing liver inflammation in a subject, comprising administering or delivering to the subject an effective amount of a conjugate, wherein the subject comprises 1024 G>A (E342K) mutation in human SERPINA1;
(6) a method for inhibiting elastase in a subject, comprising administering or delivering to the subject an effective amount of a conjugate, wherein the subject comprises 1024 G>A (E342K) mutation in human SERPINA1;
(7) a method for increasing levels and/or activities of an alpha-1 antitrypsin (A1AT) polypeptide in the serum or blood of a subject, comprising administering or delivering to the subject an effective amount of a conjugate, wherein the subject comprises 1024 G>A (E342K) mutation in human SERPINA1; and
(8) a method for decreasing levels and/or activities of a mutant alpha-1 antitrypsin (A1AT) polypeptide in the serum or blood of a subject, comprising administering or delivering to the subject an effective amount of a conjugate, wherein the subject comprises 1024 G>A (E342K) mutation in human SERPINA1;
wherein the conjugate is a conjugate of claim 96.

104. A method for preventing or treating a condition, disorder or disease, comprising administering or delivering to a subject susceptible thereto or suffering therefrom an effective amount of a conjugate of claim 96, wherein the subject comprises 1024 G>A (E342K) mutation in human SERPINA1.

105. A method for preparing an oligonucleotide of claim 96, comprising coupling a phosphoramidite comprising a chiral auxiliary with a hydroxyl group.

106. A method for delivering to a system an oligonucleotide of claim 89, comprising administering to the system a conjugate of the oligonucleotide with one or more ligands for an asialoglycoprotein receptor.

107. An oligonucleotide, wherein:
the oligonucleotide is Mod001L001mCn001RmC*SmC*SfA*SfG*Sm5CeoAeofG*SfC*SmUmUn001RmCfA*SfGn001RfUm5Ceo*SfC*SmCmUn001RmUTeofC*ST*Sb008U*SIn001SmUfC*SmG*SmAn001RmU (SEQ ID NO: 13) or a salt thereof, wherein:
Mod001 represents 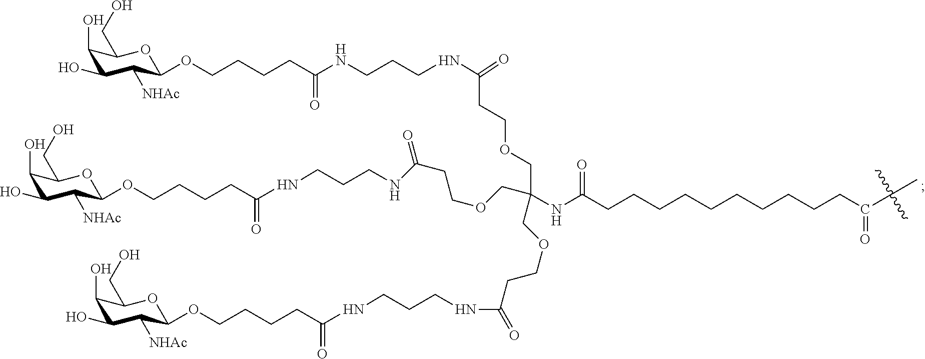
L001: —NH—(CH₂)₆—, connected to Mod001 through —NH— and the 5′-end of the oligonucleotide chain through a phosphate linkage;
f represents a 2′-F modification to a nucleoside;
m represents a 2′-OMe modification to a nucleoside;
eo represents a 2′-OCH₂CH₂OCH₃ modification to a nucleoside;
m5Ceo represents 5-methyl 2′-O-methoxyethyl C;
n001R represents a Rp n001 linkage;
n001S represents a Sp n001 linkage;
a n001 linkage is 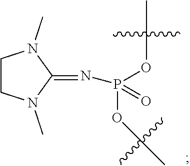 [SMILES: *OP(=O)(N=C1N(C)CCN1C)O*]
*S represents a Sp phosphorothioate linkage;
I represents the nucleobase is hypoxanthine; and
b008U represents a nucleoside whose base is

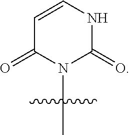 [SMILES: *n1c(=O)cc[nH]c1=O]

108. The oligonucleotide of claim 107, wherein the diastereopurity of the oligonucleotide is about or at least about (DS)^{nc}, wherein DS is about 90% and nc is the number of chiral linkage phosphorus.

109. The oligonucleotide of claim 108, wherein the oligonucleotide is a sodium salt.

110. A pharmaceutical composition, comprising an oligonucleotide of claim 109 and a pharmaceutically acceptable carrier.

111. A method selected from:
(1) a method for modifying a target adenosine in a target nucleic acid, comprising contacting the target nucleic acid with an oligonucleotide, wherein the target adenosine is 1024 G>A in human SERPINA1;
(2) a method for producing, or restoring or increasing level of a product of a particular nucleic acid, comprising contacting a target nucleic acid with an oligonucleotide, wherein the target nucleic acid comprises a target adenosine, and the particular nucleic acid differs from the target nucleic acid in that the particular nucleic acid has an I or G instead of the target adenosine, wherein the target nucleic acid is a human SERPINA1 transcript with a 1024 G>A mutation, and the target adenosine is 1024 G>A in human SERPINA1;
(3) a method for reducing level of a product of a target nucleic acid, comprising contacting a target nucleic acid with an oligonucleotide, wherein the target nucleic acid comprises a target adenosine, wherein the target nucleic acid is a human SERPINA1 transcript with a 1024 G>A mutation, and the target adenosine is 1024 G>A in human SERPINA1;
(4) a method for reducing Z-AAT in liver of a subject, comprising administering or delivering to the subject an effective amount of an oligonucleotide, wherein the subject comprises 1024 G>A (E342K) mutation in human SERPINA1;
(5) a method for reducing liver inflammation in a subject, comprising administering or delivering to the subject an effective amount of an oligonucleotide, wherein the subject comprises 1024 G>A (E342K) mutation in human SERPINA1;
(6) a method for inhibiting elastase in a subject, comprising administering or delivering to the subject an effective amount of an oligonucleotide, wherein the subject comprises 1024 G>A (E342K) mutation in human SERPINA1;
(7) a method for increasing levels and/or activities of an alpha-1 antitrypsin (A1AT) polypeptide in the serum or blood of a subject, comprising administering or delivering to the subject an effective amount of an oligonucleotide, wherein the subject comprises 1024 G>A (E342K) mutation in human SERPINA1; and
(8) a method for decreasing levels and/or activities of a mutant alpha-1 antitrypsin (A1AT) polypeptide in the serum or blood of a subject, comprising administering or delivering to the subject an effective amount of an oligonucleotide, wherein the subject comprises 1024 G>A (E342K) mutation in human SERPINA1;
wherein the oligonucleotide is an oligonucleotide of claim 109.

112. A method for preventing or treating a condition, disorder or disease, comprising administering or delivering to a subject susceptible thereto or suffering therefrom an effective amount of an oligonucleotide of claim 109, wherein the subject comprises 1024 G>A (E342K) mutation in human SERPINA1.

113. A method for preparing an oligonucleotide of claim 109, comprising coupling a phosphoramidite comprising a chiral auxiliary with a hydroxyl group.

114. A compound, wherein the compound is a compound of formula A-2 or a pharmaceutically acceptable salt thereof:

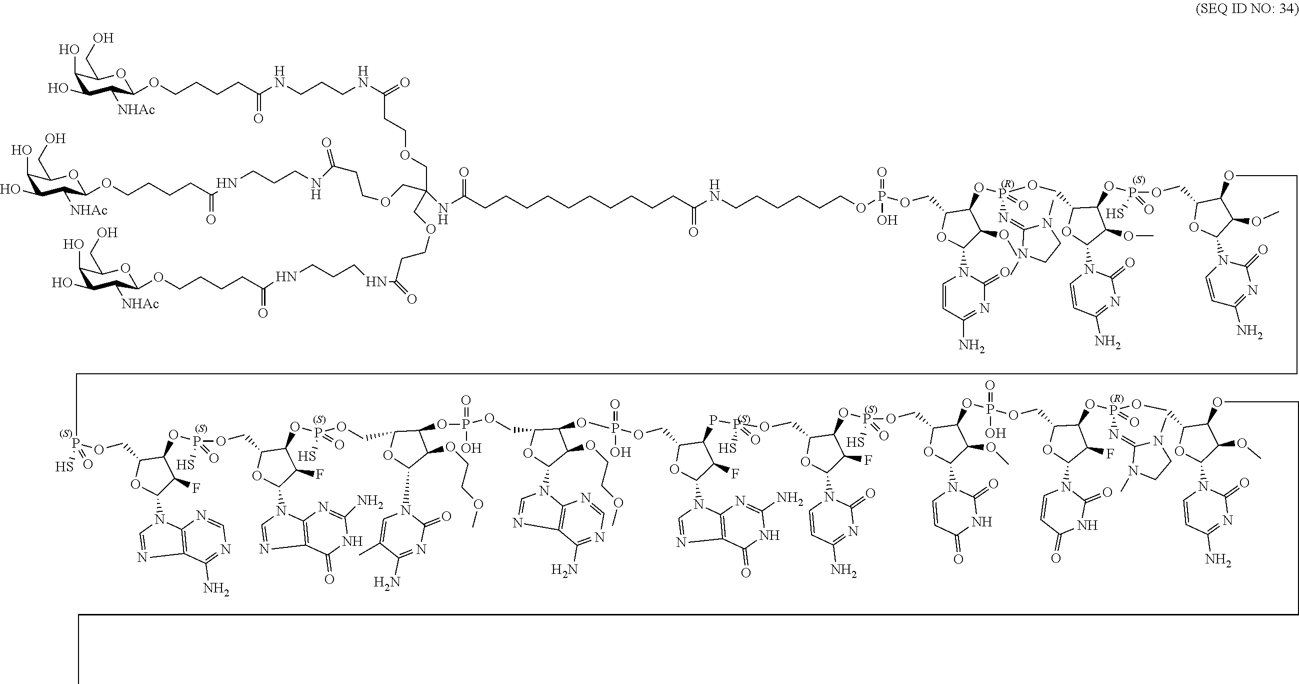

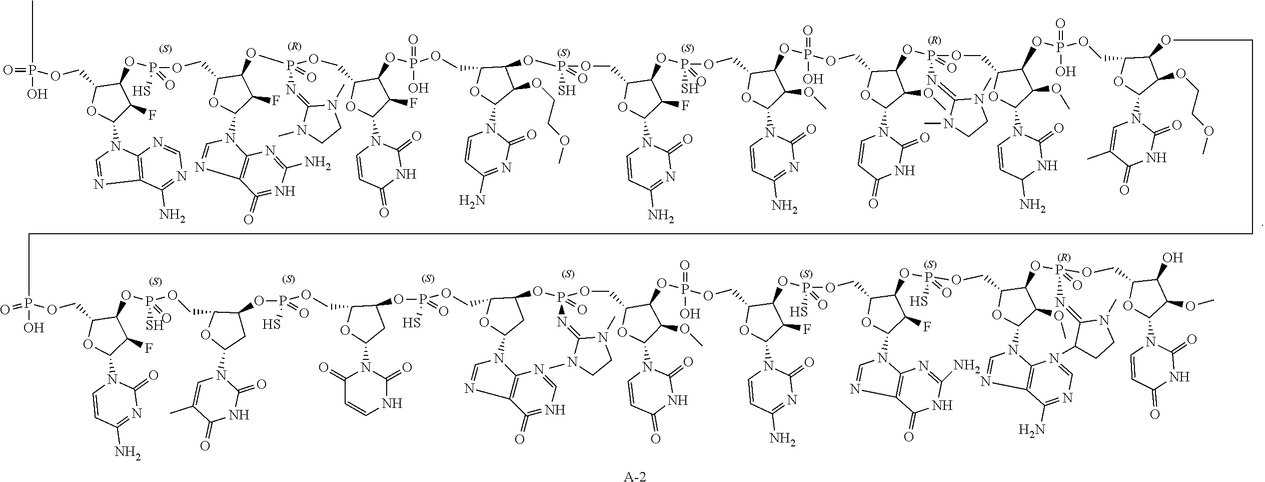

115. The compound of claim 114, wherein the compound is a sodium salt, wherein each phosphorothioate and phosphate group independently exists in its sodium salt form.

116. The compound of claim 115, wherein the diastereopurity of the compound is about or at least about (DS)^{nc}, wherein DS is about 90% and nc is the number of chiral linkage phosphorus.

117. A pharmaceutical composition comprising a compound of claim 114 and a pharmaceutically acceptable carrier.

118. A method selected from:
(1) a method for modifying a target adenosine in a target nucleic acid, comprising contacting the target nucleic acid with a compound, wherein the target adenosine is 1024 G>A in human SERPINA1;
(2) a method for producing, or restoring or increasing level of a product of a particular nucleic acid, comprising contacting a target nucleic acid with a compound, wherein the target nucleic acid comprises a target adenosine, and the particular nucleic acid differs from the target nucleic acid in that the particular nucleic acid has an I or G instead of the target adenosine, wherein the target nucleic acid is a human SERPINA1 transcript with a 1024 G>A mutation, and the target adenosine is 1024 G>A in human SERPINA1;
(3) a method for reducing level of a product of a target nucleic acid, comprising contacting a target nucleic acid with a compound, wherein the target nucleic acid comprises a target adenosine, wherein the target nucleic acid is a human SERPINA1 transcript with a 1024 G>A mutation, and the target adenosine is 1024 G>A in human SERPINA1;
(4) a method for reducing Z-AAT in liver of a subject, comprising administering or delivering to the subject an effective amount of a compound, wherein the subject comprises 1024 G>A (E342K) mutation in human SERPINA1;
(5) a method for reducing liver inflammation in a subject, comprising administering or delivering to the subject an effective amount of a compound, wherein the subject comprises 1024 G>A (E342K) mutation in human SERPINA1;
(6) a method for inhibiting elastase in a subject, comprising administering or delivering to the subject an effective amount of a compound, wherein the subject comprises 1024 G>A (E342K) mutation in human SERPINA1;
(7) a method for increasing levels and/or activities of an alpha-1 antitrypsin (A1AT) polypeptide in the serum or blood of a subject, comprising administering or delivering to the subject an effective amount of a compound, wherein the subject comprises 1024 G>A (E342K) mutation in human SERPINA1; and
(8) a method for decreasing levels and/or activities of a mutant alpha-1 antitrypsin (A1AT) polypeptide in the serum or blood of a subject, comprising administering or delivering to the subject an effective amount of a compound, wherein the subject comprises 1024 G>A (E342K) mutation in human SERPINA1;
wherein the compound is a compound of claim 114.

119. A method for preventing or treating a condition, disorder or disease, comprising administering or delivering to a subject susceptible thereto or suffering therefrom an effective amount of a compound of claim 114, wherein the subject comprises 1024 G>A (E342K) mutation in human SERPINA1.

120. A method for preparing a compound of claim 114, comprising coupling a phosphoramidite comprising a chiral auxiliary with a hydroxyl group.
